(19) United States
(12) Patent Application Publication
(10) Pub. No.: US 2010/0047807 A1
(43) Pub. Date: Feb. 25, 2010

(54) GENETIC VARIANTS ASSOCIATED WITH PERIODIC LIMB MOVEMENTS AND RESTLESS LEGS SYNDROME

(75) Inventors: Hreinn Stefansson, Gardabaer (IS); Hjorvar Petursson, Reykjavik (IS)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: deCODE Genetics ehf., Reykjavik (IS)

(21) Appl. No.: 12/595,333

(22) PCT Filed: Apr. 11, 2008
(86) PCT No.: PCT/IS2008/000010

§ 371 Date: Oct. 9, 2009

(30) Foreign Application Priority Data
Apr. 12, 2007 (IS) 8631
Jun. 22, 2007 (IS) 8655
Jul. 13, 2007 (IS) 8663

(51) Int. Cl. C12Q 1/68; G06F 19/00; C12M 1/34

(52) U.S. Cl. 435/6; 702/19; 435/287.2

(57) ABSTRACT

The present inventions discloses genetic markers and haplotypes that have been found to be associated with risk of Restless Legs Syndrome (RLS), Periodic Limb Movement Disorder (PLMD), and Periodic Limb Movements of Sleep (PLMS). Methods for determination of susceptibility of these disorders are disclosed using such markers, as are kits useful in such determination.

### BACKGROUND OF THE INVENTION

Genetic risk is conferred by subtle differences in genes among individuals in a population. Genes differ between individuals most frequently due to single nucleotide polymorphisms (SNP), although other variations are also important. SNPs are located on average every 1000 base pairs in the human genome. Accordingly, a typical human gene containing 250,000 base pairs may contain 250 different SNPS. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphisms in the human genome are caused by insertions, deletions, translocations, or inversions of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of disease are uncovered, genetic testing for such risk factors is becoming important for clinical medicine. Examples are apolipoprotein E testing to identify genetic carriers of the ApoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

Restless Legs Syndrome (RLS) is a common neurologic disorder involving both sensory and motor elements. An uncomfortable and distressing sensory urge dominates during rest/inactivity in the evening and at night and delays the onset of sleep. Sleep is often interrupted by involuntary, highly stereotyped, and regularly occurring limb movements, called periodic limb movements in sleep (PLMS). The RLS diagnosis is based on fulfilling established clinical criteria. Affected individuals have a strong urge to move their legs due to discomfort and unpleasant sensation in the legs that worsens with inactivity and affects most RLS patients in the evening or at bedtime. Diagnosis of Periodic Limb Movement Disorder (PMLD) requires the presence of periodic limb movements of sleep (PLMS) on polysomnography, sleep complaints and exclusion of other potential causes of the associated sleep complaint. PLMS (>5 movements/hour of sleep) are found in more than 80% of RLS patients (Montplaisir, J. et al. Mov Disord 12, 61-5 (1997)). However, a much smaller fraction of those with severe PLMS (>5 movements/hour of sleep) fulfill the diagnosis of RLS. The PLMS usually occur at periodic intervals, usually during the first half of the night, during non-REM sleep. It has been shown that PLMS are associated with microarousals and autonomic activation, and therefore associated with an alteration in sleep structure, and PLMS may be a marker of sleep instability. In childhood and adolescence, PLMS (>5 movements/hour of sleep) rarely occur in healthy individuals. Its prevalence, however, increases with advanced age and is observed in subjects with and without any sleep disturbance in adults and the elderly

Despite how common these conditions are there have been relatively few studies of their genetic epidemiology. Still, from the available data it is clear that genetic factors are important. Firstly, familial nature of RLS has long been recognized, with high reported rates of positive family history among RLS patients (Allen, R. P. et al. Sleep Med 4, 101-19 (2003)), and corroborative evidence from twin studies (Desai, A. V., Cherkas, L. F., Spector, T. D. & Williams, A. J. Twin Res 7, 589-95 (2004); Ondo, W. G., Vuong, K. D. & Wang, Q. Neurology 55, 1404-6 (2000)). Secondly, this distressing sensorimotor disorder is more prevalent in populations of western European ancestry (5.5-24%) than in other populations that have been studied; 0.1% in Singapore, 2% in a native population in Ecuador, 3.2% in Turkey and 4.6% in an elderly Japanese population (age 65+) (Merlino, G., Valente, M., Serafini, A. & Gigli, G. L. Neurol Sci 28 Suppl 1, S37-46 (2007); Mizuno, S., Miyaoka, T., Inagaki, T. & Horiguchi, J. Psychiatry Clin Neurosci 59, 461-5 (2005)). No population prevalence figures are, however, available for African populations, although one study suggests that the prevalence among African-Americans is similar to that of people of western European origin in the US (Lee, H. B. et al. Sleep Med 7, 642-5 (2006)). Furthermore, the onset of RLS is highly variable, spanning very young age to very old age. The variation in onset could be explained by different causative alleles, or by phenocopies. Thus, RLS may occur in an idiopathic primary form or secondary to other medical conditions, in particular peripheral neuropathies, uraemia, rheumatoid arthritis, Parkinson's disease, iron deficiency and attention-deficit hyperactivity disorder in children.

RLS is not only a subjective disorder, periodic limb movements in sleep (PLMS) commonly accompany RLS and explain in part the sleep disturbances RLS patients often suffer from (Hornyak, M., Riemann, D. & Voderholzer, U. Sleep Med 5, 597-600 (2004)). Although over 80% of RLS patients experience severe PLMS (>5 movements/h sleep) (Montplaisir, J. et al. Mov Disord 12, 61-5 (1997)), RLS and PLMS do not appear to be alternate forms of the same trait. RLS can exist in a person not showing PLMS and a person with PLMS does not always have RLS. PLMS are regarded a distinct nosologic entity and pathological if severe (periodic limb movement disorder (PLMD)).

The most important secondary factor contributing to the clinical manifestation of RLS and PLMS is iron depletion (Trenkwalder, C., et al., Lancet Neurol. 4: 465-475 (2005). Several studies have thus shown that there is a relation between low concentrations of ferritin and symptoms of RLS.

Although physiological traits by themselves may not merit much attention they can be of most importance in the identification of genes and genetic variants associating with and conferring risk to more severe human diseases. Traits such as PLMS may better correspond to genetic variants than conditions or diseases which are diagnosed by a strictly clinical diagnosis and may be secondary, or represent a mixed bag of phenocopies. The PLMS merit attention because of their association with RLS and PLMD, as well as sleep state or arousal instability, which has been shown to confer risk of hypertension and other cardiovascular problems, as well as affecting the quality of life.

There is thus a need for identifying genetic variants that associate with RLS, PLMD and PLMS. Knowledge of such variants can be utilized for the development of diagnostic methods that can be useful for the identification of those individuals at risk for developing these disorders; it can also be used in diagnostic methods of these disorders. The identification of the genetic variants, and the underlying genes, predisposing towards the development of RLS, PLMD and PLMS can be utilized in the development of new therapeutic means for preventing and/or alleviating the symptoms associated with these disorders.

### SUMMARY OF THE INVENTION

The present invention relates to methods of diagnosing a susceptibility to sleep-related movement disorders. In particular, the invention relates to methods of diagnosing or detecting a susceptibility to RLS, PLMD and/or PLMS. The invention pertains to methods of diagnosing or detecting an increased susceptibility to RLS, PLMD and PLMS, as well as methods of diagnosing or detecting a decreased susceptibility to RLS, PLMD and PLMS or diagnosing a protection against RLS, PLMD and PLMS, by evaluating certain markers or haplotypes that have been found to be associated with increased or decreased susceptibility of these disorders.

The present invention relates to a method of determining a susceptibility to a sleep-related movement disorder in a human individual, the method comprising determining the presence or absence of at least one allele of at least one polymorphic marker in a nucleic acid sample obtained from the individual, wherein the at least one polymorphic marker is selected from the group of markers associated with the C06 LD block, wherein the presence of the at least one allele is indicative of a susceptibility to a sleep-related movement disorder. These markers are tagging markers for the C06 LD block, and as such they are surrogate markers for the at-risk variants of the present invention. In one embodiment, markers associated with the C06 LD block are markers that are in linkage disequilibrium with at least one marker within the C06 LD block.

Another aspect relates to a method of determining a susceptibility to a sleep-related movement disorder in a human individual, the method comprising determining the presence or absence of at least one allele of at least one polymorphic marker in a nucleic acid sample obtained from the individual, or in a genotype dataset derived from the individual, wherein the at least one polymorphic marker is selected from the group of markers set forth in Table 4, and markers in linkage disequilibrium therewith, wherein the presence of the at least one allele is indicative of a susceptibility to a sleep-related movement disorder. In one embodiment, the method comprises determining the presence or absence of at least one allele of at least one polymorphic marker in a nucleic acid sample obtained from the individual. In another embodiment, the method comprises determining the presence or absence of at least one allele of at least one polymorphic marker in a genotype dataset derived from the individual. The genotype dataset is preferably derived from the individual in the sense that the dataset contains genetic information that has been determined using a nucleic acid sample from the individual.

In certain embodiments, the at least one polymorphic marker is associated with the BTBD9 gene, the GLO1 gene, the DNAH8 gene and/or the TEX27 gene. In certain such embodiments, the polymorphic marker is in linkage disequilibrium with at least one marker within the BTBD9 gene, the GLO1 gene, the DNAH8 gene and/or the TEX27 gene. In certain preferred embodiments, the at least one polymorphic marker is selected from the group of markers consisting of rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11). In other embodiments, the at least one polymorphic marker is marker rs3923809 (SEQ ID NO:1), or markers in linkage disequilibrium therewith. In one preferred embodiment, the at least one polymorphic marker is selected from the markers set forth in Table 5. In another preferred embodiment, the at least one polymorphic marker is rs3923809. In another preferred embodiment, the at least one polymorphic marker is rs3923809, wherein the presence of allele 1 (allele A) is indicative of increased risk of the sleep-related movement disorder.

Another method of the invention relates to assessing an individual for probability of response to a therapeutic agent used for preventing or ameliorating symptoms associated with a sleep-related movement disorder, comprising: determining the presence or absence of at least one allele of at least one polymorphic marker in a nucleic acid sample obtained from the individual, or in a dataset derived from the individual, wherein the at least one polymorphic marker is selected from the group consisting of the polymorphic markers set forth in Table 4, wherein the presence of the marker is indicative of a probability of a positive response to the therapeutic agent. In embodiment, the therapeutic agent is selected from entacapone, levodopa, carbidopa, combinations comprising entacapone, levodopa and carbidopa, rotigotine, safinamide, pramipexole, ropinirole, gabapentin, gabapentin enacarbil, istradefylline, aplindore, lisuride, radafaxine, SEP-226330, sumanirole, nitric oxide donors and dopamine D1 receptor agonists. In one preferred embodiment, the therapeutic agent is gabapentin. Other embodiments can include any one, or any combinations of, these therapeutic agents, or other therapeutic agents useful for treating a sleep-related movement disorder, or ameliorating symptoms associated with a sleep-related movement disorder.

Another aspect of the invention provides a method of determining susceptibility to abnormal iron stores in a human individual, the method comprising determining the presence or absence of at least one allele of at least one polymorphic marker in a nucleic acid sample obtained from the individual, or in a dataset derived from the individual, wherein the at least one polymorphic marker is selected from the group consisting of the polymorphic markers set forth in Table 4, and markers in linkage disequilibrium therewith, wherein the presence of the marker is indicative of susceptibility to abnormal iron stores in the individual. In one embodiment, the abnormal iron stores are characterized by decreased serum ferritin levels and/or increased ferritin index. In particular embodiments, serum ferritin levels are decreased by at least 10% for each copy of the at-risk allele for decreased iron stores and/or increased ferritin index of the present invention. In other embodiments, serum ferritin levels are decreased by at least 11%, at least 12%, or at least 13% for each copy of the at-risk allele for decreased iron stores of the present invention. In one such embodiment, the presence of rs3923809 allele A, or a marker allele in linkage disequilibrium therewith, in the individual, is indicative of decreased serum ferritin levels in the individual by at least 10% per allele copy.

In another aspect, the invention relates to a method of identification a marker for use in assessing susceptibility to a sleep-related movement disorder, the method comprising identifying at least one polymorphism in linkage disequilibrium, as determined by r2>0.2, to at least one of the polymorphisms listed in Tables 1-5, and determining the genotype status of a sample of individuals diagnosed with, or having a susceptibility to, a sleep-related movement disorder, and a control sample, wherein significant association to a sleep-related movement disorder, or a susceptibility to a sleep-related movement disorder, of at least one allele in the at least one polymorphism is indicative of the polymorphism being useful for assessing susceptibility to a sleep-related movement disorder.

In another aspect, the present invention relates to a kit for assessing susceptibility to a sleep-related movement disorder in a human individual, the kit comprising reagents necessary for selectively detecting at least one allele of at least one polymorphic marker in the genome of the individual, wherein the polymorphic marker is selected from the group consisting of the polymorphic markers associated with C06 LD block, and wherein the presence of the at least one allele is indicative of a susceptibility to a sleep-related movement disorder.

The present invention, in a further aspect, relates to a method of assessing a susceptibility to a sleep-related movement disorder in a human individual, comprising screening a nucleic acid from the individual for at least one polymorphic marker allele or haplotype within the C06 LD block, between position 37,816,141 and 38,797,853 in Build 36 of the National Center for Biotechnology (NCBI) Build 36 sequence assembly, that correlates with increased occurrence of the sleep-related movement disorder in a human population; wherein determination of the presence of an at-risk marker allele in the at least one polymorphism or an at-risk haplotype in the nucleic acid identifies the individual as having elevated susceptibility to the movement disorder, and wherein the absence of the at least one at-risk marker allele or at-risk haplotype in the nucleic acid identifies the individual as not having the elevated susceptibility. In one embodiment, the at least one polymorphic marker is selected from the markers set forth in Table 4, and markers in linkage disequilibrium therewith.

Yet another aspect of the invention provides a method of identification of a marker for use in assessing susceptibility to a sleep-related movement disorder in human individuals, the method comprising (a) identifying at least one polymorphic marker within the C06 LD block, between position 37,816,141 and 38,797,853 in Build 36 of the National Center for Biotechnology (NCBI) Build 36 sequence assembly; (b) determining the genotype status of a sample of individuals diagnosed with a sleep-related movement disorder; and (c) determining the genotype status of a sample of control individuals; wherein a significant difference in frequency of at least one allele in at least one polymorphism in individuals diagnosed with the sleep-related movement disorder as compared with the frequency of the at least one allele in the control sample is indicative of the at least one polymorphism being useful for assessing susceptibility to the sleep-related movement disorder.

In one embodiment, an increase in frequency of the at least one allele in the at least one polymorphism in individuals diagnosed with the sleep-related movement disorder, as compared with the frequency of the at least one allele in the control sample, is indicative of the at least one polymorphism being useful for assessing increased susceptibility to the sleep-related movement disorder. In another embodiment, a decrease in frequency of the at least one allele in the at least one polymorphism in individuals diagnosed with the sleep-related movement disorder, as compared with the frequency of the at least one allele in the control sample, is indicative of the at least one polymorphism being useful for assessing decreased susceptibility to, or protection against, the sleep-related movement disorder. In preferred embodiments, the significant difference in frequency is characterized by a statistical measure.

A method of genotyping a nucleic acid sample obtained from a human individual is also provided, the method comprising determining the presence or absence of at least one allele of at least one polymorphic marker predictive of increased risk of a sleep-related movement disorder in the sample, wherein the at least one marker is selected from the markers set forth in Table 4, and markers in linkage disequilibrium therewith, and wherein determination of the presence or absence of the at least one allele of the at least one polymorphic marker is predictive of increased risk of the sleep-related movement disorder in the individual. Genotyping can be performed by any convenient method known to the skilled person. In some embodiments, genotyping comprises amplifying a segment of a nucleic acid that comprises the at least one polymorphic marker, by Polymerase Chain Reaction (PCR), using a nucleotide primer pair flanking the at least one polymorphic marker. In certain embodiments, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis. In preferred embodiments, the process comprises allele-specific probe hybridization. In one such embodiment, the method comprises the steps of:
1. contacting copies of the nucleic acid with a detection oligonucleotide probe and an enhancer oligonucleotide probe under conditions for specific hybridization of the oligonucleotide probe with the nucleic acid;
   wherein
   a) the detection oligonucleotide probe is from 5-100 nucleotides in length and specifically hybridizes to a first segment of the nucleic acid whose nucleotide sequence is given by SEQ ID NO:50 that comprises at least one polymorphic site;
   b) the detection oligonucleotide probe comprises a detectable label at its 3′ terminus and a quenching moiety at its 5′ terminus;
   c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
   d) a single base gap exists between the first segment and the second segment, such that when the oligonucleotide probe and the enhancer oligonucleotide probe are both hybridized to the nucleic acid, a single base gap exists between the oligonucleotides;
2. treating the nucleic acid with an endonuclease that will cleave the detectable label from the 3′ terminus of the detection probe to release free detectable label when the detection probe is hybridized to the nucleic acid; and
   measuring free detectable label, wherein the presence of the free detectable label indicates that the detection probe specifically hybridizes to the first segment of the nucleic acid, and indicates the sequence of the polymorphic site as the complement of the detection probe.

The present invention further provides a method of predicting prognosis of an individual diagnosed with a sleep-related movement disorder, the method comprising determining the presence or absence of at least one allele of at least one polymorphic marker in a nucleic acid sample obtained from the individual, wherein the at least one polymorphic marker is selected from the markers set forth in Table 4, and markers in linkage disequilibrium therewith, wherein determination of the presence of the at least one allele is indicative of a worse prognosis of the sleep-related movement disorder in the individual.

A method of monitoring progress of a treatment of an individual undergoing treatment for a sleep-related movement disorder is also provided, the method comprising determining the presence or absence of at least one allele of at least one polymorphic marker in a nucleic acid sample obtained from the individual, wherein the at least one polymorphic marker is selected from the markers set forth in Table 4, and markers in linkage disequilibrium therewith, wherein determination of the presence of the at least one allele is indicative of the treatment outcome of the individual.

A further method of the invention pertain to the diagnosis or determination of a susceptibility to a sleep-related movement disorder in a human individual, the method comprising determining the presence or absence of at least one allele of at least one polymorphic marker in a nucleic acid sample obtained from the individual, wherein the at least one polymorphic marker is associated with the Meis1 gene, wherein the presence of the at least one allele is indicative of a susceptibility to a sleep-related movement disorder. In one embodiment, the at least one marker is located within the Meis1 LD block, between positions 66,580,000 and 66,660,000 in NCBI Build 36 of the human sequence assembly. In another embodiment, the at least marker is selected from the markers indicated in Table 12. In certain embodiments a further step is provided comprising assessing the frequency of at least one haplotype in the individual, wherein the haplotype comprises at least two markers, and wherein the presence of the at least one haplotype is indicative of increased susceptibility to a sleep-related movement disorder. In one such embodiment, the haplotype comprises rs2192954 allele A and rs2300478 allele G, or haplotypes in linkage disequilibrium therewith. In one embodiment, the haplotype is characterized by the presence of rs2192954 allele A and rs2300478 allele G. In another embodiment, the haplotype is selected from T-rs4387782 T-rs12713568 T-rs3890755 C-rs6728018 C-rs4480973 G-rs10865355 G-rs9789535 T-rs2216120 A-rs2300477 A-rs2192954 G-rs2300478 G-rs6711787 A-rs2284706 C-rs2300484 C-rs1000756; and A-rs2192954 G-rs2300478.

The invention also provides computer-implemented functions relating to the relationship between certain genetic variants and sleep-related movement disorders. For example, in one aspect of the invention, an apparatus for determining a genetic indicator for a sleep-related movement disorder in a human individual is provided, comprising:
a computer readable memory; and
a routine stored on the computer readable memory;
   wherein the routine is adapted to be executed on a processor to analyze marker and/or haplotype information for at least one human individual with respect to at least one polymorphic marker selected from the markers set forth in Table 4, and markers in linkage disequilibrium therewith, and generate an output based on the marker or haplotype information, wherein the output comprises a risk measure of the at least one marker or haplotype as a genetic indicator of the sleep-related movement disorder in the human individual.

In one embodiment, the routine further comprises an indicator of the frequency of at least one allele of at least one polymorphic marker or at least one haplotype in a plurality of individuals diagnosed with a sleep-related movement disorder, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with a sleep-related movement disorder.

Kits for use in any of the methods described herein are also provided. In one such aspect, the invention provides a kit for assessing susceptibility to a sleep-related movement disorder in a human individual, the kit comprising reagents for selectively detecting at least one allele of at least one polymorphic marker in the genome of the individual, wherein the polymorphic marker is selected from the markers set forth in Table 4, and markers in linkage disequilibrium therewith, and wherein the presence of the at least one allele is indicative of a susceptibility to a sleep-related movement disorder. In one embodiment, the at least one polymorphic marker is selected from rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO: 10), and rs13219518 (SEQ ID NO: 11). In another embodiment, the at least one polymorphic marker is selected from rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3). In a preferred embodiment, the at least one polymorphic marker is rs3923809 (SEQ ID NO:1), or markers in linkage disequilibrium therewith. In certain embodiments, the reagents comprise at least one contiguous oligonucleotide that hybridizes to a fragment of the genome of the individual comprising the at least one polymorphic marker, a buffer and a detectable label. In some embodiments, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. The at least one oligonucleotide is preferably completely complementary to the genome of the individual. The oligonucleotide can be about 18 to about 50 nucleotides in length. In certain embodiments, the oligonucleotide is 20-30 nucleotides in length. In preferred embodiments, the kit comprises:
a. a detection oligonucleotide probe that is from 5-100 nucleotides in length;
b. an enhancer oligonucleotide probe that is from 5-100 nucleotides in length; and
c. an endonuclease enzyme;
   wherein the detection oligonucleotide probe specifically hybridizes to a first segment of the nucleic acid whose nucleotide sequence is set forth in any one of SEQ ID NO:1-343;
   and wherein the detection oligonucleotide probe comprises a detectable label at its 3′ terminus and a quenching moiety at its 5′ terminus;
   wherein the enhancer oligonucleotide is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid;
   wherein a single base gap exists between the first segment and the second segment, such that when the oligonucleotide probe and the enhancer oligonucleotide probe are both hybridized to the nucleic acid, a single base gap exists between the oligonucleotides; and
   wherein treating the nucleic acid with the endonuclease will cleave the detectable label from the 3′ terminus of the detection probe to release free detectable label when the detection probe is hybridized to the nucleic acid.

The invention also relates to kits for use in other methods as described in detail herein. In such kits, reagents for detecting at least one allele of at least one polymorphic marker, as described herein in further detail, are provided, optionally with instructions about further use of the information gathered by use of the kit. This includes, for example, instructions about drug or therapeutic agent response, appropriate drug to use or administer based on the genetic status of the individual, and complications associated with a particular drug. Such use is in more detailed terms described further herein.

In some embodiments of the methods, apparatus and kits provided, the sleep-related movement disorder is selected from Restless Legs Syndrome (RLS), Periodic limb movements in sleep (PLMS) and Periodic Limb Movement Disorder (PLMD). In one embodiment, the sleep-related movement disorder is Restless Legs Syndrom (RLS). In another embodiment, the sleep-related movement disorder is Periodic limb movements in sleep (PLMS). In another embodiment, the sleep-related movement disorder is Periodic Limb Movement Disorder (PLMD). In some embodiments, the sleep-related movement disorder is RLS in the presence of PLMS (RLS+PLMS). In some other embodiments, the disorder is PLMS in the absence of RLS (PLMS−RLS). The sleep-related movement disorder may in other embodiments furthermore comprise other disorders known to be associated with RLS, including but not limited to peripheral neuropathies, hypertension, uraemia, rheumatoid arthritis, Parkinson's disease, iron deficiency and attention-deficit hyperactivity disorder in children.

Another aspect of the invention relates to the use of an oligonucleotide probe in the manufacture of a diagnostic reagent for diagnosing/assessing susceptibility to a sleep-related movement disorder, wherein the probe comprises a fragment of the genome comprising at least one polymorphism selected from the polymorphisms set forth in any of the Tables 1-5, and polymorphisms in linkage disequilibrium therewith, wherein the fragment is 15-500 nucleotides in length.

Yet another aspect of the invention relates to a method of predicting prognosis of an individual experiencing symptoms associated with, or an individual diagnosed with, a sleep-related movement disorder, the method comprising determining the presence or absence of at least one allele of at least one polymorphic marker in a nucleic acid sample obtained from the individual, wherein the at least one polymorphic marker is selected from the group consisting of the polymorphic markers listed in Tables 1-5, and markers in linkage disequilibrium therewith, wherein the presence of the at least one allele is indicative of a worse prognosis of the breast cancer in the individual.

A further aspect of the invention relates to a method of monitoring progress of a treatment of an individual undergoing treatment for symptoms associated with a sleep-related movement disorder, the method comprising determining the presence or absence of at least one allele of at least one polymorphic marker in a nucleic acid sample obtained from the individual, wherein the at least one polymorphic marker is selected from the group consisting of the polymorphic markers listed in Tables 1-5, and markers in linkage disequilibrium therewith, wherein the presence of the at least one allele is indicative of the treatment outcome of the individual.

Variants (markers and/or haplotypes comprising polymorphic markers) in linkage disequilibrium with the markers and haplotypes of the present invention are also useful for the methods and kits of the invention, as described herein. The invention therefore also pertains to markers in linkage disequilibrium with the markers and haplotypes of the invention. In certain embodiments of the methods, apparatus or kits of the invention, linkage disequilibrium is characterized by specific values or ranges of values for a quantitative measure of linkage disequilibrium. In one such embodiment, linkage disequilibrium is characterized by specific values for r². In another such embodiment, linkage disequilibrium is characterized by specific values for |D′|. In yet another embodiment, linkage disequilibrium is characterized by specific values for r² and |D′|. In one preferred embodiment, linkage disequilibrium is characterized by values for r² of greater than 0.1. In another preferred embodiment, linkage disequilibrium is characterized by values for r² of greater than 0.2. Other cutoff values for r² are also possible, including, but not limited to, 0.3, 0.4, 0.5, 0.6, 0.7, 0.8, 0.9, 0.95, 0.96, 0.97, 0.98, 0.99. In another preferred embodiment, linkage disequilibrium is characterized by values for |D′| of greater than 0.5. In another preferred embodiment, linkage disequilibrium is characterized by values for |D′| of greater than 0.8. Other cutoff values for |D′| are also possible, including, but not limited to, 0.2, 0.3, 0.4, 0.6, 0.7, 0.8, 0.9, 0.95, 0.96, 0.97, 0.98 and 0.99. In certain embodiments, linkage disequilibrium is characterized by numeric cutoff values for either |D′| and r². In one such embodiment linkage disequilibrium is characterized by numeric values for either |D′| of greater than 0.8 or r² of greater than 0.2, or both.

In certain other embodiments of the methods, uses, apparatus or kits of the invention, the individual is of a specific human ancestry. In one embodiment, the ancestry is selected from black African ancestry, Caucasian ancestry and Chinese ancestry. In another embodiment, the ancestry is black African ancestry. In another embodiment, the ancestry is African American ancestry. In another embodiment, the ancestry is European ancestry. In another embodiment, the ancestry is Caucasian ancestry. The ancestry is in certain embodiment self-reported by the individual who undergoes genetic analysis or genotyping. In other embodiments, the ancestry is determined by genetic determination comprising detecting at least one allele of at least one polymorphic marker in a nucleic acid sample from the individual, wherein the presence or absence of the allele is indicative of the ancestry of the individual.

The invention can in general be reduced to practice to in the methods, apparatus and other applications described herein using the markers and haplotypes of the invention that have in more detail been described herein in general terms as being useful for assessing susceptibility to the sleep-related movement disorders RLS, PLMS and PLMD. Thus, in certain embodiments the invention pertains to any one or more than one of the markers as listed in Tables 1-5, and markers in linkage disequilibrium therewith. In some embodiments, the at least one marker is selected from the group of markers set forth in Table 4, and markers in linkage disequilibrium therewith. In other embodiments, the at least one marker is selected from the marker set forth in Table 5. In some other embodiments, the at least one marker is selected from rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3), and markers in linkage disequilibrium therewith. In some embodiments, the at least one marker is selected from the group consisting of rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11). In particular preferred embodiments, the at least one marker is selected from marker rs3923809 (SEQ ID NO:1), and markers in linkage disequilibrium therewith. In another preferred embodiment, the marker is rs3923809. In some further embodiments, the markers are selected from markers within the C06 LD block region. Certain embodiments relate to markers associated with the Meis1 gene, including markers in linkage disequilibrium with the Meis1 gene. In one such embodiment, the markers are selected from markers within the Meis1 LD block. In a preferred embodiment, the markers are selected from the markers set forth in Table 12, and markers in linkage disequilibrium therewith. In another preferred embodiment, the markers are A-rs2192954 G-rs2300478, wherein the presence of allele A in rs2192954 and allele G in rs2300478 is indicative of increased risk of a sleep-related movement disorder. In other preferred embodiments, the markers are selected from markers in linkage disequilibrium with the A-rs2192954 G-rs2300478 haplotype.

In certain embodiments of the methods of the invention further comprise assessing the frequency of at least one haplotype in the individual, wherein the haplotype comprises at least two markers, and wherein the presence of the at least one haplotype is indicative of a susceptibility to a sleep-related movement disorder. Detecting haplotypes comprising two or more polymorphic markers can be performed using methods known to the skilled person, as further described herein.

In other embodiments of the invention, the presence of the at least one allele or haplotype is indicative of increased susceptibility to a sleep-related movement disorder. In one such embodiment, the at least one allele or haplotype comprises at least one of rs3923809 allele 1, rs9357271 allele 4, rs6923737 allele 4, rs7770868 allele 1, rs4711546 allele 1, rs6904723 allele 1, rs12208647 allele 1, rs4236060 allele 2, rs10947739 allele 2, rs6920488 allele 1, and rs13219518 allele 1. In another such embodiment, the haplotype is

### T-rs4387782 T-rs12713568 T-rs3890755 C-rs6728018 C-rs4480973 G-rs10865355 G-rs9789535 T-rs2216120 A-rs2300477 A-rs2192954 G-rs2300478 G-rs6711787 A-rs2284706 C-rs2300484 C-rs1000756; or

### A-rs2192954 G-rs2300478.

In certain embodiments, increased susceptibility is characterized by a relative risk or odds ratio of at least 1.4, including at least 1.5, at least 1.6, a at least 1.7, at least 1.8, at least 1.9 or at least 2.0.

In certain other embodiments of the methods or kits of the invention, the presence of the at least one allele or haplotype is indicative of decreased susceptibility to a sleep-related movement disorder. In certain such embodiments, the decreased susceptibility is characterized by a relative risk of less than 0.9, including a relative risk of less than 0.8, a relative risk of less than 0.7, a relative risk of less than 0.6, and a relative risk of less than 0.5.

Particular embodiments of the methods of the invention pertains to analysis of the markers and/or haplotypes associated with sleep-related movement disorder, such as RLS, PLMS and PLMD, in combination with other risk factors for sleep-related movement disorders. In one such embodiment, a further step comprising analyzing a sample comprising genomic DNA from the human individual or a genotype dataset derived from the human individual for the presence or absence of at an additional at-risk variant for the sleep-related movement disorder is included, wherein the additional variant comprises at least one at-risk allele of at least one at-risk variant for the sleep-related movement disorder that is not in linkage disequilibrium with an at-risk marker for the sleep-related movement disorder associated with the C06 LD Block. In one such embodiment, the additional at-risk variant is a variant that is not set forth in Table 4 herein. In another embodiment, the additional at-risk variant is an at-risk variant associated with the Meis1 gene. In a preferred embodiment the additional at-risk variant is a haplotype comprising rs2192954 allele A and rs2300478 allele G, or a marker or haplotype in linkage disequilibrium therewith. In another preferred embodiment of the methods of the invention, the presence or absence of at least one of rs3923809 allele 1, rs9357271 allele 4, rs6923737 allele 4, rs7770868 allele 1, rs4711546 allele 1, rs6904723 allele 1, rs12208647 allele 1, rs4236060 allele 2, rs10947739 allele 2, rs6920488 allele 1, and rs13219518 allele 1 is determined, together with the presence or absence of the haplotype comprising rs2192954 allele A and rs2300478 allele G. In another preferred embodiment, the presence or absence of rs3923809 allele 1 is determined, wherein the presence of allele 1 is indicative of increased risk of a sleep-related movement disorder, together with the determination of the presence or absence of the haplotype comprising rs2192954 allele A and rs2300478 allele G, wherein the presence of the haplotype is indicative of increased risk of the sleep-related movement disorder. The determination of multiple independent at-risk variants for a sleep-related movement disorder (i.e. variants that are not in linkage disequilibrium) can be analyzed to determine overall risk. In certain other embodiments, a further step is included, comprising analyzing non-genetic information to make risk assessment, diagnosis, or prognosis of the human individual.

The non-genetic information can for example be selected from age, gender, ethnicity, socioeconomic status, previous disease diagnosis, medical history of subject, family history of a sleep-related movement disorder, biochemical measurements, and clinical measurements. In one embodiment, the biochemical measurements represent a measure of iron stores in the individual. In a preferred embodiment, the iron stores are represented by serum ferritin levels and/or increased ferritin index in the individual.

Combined risk can be calculated based on the multiple genetic and/or non-genetic risk factors, so as to determine overall risk for the individual.

### BRIEF DESCRIPTION OF THE DRAWINGS

The foregoing and other objects, features and advantages of the invention will be apparent from the following more particular description of preferred embodiments of the invention.

FIG. 1 shows the genomic structure of the C06 LD block. The position of six of the genome-wide significant markers in association with PLMS are indicated by circles. The LD block is characterized by the presence of a region of high LD, and its the boundaries are characterized by regions of high recombination. The LD block as defined spans positions 37,755,018 and 38,736,730 of chromosome 6 (NCBI Build 34).

FIG. 2 shows linkage disequilibrium at the BTBD9 region in the CEU HapMap SNP data (Release 19). The genome-wide significant markers, rs3923809 and rs6923737, within the BTBD9 gene are indicated, as are the TEX27 gene, the GLO1 gene and the promoter region for the DNAH8 gene, all of which are within the boundaries of the LD block.

FIG. 3 shows mean expression of GLO1 in adipose tissue from 557 individuals for the three different genotypes of marker rs3923809. The number of individuals behind each estimate is indicated at the bottom of each column and the standard error of the mean is indicated with vertical bars at the top of each column. Also included is a P-value obtained by regressing the sex and age adjusted expression on the number of copies of allele A (allelic code: A=1) each individual carries.

FIG. 4 shows data for periodic limb movements and rs3923809 genotype. RLS patients and relatives were genotyped for marker rs3923809 and the frequency of periodic limb movements in sleep estimated. Although a fraction of subjects with frequency of periodic limb movements >5/h were considered not having PLMs, after ambulatory assessments based on qualitative features (Supplementary data online), they were not excluded from this analysis to avoid bias (A) Subjects were grouped into five categories based on the frequency of limb movements; 0-5 (N=271), 5-10 (N=182), 10-20 (N=212), >20 (N=278) movements. The risk of allele A of marker rs3923809 increased in step with number of PLMs from 1.0 in the group with 0-5 movements/h sleep to 2.0 in the most severe group, 20+ movements/h sleep. (B) Subjects were also grouped by genotype into AA homozygotes (N=502), AG heterozygotes (N=371) and GG homozygotes (N=70). The frequency of movements was greater in AA homozygotes than in AG heterozygotes (P=0.003) and in turn higher in AG heterozygotes than in GG homozygotes (P<0.001) for marker rs3923809. AA homozygotes moved twice as often/h sleep than non-carriers (P<0.001). Error bars indicate standard error.

FIG. 5 shows serum ferritin levels in RLS patients and their relatives. Males (A) (N=362) and females (B) (N=603). Serum ferritin was found to be decreased by 13% for each copy of the A allele of marker rs3923809 (p=0.002).

FIG. 6 shows the genomic region on chromosome 2 encompassing the Meis1 gene.

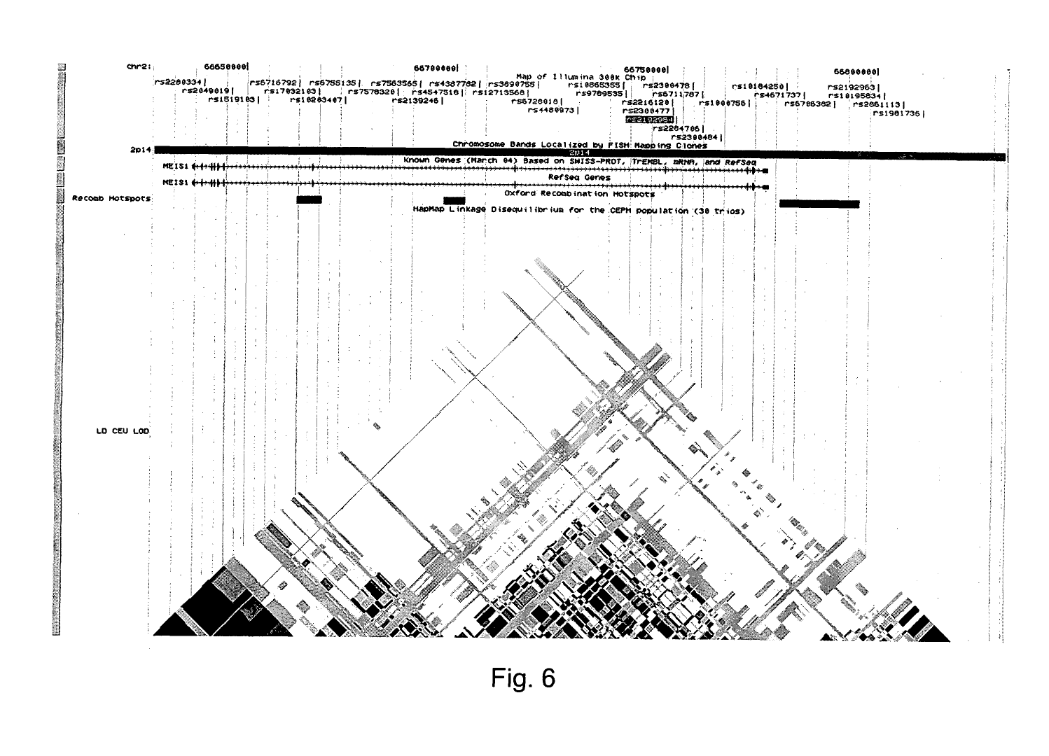

### DETAILED DESCRIPTION OF THE INVENTION

A description of preferred embodiments of the invention follows.

The present invention discloses polymorphic variants and haplotypes that have been found to be associated with RLS, PLMD and/or PLMS. Particular alleles at polymorphic markers (e.g., the markers of Tables 1-5, e.g., markers rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3), rs6920488, and rs13219518, and markers in linkage disequilibrium therewith) and haplotypes comprising such alleles have been found to be associated with RLS, PLMD and/or PLMS. Such markers and haplotypes are useful for diagnostic purposes, as described in further detail herein. Further applications of the present invention includes methods for assessing response to RLS, PLMD and/or PLMS therapeutic agents utilizing the polymorphic markers of the invention, as well as kits for assessing susceptibility of an individual to RLS, PLMD and/or PLMS.

### DEFINITIONS

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), microsatellites, insertions, deletions, duplications and translocations.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome.

Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele-1 is 1 bp shorter than the shorter allele in the CEPH sample, allele-2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, In which the number of repeat lengths varies in the general population.

An “indel” is a common form of polymorphism comprising a small insertion or deletion that is typically only a few nucleotides long.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles. Haplotypes are described herein in the context of the marker name and the allele of the marker in that haplotype, e.g., “A-rs2192954” refers to the A allele of marker rs2192954 being in the haplotype, and is equivalent to “rs2192954 allele 3”. Furthermore, allelic codes in haplotypes are as for individual markers, i.e. 1=A, 2=C, 3=G and 4=T.

The term “susceptibility”, as described herein, encompasses both increased susceptibility and decreased susceptibility. Thus, particular polymorphic markers and/or haplotypes of the invention may be characteristic of increased susceptibility (i.e., increased risk) of RLS, PLMD and/or PLMS, as characterized by a relative risk (RR) or odds ratio (OR) of greater than one. Alternatively, the markers and/or haplotypes of the invention are characteristic of decreased susceptibility (i.e., decreased risk) of RLS, PLMD and/or PLMS, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis (e.g., a sleep-related movement disorder, such as PLMS, RLS and/or PLMD), that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “nucleic acid sample” is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa, placenta, gastrointestinal tract or other organs.

The term “C06 LD block”, as described herein, refers to the Linkage Disequilibrium (LD) block on Chromosome 6 between markers rs7767440 and rs1937781, which corresponds to position 37,755,018 to 38,736,730 Mb of NCBI (National Center for Biotechnology Information) Build 34 of the human genome sequence assembly, and position 37,816,141 to 38,797,853 in the NCBI Build 36. In both these sequence builds, the C06 LD block spans 981,712 bp of sequence.

The term “Meis1 LD block”, as defined herein, refers to the LD block on Chromosome 2 between flanking recombination hotspots, and spans position 66,700,000 to 66,780,000, of NCBI Build 34 of the human genome sequence assembly. In the NCBI Build 36 sequence build, the coordinates of the Meis1 LD block are between position 66,580,000 and 66,660,000 bp. In both these sequence builds, the LD block spand 80 kb of sequence.

The term “sleep-related movement disorder”, as described herein, refers to movement disorders that adversely affect the sleep patterns of an individual. This includes Periodic Limb Movement Disorder (PLMD), Periodic limb movements in sleep (PLMS) and Restless Legs Syndrome (RLS), but can also include other related syndromes or disorders that affect sleep patterns through increased or altered movement of one or more limb, in particular the legs.

Through association analysis of a population of individuals diagnosed with RLS and/or PLMS, it has been discovered that certain alleles at certain polymorphic markers are associated with RLS and/or PLMS. A genome-wide analysis for variants associated with RLS and/or PLMS revealed association to a specific region of chromosome 6. Particular markers and haplotypes, were found to be associated with an increased risk of these disorders in this chromosomal region.

A genome-wide scan for genetic susceptibility factors associated with sleep-associated movement disorders has been performed. As shown in Table 1, several SNP markers on Chromosome 6 have been found to be associated with PLMS. These markers are all located within a region of LD flanked by the markers rs7767440 and rs1937781, which corresponds to position 37,755,018 to 38,736,730 on the chromosome (NCBI Build 34). Additional markers in LD with the markers shown in Table 1 were genotyped, leading to significant association as indicated in Table 3. The association of these markers (Table 1 and 3) is highly significant, even after correcting for the number of tests performed. These results are therefore genome-wide significant. The genetic variants identified represent common variants that confer a substantial risk of developing the disorder. Thus for marker rs3923809, the frequency of the at-risk 1 allele (A) in affected individuals is 79.1%, compared with 65.4% in the general population. This corresponds to a relative risk (RR) of 1.99 (multiplicative model), and a Population Attributable Risk (PAR) of approximately 0.63. These variants are also associated with increased risk of Restless Legs Syndrome (RLS), as indicated in Table 2. The associated risk is slightly lower at 1.79 for marker rs3923809, although still significant at the genome-wide significance level.

Further analysis has revealed that the risk conferred by rs3923809 is predominantly expressed through PLMS. As shown in Table 7, PLMS alone, or RLS with PLMS, shows significant association to the A allele of the marker, while patients diagnosed with RLS in the absence of PLMS do not have an excess of the A allele. We also found the frequency of periodic limb movements in sleep to correlate with the presence of allele A of marker rs3923809, illustrating the quantitative contribution of the at-risk A allele of the marker to the measurable PLMS phenotype. As PLMS and PLMD are very closely related clinical entities, the rs3923809 most certainly confers risk of developing PLMD, although not expressly tested.

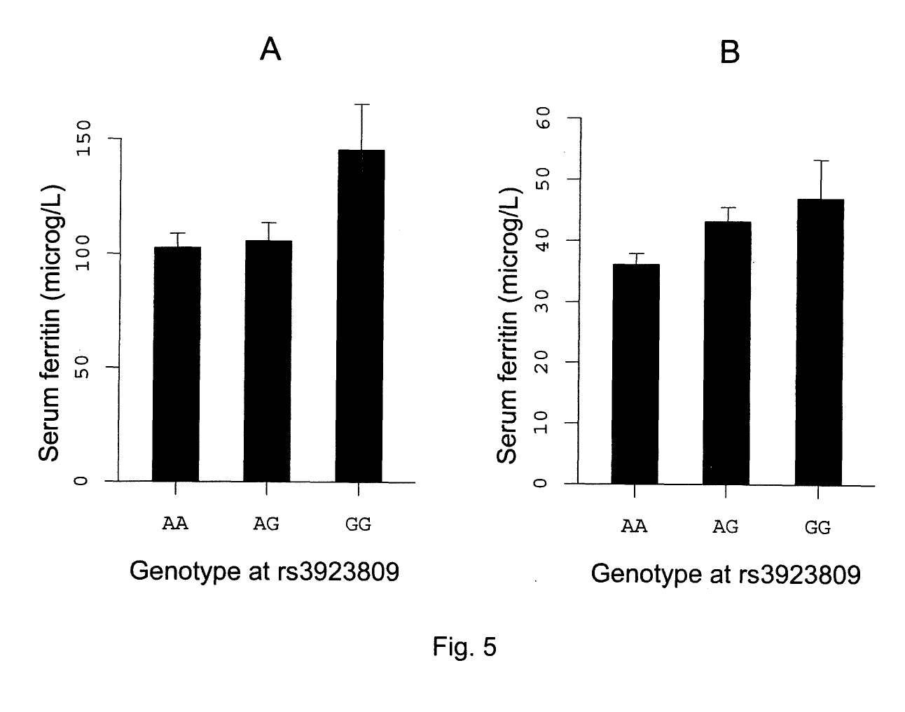

Investigation of the correlation of rs3923809 A allele with body iron stores show that the variant is significantly correlated with body iron stores. Thus, ferritin index, which is a measure inversely related to body iron stores, is increased by 5.5% for each copy of rs3923809 A allele, and serum ferritin is found to be decreased by 13% for each copy of the allele (FIG. 5).

Iron insufficiency has been investigated in the context of RLS and PLMS, due to its possible involvement in the disorders. It is now well established that iron deficiency is a significant secondary contributor to RLS and PLMS. Thus, the best established secondary causes of RLS, i.e. end stage renal disease, pregnancy, gastric surgery and blood donations, all involve compromised iron sufficiency (reviewed by Allen, R., Sleep Med 5:385-91 (2004)). It has furthermore been shown that there is increased prevalence of RLS for frequent blood donors (Silber, M. H., et al., May Clin Proc. 78:52-54 (2003)).

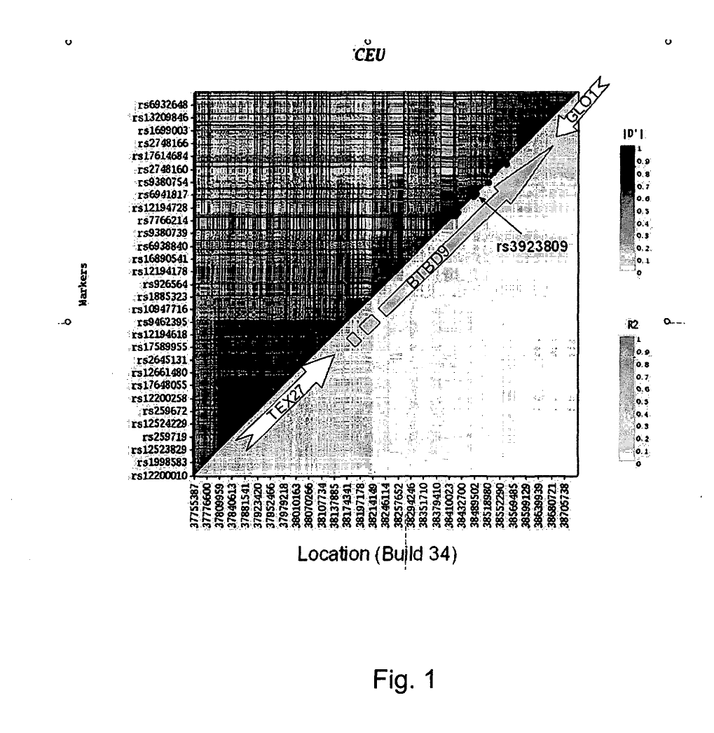

The region identified by the observed association spans a region of close to 1 Mb on chromosome 6. This region is characterized by extensive linkage disequilibrium (LD, see FIG. 1). As a consequence, a large number of polymorphic markers, in addition to those genotyped as described herein, could be used as surrogates for the markers shown in Tables 1-3, and such markers are also within the scope of the present invention. Thus, Table 4 shows all SNP markers in LD (as measured by r²>0.2, based on the public Hapmap dataset) with 7 of the markers showing the strongest association to PLMS and RLS. These markers may thus also capture the association results of the present invention, and are therefore also within scope of the present invention. These markers can therefore also be used in the methods and kits of the present invention. Other correlated markers, including known SNPs or other polymorphic markers such as microsatellites or indels, can also be used, alone or in combination, as surrogate markers for detecting the association to RLS, PLMD and/or PLMS described herein.

The sequence listing provided herein contains flanking sequence 5′ and 3′ to the corresponding polymorphic marker (299 nucleotides in each direction). The polymorphic site is indicated by the IUPAC-IUB code for the corresponding polymorphism.

The BTB (POZ) Domain Containing 9 (BTBD9), Testis Expressed Sequence 27 (TEX27) and Glyoxalase I (GLO1) Genes are in LD with the Markers Found to be Significantly Associated with PLMS and RLS

The BTBD9 gene codes for a protein with molecular weight of 61544 Da. The gene is widely expressed both in brain, including; Amygdala, cerebellum, hippocampus, Caudate nucleus and other tissue such as heart, kidney, pancreas and liver. The BTBD9 protein is not well characterized and its function has not been investigated. It contains a BTB domain also known as POZ (poxvirus and zinc finger) domain. This domain is known to be a protein-protein interaction motif found at the N-termini of several C2H2-type transcription factors as well as Shaw-type potassium channels (KCNC1-4).

The BTBD9 protein may function as a transcription factor. However, the gene contains a signal sequence indicating insertion of the protein into the plasma membrane making it less likely to have a function as a transcription factor. Possibly the BTBD9 protein interacts or operates in the same pathway as the Shaw-type potassium channels. Mouse knockout for a member of that family, Kv3.1, display a ‘strong’ mutant phenotype that includes motor dysfunction including; ataxia, myoclonus and tremor. Furthermore deficient mice are constitutively hyperactive and compared to wild type mice, double mutants display ‘restlessness’ that is particularly prominent during the light period, when mice are normally at rest, characterized by more than a doubling of ambulatory and stereotypic activity, and accompanied by a 40% sleep reduction (Espinosa, F., Marks, G., Heintz, N. & Joho, R. H. Genes Brain Behav 3, 90-100 (2004)).

Although the SNPs showing strongest association are physically located within the BTBD9 gene, the LD block spans two other genes, namely the GLO1 and TEX27 genes. The GLO1 gene is a glutathione-binding protein involved in the detoxification of methylglyoxal, a byproduct of glycolysis. In rodents, the electroencephalogram (EEG) during paradoxical sleep and exploratory behaviour is characterized by theta oscillations. Deficiency in short-chain acyl-coA dehydrogenase (Acads) in mice cause a marked slowing in the theta frequency during paradoxical sleep only. Expression of the Acads gene in brain regions involved in theta generation, notably the hippocampus. Microarray analysis of gene expression in mice with mutations in Acads indicated overexpression of Glo1. Administration of acetyl-L-carnitine (ALCAR) to mutant mice significantly recovered slow theta and GLO1 over expression (Tafti, M. et al. Nat Genet 34, 320-5 (2003)). Thus, an unappreciated metabolic pathway involving fatty acid beta-oxidation also regulates theta oscillations during sleep. Isolated rat cerebral cortex cells have been found to be able to accumulate L-carnitine and this process was competitively inhibited by 1 mM γ-aminobutyric acid (Wawrzenczyk, A., Nalecz, K. A. & Nalecz, M. J. Biochem Biophys Res Commun 213, 383-8 (1995)). Interestingly, gabapentin (1-aminomethyl cyclohexaneacetic acid), a drug structurally related to γ-aminobutiric acid (GABA), is used to treat RLS patients.

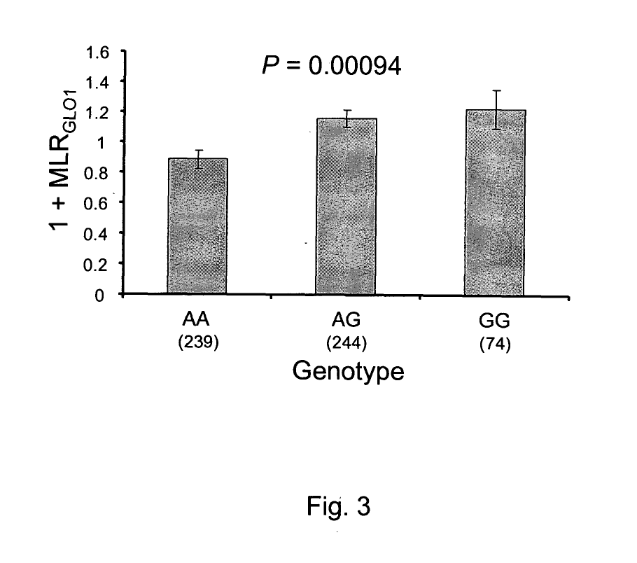

We have found that variants within C06 LD block that are associated with RLS and PLMS also affect expression of GLO1. Carriers of the at-risk allele 1 (=A) of marker rs3923809 have a decreased expression of GLO1 (FIG. 3). These findings put the genetic association findings of the present invention in a functional context, and are also consistent with the reported effects of gabapentin. The drug inhibits the reuptake of acetyl-L-carnitine, which is known to negatively affect GLO1 expression. Decreased expression of GLO1 in carriers of the at-risk carriers of the present invention may therefore be counterbalanced by the inhibitory effects of Gabapentin on acetyl-L-carnitine uptake. The polymorphic markers of the present invention may thus be predictive for the effects of therapeutic agents that exert their effect through expression levels of GLO1. The present invention therefore provides methods for predicting response to a drug that affects expression of GLO1, by assessing the presence or absence of the genetic variants of the invention in samples obtained from individuals. The absence or presence of such variants is therefore predictive for response to the drug.

The third gene within the LD block showing association to PLMS/RLS is the TEX27 gene. It has not been well characterized but may be a potential transcription factor. It is preferentially expressed in postmeiotic cells during spermatogenesis.

A fourth gene mostly residing in an adjacent LD block is the DNAH8 gene, encoding an axonemal dynein heavy chain. As promoter sequences and possible transcriptional regulatory sequences are located within the LD block of the invention, it is contemplated that the DNAH8 gene is possibly under transcriptional control by variants associated with the C06 LD block.

Investigation of haplotypes over entire haplotype blocks between recombination hotspots revealed significant association to a haplotype within the Meis1 gene (Table 10). This haplotype is significantly associated to PLMS, with OR value in excess of 2.

Further analysis revealed that a two-marker haplotype comprising rs2192954 allele A and rs2300478 allele G captures the association (Table 11). The association appears to be strongest in PLMs in the absence of RLS, but is also significant in the RLS and PLMS combined group (Table 11). The Meis1 gene is thus also implicated as a susceptibility gene for PLMS and RLS.

As a result of the discoveries disclosed herein, methods are now available for diagnosis of an increased susceptibility to sleep-related movement disorders, including RLS, PLMD and/or PLMS, as well as for diagnosis of a decreased susceptibility to and/or a protection against these disorders. In preferred embodiments of the invention, diagnostic assays are used to identify the presence of particular alleles at the chromosomal region 6p21.2, in particular the region defined by C06 LD block as described herein. In additional embodiments of the invention, other markers or SNPs, identified using the methods described herein, can be used for diagnosis of an increased susceptibility to RLS, PLMD and/or PLMS, and also for diagnosis of a decreased susceptibility to RLS, PLMD and/or PLMS or for identification of an allele that is protective against RLS, PLMD and/or PLMS. The diagnostic assays presented below can be used to identify the presence or absence of these particular alleles.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNP site; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) containing an A/G polymorphism, the assay employed may either measure the percentage or ratio of the two bases possible, i.e. A and G. Alternatively, by designing an assay that determines the opposite strand on the double-stranded DNA template, the percentage or ratio of the complementary bases T/C can be measured. Quantitatively (for example, in terms of relative risk), identical results would be obtained from measurement of either DNA strand (+ strand or − strand).

Polymorphic sites (polymorphic markers) can allow for differences in sequences based on substitutions, insertions or deletions. For example, a polymorphic microsatellite has multiple small repeats of bases (such as CA repeats) at a particular site in which the number of repeat lengths varies in the general population. Each version of the sequence with respect to the polymorphic site represents a specific allele of the polymorphic site.

Typically, a reference sequence is referred to for a particular sequence. Alleles that differ from the reference are referred to as “variant” alleles. For example, the genomic DNA sequence from position 37,755,018 to position 38,736,730 bp on Chromosome 6 of NCBI Build 34 (“C06 LD block”) represents a reference sequence. Other reference sequences related to the present invention include the genomic DNA sequence of the Meis1 LD block, the genomic DNA sequence encompassed by the BTBD9 gene, the genomic sequence encompassed by the TEX27 gene, the genomic sequence encompassed by the DNAH8 gene and the genomic sequence encompassed by the GLO1 gene. A variant sequence, as used herein, refers to a sequence that differs from the reference sequence but is otherwise substantially similar. Alleles at the polymorphic genetic markers that define the haplotypes described herein are variants. Additional variants can include changes that affect a polypeptide, e.g., a polypeptide encoded by the reference sequences of the present invention. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail herein. Such sequence changes alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with RLS, PLMD and/or PLMS or a susceptibility to RLS, PLMD and/or PLMS can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999); Kutyavin et al., Nucleic Acid Res. 34:e128 (2006)), including PCR, LCR, Nested PCR and other techniques for nucleic acid amplification. Specific methodologies available for SNP genotyping include, but are not limited to, TaqMan genotyping assays and SNPIex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), and BeadArray Technologies (e.g., Illumina GoldenGate and Infinium assays). Haplotypes of the invention (at-risk haplotypes and protective haplotypes) define genomic regions found to be associated with a susceptibility to RLS, PLMD and/or PLMS. By these or other methods available to the person skilled in the art, one or more alleles at polymorphic markers, including microsatellites, SNPs or other types of polymorphic markers, can be identified.

In certain methods described herein, an individual who is at an increased susceptibility (i.e., at risk) for a sleep-related movement disorder such as RLS, PLMD and/or PLMS is an individual in whom at least one specific allele at one or more polymorphic marker or haplotype conferring increased susceptibility for RLS, PLMD and/or PLMS is identified (i.e., at-risk marker alleles or haplotypes). In one aspect, the at-risk marker or haplotype is one that confers a significant increased risk (or susceptibility) of RLS, PLMD and/or PLMS. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least 1.5 is significant. In another further embodiment, a significant increase in risk is at least 1.7 is significant. However, other cutoffs are also contemplated, e.g., at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype of the present invention is one where at least one allele of at least one marker or haplotype is more frequently present in an individual at risk for a sleep-related movement disorder such as RLS, PLMD and/or PLMS (affected), compared to the frequency of its presence in a healthy individual (control), and wherein the presence of the marker or haplotype is indicative of increased susceptibility to RLS, PLMD and/or PLMS. The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms, i.e. free of symptoms associated with RLS and/or PLMS. In another embodiment, the disease-free control group is characterized by the absence of one or more disease-specific risk factors. Such risk factors are in one embodiment at least one environmental risk factor. In another embodiment, the risk factors comprise at least one additional genetic risk factor. As an example of a simple test for correlation would be a Fisher-exact test on a two by two table. Given a cohort of chromosomes the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for RLS, PLMD and/or PLMS is an individual in whom at least one specific allele at one or more polymorphic marker or haplotype conferring decreased susceptibility for RLS, PLMD and/or PLMS is identified. The marker alleles and/or haplotypes conferring decreased risk are also said to be protective. In one aspect, the protective marker or haplotype is one that confers a significant decreased risk (or susceptibility) of RLS, PLMD and/or PLMS. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPS), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

A genetic variant associated with a sleep-related movement disorder (e.g., RLS, PLMS and/or PLMD) can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., et al., Nat Genet 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with RLS, PLMS and/or PLMD may be assessed. In one such embodiment, variants associated with the C06 LD block and the Meis1 LD block are assessed, e.g., marker rs3923809 (or markers in LD therewith) and the A-rs2192954 G-rs2300478 haplotype (or markers or haplotypes in LD therewith).

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2.

Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. In one preferred embodiment, the significant r² value can be at least 0.1. In another preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (Caucasian, african, Japanese, chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Some representative methods for identification of haplotype blocks are set forth, for example, in U.S. Published Patent Application Nos. 20030099964, 20030170665, 20040023237 and 20040146870. Haplotype blocks can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. Such variants may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a linkage region, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Linkage Disequilibrium Using NEMO

LD between pairs of markers can be calculated using the standard definition of D′ and r² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and r² are extended to include microsatellites by averaging over the values for all possible allele combination of the two markers weighted by the marginal allele probabilities. For illustration purposes, when plotting all marker combination to elucidate the LD structure in a particular region, it may be appropriate to plot D′ in the upper left corner and the p-value or r² in the lower right corner. In the LD plots the markers can be plotted equidistant rather than according to their physical location, if desired.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The Risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

As described herein, certain markers and haplotypes comprising such markers are found to be useful for determination of susceptibility to sleep-related movement disorders, such as RLS, PLMD and/or PLMS—i.e., they are found to be useful for diagnosing a susceptibility to a sleep-related movement disorder, such as RLS, PLMD and/or PLMS. Particular alleles of polymorphic markers and haplotypes are found more frequently in individuals with the sleep-related movement disorder than in individuals without the sleep-related movement disorder. Therefore, these marker alleles and haplotypes have predictive value for detecting the sleep-related movement disorder, or a susceptibility to the sleep-related movement disorder, in an individual. Tagging markers within haplotype blocks or LD blocks comprising at-risk markers, such as the markers of the present invention, can be used as surrogates for other markers within the haplotype block or LD block. Therefore, “at-risk” tagging markers within the haplotype or LD block also have predictive value for detecting the sleep-related movement disorder, or a susceptibility to the sleep-related movement disorder, in an individual. “At-risk” tagging markers within haplotypes or LD blocks can also include other markers that distinguish among the haplotypes, as these similarly have predictive value for detecting the sleep-related movement disorder or a susceptibility to the sleep-related movement disorder.

Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high or possibly even higher than the at-risk variant. The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to sleep-related movement disorders, such as RLS, PLMD and/or PLMS, or a susceptibility to these disorders, in an individual. These tagging or surrogate markers that are in LD with the markers of the present invention can also include other markers that distinguish among haplotypes, as these similarly have predictive value for detecting susceptibility to RLS, PLMD and/or PLMS. The tagging and/or surrogate markers include markers that are described herein (e.g., markers as listed in Tables 1-5), but may also include other markers that are in LD with one or more of the markers listed in Tables 1-5.

The present invention can in certain embodiments be practiced by assessing a sample comprising genomic DNA from an individual for the presence of variants described herein to be associated with sleep-related movement disorders (e.g., RLS, PLMS and/or PLMD). Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of a sleep-related movement disorder. Alternatively, the invention can be practiced utilizing a dataset comprising information about the genotype status of at least one polymorphic marker described herein to be associated with sleep-related movement disorders (or markers in linkage disequilibrium with at least one marker shown herein to be associated with sleep-related movement disorders). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with sleep-related movement disorders. A positive result for a variant (e.g., marker allele) associated with a sleep-related movement disorder (e.g., RLS, PLMS and/or PLMD), as shown herein, is indicative of the individual from which the dataset is derived is at increased susceptibility (increased risk) of the sleep-related movement disorder.

In certain embodiments of the invention, a polymorphic marker is correlated to a sleep-related movement disorder by referencing genotype data for the polymorphic marker to a look-up table that comprises correlations between at least one allele of the polymorphism and the sleep-related movement disorder. In some embodiments, the table comprises a correlation for one polymorphism. In other embodiments, the table comprises a correlation for a plurality of polymorphisms. In both scenarios, by referencing to a look-up table that gives an indication of a correlation between a marker and the sleep-related movement disorder, a risk for the sleep-related movement disorder, or a susceptibility to the sleep-related movement disorder, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers and haplotypes of the invention, e.g., the markers presented in Tables 1-5, may be useful for risk assessment and diagnostic purposes, either alone or in combination. Thus, even in cases where the increase in risk by individual markers is relatively modest, e.g. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in one embodiment of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to a sleep-related movement disorder. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods, uses and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the sleep-related movement disorder. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g.; Tables 1-5), but may also include other markers that are in strong LD (e.g., characterized by r² greater than 0.1 or 0.2 and/or |D′|>0.8) with one or more of the markers listed in Tables 1-5.

For the SNP markers described herein, the opposite (alternate) allele to the allele found to be in excess in patients (at-risk allele) is found in decreased frequency in patients. These markers and haplotypes in LD and/or comprising such markers, are thus protective for a sleep-related movement disorder (e.g., PLMS, RLS and/or PLMD), i.e. they confer a decreased risk or susceptibility of individuals carrying these markers and/or haplotypes developing the sleep-related movement disorder.

The haplotypes and markers described herein are, in some cases, a combination of various genetic markers, e.g., SNPs and microsatellites. Therefore, detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker or haplotype associated with RLS, PLMD and/or PLMS (e.g., markers associated with C06 LD block, e.g. markers as listed in Tables 1-5) is one in which a certain marker allele or haplotype is more frequently present in an individual at risk for RLS, PLMD and/or PLMS (affected), compared to the frequency of its presence in a healthy individual (control), wherein the presence of the marker allele or haplotype is indicative of RLS, PLMD and/or PLMS or a susceptibility to RLS, PLMD and/or PLMS. In other embodiments, at-risk markers in linkage disequilibrium with one or more markers found to be associated with RLS, PLMD and/or PLMS (e.g., markers as listed in Tables 1-5) are tagging markers for which certain alleles are more frequently present in an individual at risk for RLS, PLMD and/or PLMS (affected), compared to the frequency of their presence in a healthy individual (control), wherein the presence of the tagging marker allele is indicative of increased susceptibility to RLS, PLMD and/or PLMS. In a further embodiment, at-risk marker alleles (i.e. conferring increased susceptibility) in linkage disequilibrium with one or more markers found to be associated with RLS, PLMD and/or PLMS (e.g., marker alleles as listed in Tables 1-5), are one or more alleles of specific markers that are more frequently present in an individual at risk for RLS, PLMD and/or PLMS, compared to the frequency of their presence in a healthy individual (control), wherein the presence of the marker alleles is indicative of increased susceptibility to RLS, PLMD and/or PLMS.

### Study Population

In a general sense, the methods and kits of the invention can be utilized from samples containing genomic DNA from any source, i.e. any individual. In preferred embodiments, the individual is a human individual. The individual can be an adult, child, or fetus. The present invention also provides for assessing markers and/or haplotypes in individuals who are members of a target population. Such a target population is in one embodiment a population or group of individuals at risk of developing RLS, PLMD and/or PLMS, based on other genetic factors, biomarkers, biophysical parameters (e.g., weight, BMD, blood pressure), or general health and/or lifestyle parameters (e.g., history of sleep-related movement disorder, including RLS, PLMD and/or PLMS, previous diagnosis of a sleep-related movement disorder, family history of a sleep-related movement disorder).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the disease in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either sex, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Stacey, S. N., et al., Nat Genet. May 27, 2007 (Epub ahead of print; Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including other Caucasian populations (European and/or North-American), African American populations, and populations from Africa and Asia.

The markers of the present invention found to be associated with sleep-related movement disorders (e.g., PLMS, PLMD and/or RLS) are believed to show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portugues, Italian, Polish, Bulgarian, Slavic, Serbian, Bosnian, Chech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Oroqen, She, Tujia, Tu, Xibo, Yi, Mongolan, Naxi, Cambodian, Japanese, Yakut, Melanesian, Papuan, Karitianan, Surui, Colmbian, Maya and Pima.

In one preferred embodiment, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry. In another embodiment, the invention relates to individuals of Caucasian origin.

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop a particular disease (e.g., RLS, PLMS and/or PLMD). The variants described herein do however indicate increased and/or decreased likelihood that individuals carrying the at-risk or protective variants of the invention will develop symptoms associated with the disease. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify early symptoms, so as to be able to apply treatment at an early stage.

The knowledge of a genetic variant that confers a risk of developing RLS, PLMS and/or PLMD offers the opportunity to apply a genetic-test to distinguish between individuals with increased risk of developing these diseases (i.e. carriers of the at-risk variant) and those with decreased risk of developing the diseases (i.e. carriers of the protective variants, or non-carriers of the at-risk variants). The core values of genetic testing, for individuals belonging to both of the above mentioned groups, are the possibilities of being able to diagnose the disease at an early stage and provide information to the clinician about prognosis and/or aggressiveness of the disease in order to be able to apply the most appropriate treatment, or to initiate treatment at an early stage. For example, the application of a genetic test for RLS, PLMD and/or PLMS can provide an opportunity for the detection of the disorders at an earlier stage which may lead to the application of therapeutic measures at an earlier stage, and thus can minimize the deleterious effects of the symptoms and adverse health consequences.

Individuals with a family history of a sleep-related movement disorder, such as RLS, PLMS and/or PLMD, and carriers of at-risk variants may benefit from genetic testing since the knowledge of the presence of a genetic risk factor, or evidence for increased risk of being a carrier of one or more risk factors, may provide increased incentive to apply measures to reduce complications accompanying the development of the disease.

Thus, the polymorphic markers of the present invention can be used alone or in combination, as well as in combination with other factors, including other genetic risk factors or biomarkers, for risk assessment of an individual for a sleep-related movement disorder, such as RLS, PLMD and/or PLMS. Common risk factors known to contribute to the susceptibility for sleep-related movement disorders include age, iron deficiency, blood donation. Females are also at increased risk of developing sleep-related movement disorders, such as RLS, PLMS and PLMD. Methods known in the art can be used for risk assessment of multiple risk factors, including multivariate analyses or logistic regression.

The finding that the rs3923809 A allele correlates with decreased iron stores in the body has implications for genetic testing. Thus, screening for the presence of rs3923809 A allele can be performed to identify individuals who are at particular risk of developing symptoms associated with decreased iron stores. In one such embodiment, women who are carriers of rs3923809 A allele could, for example, be directed to take iron supplements as a precautionary measure during pregnancy. Another embodiment relates to the assessment of blood donors. Blood donors who are carriers of rs3923809 A allele, in particular homozygous carriers, can be instructed to wait longer between blood donations than what is customary. In one such embodiment, blood donors are instructed to wait 4 months or longer between donations, while in other embodiments, blood donors are instructed to wait 5 months or longer, 6 months or longer, 7 months or longer, 8 months or longer or 12 months or longer, between blood donations.

Another utility of the assessment of rs3923809 A allele presence in samples from individuals is frequent measurement of human iron stores, e.g., measurements of serum ferritin levels, in those individuals, i.e. frequent assessment of the need for iron supplements for those individuals. Thus, assessment for rs3923809 A allele can be included in genetic profiling of an individual, and used in overall assessment of health risks the individual may in particular be susceptible to.

### Methods

Methods for risk assessment sleep-related movement disorders, including RLS, PLMS and PLMD, are described herein and are encompassed by the invention. The invention also encompasses methods of assessing an individual for probability of response to a therapeutic agent for sleep-related movement disorders, methods for predicting the effectiveness of a therapeutic agent for the sleep-related movement disorder, nucleic acids, polypeptides and antibodies and computer-implemented functions. Kits for assaying a sample from a subject to detect susceptibility to the sleep-related movement disorder are also encompassed by the invention.

### Diagnostic and Screening Methods

In certain embodiments, the present invention pertains to methods of diagnosing, or aiding in the diagnosis of, RLS, PLMD and/or PLMS or a susceptibility to RLS, PLMD and/or PLMS, by detecting particular alleles at genetic markers that appear more frequently in RLS, PLMD and/or PLMS subjects or subjects who are susceptible to RLS, PLMD and/or PLMS. In a particular embodiment, the invention is a method of diagnosing a susceptibility to RLS, PLMD and/or PLMS by detecting at least one allele of at least one polymorphic marker (e.g., the markers described herein). The present invention describes methods whereby detection of particular alleles of particular markers or haplotypes is indicative of a susceptibility to RLS, PLMD and/or PLMS. Such prognostic or predictive assays can also be used to determine prophylactic treatment of a subject prior to the onset of symptoms of RLS, PLMD and/or PLMS. The present invention pertains in some embodiments to methods of clinical applications of diagnosis, e.g., diagnosis performed by a medical professional. In other embodiments, the invention pertains to methods of diagnosis or determination of a susceptibility performed by a layman. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, made available to the individual can be compared to information from the public literature about disease or trait risk associated with various SNPs. The diagnostic application of disease-associated alleles as described herein, can thus be performed either by the individual, through analysis of his/her genotype data, or by a health professional based on results of a clinical test. In other words, the diagnosis or assessment of a susceptibility based on genetic risk can be made by health professionals, genetic counselors or by the layman, based on information about his/her genotype and publications on various risk factors. In the present context, the term “diagnosing”, “diagnose a susceptibility” and “determine a susceptibility” is meant to refer to any available diagnostic method, including those mentioned above.

In addition, in certain other embodiments, the present invention pertains to methods of diagnosing, or aiding in the diagnosis of, a decreased susceptibility to RLS, PLMD and/or PLMS, by detecting particular genetic marker alleles or haplotypes that appear less frequently in RLS, PLMD and/or PLMS patients than in individual not diagnosed with RLS, PLMD and/or PLMS or in the general population.

As described and exemplified herein, particular marker alleles or haplotypes (e.g. markers associated with C06 LD block, the markers and haplotypes as listed in Tables 1-4, and markers in linkage disequilibrium therewith) are associated with RLS, PLMD and/or PLMS. In one embodiment, the marker allele or haplotype is one that confers a significant risk or susceptibility to RLS, PLMD and/or PLMS. In another embodiment, the invention relates to a method of diagnosing a susceptibility to RLS, PLMD and/or PLMS in a human individual, the method comprising determining the presence or absence of at least one allele of at least one polymorphic marker in a nucleic acid sample obtained from the individual, wherein the at least one polymorphic marker is selected from the group consisting of the polymorphic markers associated with C06 LD block, the markers as listed in Tables 1-5, and markers in linkage disequilibrium therewith. In another embodiment, the invention pertains to methods of diagnosing a susceptibility to RLS, PLMD and/or PLMS in a human individual, by screening for at least one marker allele or haplotype, e.g. markers associated with C06 LD block, the markers and haplotypes as listed in Tables 1-5, and markers in linkage disequilibrium therewith. In another embodiment, the marker allele or haplotype is more frequently present in a subject having, or who is susceptible to, RLS, PLMD and/or PLMS (affected), as compared to the frequency of its presence in a healthy subject (control, such as population controls). In another embodiment, the invention relates to a method of diagnosing a susceptibility to RLS, PLMD and/or PLMS in a human individual, the method comprising determining the presence or absence of at least one allele of at least one polymorphic marker in a nucleic acid sample obtained from the individual, wherein the at least one polymorphic marker is selected from the group consisting of markers rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11), and markers in linkage disequilibrium therewith. In certain embodiments, the significance of association of the at least one marker allele or haplotype is characterized by a p value <0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to RLS, PLMD and/or PLMS. These diagnostic methods involve detecting the presence or absence of at least one marker allele or haplotype that is associated with RLS, PLMD and/or PLMS. The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a RLS, PLMD and/or PLMS-associated nucleic acid (e.g., by protein sequencing or by immunoassays using antibodies that recognize such a protein). The marker alleles or haplotypes of the present invention correspond to fragments of a genomic DNA sequence associated with RLS, PLMD and/or PLMS. Such fragments encompass the DNA sequence of the polymorphic marker or haplotype in question, but may also include DNA segments in strong LD (linkage disequilibrium) with the marker or haplotype (for example, as determined by a value of r² greater than 0.1 and/or |D′| greater than 0.8).

In one embodiment, diagnosis of a susceptibility to RLS, PLMD and/or PLMS can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject suspected of having, being susceptible to, or predisposed for RLS, PLMD and/or PLMS (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to RLS, PLMD and/or PLMS, a hybridization sample is formed by contacting the test sample containing an RLS, PLMD and/or PLMS-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the C06 LD block, the Meis1 LD block, the BTBD9 gene, the TEX27 gene, the GLO1 gene, the DNAH8 gene, as described herein, optionally comprising at least one allele of a marker described herein, or at least one haplotype comprising at least two of the markers described herein (e.g., the markers and haplotypes as listed in Tables 1-5, and markers in linkage disequilibrium therewith), or the probe can be the complementary sequence of such a sequence. In a particular embodiment, the nucleic acid probe is a portion of the nucleotide sequence of the C06 LD block, the Meis1 LD block, the DNAH8 gene, the BTBD9 gene, the TEX27 gene and the GLO1 gene, as described herein, optionally comprising at least one allele of a marker described herein (e.g., the markers as listed in Tables 1-5, markers in linkage disequilibrium therewith, and haplotypes comprising such markers), or the probe can be the complementary sequence of such a sequence. Other suitable probes for use in the diagnostic assays of the invention are described herein.

The hybridization sample is maintained under conditions that are sufficient to allow specific hybridization of the nucleic acid probe to the RLS, PLMD and/or PLMS-associated nucleic acid. “Specific hybridization”, as used herein, indicates exact hybridization (e.g., with no mismatches). Specific hybridization can be performed under high stringency conditions or moderate stringency conditions as described herein. In one embodiment, the hybridization conditions for specific hybridization are high stringency (e.g., as described herein).

Specific hybridization, if present, is then detected using standard methods. If specific hybridization occurs between the nucleic acid probe and the RLS, PLMD and/or PLMS-associated nucleic acid in the test sample, then the sample contains the allele that is complementary to the nucleotide that is present in the nucleic acid probe. The process can be repeated for other markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to RLS, PLMD and/or PLMS.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with RLS, PLMD and/or PLMS. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with RLS, PLMD and/or PLMS. Hybridization of the PNA probe is thus diagnostic for RLS, PLMD and/or PLMS or a susceptibility to RLS, PLMD and/or PLMS.

In one embodiment of the invention, diagnosis of RLS, PLMD and/or PLMS or a susceptibility to RLS, PLMD and/or PLMS is accomplished through enzymatic amplification of a nucleic acid from the subject. For example, a test sample containing genomic DNA can be obtained from the subject and the polymerase chain reaction (PCR) can be used to amplify a fragment comprising one or more markers or haplotypes of the present invention found to be associated with RLS, PLMD and/or PLMS. As described herein, identification of a particular marker allele or haplotype associated with RLS, PLMD and/or PLMS can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.), to allow the identification of polymorphisms and haplotypes. The technique can assess the presence of an alteration in the expression or composition of a polypeptide or splicing variant(s) that is encoded by a RLS, PLMD and/or PLMS-associated nucleic acid. Further, the expression of the variant(s) can be quantified as physically or functionally different.

In another method of the invention, analysis by restriction digestion can be used to detect a particular allele if the allele results in the creation or elimination of a restriction site relative to a reference sequence. A test sample containing genomic DNA is obtained from the subject. PCR can be used to amplify particular regions that are associated with RLS, PLMD and/or PLMS (e.g. region containing at least one of the polymorphic markers of the invention, e.g. the markers as listed in Tables 1-5 and markers in linkage disequilibrium therewith). Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with RLS, PLMD and/or PLMS (e.g. the polymorphic markers of Tables 1-5 and markers in linkage disequilibrium therewith). Therefore, in one embodiment, determination of the presence or absence of a particular marker alleles or haplotypes comprises sequence analysis. For example, a test sample of DNA or RNA can be obtained from the test subject. PCR or other appropriate methods can be used to amplify a portion of a RLS, PLMD and/or PLMS-associated nucleic acid, and the presence of a specific allele can then be detected directly by sequencing the polymorphic site (or multiple polymorphic sites) of the genomic DNA in the sample.

Allele-specific oligonucleotides can also be used to detect the presence of a particular allele at a RLS, PLMD and/or PLMS-associated nucleic acid (e.g. the polymorphic markers and haplotypes of Tables 1-5 and markers in linkage disequilibrium therewith), through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a RLS, PLMD and/or PLMS-associated nucleic acid, and which contains a specific allele at a polymorphic site (e.g., a polymorphism as described herein, e.g., the polymorphisms listed in Tables 1-5). An allele-specific oligonucleotide probe that is specific for one or more particular a RLS, PLMD and/or PLMS-associated nucleic acid can be prepared using standard methods (see, e.g., Current Protocols in Molecular Biology, supra). PCR can be used to amplify the desired region a RLS, PLMD and/or PLMS-associated nucleic acid. The DNA containing the amplified region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with RLS, PLMD and/or PLMS (see, e.g., Gibbs, R. et al., Nucleic Acids Res., 17:2437-2448 (1989) and WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a RLS, PLMD and/or PLMS-associated nucleic acid (e.g. the polymorphic markers and haplotypes of Tables 1-5 and markers in linkage disequilibrium therewith). For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods or light directed synthesis methods that incorporate a combination of photolithographic methods and solid phase oligonucleotide synthesis methods (Fodor, S. et al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein and also in, e.g., published PCT Application Nos. WO 92/10092 and WO 95/11995, and U.S. Pat. No. 5,424,186, the entire teachings of each of which are incorporated by reference herein. In brief, a target nucleic acid sequence, which includes one or more previously identified polymorphic markers, is amplified by well-known amplification techniques (e.g., PCR). Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms (e.g., multiple polymorphisms of a particular haplotype). In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of both of which are incorporated by reference herein.

Other methods of nucleic acid analysis can be used to detect a particular allele at a polymorphic site associated with a sleep-related movement disorder, such as RLS, PLMD and/or PLMS (e.g. the polymorphic markers and haplotypes of Tables 1-5 and markers in linkage disequilibrium therewith). Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Nat. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of RLS, PLMD and/or PLMS or a susceptibility to RLS, PLMD and/or PLMS can be made by examining expression and/or composition of a polypeptide encoded by RLS, PLMD and/or PLMS-associated nucleic acid in those instances where the genetic marker(s) or haplotype(s) of the present invention result in a change in the composition or expression of the polypeptide. Thus, diagnosis of a susceptibility to RLS, PLMD and/or PLMS can be made by examining expression and/or composition of one of these polypeptides, or another polypeptide encoded by a RLS, PLMD and/or PLMS-associated nucleic acid, in those instances where the genetic marker or haplotype of the present invention results in a change in the composition or expression of the polypeptide. The haplotypes and markers of the present invention that show association to RLS, PLMD and/or PLMS may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used to make such a detection, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a RLS, PLMD and/or PLMS-associated nucleic acid. An alteration in expression of a polypeptide encoded by a RLS, PLMD and/or PLMS-associated nucleic acid can be, for example, an alteration in the quantitative polypeptide expression (i.e., the amount of polypeptide produced). An alteration in the composition of a polypeptide encoded by a RLS, PLMD and/or PLMS-associated nucleic acid is an alteration in the qualitative polypeptide expression (e.g., expression of a mutant polypeptide or of a different splicing variant). In one embodiment, diagnosis of a susceptibility to RLS, PLMD and/or PLMS is made by detecting a particular splicing variant encoded by a RLS, PLMD and/or PLMS-associated nucleic acid, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of polypeptide encoded by a RLS, PLMD and/or PLMS-associated nucleic acid in a control sample. A control sample is a sample that corresponds to the test sample (e.g., is from the same type of cells), and is from a subject who is not affected by, and/or who does not have a susceptibility to, RLS, PLMD and/or PLMS (e.g., a subject that does not possess a marker allele or haplotype as described herein). Similarly, the presence of one or more different splicing variants in the test sample, or the presence of significantly different amounts of different splicing variants in the test sample, as compared with the control sample, can be indicative of a susceptibility to RLS, PLMD and/or PLMS. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a RLS, PLMD and/or PLMS-associated nucleic acid can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a RLS, PLMD and/or PLMS-associated nucleic acid (e.g, a nucleic acid encoding one or more of the GLO1 polypeptide, TEX27 polypeptide, BTBD9 polypeptide and the DNAH8 polypeptide) can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a RLS, PLMD and/or PLMS-associated nucleic acid in a test sample is compared with the level or amount of the polypeptide encoded by a RLS, PLMD and/or PLMS-associated nucleic acid in a control sample. A level or amount of the polypeptide in the test sample that is higher or lower than the level or amount of the polypeptide in the control sample, such that the difference is statistically significant, is indicative of an alteration in the expression of the polypeptide encoded by the RLS, PLMD and/or PLMS-associated nucleic acid, and is diagnostic for a particular allele or haplotype responsible for causing the difference in expression. Alternatively, the composition of the polypeptide encoded by a RLS, PLMD and/or PLMS-associated nucleic acid in a test sample is compared with the composition of the polypeptide encoded by a RLS, PLMD and/or PLMS-associated nucleic acid in a control sample. In another embodiment, both the level or amount and the composition of the polypeptide can be assessed in the test sample and in the control sample.

In another embodiment, the diagnosis of a susceptibility to RLS, PLMD and/or PLMS is made by detecting at least one RLS, PLMD and/or PLMS-associated marker allele or haplotype (e.g., the polymorphic markers and haplotypes of Tables 1-5 and markers in linkage disequilibrium therewith) in combination with an additional protein-based, RNA-based or DNA-based assay. The methods of the invention can also be used in combination with an analysis of a subject's family history and risk factors (e.g., environmental risk factors, lifestyle risk factors).

### Kits

Kits useful in the methods of diagnosis comprise components useful in any of the methods described herein, including for example, hybridization probes, restriction enzymes (e.g., for RFLP analysis), allele-specific oligonucleotides, antibodies that bind to an altered polypeptide encoded by RLS, PLMD and/or PLMS-associated nucleic acid (e.g., antibodies that bind to a polypeptide encoded by a genomic segment comprising at least one polymorphic marker and/or haplotype of the present invention) or to a non-altered (native) polypeptide encoded by a RLS, PLMD and/or PLMS-associated nucleic acid, means for amplification of a RLS, PLMD and/or PLMS-associated nucleic acid, means for analyzing the nucleic acid sequence of RLS, PLMD and/or PLMS-associated nucleic acid, means for analyzing the amino acid sequence of a polypeptide encoded by a RLS, PLMD and/or PLMS-associated nucleic acid, etc. Additionally, kits can provide reagents for assays to be used in combination with the methods of the present invention, e.g., reagents for use with other RLS, PLMD and/or PLMS diagnostic assays.

In one embodiment, the invention is a kit for assaying a sample from a subject to detect RLS, PLMD and/or PLMS or a susceptibility to RLS, PLMD and/or PLMS in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention (e.g., the polymorphic markers and haplotypes of Tables 1-5 and markers in linkage disequilibrium therewith). In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes one polymorphism, wherein the polymorphism is selected from the group consisting of the polymorphisms as defined in Tables 1-5, and polymorphisms in linkage disequilibrium therewith. In yet another embodiment the fragment is at least 20 base pairs in size. Such oligonucleotides or nucleic acids (e.g., oligonucleotide primers) can be designed using portions of the nucleic acids flanking polymorphisms (e.g., SNPs or microsatellites) that are indicative of RLS, PLMD and/or PLMS. In another embodiment, the kit comprises one or more labeled nucleic acids capable of detecting one or more specific polymorphic markers or haplotypes associated with RLS, PLMD and/or PLMS, and reagents for detection of the label. Suitable labels include, e.g., a radioisotope, a fluorescent label, an enzyme label, an enzyme co-factor label, a magnetic label, a spin label, an epitope label.

In another embodiment, the kit for assessing susceptibility to PLMD and/or PLMS comprises at least one oligonucleotide probe that is from 5-100 nucleotides in length and specifically hybridizes (under stringent conditions) to all or a portion of C06 LD block, and wherein at least one of said at least one oligonucleotide probes comprises a polymorphism selected from the group of polymorphisms of the invention (e.g., the polymorphisms of Tables 1-5, and polymorphisms in linkage disequilibrium therewith). In one embodiment, the kit further comprises at least one oligonucleotide pair for amplifying a genomic fragment to which the at least one oligonucleotide probe hybridizes, the segment being from 40-500 nucleotides in length. In one such embodiment, the oligonucleotide probe comprises a detectable label. In one particular embodiment, the kit comprises two oligonucleotide probes, wherein one of said probes comprises at least one detectable label and a polymorphism as listed in any of the Tables 1-5. In another embodiment, at least one of said probes comprises one detectable label, a quencher and a polymorphism selected from the polymorphisms listed in Tables 1-5.

In particular embodiments, the polymorphic marker or haplotype to be detected by the reagents of the kit comprises one or more markers, two or more markers, three or more markers, four or more markers or five or more markers selected from the group consisting of the markers in Tables 1-5 (SEQ ID NO: 1-328). In another embodiment, the marker to be detected is selected from markers rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11), and markers in linkage disequilibrium therewith. In another embodiment, the marker to be detected is selected from markers rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3), and markers in linkage disequilibrium therewith. In yet another embodiment, the marker to be detected is marker rs3923809, or markers in linkage disequilibrium therewith. In yet another embodiment, the marker to be detected is selected from the markers set forth in Table 5. In yet another embodiment, the marker to be detected is marker rs3923809. In a further embodiment, the marker to be detected is selected from the marker set forth in Table 12, or markers in linkage disequilibrium therewith. In yet a further embodiment, the markers to be detected are markers rs2192954 and rs2300478. In another embodiment, the marker or haplotype comprises marker rs3923809 allele 1, rs9357271 allele 4, rs6923737 allele 4, rs7770868 allele 1, rs4711546 allele 1, rs6904723 allele 1, rs12208647 allele 1, rs4236060 allele 2, rs10947739 allele 2, rs6920488 allele 1, and rs13219518 allele 1. In such embodiments, the presence of the marker or haplotype is indicative of a susceptibility (increased susceptibility or decreased susceptibility) to RLS, PLMD and/or PLMS.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one of such embodiments, the presence of the marker or haplotype is indicative of a susceptibility (increased susceptibility or decreased susceptibility) to a sleep-related movement disorder (e.g., RLS, PLMS and/or PLMD). In another embodiment, the presence of the marker or haplotype is indicative of response to a therapeutic agent for a sleep-related movement disorder. In another embodiment, the presence of the marker or haplotype is indicative of prognosis of a sleep-related movement disorder. In yet another embodiment, the presence of the marker or haplotype is indicative of progress of treatment of a sleep-related movement disorder. Such treatment may include intervention by medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents

Treatment of prevention of sleep-related movement disorders, including RLS, PLMS and PLMD includes dietary supplements and pharmaceutical agents. Some guidelines suggest that all people with RLS or PLMS should have ferritin levels checked; those with low levels of ferritin can be prescribed oral iron supplements. Treatment with iron IV is being tested.

Pharmaceutical agents for RLS, PLMS and/or PLMD include (1) dopamine agonists, including dextropropoxyphene, ropinirole, pramipexile, carbidopa/levodopa, co-careldopa, co-beneldopa, lisuride or pergolide; (2) opioids, including propoxyphen, oxycodone and methadone; (3) benzidazepines, which often assist people in staying asleep and reduce awakening from symptoms; and (4) anticonvulsants, including gabapentin, which may help people who experience painful symptoms.

Variants of the present invention (e.g., the markers and/or haplotypes of the invention, e.g., the markers listed in Tables 1-5) can be used to identify novel therapeutic targets for RLS, PLMD and/or PLMS. For example, genes containing, or in linkage disequilibrium with, variants (markers and/or haplotypes) associated with RLS, PLMD and/or PLMS, e.g. the GLO1, TEX27, BTBD9 and/or DNAH8 genes, or their products, as well as genes or their products that are directly or indirectly regulated by or interact with these variant genes or their products, can be targeted for the development of therapeutic agents to treat RLS, PLMD and/or PLMS, or prevent or delay onset of symptoms associated with RLS, PLMD and/or PLMS. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat a disease or disorder, such as RLS, PLMD and/or PLMS. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., the markers and haplotypes set forth in Tables 1-5) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a sleep-related movement disorder, including RLS, PLMD and/or PLMS, or a defect causing the disorder, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the administered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat RLS, PLMD and/or PLMS. Thus, the variants of the invention are useful as targets for the identification and/or development of therapeutic agents. Such methods may include assaying the ability of an agent or compound to modulate the activity and/or expression of a nucleic acid that includes at least one of the variants (markers and/or haplotypes) of the present invention, or the encoded product of the nucleic acid, e.g. the DNAH8 gene, the TEX27 gene, the GLO1 gene and/or the BTBD9 gene, or their gene products. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. One example is provided by the GLO1 expression data shown in FIG. 3 herein. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating RLS, PLMD and/or PLMS can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different, e.g. a different response rate, to a particular treatment modality, e.g. any of the therapeutic agents used for treating or preventing RLS, PLMD and/or PLMS, as described above. This means that a patient diagnosed with a sleep-related movement disorder, such as RLS, PLMD and/or PLMS, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype at (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

The present invention also relates to methods of monitoring progress or effectiveness of a treatment for RLS, PLMD and/or PLMS, including any one or combinations of the therapeutic agents described above. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for RLS, PLMD and/or PLMS as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention, i.e. individuals who are carriers of at least one allele of at least one polymorphic marker conferring increased risk of developing RLS, PLMD and/or PLMS may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with RLS, PLMD and/or PLMS when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Uses in Forensic and Paternity Testing

In addition to the diagnostic and therapeutic uses of the variants of the present invention, the variants (markers and haplotypes) can also be useful markers for human identification, and as such be useful in forensics, paternity testing and in biometrics. The specific use of SNPs for forensic purposes is reviewed by Gill (Int. J. Legal Med. 114:204-10 (2001)). Genetic variations in genomic DNA between individuals can be used as genetic markers to identify individuals and to associated a biological sample with an individual. Genetic markers, including SNPs and microsatellites, can be useful to distinguish individuals. The more markers that are analyzed, the lower the probability that the allelic combination of the markers in any given individual is the same as in an unrelated individual (assuming that the markers are unrelated, i.e. that the markers are in perfect linkage equilibrium). Thus, the variants used for these purposes are preferably unrelated, i.e. they are inherited independently. Thus, preferred markers can be selected from available markers, such as the markers of the present invention, and the selected markers may comprise markers from different regions in the human genome, including markers on different chromosomes.

In certain applications, the SNPs useful for forensic testing are from degenerate codon positions (i.e., the third position in certain codons such that the variation of the SNP does not affect the amino acid encoded by the codon). In other applications, such for applications for predicting phenotypic characteristics including race, ancestry or physical characteristics, it may be more useful and desirable to utilize SNPs that affect the amino acid sequence of the encoded protein. In other such embodiments, the variant (SNP or other polymorphic marker) affects the expression level of a nearby gene, thus leading to altered protein expression.

### Computer-Implemented Aspects

The present invention also relates to computer-implemented applications of the polymorphic markers and haplotypes described herein to be associated with sleep-related movement disorders. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual), or for deriving information from the genotype data, e.g., by comparing the genotype data to information about genetic risk factors contributing to increased susceptibility to a sleep-related movement disorder, and reporting results based on such comparison.

One such aspect relates to computer-readable media. In general terms, such medium has capabilities of storing (i) identifier information for at least one polymorphic marker or a haplotype; (ii) an indicator of the frequency of at least one allele of said at least one marker, or the frequency of a haplotype, in individuals with a sleep-related movement disorder (e.g, RLS, PLMS and/or PLMD; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

Additional information about the individual can be stored on the medium, such as ancestry information, information about sex, physical attributes or characteristics (including height and weight), biochemical measurements (such as blood pressure, blood lipid levels, fasting glucose levels, ferritin levels, ferritin index values), or other useful information that is desirable to store or manipulate in the context of the genotype status of a particular individual.

The invention furthermore relates to an apparatus that is suitable for determination or manipulation of genetic data useful for determining a susceptibility to a sleep-related movement disorders (e.g. PLMS, PLMD and/or RLS) in a human individual. Such an apparatus can include a computer-readable memory, a routine for manipulating data stored on the computer-readable memory, and a routine for generating an output that includes a measure of the genetic data. Such measure can include values such as allelic or haplotype frequencies, genotype counts, sex, age, phenotype information, values for odds ratio (OR) or relative risk (RR), population attributable risk (PAR), or other useful information that is either a direct statistic of the original genotype data or based on calculations based on the genetic data.

The markers and haplotypes shown herein to be associated with increased susceptibility (e.g., increased risk) of a sleep-related movement disorder, are in certain embodiments useful for interpretation and/or analysis of genotype data. Thus in certain embodiments, an identification of an at-risk allele for a sleep-related movement disorder, as shown herein, or an allele at a polymorphic marker in LD with any one of the markers shown herein to be associated with a sleep-related movement disorder, is indicative of the individual from whom the genotype data originates is at increased risk of the sleep-related movement disorder. In one such embodiment, genotype data is generated for at least one polymorphic marker shown herein to be associated with the sleep-related movement disorder, or a marker in linkage disequilibrium therewith. The genotype data is subsequently made available to the individual from whom the data originates, for example via a user interface accessable over the internet, together with an interpretation of the genotype data, e.g., in the form of a risk measure (such as an absolute risk (AR), risk ratio (RR) or odds ratio (OR)) for the sleep-related movement disorder (e.g., PLMS, PLMD and/or RLS). In another embodiment, at-risk markers identified in a genotype dataset derived from an individual are assessed and results from the assessment of the risk conferred by the presence of such at-risk varians in the dataset are made available to the individual, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived. In particular embodiments, the results of risk assessment is made available to a third party, e.g., a physician, other healthcare worker or genetic counselor.

### Markers Useful in Various Aspects of the Invention

The above-described methods and applications relating to sleep-related movements can in general be practiced with the markers and haplotypes of the invention that have in more detail been described herein in general terms as being useful for assessing susceptibility to the sleep-related movement disorders RLS, PLMS and PLMD. Thus, these applications can in general be reduced to practice using the markers as listed in Tables 1-5 (SEQ ID NO: 1-328) and Tables 10-12 (SEQ ID NO: 329-343), and markers in linkage disequilibrium therewith. In some embodiments, the markers are selected from the group of markers set forth in Table 4, and markers in linkage disequilibrium therewith. In some other embodiments, the markers are selected from rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3), and markers in linkage disequilibrium therewith. In some embodiments, the markers are selected from the group consisting of rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11). In particular preferred embodiments, the markers are selected from marker rs3923809 (SEQ ID NO:1), and markers in linkage disequilibrium therewith. In some other preferred embodiments, the markers are selected from the markers set forth in Table 5. In some further embodiments, the markers are selected from markers within the C06 LD block region. Certain embodiments relate to markers associated with the Meis1 gene, including markers in linkage disequilibrium with the Meis1 gene. In one such embodiment, the markers are selected from markers within the Meis1 LD block. In a preferred embodiment, the markers are selected from the markers set forth in Table 12. In another preferred embodiment, the markers are A-rs2192954 G-rs2300478, wherein the presence of allele A in rs2192954 and allele G in rs2300478 is indicative of increased risk of a sleep-related movement disorder. In other preferred embodiments, the markers are selected from markers in linkage disequilibrium with the A-rs2192954 G-rs2300478 haplotype.

### Nucleic Acids and Polypeptides

The nucleic acids and polypeptides described herein can be used in methods and kits of the present invention, as described in the above.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Nat. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, nucleotide sequence of the C06 LD block, the nucleotide sequence of the Meis1 LD block, the nucleotide sequence of the BTBD9 gene, the nucleotide sequence of the TEX27 gene, the nucleotide sequence of the GLO1 gene, the nucleotide sequence of the DNAH8 gene, or a nucleotide sequence comprising, or consisting of, the complement of the nucleotide sequence of C06 LD block, the Meis1 LD block, the nucleotide sequence of the BTBD9 gene, the nucleotide sequence of the TEX27 gene, the nucleotide sequence of the GLO1 gene, the nucleotide sequence of the DNAH8 gene, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify RLS, PLMD and/or PLMS, and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular RLS, PLMD and/or PLMS. Antibodies specific for a variant protein of the present invention that is encoded by a nucleic acid that comprises at least one polymorphic marker or haplotype as described herein can be used to screen for the presence of the variant protein, for example to screen for a predisposition to RLS, PLMD and/or PLMS as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labeled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting examples.

### Example 1

### Identification of Genetic Variants on Chromosome 6p21.2 Found to be Associated with RLS and PLMS

### Material and Methods

### Patient Recruitment

Approval was obtained from the National Bioethics Committee (02-057) and the Data Protection Commission (DPC) of Iceland. A list of 514 subjects answering to a single newspaper advertisement highlighting the essential clinical symptoms of RLS was generated in a single week in February 2003. A Nurse Practitioner (IE) from the blood collection clinic under the auspices of the DPC then contacted subjects willing to participate. During an initial clinic visit, formal written informed consent was obtained for additional clinical, genetic, and electrophysiological analysis. The DPC encrypts all personal identifiers associated to information or blood samples using the third-party encryption system developed by deCODE Genetics Inc. in collaboration with the DPC¹⁵. Having obtained consent, the nurse practitioner 1) obtained a general medical and medication history; 2) administered a second 80-item questionnaire that contained a set of questions from several other validated, quantifiable sleep-related questionnaires (e.g., the International Restless Legs Syndrome Study Group (IRLSSG) rating scale (Walters, A. S. et al. Sleep Med 4, 121-32 (2003)); Epworth Sleepiness Scale (Johns, M. W. Sleep 14, 540-5 (1991)); and Basic Nordic Sleep Questionnaire (Partinen, M. & Gislason, T. J Sleep Res 4, 150-155 (1995)), and that assessed clinical features, potential comorbid conditions, and quality of life; 3) collected venous blood in three 10 cc vials containing anticoagulant for DNA extraction, and two 10 cc red-top vials for analysis of measures of iron metabolism; and 4) instructed the patient on use of PAM-RL monitors for phenotypic characterization of PLMS (see below). During this interview subjects were asked about RLS symptoms in family members.

Regardless, however, of positive or negative family history, subjects were asked to distribute a screening questionnaire containing the 1995 IRLSSG consensus criteria (Walters, A. S. Mov Disord 10, 634-42 (1995)) to both affected and unaffected family members. If verbal consent was obtained from the patient to contact these family members, they were contacted by telephone and asked about their RLS symptomatology and willingness to participate in the study.

### Phenotypic Characterization

The diagnosis of RLS remains a clinical one, with modified criteria published in 2003 by the IRLSSG (Allen, R. P. et al. Sleep Med 4, 101-19 (2003)). Because concept and design, and recruitment for this study occurred in 2002 and early 2003, respectively, subjects were required to answer questions that captured the 4 essential diagnostic criteria for RLS established in 1995 (Walters, A. S. Mov Disord 10, 634-42 (1995)). The specific questions and possible responses included:
1. When sitting or lying down, do you have a strong urge to move your legs (never, rarely (once a month or less), sometimes (2-4 times/month), often (5-15 times/month), and very often (more than 15 times/month).
2. Is your urge to move accompanied by a discomfort (unpleasant sensation) in your legs, for example, creepy-crawly or tingly feelings (yes, no, don't know)?
3. Is the discomfort in your legs relieved in any way, even for a short time, by walking or moving your legs (yes, no, don't know)?
4. At what times is the discomfort in your legs and urge to move most bothersome to you (in the morning (after breakfast and before noon), in the afternoon (before supper), in the evening (after supper), at bedtime, no difference by time of day)?

As the 2003 revised criteria split question #1 into two components (i.e. an imperative urge and worsening with inactivity) and excluded question #2, 43 subjects were considered to be affected (the RLScc group) if they reported symptoms at least 2-4 times/month that were relieved by movement, and that occurred in the evening or at bedtime. To complement this subjective assessment, subjects were instructed by the NP on use of a small (65 gm), wristwatch-sized tri-axial accelerometer with 10 Hz sampling (PAM-RL detector, IM Systems, Baltimore, Mass.) affixed via a Velcro strip to their most affected ankle (default: non-dominant ankle) for 5 consecutive nights in order to derive: 1) a mean, maximum and minimum PLM index (PLMi; # of PLMS/hour); and 2) an empirically derived assessment of PLMS status. The PAM-RL provides an accurate naturalistic assessment of polysomnographically derived PLMi (Pearson's correlation r=0.87, p<0.0001) (Sforza, E., Johannes, M. & Claudio, B. Sleep Med 6, 407-13 (2005)), has been reported to differentiate and discriminate between RLS-related motor symptoms and normal nocturnal motor activity (Tuisku, K., Holi, M. M., Wahlbeck, K., Ahigren, A. J. & Lauerma, H. Mov Disord 18, 442-8 (2003)), and now is routinely used to confirm treatment efficacy in RLS subjects (Tuisku, K., Holi, M. M., Wahlbeck, K., Ahlgren, A. J. & Lauerma, H. Eur J Neurol 12, 385-7 (2005); Rye D, A. R., Carson S and Ritchie S. Sleep 28, A270 (2005)). Encrypted data conveying the temporal pattern and quantity of PLMS was uploaded to a secure FTP site, and then downloaded weekly for off-line analysis by DBR and JMB to ensure data integrity and consistency. Quantification of PLMS was performed through use of a software algorithm based upon standards of practice for PLMS scoring that accompanied the PAM-RL (version 7.5.70) (The Atlas Task Force Sleep 16, 748-59 (1993)). This required a user-generated start (beginning of sleep period) and stop (end of sleep period) time guided by reference to an up-down body position function/marker. Independent of the absolute number of PLMS/hour, other qualitative features including time of night (Culpepper, W. J., Badia, P. & Shaffer, J. I. Sleep 15, 306-11 (1992)), and sleep stage and periodicity (Pollmacher, T., Mullington, J. & Lauer, C. J. Biol Psychiatry 42, 713-20 (1997)) characterize PLMS in RLS subjects. Moreover, some PLMS went unscored by the software algorithm due to its reliance upon a pre-determined minimal threshold for detection of a leg movement. Thus, a single observer (DBR) blind to each subject's clinical features empirically derived a subject's PLMS status as: positive, probably positive, probably negative, and negative. In Table I Positive and probably positive subjects are analysed. Classification as positive required that the quantity or pattern of leg movements emulated PLMS on at least 2 of 5 nights of recording, while at least one of five nights of similar PLM activity was required for categorization as probably positive. When leg movements consisted of only short intervals (e.g., <20 minutes) that were periodic in nature, were aperiodic, modest in number, separated by fairly long intermovement intervals (e.g., 50-90 seconds), or occurred later after apparent sleep onset, subjects were classified as probably negative. In subjects categorized as negative, leg movements were typically absent on all 5 recording nights. The accuracy of this diagnostic scheme was determined by comparison to a consensus diagnosis (only ‘positive’ or ‘negative’ allowed) derived from face-to-face personal interviews and physical examinations of 123 of the 514 ‘probands’ by 5 clinicians experienced in the diagnosis and care of RLS patients (Rye D, B. D., Iranzo A, et al. Sleep 27, 306-07 (2004)).

The Icelandic controls were chosen at random from individuals that have participated in other genetic studies at deCODE, excluding first-degree relatives of patients and controls.

### Association Analysis

Samples were genotyped using the Illumina Human Hap300 and Human Hap300-duo+ Bead Arrays. In total 311398 SNP markers were common to both CHIPs, thereof 2176 were excluded due to problems with yield or markers were not in Hardy-Weinberg equilibrium in controls. Thus, 309,222 SNP markers gave good quality genotypes and were used in the analysis. The 390 cases were tested for association to the 309,222 SNPs using genotypes from 15,600 Icelandic controls. Samples with a call rate below 98% were excluded from the analysis.

A likelihood procedure described in a previous publication (Gretarsdottir, S. et al. Nat Genet 35, 131-8 (2003)), and implemented in the NEMO software, was used for the association analyses. Allele-specific RR was calculated assuming a multiplicative model for the two chromosomes of an individual (Falk, C. T. & Rubinstein, P. Ann Hum Genet 51, 227-33 (1987)).

In all tables, the RRs and P values for the case-control comparisons of all SNPs were corrected for relatedness of patients.

### Expression Analysis

Peripheral blood was used for RNA extraction, the samples being collected from participating individuals, who gave their informed consent in accordance with regulations of the Icelandic Data Protection Authority and the National Bioethics Committee, between 8 and 10 in the morning, after fasting overnight. RNA was extracted within 2 hrs by Erythrocyte Lysis buffer using the RNeasy Midi Kit (Qiagen).

All RNA was amplified as described previously (van't Leer L. J., et al., Nature 415, 530-536 (2002)). The samples were hybridized to a Human 25K array manufactured by Agilent Tecnologies, containing 23,720 probes (Johnson, J. M. et al., Science 302, 2141-2144 (2003)). Array images were processed as described previously (van't Leer et al., Nature 415, 530-536 (2002)). Expression changes between two samples were quantified as mean logarithmic (log10) expression ratio (MLR), i.e. expression ratios between background corrected intensity values for the two channels for each spot on the array.

### Results

### Identification of a PLMS/RLS Locus in an Icelandic Sample.

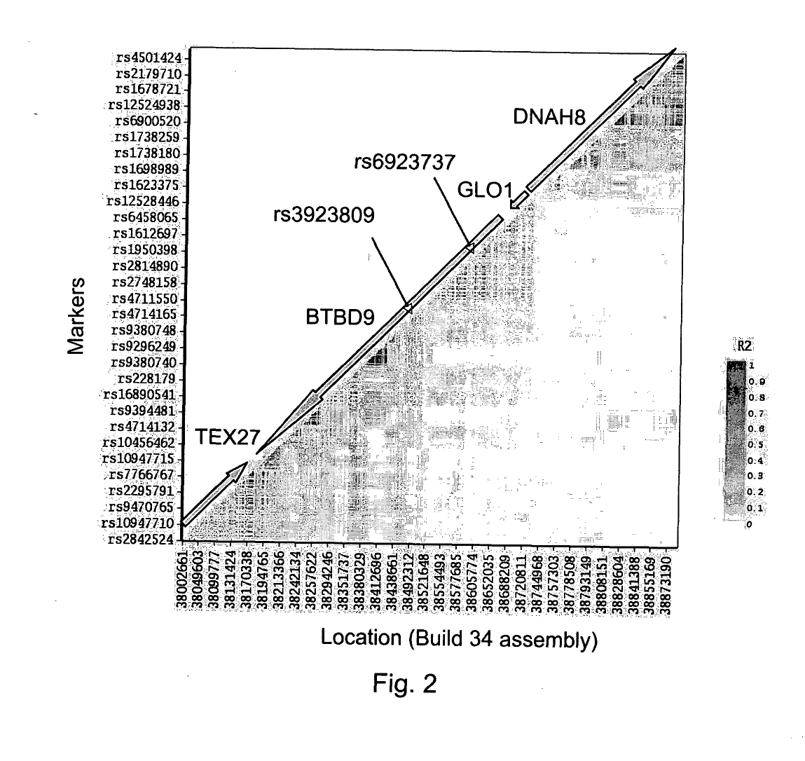

In the discovery phase we obtained genotypes from 390 well phenotyped Icelandic subjects diagnosed either with RLS, PLMS or both phenotypes (detailed description of the phenotypic characterization can be found in the Method section). Of the 390 cases 349 were diagnosed with PLMS, 290 with RLS and 249 with both diagnoses. Samples were genotyped using the Illumina Human Hap300 and Human Hap300-duo+ Bead Arrays. In total 311398 SNP markers were common to both CHIPs, thereof 2176 were excluded due to problems with yield or markers were not in Hardy-Weinberg equilibrium in controls. Thus, 309,222 SNP markers gave good quality genotypes and were used in the analysis. The 390 cases were tested for association to the 309,222 SNPs using genotypes from 15,600 Icelandic controls. Six markers; rs3923809, rs7770868, rs9357271, rs6923737, rs6904723 and rs4711546 in a single linkage disequilibrium (LD) block show genome-wide association with PLMS. These markers are located in the BTB (POZ) domain containing 9 (BTBD9) gene on chromosome 6p21 (Table 1 and FIGS. 1 and 2). One of the markers, rs3923809, furthermore shows genome-wide significant association to RLS (Table 2). For all six markers the rr is significantly higher for the PLMS group than for the RLS group. The most significant marker for both phenotypes is, rs3923809 (r=1.99; P=5.5×10⁻¹³), is found in 79.1% frequency in the PLMS group and 65.4% frequency in the control group. Thus, this variant is accounting for 630% of the population attributable risk for PLMS. Similarly this variant is found in 77.2% frequency in the RLS group and accounting for 57% of the population attributable risk. These results have been adjusted for the relatedness among the subjects.

TABLE 1
Six markers in an LD block on chromosome 6p21.2 show genome-wide
significant association with PLMS. (349 affecteds with >98% yield,
15600 controls with >98% yield, allele 1 =
A, allele 2 = C, allele 3 = G, allele 4 = T)

| corr. p- | | | | | | | | | |
|---|---|---|---|---|---|---|---|---|---|
| val | r | #aff | afffrq | #con | con. freq | corr. X2 | PAR | allele | SNP |
| 5.5 * 10⁻¹³ | 0.50 | 349 | 0.209 | 15575 | 0.345 | 52.0 | | 3 | rs3923809 |
| 5.5 * 10⁻¹³ | 1.99 | 349 | 0.791 | 15575 | 0.654 | 52.0 | 0.63 | 1 | rs3923809 |
| 1.6 * 10⁻⁸ | 0.53 | 349 | 0.143 | 15598 | 0.237 | 31.9 | | 2 | rs9357271 |
| 1.6 * 10⁻⁸ | 1.86 | 349 | 0.857 | 15598 | 0.762 | 31.9 | 0.63 | 4 | rs9357271 |
| 2.1 * 10⁻⁸ | 0.59 | 349 | 0.241 | 15596 | 0.348 | 31.4 | | 2 | rs6923737 |
| 2.1 * 10⁻⁸ | 1.68 | 349 | 0.759 | 15596 | 0.652 | 31.4 | 0.52 | 4 | rs6923737 |
| 2.4 * 10⁻⁸ | 0.61 | 329 | 0.307 | 15351 | 0.423 | 31.2 | | 2 | rs7770868 |
| 2.4 * 10⁻⁸ | 1.65 | 329 | 0.693 | 15351 | 0.577 | 31.2 | 0.47 | 1 | rs7770868 |
| 5.8 * 10⁻⁸ | 0.56 | 349 | 0.150 | 15599 | 0.242 | 29.4 | | 3 | rs4711546 |
| 5.8 * 10⁻⁸ | 1.80 | 349 | 0.850 | 15599 | 0.758 | 29.4 | 0.61 | 1 | rs4711546 |
| 1.6 * 10⁻⁷ | 0.64 | 349 | 0.367 | 15519 | 0.474 | 27.5 | | 2 | rs6904723 |
| 1.6 * 10⁻⁷ | 1.56 | 349 | 0.633 | 15519 | 0.526 | 27.5 | 0.40 | 1 | rs6904723 |
| 3.1 * 10⁻⁵ | 0.62 | 349 | 0.822 | 15598 | 0.882 | 17.3 | | 3 | rs12208647 |
| 3.1 * 10⁻⁵ | 1.61 | 349 | 0.178 | 15598 | 0.118 | 17.3 | 0.13 | 1 | rs12208647 |

TABLE 2
The top marker from the PLMS scan, rs3923809, also shows genome-wide
significant association with RLS. (290 affecteds with >98% yield,
15600 controls with >98% yield, allele 1 =
A, allele 2 = C, allele 3 = G, allele 4 = T)

| corr. p- | | | | | | | | | |
|---|---|---|---|---|---|---|---|---|---|
| val | r | #aff | afffrq | #con | con. freq | corr. X2 | PAR | allele | SNP |
| 1.3 * 10⁻⁸ | 0.56 | 290 | 0.228 | 15575 | 0.345 | 32.3 | | 3 | rs3923809 |
| 1.3 * 10⁻⁸ | 1.79 | 290 | 0.772 | 15575 | 0.654 | 32.3 | 0.57 | 1 | rs3923809 |
| 1.9 * 10⁻⁶ | 0.62 | 290 | 0.248 | 15596 | 0.348 | 22.7 | | 2 | rs6923737 |
| 1.9 * 10⁻⁶ | 1.62 | 290 | 0.752 | 15596 | 0.652 | 22.7 | 0.49 | 4 | rs6923737 |
| 5.5 * 10⁻⁶ | 0.65 | 279 | 0.321 | 15351 | 0.423 | 20.7 | | 2 | rs7770868 |
| 5.5 * 10⁻⁶ | 1.55 | 279 | 0.679 | 15351 | 0.577 | 20.7 | 0.42 | 1 | rs7770868 |
| 1.8 * 10⁻⁵ | 0.61 | 290 | 0.159 | 15598 | 0.237 | 18.4 | | 2 | rs9357271 |
| 1.8 * 10⁻⁵ | 1.65 | 290 | 0.841 | 15598 | 0.762 | 18.4 | 0.55 | 4 | rs9357271 |
| 3.9 * 10⁻⁵ | 0.56 | 290 | 0.166 | 15599 | 0.242 | 16.9 | | 3 | rs4711546 |
| 3.9 * 10⁻⁵ | 1.61 | 290 | 0.835 | 15599 | 0.758 | 16.9 | 0.53 | 1 | rs4711546 |
| 1.1 * 10⁻⁴ | 0.70 | 289 | 0.388 | 15519 | 0.474 | 15.0 | | 2 | rs6904723 |
| 1.1 * 10⁻⁴ | 1.43 | 289 | 0.612 | 15519 | 0.526 | 15.0 | 0.33 | 1 | rs6904723 |
| 4.2 * 10⁻³ | 0.69 | 290 | 0.837 | 15598 | 0.882 | 8.2 | | 3 | rs12208647 |
| 4.2 * 10⁻³ | 1.44 | 290 | 0.162 | 15598 | 0.118 | 8.2 | 0.10 | 1 | rs12208647 |

We genotyped an additional 4 markers in the C06 LD block region, selected from the Hapmap dataset to be correlated with marker rs3923809. Table 3 shows association to PLMS for these markers. All four markers show genome-wide significant association to PLMS, with RR values comparable to those originally measured for rs3923809.

TABLE 3
Results of association to PLMS for markers correlated with marker
rs3923809 (allele 1 = A, allele 2 = C, allele 3 = G, allele 4 = T).

| corr. p- | | | | | | | | |
|---|---|---|---|---|---|---|---|---|
| val | R | #aff | afffrq | #con | con. freq | corr. X2 | allele | SNP |
| 3.4 * 10⁻¹⁴ | 0.54 | 526 | 0.202 | 1702 | 0.321 | 57.5 | 4 | rs4236060 |
| 3.4 * 10⁻¹⁴ | 1.84 | 526 | 0.798 | 1702 | 0.679 | 57.5 | 2 | rs4236060 |
| 1.8 * 10⁻¹⁵ | 0.49 | 526 | 0.212 | 829 | 0.353 | 63.3 | 4 | rs10947739 |
| 1.8 * 10⁻¹⁵ | 2.03 | 526 | 0.788 | 829 | 0.647 | 63.3 | 2 | rs10947739 |
| 1.3 * 10⁻¹⁵ | 0.49 | 530 | 0.212 | 832 | 0.354 | 63.9 | 3 | rs6920488 |
| 1.3 * 10⁻¹⁵ | 2.03 | 530 | 0.788 | 832 | 0.646 | 63.9 | 1 | rs6920488 |
| 3.8 * 10⁻¹⁵ | 0.49 | 529 | 0.209 | 822 | 0.348 | 61.8 | 4 | rs13219518 |
| 3.8 * 10⁻¹⁵ | 2.02 | 529 | 0.791 | 822 | 0.652 | 61.8 | 1 | rs13219518 |

It is well documented that prevalence of PLMS increases with age (Bixler, E. O. et al. Res Commun Chem Pathol Pharmacol 36, 129-40 (1982)). Estimates show that PLMS are found in 4-5% of healthy adults (Bixler, E. O. et al. Res Commun Chem Pathol Pharmacol 36, 129-40 (1982); Ohayon, M. M. & Roth, T. J Psychosom Res 53, 547-54 (2002) while it may be as high as 45% in those that are over 65 years of age (Ancoli-Israel, S. et al. Sleep 14, 496-500 (1991)). We have looked at the rr for the PLMS and RLS with respect to age at PLMs assessment and age at RLS onset. We do not see significant decline in rr with age at PLMS assessment (data not shown). However, in the RLS group the rr for marker rs3923809 is lower in those reporting RLS at young age (under 20) and old age (over 50).

The lower risk in the early onset group may possibly be explained by RLS phenocopies such as growth-pain while in the late onset group there may exist RLS phenocopies explained by older age and worse health condition or medication. Our results show unequivocally that PLM and RLS share a genetic risk factor. PLMS are though showing considerably tighter association to rs3923809 and other markers in the LD block compared to RLS. Thus, PLMS are probably less influenced by phenocopies than RLS.

HapMap data reveals that the frequencies of the A allele of rs3923809 is lower in Asian CHB and JPT populations compared to the Caucasian CEU population and the African YRI population, 0.35 and 0.36 vs. 0.74 and 0.66 respectively. This is particularly interesting in the light of the fact that the prevalence of RLS is lower is Asian populations.

Three BTB (POZ) Domain Containing 9 Gene (BTBD9), the Testis Expressed Sequence 27 (TEX27) and Glyoxalase I (GLO1) are in LD with the Markers Significantly Associating with PLMS and RLS

The BTBD9 gene codes for a protein with molecular weight of 61544 Da. The gene is widely expressed both in brain, including; Amygdala, cerebellum, hippocampus, Caudate nucleus and other tissue such as heart, kidney, pancreas and liver. The BTBD9 protein is not well characterized and its function has not been investigated. It contains a BTB domain also known as POZ (poxvirus and zinc finger) domain. This domain is known to be a protein-protein interaction motif found at the N-termini of several C2H2-type transcription factors as well as Shaw-type potassium channels (KCNC1-4).

The BTBD9 protein may function as a transcription factor. However, the gene contains a signal sequence indicating insertion of the protein into the plasma membrane making it less likely to have a function as a transcription factor. Possibly the BTBD9 protein interacts or operates in the same pathway as the Shaw-type potassium channels. Mouse knockout for a member of that family, Kv3.1, display a ‘strong’ mutant phenotype that includes motor dysfunction including; ataxia, myoclonus and tremor. Furthermore deficient mice are constitutively hyperactive and compared to wild type mice, double mutants display ‘restlessness’ that is particularly prominent during the light period, when mice are normally at rest, characterized by more than a doubling of ambulatory and stereotypic activity, and accompanied by a 40% sleep reduction (Espinosa, F., et al. Genes Brain Behav 3, 90-100 (2004)).

Although the SNPs showing strongest association are found in an intron of the BTBD9 gene the LD block spans two other genes, namely the GLO1 and TEX27 genes. The GLO1 gene is a glutathione-binding protein involved in the detoxification of methylglyoxal, a byproduct of glycolysis. In rodents, the electroencephalogram (EEG) during paradoxical sleep and exploratory behavior is characterized by theta oscillations. Deficiency in short-chain acyl-coA dehydrogenase (Acads) in mice cause a marked slowing in the theta frequency during paradoxical sleep only. Expression of the Acads gene in brain regions involved in theta generation, notably the hippocampus. Microarray analysis of gene expression in mice with mutations in Acads indicated overexpression of Glo1. Administration of acetyl-L-carnitine (ALCAR) to mutant mice significantly recovered slow theta and Glo1 over expression (Tafti, M. et al. Nat Genet 34, 320-5 (2003)). Thus, an unappreciated metabolic pathway involving fatty acid beta-oxidation also regulates theta oscillations during sleep.

Isolated rat cerebral cortex cells were able to accumulate L-carnitine and this process was competitively inhibited by 1 mM γ-aminobutyric acid¹⁴. Interestingly, gabapentin (1-aminomethyl cyclohexaneacetic acid), a drug structurally related to γ-aminobutiric acid (GABA), is used to treat RLS patients.

The third gene within the LD block showing association to PLMS/RLS is the TEX27 gene. It has not been well characterized but may be a potential transcription factor. It is preferentially expressed in postmeiotic cells during spermatogenesis.

A fourth gene which is mostly residing in an adjacent LD block is the DNAH8 gene, encoding an axonemal dynein heavy chain. As promoter sequences and possible transcriptional regulatory sequences are located within the LD block of the invention, it is contemplated that the DNAH8 gene is possibly under transcriptional control by variants associated with the C06 LD block.

Association of Marker with Expression of the GLO1 Gene

FIG. 3 shows results of association of marker rs3923809 with expression of GLO1. The expression was measured by a probe-based assay utilizing an nucleic-acid based expression array. The array contains probes for 23,720 transcripts, and can thus be utilized in combination with case-control association studies to investigate whether individual genes are under expression control by individual SNPs. Mean expression of GLO1 in adipose tissue from 557 individuals for the three different genotypes of rs3923809 was determined. Expression changes between two samples were quantified as mean logarithm (log10) expression ratios (MLR) and the values have been adjusted for age and sex of the individuals. The number of individuals behind each estimate in indicated at the bottom of each column and the standard error of the mean is indicated with vertical bars at the top of each column. Also include is a P-value obtained by regressing the sex and age adjusted expression on the number of copies of allele A an individual carries.

The results show that expression of GLO1 is decreased in carriers of the at-risk variant A of marker rs3923809. This indicates a direct functional consequence of the variants of the present invention found to correlate with RLS and PLMS.

TABLE 4
Surrogate markers in linkage disequilibrium (LD) with at least one of the markers
giving significant association to RLS and PLMS. The markers were selected using HapMap CEU
data. Markers with r2 > 0.2 with at least one of rs9357271, rs4711546, rs12208647,
rs6904723, rs3923809, rs7770868 and rs6923737 were selected from a search of all markers in
a 2Mb interval (1Mb upstream and 1Mb downstream) around marker rs3923809. The columns
(left to right) indicate surrogate marker name, position in NCBI Build 36, values of D′ to
rs3923809, value of highest r² to any of the six additional markers (i.e., excluding rs3923809),
the name of the marker to which the surrogate has the highest value of r² to, and the SEQ ID for
the flanking sequence of the surrogate marker.

| | | | | | | SEQ ID |
|---|---|---|---|---|---|---|
| Marker | Position | D′ w/rs3923809 | R² w/rs3923809 | Max (R²) | Marker w/max (R²) | NO: |
| rs1041037 | 37870532 | 0.725 | 0.029 | 0.528 | rs12208647 | 12 |
| rs7762052 | 37875507 | 0.671 | 0.043 | 0.309 | rs12208647 | 13 |
| rs6936260 | 37882300 | 0.655 | 0.042 | 0.325 | rs12208647 | 14 |
| rs11755559 | 37883026 | 0.725 | 0.029 | 0.530 | rs12208647 | 15 |
| rs6910715 | 37884940 | 0.819 | 0.063 | 0.345 | rs12208647 | 16 |
| rs6908106 | 37893468 | 0.819 | 0.063 | 0.345 | rs12208647 | 17 |
| rs11759458 | 37894737 | 0.819 | 0.063 | 0.345 | rs12208647 | 18 |
| rs11752692 | 37898187 | 0.725 | 0.029 | 0.530 | rs12208647 | 19 |
| rs10485030 | 37899874 | 0.819 | 0.063 | 0.345 | rs12208647 | 20 |
| rs2274091 | 37901719 | 0.819 | 0.063 | 0.345 | rs12208647 | 21 |
| rs16890185 | 37903257 | 0.819 | 0.063 | 0.345 | rs12208647 | 22 |
| rs12661378 | 37903924 | 0.825 | 0.068 | 0.336 | rs12208647 | 23 |
| rs7761561 | 37905283 | 0.818 | 0.064 | 0.342 | rs12208647 | 24 |
| rs11759721 | 37907212 | 0.725 | 0.029 | 0.530 | rs12208647 | 25 |
| rs10947702 | 37910654 | 0.819 | 0.063 | 0.345 | rs12208647 | 26 |
| rs7764130 | 37912241 | 0.819 | 0.063 | 0.345 | rs12208647 | 27 |
| rs12662426 | 37913088 | 0.805 | 0.059 | 0.331 | rs12208647 | 28 |
| rs11759574 | 37917147 | 0.748 | 0.035 | 0.480 | rs12208647 | 29 |
| rs7742226 | 37924180 | 1.000 | 0.096 | 0.347 | rs12208647 | 30 |
| rs10947704 | 37925087 | 0.819 | 0.063 | 0.345 | rs12208647 | 31 |
| rs9357257 | 37929675 | 0.428 | 0.037 | 0.208 | rs9357271 | 32 |
| rs13205214 | 37932109 | 0.836 | 0.063 | 0.343 | rs12208647 | 33 |
| rs7773419 | 37935353 | 0.819 | 0.063 | 0.345 | rs12208647 | 34 |
| rs12664768 | 37940469 | 1.000 | 0.097 | 0.345 | rs12208647 | 35 |
| rs12524229 | 37942664 | 0.424 | 0.036 | 0.208 | rs9357271 | 36 |
| rs11758035 | 37948091 | 0.725 | 0.029 | 0.530 | rs12208647 | 37 |
| rs11755629 | 37948228 | 0.824 | 0.066 | 0.340 | rs12208647 | 38 |
| rs11753118 | 37965847 | 0.750 | 0.034 | 0.485 | rs12208647 | 39 |
| rs7746266 | 37967834 | 0.819 | 0.063 | 0.345 | rs12208647 | 40 |
| rs6458032 | 37968048 | 0.819 | 0.063 | 0.345 | rs12208647 | 41 |
| rs6911716 | 37973388 | 1.000 | 0.097 | 0.343 | rs12208647 | 42 |
| rs11756713 | 37976113 | 0.819 | 0.063 | 0.345 | rs12208647 | 43 |
| rs12664091 | 37977689 | 0.825 | 0.068 | 0.336 | rs12208647 | 44 |
| rs3844314 | 37983005 | 0.819 | 0.063 | 0.345 | rs12208647 | 45 |
| rs11752521 | 37989202 | 0.819 | 0.063 | 0.345 | rs12208647 | 46 |
| rs11752567 | 37989423 | 0.725 | 0.029 | 0.530 | rs12208647 | 47 |
| rs7748312 | 38002867 | 0.788 | 0.056 | 0.366 | rs12208647 | 48 |
| rs16890270 | 38005011 | 0.797 | 0.059 | 0.357 | rs12208647 | 49 |
| rs2273441 | 38005817 | 0.802 | 0.055 | 0.354 | rs12208647 | 50 |
| rs11759486 | 38006864 | 0.728 | 0.030 | 0.527 | rs12208647 | 51 |
| rs13206035 | 38008100 | 0.810 | 0.059 | 0.366 | rs12208647 | 52 |
| rs7775794 | 38011638 | 0.819 | 0.063 | 0.345 | rs12208647 | 53 |
| rs11757359 | 38014029 | 0.629 | 0.020 | 0.554 | rs12208647 | 54 |
| rs2842510 | 38017062 | 0.824 | 0.062 | 0.334 | rs12208647 | 55 |
| rs11759826 | 38030296 | 0.638 | 0.038 | 0.345 | rs12208647 | 56 |
| rs7759792 | 38032884 | 0.801 | 0.058 | 0.362 | rs12208647 | 57 |
| rs11751781 | 38037719 | 0.638 | 0.038 | 0.345 | rs12208647 | 58 |
| rs11753267 | 38041299 | 0.490 | 0.014 | 0.488 | rs12208647 | 59 |
| rs11752863 | 38044484 | 0.503 | 0.016 | 0.483 | rs12208647 | 60 |
| rs11756030 | 38048938 | 0.490 | 0.014 | 0.489 | rs12208647 | 61 |
| rs2842521 | 38057146 | 0.596 | 0.039 | 0.310 | rs12208647 | 62 |
| rs11758540 | 38058339 | 0.490 | 0.014 | 0.489 | rs12208647 | 63 |
| rs752110 | 38061966 | 0.490 | 0.014 | 0.489 | rs12208647 | 64 |
| rs11754980 | 38065530 | 0.490 | 0.014 | 0.488 | rs12208647 | 65 |
| rs12661480 | 38071286 | 0.503 | 0.016 | 0.483 | rs12208647 | 66 |
| rs11752623 | 38086838 | 0.589 | 0.011 | 0.513 | rs12208647 | 67 |
| rs11750950 | 38087158 | 0.409 | 0.010 | 0.488 | rs12208647 | 68 |
| rs11753054 | 38099778 | 0.409 | 0.010 | 0.488 | rs12208647 | 69 |
| rs10947710 | 38110726 | 0.501 | 0.015 | 0.486 | rs12208647 | 70 |
| rs11757072 | 38126606 | 0.503 | 0.016 | 0.483 | rs12208647 | 71 |
| rs9296234 | 38127698 | 0.428 | 0.037 | 0.208 | rs9357271 | 72 |
| rs12664900 | 38133981 | 0.503 | 0.016 | 0.483 | rs12208647 | 73 |
| rs12183620 | 38135700 | 0.428 | 0.037 | 0.298 | rs9357271 | 74 |
| rs9357261 | 38145274 | 0.414 | 0.035 | 0.207 | rs9357271 | 75 |
| rs11751153 | 38149091 | 0.501 | 0.015 | 0.486 | rs12208647 | 76 |
| rs9380717 | 38150880 | 0.428 | 0.037 | 0.208 | rs9357271 | 77 |
| rs9470765 | 38160900 | 0.503 | 0.016 | 0.483 | rs12208647 | 78 |
| rs11755439 | 38166492 | 0.486 | 0.014 | 0.488 | rs12208647 | 79 |
| rs9470772 | 38171048 | 0.490 | 0.014 | 0.489 | rs12208647 | 80 |
| rs11756626 | 38173823 | 0.490 | 0.014 | 0.489 | rs12208647 | 81 |
| rs9470777 | 38179433 | 0.490 | 0.014 | 0.489 | rs12208647 | 82 |
| rs1885324 | 38180805 | 0.045 | 0.001 | 0.280 | rs12208647 | 83 |
| rs9470779 | 38181800 | 0.490 | 0.014 | 0.489 | rs12208647 | 84 |
| rs11753563 | 38188196 | 0.490 | 0.014 | 0.489 | rs12208647 | 85 |
| rs9470784 | 38191618 | 0.490 | 0.014 | 0.489 | rs12208647 | 86 |
| rs11758095 | 38193427 | 0.496 | 0.015 | 0.486 | rs12208647 | 87 |
| rs11759483 | 38195657 | 0.490 | 0.014 | 0.489 | rs12208647 | 88 |
| rs2145887 | 38205752 | 0.522 | 0.017 | 0.453 | rs12208647 | 89 |
| rs11759816 | 38208619 | 0.696 | 0.025 | 0.509 | rs12208647 | 90 |
| rs11751094 | 38208975 | 0.490 | 0.014 | 0.489 | rs12208647 | 91 |
| rs9357262 | 38214323 | 0.428 | 0.037 | 0.208 | rs9357271 | 92 |
| rs9470794 | 38214822 | 0.490 | 0.014 | 0.489 | rs12208647 | 93 |
| rs11757654 | 38215198 | 0.490 | 0.014 | 0.489 | rs12208647 | 94 |
| rs11755600 | 38215843 | 0.490 | 0.014 | 0.489 | rs12208647 | 95 |
| rs1923280 | 38216871 | 0.487 | 0.014 | 0.483 | rs12208647 | 96 |
| rs9470798 | 38219317 | 0.490 | 0.014 | 0.489 | rs12208647 | 97 |
| rs9349056 | 38222054 | 0.419 | 0.036 | 0.207 | rs9357271 | 98 |
| rs11755566 | 38224647 | 0.490 | 0.014 | 0.489 | rs12208647 | 99 |
| rs11756902 | 38226616 | 0.490 | 0.014 | 0.489 | rs12208647 | 100 |
| rs7748392 | 38226733 | 0.490 | 0.014 | 0.489 | rs12208647 | 101 |
| rs876818 | 38228638 | 0.507 | 0.016 | 0.485 | rs12208647 | 102 |
| rs3800364 | 38229189 | 0.490 | 0.014 | 0.489 | rs12208647 | 103 |
| rs2395692 | 38232057 | 0.490 | 0.014 | 0.489 | rs12208647 | 104 |
| rs2395693 | 38232197 | 0.490 | 0.014 | 0.489 | rs12208647 | 105 |
| rs12182468 | 38233522 | 1.000 | 0.048 | 0.506 | rs12208647 | 106 |
| rs9462394 | 38235180 | 0.490 | 0.014 | 0.489 | rs12208647 | 107 |
| rs9462395 | 38235464 | 0.490 | 0.014 | 0.489 | rs12208647 | 108 |
| rs11755190 | 38236221 | 0.490 | 0.014 | 0.489 | rs12208647 | 109 |
| rs11753067 | 38236289 | 0.496 | 0.015 | 0.486 | rs12208647 | 110 |
| rs11753160 | 38236796 | 0.474 | 0.011 | 0.490 | rs12208647 | 111 |
| rs11751937 | 38238431 | 1.000 | 0.015 | 0.276 | rs12208647 | 112 |
| rs2073021 | 38239420 | 0.712 | 0.028 | 0.527 | rs12208647 | 113 |
| rs11752799 | 38241008 | 0.108 | 0.001 | 0.421 | rs12208647 | 114 |
| rs9462399 | 38249300 | 0.551 | 0.020 | 0.508 | rs12208647 | 115 |
| rs11756301 | 38250294 | 0.724 | 0.034 | 0.541 | rs12208647 | 116 |
| rs9369034 | 38254210 | 0.522 | 0.017 | 0.545 | rs12208647 | 117 |
| rs4336442 | 38254424 | 0.574 | 0.021 | 0.495 | rs12208647 | 118 |
| rs10947715 | 38255888 | 0.576 | 0.024 | 0.565 | rs12208647 | 119 |
| rs9462403 | 38260932 | 0.556 | 0.021 | 0.555 | rs12208647 | 120 |
| rs10947717 | 38267851 | 0.551 | 0.020 | 0.508 | rs12208647 | 121 |
| rs10456461 | 38274417 | 0.419 | 0.021 | 0.384 | rs12208647 | 122 |
| rs10456462 | 38274489 | 0.527 | 0.030 | 0.424 | rs12208647 | 123 |
| rs12176364 | 38300971 | 0.827 | 0.156 | 0.335 | rs9357271 | 124 |
| rs1931760 | 38309423 | 0.638 | 0.034 | 0.645 | rs12208647 | 125 |
| rs1931761 | 38310800 | 0.638 | 0.034 | 0.645 | rs12208647 | 126 |
| rs12202202 | 38312281 | 0.635 | 0.034 | 0.643 | rs12208647 | 127 |
| rs7356829 | 38313830 | 0.827 | 0.156 | 0.335 | rs9357271 | 128 |
| rs9462415 | 38314213 | 0.827 | 0.156 | 0.335 | rs9357271 | 129 |
| rs9296239 | 38315614 | 0.827 | 0.156 | 0.335 | rs9357271 | 130 |
| rs9470834 | 38315642 | 0.829 | 0.161 | 0.335 | rs9357271 | 131 |
| rs9470835 | 38315651 | 0.825 | 0.156 | 0.335 | rs9357271 | 132 |
| rs9366949 | 38316836 | 0.827 | 0.156 | 0.335 | rs9357271 | 133 |
| rs9380726 | 38316961 | 0.827 | 0.156 | 0.335 | rs9357271 | 134 |
| rs873316 | 38317068 | 0.162 | 0.006 | 0.217 | rs12208647 | 135 |
| rs7745176 | 38317441 | 0.827 | 0.156 | 0.335 | rs9357271 | 136 |
| rs9357265 | 38318032 | 0.827 | 0.156 | 0.335 | rs9357271 | 137 |
| rs9462416 | 38318287 | 0.101 | 0.002 | 0.216 | rs12208647 | 138 |
| rs9357266 | 38318432 | 0.827 | 0.156 | 0.335 | rs9357271 | 139 |
| rs9296240 | 38318497 | 0.827 | 0.156 | 0.335 | rs9357271 | 140 |
| rs9296241 | 38318553 | 0.827 | 0.156 | 0.335 | rs9357271 | 141 |
| rs9296242 | 38318690 | 0.827 | 0.156 | 0.335 | rs9357271 | 142 |
| rs9394481 | 38318745 | 0.827 | 0.156 | 0.335 | rs9357271 | 143 |
| rs9357267 | 38318984 | 0.827 | 0.156 | 0.335 | rs9357271 | 144 |
| rs9394482 | 38319053 | 0.827 | 0.156 | 0.335 | rs9357271 | 145 |
| rs7755820 | 38321292 | 0.825 | 0.156 | 0.335 | rs9357271 | 146 |
| rs7756174 | 38321497 | 0.824 | 0.167 | 0.351 | rs9357271 | 147 |
| rs17757975 | 38322128 | 0.638 | 0.034 | 0.645 | rs12208647 | 148 |
| rs9349061 | 38322423 | 0.804 | 0.133 | 0.309 | rs9357271 | 149 |
| rs9349062 | 38322441 | 0.804 | 0.134 | 0.309 | rs9357271 | 150 |
| rs9349063 | 38322495 | 0.827 | 0.156 | 0.335 | rs9357271 | 151 |
| rs9366950 | 38324672 | 0.618 | 0.120 | 0.289 | rs9357271 | 152 |
| rs726160 | 38329617 | 0.013 | 0.000 | 0.208 | rs12208647 | 153 |
| rs4714136 | 38336104 | 0.552 | 0.223 | 0.259 | rs7770868 | 154 |
| rs13194038 | 38338207 | 1.000 | 0.221 | 0.221 | rs3923809 | 155 |
| rs9470847 | 38346529 | 0.555 | 0.229 | 0.264 | rs7770868 | 156 |
| rs9470848 | 38348063 | 0.555 | 0.229 | 0.264 | rs7770868 | 157 |
| rs1475761 | 38348396 | 0.555 | 0.229 | 0.264 | rs7770868 | 158 |
| rs12201418 | 38354349 | 0.646 | 0.036 | 0.641 | rs12208647 | 159 |
| rs9462426 | 38354751 | 0.577 | 0.062 | 0.284 | rs12208647 | 160 |
| rs7739762 | 38355698 | 0.638 | 0.034 | 0.645 | rs12208647 | 161 |
| rs1931767 | 38355823 | 0.555 | 0.229 | 0.264 | rs7770868 | 162 |
| rs9470850 | 38356173 | 0.577 | 0.062 | 0.284 | rs12208647 | 163 |
| rs9470851 | 38357606 | 0.555 | 0.229 | 0.264 | rs7770868 | 164 |
| rs12191714 | 38361778 | 0.649 | 0.166 | 0.322 | rs6904723 | 165 |
| rs4714140 | 38362904 | 0.505 | 0.191 | 0.257 | rs7770868 | 166 |
| rs12206324 | 38365813 | 0.649 | 0.166 | 0.322 | rs6904723 | 167 |
| rs12215850 | 38372831 | 0.649 | 0.166 | 0.322 | rs6904723 | 168 |
| rs4714144 | 38381069 | 0.503 | 0.198 | 0.218 | rs7770868 | 169 |
| rs6905637 | 38385888 | 0.526 | 0.209 | 0.246 | rs7770868 | 170 |
| rs871183 | 38396146 | 0.598 | 0.160 | 0.261 | rs6904723 | 171 |
| rs228184 | 38400595 | 0.541 | 0.211 | 0.279 | rs4711546 | 172 |
| rs228182 | 38407846 | 0.497 | 0.184 | 0.219 | rs7770868 | 173 |
| rs228181 | 38408118 | 0.506 | 0.198 | 0.240 | rs7770868 | 174 |
| rs228179 | 38412860 | 0.643 | 0.163 | 0.320 | rs6904723 | 175 |
| rs228185 | 38423194 | 0.551 | 0.226 | 0.261 | rs7770868 | 176 |
| rs228186 | 38425664 | 0.634 | 0.153 | 0.304 | rs6904723 | 177 |
| rs228187 | 38425781 | 0.551 | 0.226 | 0.261 | rs7770868 | 178 |
| rs228188 | 38426351 | 0.643 | 0.163 | 0.320 | rs6904723 | 179 |
| rs7763775 | 38426652 | 0.643 | 0.163 | 0.320 | rs6904723 | 180 |
| rs2179532 | 38426685 | 0.586 | 0.145 | 0.274 | rs6904723 | 181 |
| rs2179533 | 38426699 | 0.584 | 0.141 | 0.266 | rs6904723 | 182 |
| rs1033480 | 38428921 | 0.636 | 0.139 | 0.336 | rs6904723 | 183 |
| rs6922620 | 38428988 | 0.539 | 0.220 | 0.271 | rs7770868 | 184 |
| rs9380737 | 38429760 | 0.750 | 0.369 | 0.477 | rs7770868 | 185 |
| rs4714148 | 38431277 | 0.737 | 0.160 | 0.203 | rs4711546 | 186 |
| rs6940319 | 38437807 | 0.772 | 0.189 | 0.219 | rs4711546 | 187 |
| rs9349073 | 38440346 | 0.772 | 0.189 | 0.219 | rs4711546 | 188 |
| rs9380739 | 38440533 | 0.415 | 0.118 | 0.482 | rs4711546 | 189 |
| rs9394492 | 38440588 | 0.415 | 0.118 | 0.482 | rs4711546 | 190 |
| rs9380740 | 38441452 | 0.763 | 0.415 | 1.000 | rs9357271 | 191 |
| rs1883610 | 38448077 | 0.409 | 0.108 | 0.458 | rs4711546 | 192 |
| rs4711542 | 38450140 | 0.772 | 0.189 | 0.219 | rs4711546 | 193 |
| rs4714152 | 38451319 | 0.765 | 0.416 | 1.000 | rs9357271 | 194 |
| rs12212820 | 38453820 | 0.809 | 0.053 | 0.683 | rs12208647 | 195 |
| rs742515 | 38455669 | 0.765 | 0.416 | 1.000 | rs9357271 | 196 |
| rs4140443 | 38455930 | 0.766 | 0.434 | 1.000 | rs9357271 | 197 |
| rs9394493 | 38458782 | 0.719 | 0.362 | 1.000 | rs9357271 | 198 |
| rs7759078 | 38459269 | 0.763 | 0.415 | 1.000 | rs9357271 | 199 |
| rs7759629 | 38459478 | 0.765 | 0.416 | 1.000 | rs9357271 | 200 |
| rs9394494 | 38459959 | 0.746 | 0.388 | 1.000 | rs9357271 | 201 |
| rs9380741 | 38460057 | 0.761 | 0.403 | 1.000 | rs9357271 | 202 |
| rs4711544 | 38460261 | 0.765 | 0.416 | 1.000 | rs9357271 | 203 |
| rs1983605 | 38463918 | 0.765 | 0.416 | 1.000 | rs9357271 | 204 |
| rs7749685 | 38465868 | 0.765 | 0.416 | 1.000 | rs9357271 | 205 |
| rs9380742 | 38465910 | 0.765 | 0.416 | 1.000 | rs9357271 | 206 |
| rs9380743 | 38466780 | 0.765 | 0.416 | 1.000 | rs9357271 | 207 |
| rs9296248 | 38466812 | 0.765 | 0.416 | 1.000 | rs9357271 | 208 |
| rs9470867 | 38466838 | 0.764 | 0.414 | 1.000 | rs9357271 | 209 |
| rs9470868 | 38467055 | 0.765 | 0.416 | 1.000 | rs9357271 | 210 |
| rs7756267 | 38467577 | 0.765 | 0.416 | 1.000 | rs9357271 | 211 |
| rs4714155 | 38468878 | 0.741 | 0.391 | 1.000 | rs9357271 | 212 |
| rs4714156 | 38469090 | 0.703 | 0.363 | 0.848 | rs4711546 | 213 |
| rs7766214 | 38471146 | 0.765 | 0.416 | 1.000 | rs9357271 | 214 |
| rs7748599 | 38471191 | 0.763 | 0.415 | 1.000 | rs9357271 | 215 |
| rs9349077 | 38473574 | 0.816 | 0.449 | 0.948 | rs4711546 | 216 |
| rs9296249 | 38473819 | 0.765 | 0.416 | 1.000 | rs9357271 | 217 |
| rs9357271 | 38473851 | 0.769 | 0.430 | 1.000 | rs9357271 | 2 |
| rs7764502 | 38474115 | 0.763 | 0.415 | 1.000 | rs9357271 | 218 |
| rs4711546 | 38474164 | 0.765 | 0.416 | 1.000 | rs4711546 | 5 |
| rs4714157 | 38474363 | 0.765 | 0.416 | 1.000 | rs9357271 | 219 |
| rs9357272 | 38478330 | 0.765 | 0.416 | 1.000 | rs9357271 | 220 |
| rs9394495 | 38479170 | 0.766 | 0.179 | 0.212 | rs4711546 | 221 |
| rs9380745 | 38481206 | 0.806 | 0.427 | 0.946 | rs4711546 | 222 |
| rs6903611 | 38482687 | 0.765 | 0.416 | 1.000 | rs9357271 | 223 |
| rs9369057 | 38482744 | 0.815 | 0.448 | 0.948 | rs4711546 | 224 |
| rs6903767 | 38482790 | 0.765 | 0.416 | 1.000 | rs9357271 | 225 |
| rs4714158 | 38483312 | 0.765 | 0.416 | 1.000 | rs9357271 | 226 |
| rs9380747 | 38483947 | 0.765 | 0.416 | 1.000 | rs9357271 | 227 |
| rs2814896 | 38488782 | 0.926 | 0.271 | 0.406 | rs7770868 | 228 |
| rs2745379 | 38489086 | 0.852 | 0.242 | 0.373 | rs7770868 | 229 |
| rs2745380 | 38489311 | 0.924 | 0.259 | 0.392 | rs7770868 | 230 |
| rs2814888 | 38489334 | 0.931 | 0.293 | 0.438 | rs7770868 | 231 |
| rs2745381 | 38490914 | 1.000 | 0.296 | 0.423 | rs7770868 | 232 |
| rs2745382 | 38492570 | 0.926 | 0.271 | 0.406 | rs7770868 | 233 |
| rs12194728 | 38493823 | 0.926 | 0.269 | 0.407 | rs7770868 | 234 |
| rs2745384 | 38497676 | 1.000 | 0.307 | 0.422 | rs7770868 | 235 |
| rs2745385 | 38498207 | 1.000 | 0.305 | 0.436 | rs7770868 | 236 |
| rs9380748 | 38499784 | 0.926 | 0.271 | 0.406 | rs7770868 | 237 |
| rs4591850 | 38506781 | 0.926 | 0.271 | 0.406 | rs7770868 | 238 |
| rs4131034 | 38507938 | 0.925 | 0.268 | 0.402 | rs7770868 | 239 |
| rs4591851 | 38508970 | 0.926 | 0.271 | 0.406 | rs7770868 | 240 |
| rs9470878 | 38514714 | 0.486 | 0.094 | 0.222 | rs6904723 | 241 |
| rs12208647 | 38516201 | 1.000 | 0.063 | 1.000 | rs12208647 | 7 |
| rs6899746 | 38520766 | 0.946 | 0.510 | 0.965 | rs6904723 | 242 |
| rs9470879 | 38523833 | 0.486 | 0.094 | 0.222 | rs6904723 | 243 |
| rs10947737 | 38527436 | 1.000 | 0.113 | 0.207 | rs6904723 | 244 |
| rs12662621 | 38528162 | 0.553 | 0.112 | 0.205 | rs6904723 | 245 |
| rs11751154 | 38531321 | 1.000 | 0.029 | 0.459 | rs12208647 | 246 |
| rs13199426 | 38536693 | 0.482 | 0.092 | 0.222 | rs6904723 | 247 |
| rs6900510 | 38538076 | 0.482 | 0.092 | 0.222 | rs6904723 | 248 |
| rs6901086 | 38538387 | 0.947 | 0.517 | 0.965 | rs6904723 | 249 |
| rs6904723 | 38544295 | 1.000 | 0.557 | 1.000 | rs6904723 | 6 |
| rs3923809 | 38548948 | 1.000 | 1.000 | 1.000 | rs3923809 | 1 |
| rs4236058 | 38550426 | 1.000 | 0.217 | 0.386 | rs6904723 | 250 |
| rs6920488 | 38552018 | 1.000 | 1.000 | 1.000 | rs3923809 | 10 |
| rs9470884 | 38552741 | 0.581 | 0.137 | 0.227 | rs6904723 | 251 |
| rs9470885 | 38553377 | 1.000 | 0.557 | 1.000 | rs6904723 | 252 |
| rs4714165 | 38553435 | 1.000 | 0.557 | 1.000 | rs6904723 | 253 |
| rs6933268 | 38554328 | 1.000 | 0.644 | 1.000 | rs7770868 | 254 |
| rs6933758 | 38554349 | 1.000 | 0.600 | 1.000 | rs7770868 | 255 |
| rs12200371 | 38554612 | 0.557 | 0.113 | 0.232 | rs7770868 | 256 |
| rs13219518 | 38555848 | 1.000 | 0.957 | 0.957 | rs3923809 | 11 |
| rs9470887 | 38558410 | 0.557 | 0.113 | 0.232 | rs7770868 | 257 |
| rs9470888 | 38561015 | 0.948 | 0.601 | 0.962 | rs7770868 | 258 |
| rs9462435 | 38570041 | 0.557 | 0.113 | 0.232 | rs7770868 | 259 |
| rs17650092 | 38571627 | 1.000 | 0.134 | 0.214 | rs7770868 | 260 |
| rs7770868 | 38572604 | 1.000 | 0.638 | 1.000 | rs7770868 | 4 |
| rs9470891 | 38575487 | 1.000 | 0.226 | 0.364 | rs6904723 | 261 |
| rs12206905 | 38575642 | 1.000 | 0.217 | 0.342 | rs6904723 | 262 |
| rs10947739 | 38577301 | 0.867 | 0.746 | 0.746 | rs3923809 | 9 |
| rs4236060 | 38578065 | 0.955 | 0.872 | 0.872 | rs3923809 | 8 |
| rs17590501 | 38578215 | 1.000 | 0.016 | 0.246 | rs12208647 | 263 |
| rs10947740 | 38584991 | 0.553 | 0.246 | 0.433 | rs6923737 | 264 |
| rs7740763 | 38586245 | 1.000 | 0.202 | 0.264 | rs6923737 | 265 |
| rs12199427 | 38587038 | 0.644 | 0.067 | 0.210 | rs6923737 | 266 |
| rs4074421 | 38589931 | 0.672 | 0.079 | 0.223 | rs6923737 | 267 |
| rs6923737 | 38591542 | 0.858 | 0.572 | 1.000 | rs6923737 | 3 |
| rs9394508 | 38594615 | 0.550 | 0.108 | 0.278 | rs6923737 | 268 |
| rs6924443 | 38594856 | 0.590 | 0.075 | 0.227 | rs6923737 | 269 |
| rs10807196 | 38602589 | 0.656 | 0.260 | 0.538 | rs6923737 | 270 |
| rs1073900 | 38609285 | 0.577 | 0.063 | 0.208 | rs6923737 | 271 |
| rs12193868 | 38619093 | 0.665 | 0.081 | 0.232 | rs6923737 | 272 |
| rs2748156 | 38621152 | 0.607 | 0.079 | 0.239 | rs6923737 | 273 |
| rs2092797 | 38622362 | 0.510 | 0.077 | 0.219 | rs6923737 | 274 |
| rs6931131 | 38623973 | 0.507 | 0.077 | 0.223 | rs6923737 | 275 |
| rs6932235 | 38624556 | 0.510 | 0.077 | 0.219 | rs6923737 | 276 |
| rs2814887 | 38626857 | 0.592 | 0.077 | 0.239 | rs6923737 | 277 |
| rs2814889 | 38627739 | 0.671 | 0.269 | 0.530 | rs6923737 | 278 |
| rs2748173 | 38638120 | 0.607 | 0.079 | 0.239 | rs6923737 | 279 |
| rs2748172 | 38638278 | 0.657 | 0.256 | 0.514 | rs6923737 | 280 |
| rs2814890 | 38638808 | 0.605 | 0.080 | 0.241 | rs6923737 | 281 |
| rs17620389 | 38642407 | 0.568 | 0.012 | 0.456 | rs12208647 | 282 |
| rs1321056 | 38643554 | 0.613 | 0.224 | 0.476 | rs6923737 | 283 |
| rs2252550 | 38649462 | 0.606 | 0.211 | 0.456 | rs6923737 | 284 |
| rs2814893 | 38651824 | 0.652 | 0.104 | 0.278 | rs6923737 | 285 |
| rs2814894 | 38652475 | 0.613 | 0.224 | 0.476 | rs6923737 | 286 |
| rs2748168 | 38653920 | 0.652 | 0.104 | 0.278 | rs6923737 | 287 |
| rs2748166 | 38660252 | 0.652 | 0.104 | 0.278 | rs6923737 | 288 |
| rs10807198 | 38662250 | 0.613 | 0.224 | 0.476 | rs6923737 | 289 |
| rs7769186 | 38663490 | 0.652 | 0.104 | 0.278 | rs6923737 | 290 |
| rs6458059 | 38665577 | 0.600 | 0.222 | 0.461 | rs6923737 | 291 |
| rs6458060 | 38665677 | 0.588 | 0.067 | 0.211 | rs6923737 | 292 |
| rs1739632 | 38669200 | 0.613 | 0.224 | 0.476 | rs6923737 | 293 |
| rs910516 | 38672926 | 0.613 | 0.224 | 0.476 | rs6923737 | 294 |
| rs2144328 | 38673369 | 0.591 | 0.187 | 0.417 | rs6923737 | 295 |
| rs1699018 | 38675991 | 0.588 | 0.067 | 0.211 | rs6923737 | 296 |
| rs1699020 | 38678691 | 0.652 | 0.104 | 0.278 | rs6923737 | 297 |
| rs1699015 | 38680568 | 0.588 | 0.067 | 0.211 | rs6923737 | 298 |
| rs1699007 | 38691346 | 0.612 | 0.224 | 0.464 | rs6923737 | 299 |
| rs1699002 | 38703379 | 0.613 | 0.224 | 0.476 | rs6923737 | 300 |
| rs1739626 | 38707544 | 0.613 | 0.224 | 0.476 | rs6923737 | 301 |
| rs1781708 | 38712732 | 0.613 | 0.224 | 0.476 | rs6923737 | 302 |
| rs1781742 | 38713353 | 0.601 | 0.072 | 0.220 | rs6923737 | 303 |
| rs1699013 | 38718697 | 0.652 | 0.104 | 0.278 | rs6923737 | 304 |
| rs1781741 | 38720498 | 0.652 | 0.104 | 0.278 | rs6923737 | 305 |
| rs1781740 | 38722176 | 0.578 | 0.065 | 0.210 | rs6923737 | 306 |
| rs1781738 | 38735257 | 0.503 | 0.182 | 0.430 | rs6923737 | 307 |
| rs12198690 | 38736696 | 0.606 | 0.112 | 0.304 | rs6923737 | 308 |
| rs6458063 | 38740555 | 0.465 | 0.100 | 0.298 | rs6923737 | 309 |
| rs7747615 | 38740681 | 0.596 | 0.105 | 0.252 | rs6923737 | 310 |
| rs17544777 | 38742721 | 0.596 | 0.105 | 0.290 | rs6923737 | 311 |
| rs4546477 | 38743705 | 0.414 | 0.079 | 0.297 | rs6923737 | 312 |
| rs7739414 | 38744177 | 0.490 | 0.105 | 0.293 | rs6923737 | 313 |
| rs9380765 | 38745479 | 0.463 | 0.088 | 0.272 | rs6923737 | 314 |
| rs6458064 | 38746487 | 0.644 | 0.119 | 0.319 | rs6923737 | 315 |
| rs9462448 | 38748222 | 0.442 | 0.083 | 0.264 | rs6923737 | 316 |
| rs6458065 | 38749332 | 0.399 | 0.073 | 0.251 | rs6923737 | 317 |
| rs17622097 | 38750349 | 0.567 | 0.096 | 0.288 | rs6923737 | 318 |
| rs937662 | 38754347 | 0.555 | 0.084 | 0.268 | rs6923737 | 319 |
| rs3799703 | 38754608 | 0.442 | 0.083 | 0.264 | rs6923737 | 320 |
| rs12214815 | 38755830 | 0.596 | 0.105 | 0.292 | rs6923737 | 321 |
| rs2736655 | 38756772 | 0.499 | 0.122 | 0.381 | rs6923737 | 322 |
| rs2736654 | 38758606 | 0.625 | 0.127 | 0.328 | rs6923737 | 323 |
| rs4714175 | 38758819 | 0.472 | 0.105 | 0.306 | rs6923737 | 324 |
| rs10947755 | 38759800 | 0.472 | 0.105 | 0.306 | rs6923737 | 325 |
| rs2471999 | 38760434 | 0.472 | 0.105 | 0.306 | rs6923737 | 326 |
| rs10447398 | 38764307 | 0.613 | 0.115 | 0.340 | rs6923737 | 327 |
| rs1699012 | 38788921 | 0.503 | 0.182 | 0.430 | rs6923737 | 328 |

TABLE 5
Markers in linkage disequilibrium (LD) with rs3923809. The markers were selected
from a search of all markers in a 2Mb interval (1Mb upstream and 1Mb downstream) around
marker rs3923809. Shown are marker with r² > 0.1 to rs3923809 in the HapMap CEU
(Caucasian) sample set, ordered by value of r² values. The columns (left to right) indicate
surrogate marker name, position in NCBI Build 36, values of D′ to rs3923809, value of highest r²
to any of the six additional markers used to select the surrogates indicated in Table 4, the name
of the marker to which the surrogate has the highest value of r² to, and the SEQ ID for the
surrogate marker.

| | | | | | Marker | SEQ ID |
|---|---|---|---|---|---|---|
| Marker | Position | D′ w/rs3923809 | R2 w/rs3923809 | Max (R2) | w/max (R2) | NO |
| rs6458063 | 38740555 | 0.465 | 0.1 | 0.298 | rs6923737 | 309 |
| rs2814893 | 38651824 | 0.652 | 0.104 | 0.278 | rs6923737 | 285 |
| rs2748168 | 38653920 | 0.652 | 0.104 | 0.278 | rs6923737 | 287 |
| rs2748166 | 38660252 | 0.652 | 0.104 | 0.278 | rs6923737 | 288 |
| rs7769186 | 38663490 | 0.652 | 0.104 | 0.278 | rs6923737 | 290 |
| rs1699020 | 38678691 | 0.652 | 0.104 | 0.278 | rs6923737 | 297 |
| rs1699013 | 38718697 | 0.652 | 0.104 | 0.278 | rs6923737 | 304 |
| rs1781741 | 38720498 | 0.652 | 0.104 | 0.278 | rs6923737 | 305 |
| rs7747615 | 38740681 | 0.596 | 0.105 | 0.252 | rs6923737 | 310 |
| rs17544777 | 38742721 | 0.596 | 0.105 | 0.29 | rs6923737 | 311 |
| rs7739414 | 38744177 | 0.49 | 0.105 | 0.293 | rs6923737 | 313 |
| rs12214815 | 38755830 | 0.596 | 0.105 | 0.292 | rs6923737 | 321 |
| rs4714175 | 38758819 | 0.472 | 0.105 | 0.306 | rs6923737 | 324 |
| rs10947755 | 38759800 | 0.472 | 0.105 | 0.306 | rs6923737 | 325 |
| rs2471999 | 38760434 | 0.472 | 0.105 | 0.306 | rs6923737 | 326 |
| rs1883610 | 38448077 | 0.409 | 0.108 | 0.458 | rs4711546 | 192 |
| rs9394508 | 38594615 | 0.55 | 0.108 | 0.278 | rs6923737 | 268 |
| rs12662621 | 38528162 | 0.553 | 0.112 | 0.205 | rs6904723 | 245 |
| rs12198690 | 38736696 | 0.606 | 0.112 | 0.304 | rs6923737 | 308 |
| rs10947737 | 38527436 | 1 | 0.113 | 0.207 | rs6904723 | 244 |
| rs12200371 | 38554612 | 0.557 | 0.113 | 0.232 | rs7770868 | 256 |
| rs9470887 | 38558410 | 0.557 | 0.113 | 0.232 | rs7770868 | 257 |
| rs9462435 | 38570041 | 0.557 | 0.113 | 0.232 | rs7770868 | 259 |
| rs10447398 | 38764307 | 0.613 | 0.115 | 0.34 | rs6923737 | 327 |
| rs9380739 | 38440533 | 0.415 | 0.118 | 0.482 | rs4711546 | 189 |
| rs9394492 | 38440588 | 0.415 | 0.118 | 0.482 | rs4711546 | 190 |
| rs6458064 | 38746487 | 0.644 | 0.119 | 0.319 | rs6923737 | 315 |
| rs9366950 | 38324672 | 0.618 | 0.12 | 0.289 | rs9357271 | 152 |
| rs2736655 | 38756772 | 0.499 | 0.122 | 0.381 | rs6923737 | 322 |
| rs2736654 | 38758606 | 0.625 | 0.127 | 0.328 | rs6923737 | 323 |
| rs9349061 | 38322423 | 0.804 | 0.133 | 0.309 | rs9357271 | 149 |
| rs9349062 | 38322441 | 0.804 | 0.134 | 0.309 | rs9357271 | 150 |
| rs17650092 | 38571627 | 1 | 0.134 | 0.214 | rs7770868 | 260 |
| rs9470884 | 38552741 | 0.581 | 0.137 | 0.227 | rs6904723 | 251 |
| rs1033480 | 38428921 | 0.636 | 0.139 | 0.336 | rs6904723 | 183 |
| rs2179533 | 38426699 | 0.584 | 0.141 | 0.266 | rs6904723 | 182 |
| rs2179532 | 38426685 | 0.586 | 0.145 | 0.274 | rs6904723 | 181 |
| rs228186 | 38425664 | 0.634 | 0.153 | 0.304 | rs6904723 | 177 |
| rs12176364 | 38300971 | 0.827 | 0.156 | 0.335 | rs9357271 | 124 |
| rs7356829 | 38313830 | 0.827 | 0.156 | 0.335 | rs9357271 | 128 |
| rs9462415 | 38314213 | 0.827 | 0.156 | 0.335 | rs9357271 | 129 |
| rs9296239 | 38315614 | 0.827 | 0.156 | 0.335 | rs9357271 | 130 |
| rs9470835 | 38315651 | 0.825 | 0.156 | 0.335 | rs9357271 | 132 |
| rs9366949 | 38316836 | 0.827 | 0.156 | 0.335 | rs9357271 | 133 |
| rs9380726 | 38316961 | 0.827 | 0.156 | 0.335 | rs9357271 | 134 |
| rs7745176 | 38317441 | 0.827 | 0.156 | 0.335 | rs9357271 | 136 |
| rs9357265 | 38318032 | 0.827 | 0.156 | 0.335 | rs9357271 | 137 |
| rs9357266 | 38318432 | 0.827 | 0.156 | 0.335 | rs9357271 | 139 |
| rs9296240 | 38318497 | 0.827 | 0.156 | 0.335 | rs9357271 | 140 |
| rs9296241 | 38318553 | 0.827 | 0.156 | 0.335 | rs9357271 | 141 |
| rs9296242 | 38318690 | 0.827 | 0.156 | 0.335 | rs9357271 | 142 |
| rs9394481 | 38318745 | 0.827 | 0.156 | 0.335 | rs9357271 | 143 |
| rs9357267 | 38318984 | 0.827 | 0.156 | 0.335 | rs9357271 | 144 |
| rs9394482 | 38319053 | 0.827 | 0.156 | 0.335 | rs9357271 | 145 |
| rs7755820 | 38321292 | 0.825 | 0.156 | 0.335 | rs9357271 | 146 |
| rs9349063 | 38322495 | 0.827 | 0.156 | 0.335 | rs9357271 | 151 |
| rs871183 | 38396146 | 0.598 | 0.16 | 0.261 | rs6904723 | 171 |
| rs4714148 | 38431277 | 0.737 | 0.16 | 0.203 | rs4711546 | 186 |
| rs9470834 | 38315642 | 0.829 | 0.161 | 0.335 | rs9357271 | 131 |
| rs228179 | 38412860 | 0.643 | 0.163 | 0.32 | rs6904723 | 175 |
| rs228188 | 38426351 | 0.643 | 0.163 | 0.32 | rs6904723 | 179 |
| rs7763775 | 38426652 | 0.643 | 0.163 | 0.32 | rs6904723 | 180 |
| rs12191714 | 38361778 | 0.649 | 0.166 | 0.322 | rs6904723 | 165 |
| rs12206324 | 38365813 | 0.649 | 0.166 | 0.322 | rs6904723 | 167 |
| rs12215850 | 38372831 | 0.649 | 0.166 | 0.322 | rs6904723 | 168 |
| rs7756174 | 38321497 | 0.824 | 0.167 | 0.351 | rs9357271 | 147 |
| rs9394495 | 38479170 | 0.766 | 0.179 | 0.212 | rs4711546 | 221 |
| rs1781738 | 38735257 | 0.503 | 0.182 | 0.43 | rs6923737 | 307 |
| rs1699012 | 38788921 | 0.503 | 0.182 | 0.43 | rs6923737 | 328 |
| rs228182 | 38407846 | 0.497 | 0.184 | 0.219 | rs7770868 | 173 |
| rs2144328 | 38673369 | 0.591 | 0.187 | 0.417 | rs6923737 | 295 |
| rs6940319 | 38437807 | 0.772 | 0.189 | 0.219 | rs4711546 | 187 |
| rs9349073 | 38440346 | 0.772 | 0.189 | 0.219 | rs4711546 | 188 |
| rs4711542 | 38450140 | 0.772 | 0.189 | 0.219 | rs4711546 | 193 |
| rs4714140 | 38362904 | 0.505 | 0.191 | 0.257 | rs7770868 | 166 |
| rs4714144 | 38381069 | 0.503 | 0.198 | 0.218 | rs7770868 | 169 |
| rs228181 | 38408118 | 0.506 | 0.198 | 0.24 | rs7770868 | 174 |
| rs7740763 | 38586245 | 1 | 0.202 | 0.264 | rs6923737 | 265 |
| rs6905637 | 38385888 | 0.526 | 0.209 | 0.246 | rs7770868 | 170 |
| rs228184 | 38400595 | 0.541 | 0.211 | 0.279 | rs4711546 | 172 |
| rs2252550 | 38649462 | 0.606 | 0.211 | 0.456 | rs6923737 | 284 |
| rs4236058 | 38550426 | 1 | 0.217 | 0.386 | rs6904723 | 250 |
| rs12206905 | 38575642 | 1 | 0.217 | 0.342 | rs6904723 | 262 |
| rs6922620 | 38428988 | 0.539 | 0.22 | 0.271 | rs7770868 | 184 |
| rs13194038 | 38338207 | 1 | 0.221 | 0.221 | rs3923809 | 155 |
| rs6458059 | 38665577 | 0.6 | 0.222 | 0.461 | rs6923737 | 291 |
| rs4714136 | 38336104 | 0.552 | 0.223 | 0.259 | rs7770868 | 154 |
| rs1321056 | 38643554 | 0.613 | 0.224 | 0.476 | rs6923737 | 283 |
| rs2814894 | 38652475 | 0.613 | 0.224 | 0.476 | rs6923737 | 286 |
| rs10807198 | 38662250 | 0.613 | 0.224 | 0.476 | rs6923737 | 289 |
| rs1739632 | 38669200 | 0.613 | 0.224 | 0.476 | rs6923737 | 293 |
| rs910516 | 38672926 | 0.613 | 0.224 | 0.476 | rs6923737 | 294 |
| rs1699007 | 38691346 | 0.612 | 0.224 | 0.464 | rs6923737 | 299 |
| rs1699002 | 38703379 | 0.613 | 0.224 | 0.476 | rs6923737 | 300 |
| rs1739626 | 38707544 | 0.613 | 0.224 | 0.476 | rs6923737 | 301 |
| rs1781708 | 38712732 | 0.613 | 0.224 | 0.476 | rs6923737 | 302 |
| rs228185 | 38423194 | 0.551 | 0.226 | 0.261 | rs7770868 | 176 |
| rs228187 | 38425781 | 0.551 | 0.226 | 0.261 | rs7770868 | 178 |
| rs9470891 | 38575487 | 1 | 0.226 | 0.364 | rs6904723 | 261 |
| rs9470847 | 38346529 | 0.555 | 0.229 | 0.264 | rs7770868 | 156 |
| rs9470848 | 38348063 | 0.555 | 0.229 | 0.264 | rs7770868 | 157 |
| rs1475761 | 38348396 | 0.555 | 0.229 | 0.264 | rs7770868 | 158 |
| rs1931767 | 38355823 | 0.555 | 0.229 | 0.264 | rs7770868 | 162 |
| rs9470851 | 38357606 | 0.555 | 0.229 | 0.264 | rs7770868 | 164 |
| rs2745379 | 38489086 | 0.852 | 0.242 | 0.373 | rs7770868 | 229 |
| rs10947740 | 38584991 | 0.553 | 0.246 | 0.433 | rs6923737 | 264 |
| rs2748172 | 38638278 | 0.657 | 0.256 | 0.514 | rs6923737 | 280 |
| rs2745380 | 38489311 | 0.924 | 0.259 | 0.392 | rs7770868 | 230 |
| rs10807196 | 38602589 | 0.656 | 0.26 | 0.538 | rs6923737 | 270 |
| rs4131034 | 38507938 | 0.925 | 0.268 | 0.402 | rs7770868 | 239 |
| rs12194728 | 38493823 | 0.926 | 0.269 | 0.407 | rs7770868 | 234 |
| rs2814889 | 38627739 | 0.671 | 0.269 | 0.53 | rs6923737 | 278 |
| rs2814896 | 38488782 | 0.926 | 0.271 | 0.406 | rs7770868 | 228 |
| rs2745382 | 38492570 | 0.926 | 0.271 | 0.406 | rs7770868 | 233 |
| rs9380748 | 38499784 | 0.926 | 0.271 | 0.406 | rs7770868 | 237 |
| rs4591850 | 38506781 | 0.926 | 0.271 | 0.406 | rs7770868 | 238 |
| rs4591851 | 38508970 | 0.926 | 0.271 | 0.406 | rs7770868 | 240 |
| rs2814888 | 38489334 | 0.931 | 0.293 | 0.438 | rs7770868 | 231 |
| rs2745381 | 38490914 | 1 | 0.296 | 0.423 | rs7770868 | 232 |
| rs2745385 | 38498207 | 1 | 0.305 | 0.436 | rs7770868 | 236 |
| rs2745384 | 38497676 | 1 | 0.307 | 0.422 | rs7770868 | 235 |
| rs9394493 | 38458782 | 0.719 | 0.362 | 1 | rs9357271 | 198 |
| rs4714156 | 38469090 | 0.703 | 0.363 | 0.848 | rs4711546 | 213 |
| rs9380737 | 38429760 | 0.75 | 0.369 | 0.477 | rs7770868 | 185 |
| rs9394494 | 38459959 | 0.746 | 0.388 | 1 | rs9357271 | 201 |
| rs4714155 | 38468878 | 0.741 | 0.391 | 1 | rs9357271 | 212 |
| rs9380741 | 38460057 | 0.761 | 0.403 | 1 | rs9357271 | 202 |
| rs9470867 | 38466838 | 0.764 | 0.414 | 1 | rs9357271 | 209 |
| rs9380740 | 38441452 | 0.763 | 0.415 | 1 | rs9357271 | 191 |
| rs7759078 | 38459269 | 0.763 | 0.415 | 1 | rs9357271 | 199 |
| rs7748599 | 38471191 | 0.763 | 0.415 | 1 | rs9357271 | 215 |
| rs7764502 | 38474115 | 0.763 | 0.415 | 1 | rs9357271 | 218 |
| rs4714152 | 38451319 | 0.765 | 0.416 | 1 | rs9357271 | 194 |
| rs742515 | 38455669 | 0.765 | 0.416 | 1 | rs9357271 | 196 |
| rs7759629 | 38459478 | 0.765 | 0.416 | 1 | rs9357271 | 200 |
| rs4711544 | 38460261 | 0.765 | 0.416 | 1 | rs9357271 | 203 |
| rs1983605 | 38463918 | 0.765 | 0.416 | 1 | rs9357271 | 204 |
| rs7749685 | 38465868 | 0.765 | 0.416 | 1 | rs9357271 | 205 |
| rs9380742 | 38465910 | 0.765 | 0.416 | 1 | rs9357271 | 206 |
| rs9380743 | 38466780 | 0.765 | 0.416 | 1 | rs9357271 | 207 |
| rs9296248 | 38466812 | 0.765 | 0.416 | 1 | rs9357271 | 208 |
| rs9470868 | 38467055 | 0.765 | 0.416 | 1 | rs9357271 | 210 |
| rs7756267 | 38467577 | 0.765 | 0.416 | 1 | rs9357271 | 211 |
| rs7766214 | 38471146 | 0.765 | 0.416 | 1 | rs9357271 | 214 |
| rs9296249 | 38473819 | 0.765 | 0.416 | 1 | rs9357271 | 217 |
| rs4711546 | 38474164 | 0.765 | 0.416 | 1 | rs4711546 | 5 |
| rs4714157 | 38474363 | 0.765 | 0.416 | 1 | rs9357271 | 219 |
| rs9357272 | 38478330 | 0.765 | 0.416 | 1 | rs9357271 | 220 |
| rs6903611 | 38482687 | 0.765 | 0.416 | 1 | rs9357271 | 223 |
| rs6903767 | 38482790 | 0.765 | 0.416 | 1 | rs9357271 | 225 |
| rs4714158 | 38483312 | 0.765 | 0.416 | 1 | rs9357271 | 226 |
| rs9380747 | 38483947 | 0.765 | 0.416 | 1 | rs9357271 | 227 |
| rs9380745 | 38481206 | 0.806 | 0.427 | 0.946 | rs4711546 | 222 |
| rs9357271 | 38473851 | 0.769 | 0.43 | 1 | rs9357271 | 2 |
| rs4140443 | 38455930 | 0.766 | 0.434 | 1 | rs9357271 | 197 |
| rs9369057 | 38482744 | 0.815 | 0.448 | 0.948 | rs4711546 | 224 |
| rs9349077 | 38473574 | 0.816 | 0.449 | 0.948 | rs4711546 | 216 |
| rs6899746 | 38520766 | 0.946 | 0.51 | 0.965 | rs6904723 | 242 |
| rs6901086 | 38538387 | 0.947 | 0.517 | 0.965 | rs6904723 | 249 |
| rs6904723 | 38544295 | 1 | 0.557 | 1 | rs6904723 | 6 |
| rs9470885 | 38553377 | 1 | 0.557 | 1 | rs6904723 | 252 |
| rs4714165 | 38553435 | 1 | 0.557 | 1 | rs6904723 | 253 |
| rs6923737 | 38591542 | 0.858 | 0.572 | 1 | rs6923737 | 3 |
| rs6933758 | 38554349 | 1 | 0.6 | 1 | rs7770868 | 255 |
| rs9470888 | 38561015 | 0.948 | 0.601 | 0.962 | rs7770868 | 258 |
| rs7770868 | 38572604 | 1 | 0.638 | 1 | rs7770868 | 4 |
| rs6933268 | 38554328 | 1 | 0.644 | 1 | rs7770868 | 254 |
| rs10947739 | 38577301 | 0.867 | 0.746 | 0.746 | rs3923809 | 9 |
| rs4236060 | 38578065 | 0.955 | 0.872 | 0.872 | rs3923809 | 8 |
| rs13219518 | 38555848 | 1 | 0.957 | 0.957 | rs3923809 | 11 |
| rs3923809 | 38548948 | 1 | 1 | 1 | rs3923809 | 1 |
| rs6920488 | 38552018 | 1 | 1 | 1 | rs3923809 | 10 |

### Example 2

### Analysis of Phenotypic Contributions to the C06 Association Signal and Replication in Icelandic and US Cohorts

The association to RLS and PLMS observed in the Icelandic sample was investigated in a second Icelandic sample, as well as in a cohort from the US. The US replication cohort was recruited through the Emory Program in Sleep Medicine in Atlanta, Ga. Consent for inclusion and blood draws for the proposed analyses occurred under the auspices of an IRB-HIC approved protocol (HIC ID 133-98) Clinical Research in Neurology (CRIN). Controls were drawn from spouses and patients presenting to other general and subspecialty Neurology clinics.

Table 6 shows results of association analysis of marker rs6923737 in the various cohorts for individuals that present with symptoms that fulfill both RLS and PLMS diagnostic criteria. The original result in the Icelandic discovery sample is replicated in the second Icelandic sample, with comparable OR values, and the signal replicates in the US sample, although with a slightly lower risk. Combined analysis illustrates the significance of the finding (OR=1.7, p-value 3×10⁻¹⁴).

Table 7 shows results from analysis of SNP markers in a 600 kb region surrounding rs3923809 for the RLS+PLMS diagnostic criteria. The table illustrates that many markers in the region show significant association. After correction for the signal due to rs3923809, however, the signal for almost all of the markers is non-significant at the nominal p-value level of 0.05. This illustrates that the markers are surrogates for the association signal detected originally by rs3923809.

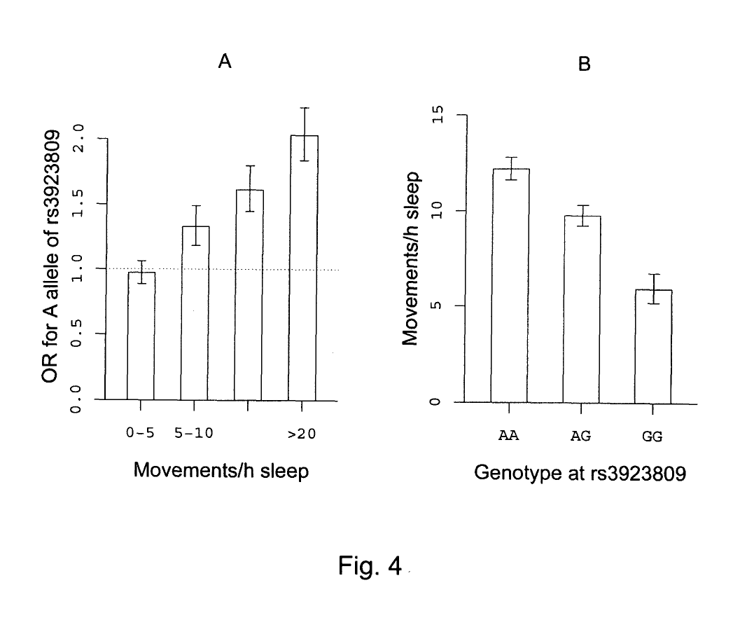

We also found the frequency of periodic limb movements in sleep to correlate with the presence of allele A of marker rs3923809 (FIG. 4A) and to be nearly twice as frequent in AA homozygotes as it was in the in non-carriers (GG) (P<0.001, FIG. 4B). The OR of the most severe group, 20+ movements/h sleep, is 2.0, while for the group with 0-5 movements/h sleep the OR is 1.0 (FIG. 4A). No significant correlation was observed between allele A and RLS severity as assessed by the IRLSSG rating scale (P=0.35) or the self-reported age at onset of RLS symptoms (P=0.73).

TABLE 6
Genome-wide significant association to RLS + PLMs was detected
to allele A of SNP rs3923809 in an Icelandic discovery sample. This
association was replicated in a second Icelandic
sample and a sample from U.S.

| | | | | Allele | |
|---|---|---|---|---|---|
| Population | | | | frequency | |
| (N cases/N | P- | | | Cases | |
| controls) | value | OR (95% CI) | PAR | Controls | |
| RLS + PLMs | | | | | |
| Iceland | 2 × 10⁻⁹ | 1.8 (1.5, 2.2) | 0.57 | 0.775 | 0.656 |
| Discovery | | | | | |
| (306/15,634) | | | | | |
| Replication I | 0.0004 | 1.8 (1.3, 2.4) | 0.56 | 0.772 | 0.657 |
| (123/1,233) | | | | | |
| Combined | 2 × 10⁻¹² | 1.8 (1.5, 2.1) | 0.57 | 0.774 | 0.656 |
| (429/16,866) | | | | | |
| US | 0.004 | 1.5 (1.2, 2.0) | 0.46 | 0.766 | 0.681 |
| Replication II | | | | | |
| (188/662) | | | | | |
| Iceland and US | 3 × 10⁻¹⁴ | 1.7 (1.5, 2.0) | 0.54 | — | — |
| Combined | | | | | |

TABLE 7
The 70 SNPs in a 600kb region around rs3923809 that satisfied quality criteria in the
discovery genome-wide scan. All P values have been corrected by genomic controls.

| | | | | | P value for | P value for | | | | | | |
|---|---|---|---|---|---|---|---|---|---|---|---|---|
| | | | | | SNP | rs3923809 | Genotype counts | | | | | |
| | | Control | 17 | P value | corrected for | corrected for | Cases | | | Controls | | |
| SNP | Position | frequency | OR (95% CI) | for SNP | rs3923809 | SNP | aa | aA | AA | aa | aA | AA |
| rs3800358 | 38249982 | 0.53 | 1.0 (0.8, 1.2) | 0.96 | 0.88 | 2 × 10⁻⁹ | 70 | 149 | 87 | 3554 | 7659 | 4385 |
| rs2235711 | 38256748 | 0.76 | 1.1 (0.9, 1.4) | 0.23 | 0.34 | 3 × 10⁻⁹ | 13 | 107 | 186 | 936 | 5625 | 9102 |
| rs10807192 | 38267796 | 0.48 | 1.0 (0.8, 1.2) | 0.96 | 0.73 | 2 × 10⁻⁹ | 83 | 149 | 74 | 4227 | 7699 | 3736 |
| rs742538 | 38268605 | 0.66 | 1.0 (0.8, 1.2) | 0.96 | 0.79 | 2 × 10⁻⁹ | 40 | 126 | 140 | 1852 | 6869 | 6936 |
| rs13206817 | 38281343 | 0.48 | 1.0 (0.9, 1.2) | 0.62 | 0.65 | 2 × 10⁻⁹ | 76 | 162 | 68 | 4277 | 7848 | 3536 |
| rs1931765 | 38282799 | 0.8 | 1.2 (1.0, 1.5) | 0.12 | 0.48 | 6 × 10⁻⁹ | 7 | 95 | 204 | 692 | 5036 | 9935 |
| rs4299828 | 38285645 | 0.8 | 1.2 (1.0, 1.5) | 0.1 | 0.42 | 6 × 10⁻⁹ | 7 | 94 | 205 | 692 | 5034 | 9934 |
| rs926564 | 38307237 | 0.72 | 1.2 (1.0, 1.5) | 0.04 | 0.73 | 2 × 10⁻⁸ | 16 | 113 | 177 | 1220 | 6183 | 8193 |
| rs909997 | 38310342 | 0.07 | 1.1 (0.8, 1.6) | 0.43 | 0.66 | 3 × 10⁻⁹ | 262 | 42 | 2 | 13663 | 1914 | 85 |
| rs12202202 | 38312281 | 0.12 | 1.2 (0.9, 1.5) | 0.23 | 0.55 | 4 × 10⁻⁹ | 227 | 70 | 8 | 12024 | 3375 | 246 |
| rs9296239 | 38315614 | 0.92 | 1.3 (0.9, 1.9) | 0.12 | 0.97 | 7 × 10⁻⁹ | 2 | 32 | 272 | 104 | 2170 | 13389 |
| rs9366950 | 38324672 | 0.87 | 1.2 (0.9, 1.6) | 0.16 | 0.89 | 6 × 10⁻⁹ | 2 | 66 | 238 | 298 | 3614 | 11750 |
| rs726160 | 38329617 | 0.65 | 1.1 (0.9, 1.4) | 0.18 | 0.51 | 4 × 10⁻⁹ | 30 | 138 | 137 | 1937 | 7138 | 6578 |
| rs13194038 | 38338207 | 0.92 | 1.5 (1.1, 2.2) | 0.02 | 0.45 | 3 × 10⁻⁸ | 0 | 34 | 272 | 104 | 2372 | 13187 |
| rs9462426 | 38354751 | 0.37 | 1.2 (1.0, 1.4) | 0.04 | 0.61 | 2 × 10⁻⁸ | 107 | 142 | 56 | 6167 | 7289 | 2208 |
| rs6905637 | 38385888 | 0.67 | 1.4 (1.2, 1.7) | 0.0002 | 0.23 | 1 × 10⁻⁶ | 17 | 121 | 168 | 1671 | 6911 | 7082 |
| rs228181 | 38408118 | 0.67 | 1.5 (1.2, 1.8) | 8 × 10⁻⁵ | 0.30 | 4 × 10⁻⁶ | 16 | 123 | 167 | 1704 | 6978 | 6975 |
| rs12660215 | 38413748 | 0.18 | 1.1 (0.9, 1.4) | 0.25 | 0.99 | 4 × 10⁻⁹ | 193 | 103 | 10 | 10505 | 4632 | 526 |
| rs228188 | 38426351 | 0.47 | 1.2 (1.1, 1.5) | 0.01 | 0.67 | 6 × 10⁻⁸ | 66 | 161 | 79 | 4470 | 7783 | 3409 |
| rs4573069 | 38431723 | 0.33 | 1.2 (1.0, 1.4) | 0.05 | 0.36 | 1 × 10⁻⁸ | 124 | 135 | 47 | 6955 | 6980 | 1729 |
| rs13200372 | 38436415 | 0.88 | 1.2 (0.9, 1.6) | 0.12 | 0.50 | 6 × 10⁻⁹ | 2 | 56 | 248 | 217 | 3293 | 12153 |
| rs6907434 | 38444627 | 0.79 | 1.1 (0.9, 1.4) | 0.37 | 0.30 | 2 × 10⁻⁹ | 14 | 92 | 200 | 706 | 5225 | 9731 |
| rs1883610 | 38448077 | 0.64 | 1.1 (0.9, 1.3) | 0.18 | 0.09 | 1 × 10⁻⁹ | 29 | 140 | 124 | 2082 | 7181 | 6289 |
| rs4711542 | 38450140 | 0.43 | 1.2 (1.0, 1.4) | 0.03 | 0.62 | 2 × 10⁻⁸ | 86 | 151 | 69 | 5123 | 7732 | 2809 |
| rs9357271 | 38473851 | 0.76 | 1.8 (1.4, 2.3) | 2 × 10⁻⁷ | 0.28 | 0.001 | 5 | 80 | 221 | 896 | 5632 | 9135 |
| rs4711546 | 38474164 | 0.76 | 1.7 (1.4, 2.1) | 2 × 10⁻⁶ | 0.65 | 0.0003 | 7 | 82 | 217 | 931 | 5696 | 9036 |
| rs4565302 | 38508117 | 0.12 | 1.6 (1.2, 2.0) | 0.0002 | 0.03 | 2 × 10⁻⁷ | 211 | 85 | 10 | 12214 | 3229 | 216 |
| rs9470878 | 38514714 | 0.89 | 1.2 (0.9, 1.6) | 0.22 | 0.96 | 5 × 10⁻⁹ | 3 | 54 | 249 | 202 | 3189 | 12272 |
| rs12208647 | 38516201 | 0.12 | 1.6 (1.2, 2.0) | 0.0002 | 0.02 | 2 × 10⁻⁷ | 210 | 85 | 11 | 12157 | 3278 | 227 |
| rs6904723 | 38544295 | 0.53 | 1.5 (1.3, 1.8) | 7 × 10⁻⁷ | 0.45 | 0.0006 | 37 | 151 | 118 | 3517 | 7716 | 4346 |
| rs3923809 | 38548948 | 0.66 | 1.8 (1.5, 2.2) | 2 × 10⁻⁹ | — | — | 12 | 114 | 180 | 1816 | 7120 | 6698 |
| rs4236058 | 38550426 | 0.33 | 1.2 (1.0, 1.5) | 0.02 | 0.53 | 3 × 10⁻⁸ | 118 | 144 | 43 | 7013 | 6987 | 1661 |
| rs9470887 | 38558410 | 0.89 | 1.1 (0.9, 1.5) | 0.33 | 0.40 | 2 × 10⁻⁹ | 3 | 54 | 249 | 187 | 3103 | 12374 |
| rs9380755 | 38580104 | 0.83 | 1.1 (0.8, 1.3) | 0.65 | 0.94 | 2 × 10⁻⁹ | 10 | 77 | 219 | 408 | 4376 | 10880 |
| rs6923737 | 38591542 | 0.65 | 1.7 (1.4, 2.0) | 1 × 10⁻⁷ | 0.16 | 0.002 | 16 | 116 | 174 | 1885 | 7096 | 6679 |
| rs10498741 | 38594688 | 0.14 | 1.4 (1.1, 1.7) | 0.008 | 0.29 | 5 × 10⁻⁸ | 206 | 86 | 14 | 11424 | 3926 | 293 |
| rs2748156 | 38621152 | 0.32 | 1.2 (1.0, 1.4) | 0.06 | 1.00 | 1 × 10⁻⁸ | 120 | 150 | 36 | 7129 | 6890 | 1644 |
| rs6931131 | 38623973 | 0.45 | 1.1 (1.0, 1.4) | 0.12 | 0.93 | 7 × 10⁻⁹ | 82 | 153 | 71 | 4706 | 7840 | 3105 |
| rs2748173 | 38638120 | 0.32 | 1.2 (1.0, 1.4) | 0.07 | 0.99 | 1 × 10⁻⁸ | 120 | 151 | 35 | 7151 | 6881 | 1631 |
| rs1321056 | 38643554 | 0.6 | 1.5 (1.2, 1.7) | 4 × 10⁻⁵ | 0.12 | 4 × 10⁻⁶ | 27 | 137 | 142 | 2394 | 7641 | 5597 |
| rs2814891 | 38644949 | 0.07 | 1.3 (1.0, 1.8) | 0.06 | 0.05 | 2 × 10⁻⁹ | 256 | 47 | 3 | 13673 | 1909 | 73 |
| rs17543178 | 38651355 | 0.14 | 1.5 (1.2, 1.9) | 0.0003 | 0.03 | 2 × 10⁻⁷ | 198 | 98 | 10 | 11655 | 3731 | 275 |
| rs2814894 | 38652475 | 0.6 | 1.5 (1.2, 1.7) | 3 × 10⁻⁵ | 0.11 | 4 × 10⁻⁶ | 27 | 136 | 142 | 2388 | 7661 | 5604 |
| rs2748166 | 38660252 | 0.38 | 1.2 (1.0, 1.4) | 0.09 | 0.87 | 1 × 10⁻⁸ | 104 | 152 | 50 | 6092 | 7368 | 2202 |
| rs2180106 | 38668193 | 0.23 | 1.3 (1.1, 1.6) | 0.005 | 0.14 | 4 × 10⁻⁸ | 158 | 125 | 22 | 9380 | 5482 | 790 |
| rs1739626 | 38707544 | 0.6 | 1.4 (1.2, 1.7) | 5 × 10⁻⁵ | 0.13 | 3 × 10⁻⁶ | 27 | 137 | 142 | 2392 | 7553 | 5609 |
| rs1739633 | 38725698 | 0.74 | 1.3 (1.1, 1.6) | 0.009 | 0.57 | 6 × 10⁻⁸ | 13 | 102 | 190 | 970 | 6143 | 8550 |
| rs1781738 | 38735257 | 0.64 | 1.5 (1.2, 1.8) | 6 × 10⁻⁵ | 0.12 | 3 × 10⁻⁶ | 24 | 122 | 160 | 1956 | 7339 | 6369 |
| rs13210334 | 38736260 | 0.91 | 1.1 (0.8, 1.5) | 0.62 | 0.57 | 2 × 10⁻⁹ | 2 | 48 | 255 | 135 | 2592 | 12921 |
| rs1937782 | 38737208 | 0.91 | 1.1 (0.8, 1.4) | 0.67 | 0.62 | 2 × 10⁻⁹ | 2 | 49 | 255 | 135 | 2608 | 12920 |
| rs6458065 | 38749332 | 0.55 | 1.3 (1.1, 1.5) | 0.01 | 0.77 | 6 × 10⁻⁸ | 50 | 142 | 114 | 3141 | 7812 | 4709 |
| rs3778443 | 38762466 | 0.06 | 1.4 (1.0, 1.9) | 0.04 | 0.09 | 4 × 10⁻⁹ | 254 | 50 | 1 | 13760 | 1835 | 61 |
| rs6932648 | 38766861 | 0.92 | 1.0 (0.8, 1.4) | 0.90 | 0.85 | 2 × 10⁻⁹ | 2 | 47 | 257 | 104 | 2449 | 13103 |
| rs10484854 | 38772572 | 0.31 | 1.0 (0.9, 1.2) | 0.78 | 0.33 | 1 × 10⁻⁹ | 141 | 136 | 28 | 7497 | 6655 | 1512 |
| rs1781735 | 38780057 | 0.48 | 1.1 (1.0, 1.4) | 0.12 | 0.78 | 7 × 10⁻⁹ | 69 | 158 | 79 | 4143 | 7926 | 3593 |
| rs1937780 | 38782428 | 0.4 | 1.0 (0.8, 1.2) | 0.99 | 0.33 | 1 × 10⁻⁹ | 115 | 139 | 52 | 5666 | 7548 | 2439 |
| rs1781731 | 38790310 | 0.07 | 1.4 (1.0, 1.9) | 0.03 | 0.07 | 5 × 10⁻⁹ | 253 | 51 | 2 | 13685 | 1903 | 72 |
| rs1937781 | 38797853 | 0.33 | 1.2 (1.0, 1.4) | 0.07 | 0.10 | 3 × 10⁻⁹ | 128 | 130 | 48 | 7025 | 6886 | 1753 |
| rs9296260 | 38800415 | 0.76 | 1.1 (0.9, 1.4) | 0.30 | 0.43 | 3 × 10⁻⁹ | 16 | 106 | 184 | 896 | 5862 | 8904 |
| rs1698998 | 38804646 | 0.83 | 1.2 (1.0, 1.5) | 0.10 | 0.20 | 4 × 10⁻⁹ | 10 | 69 | 227 | 435 | 4503 | 10723 |
| rs1623375 | 38806091 | 0.53 | 1.1 (0.9, 1.3) | 0.38 | 0.87 | 3 × 10⁻⁹ | 73 | 133 | 100 | 3619 | 7639 | 4405 |
| rs1626976 | 38806558 | 0.77 | 1.0 (0.9, 1.3) | 0.70 | 0.29 | 1 × 10⁻⁹ | 19 | 101 | 185 | 916 | 5527 | 9221 |
| rs17552381 | 38806755 | 0.26 | 1.2 (1.0, 1.4) | 0.13 | 0.18 | 3 × 10⁻⁹ | 158 | 116 | 31 | 8539 | 5997 | 1110 |
| rs1781726 | 38812327 | 0.35 | 1.1 (0.9, 1.3) | 0.17 | 0.18 | 2 × 10⁻⁹ | 124 | 132 | 50 | 6636 | 7031 | 1969 |
| rs1937777 | 38812483 | 0.06 | 1.1 (0.8, 1.6) | 0.42 | 0.69 | 3 × 10⁻⁹ | 265 | 37 | 4 | 13711 | 1874 | 77 |
| rs756405 | 38814051 | 0.26 | 1.1 (0.9, 1.3) | 0.36 | 0.41 | 2 × 10⁻⁹ | 161 | 120 | 25 | 8572 | 6022 | 1066 |
| rs6458068 | 38820666 | 0.17 | 1.1 (0.9, 1.3) | 0.59 | 0.52 | 2 × 10⁻⁹ | 205 | 92 | 9 | 10767 | 4441 | 456 |
| rs2206860 | 38831139 | 0.47 | 1.0 (0.9, 1.2) | 0.83 | 0.83 | 2 × 10⁻⁹ | 82 | 157 | 65 | 4411 | 7759 | 3400 |
| rs1678677 | 38836120 | 0.79 | 1.0 (0.8, 1.3) | 0.69 | 0.48 | 2 × 10⁻⁹ | 11 | 101 | 194 | 695 | 5126 | 9841 |
| rs13219077 | 38847145 | 0.74 | 1.0 (0.8, 1.2) | 0.95 | 0.75 | 2 × 10⁻⁹ | 18 | 122 | 165 | 1040 | 6068 | 8550 |

Also shown in Table 8 is the analysis for various phenotypic entities based on the RLS and PLMS phenotypes, i.e. RLS in the presence of PLMS (RLS+PLMS), RLS in the absence of PLMS (RLS−PLMS), PLMS in the absence of RLS (PLMS−RLS), as well as for RLS combined (RLS) and PLMS combined (PLMS).

TABLE 8
RLS phenotypes in the Icelandic sample. Genome-wide significant
association to RLS + PLMS, to RLS and to PLM was detected to
allele A of SNP rs3923809. For the group of subjects reporting RLS
symptoms without PLMS (RLS − PLMS) no association to rs3923809
was detected. Conversely, subjects with PLMs who did not meet the
RLS consensus criteria (PLMS − RLS) had an OR indistinguishable
from that in the RLS + PLMs group (P = 0.19). Note that the number
of subjects in the combined PLMS (N = 546) is greater than the sum of
RLS + PLMS and PLMS − RLS (534). The 12 additional subjects
with PLMS have incomplete answers to the RLS questionnaire and
therefore do not belong to the RLS + PLMs or the PLMS-RLS groups.

| | | | Allele | |
|---|---|---|---|---|
| Phenotype | | | frequency | |
| (N cases/N | P- | | Cases | |
| controls) | value | OR (95% CI) | Controls | |
| RLS + PLMS | 2 × 10⁻¹² | 1.8 (1.5, 2.1) | 0.774 | 0.656 |
| (429/16,866) | | | | |
| RLS − PLMs | 0.81 | 1.0 (0.8, 1.2) | 0.651 | 0.656 |
| (229/16,866) | | | | |
| PLMS − RLS | 2 × 10⁻⁶ | 2.3 (1.6, 3.2) | 0.814 | 0.656 |
| (105/16,866) | | | | |
| RLS | 6 × 10⁻⁸ | 1.4 (1.2, 1.6) | 0.731 | 0.656 |
| (658/16,866) | | | | |
| PLMS | 1 × 10⁻¹⁷ | 1.9 (1.5, 2.2) | 0.783 | 0.656 |
| (546/16,866) | | | | |

### Example 3

### Genetic Risk Factor for RLS and PLMS is a Marker for Body Iron Stores

Female sex, advanced age, depletion of body iron stores and western European ancestry are risk factors for RLS (Allen, R., et al Sleep Med 5:385-91 2004; Trenkwalder C, et al., Lancet Neurol 4:465-75 2005. To determine whether these factors interact with the at-risk variant we analysed them as covariates in conferring risk of RLS. The risk of RLS+PLMs conferred by allele A of rs3923809 in males (OR=2.0) is greater than that for females (OR=1.7), although the difference is not significant (P=0.28). A similar insignificant trend (P=0.09) is observed for the combined PLMs groups (OR=2.3 in males, OR=1.7 in females). We observed a significantly greater number of PLMs after the age of 50 (P<0.001 for both sexes), consistent with a previous study²³ The increase was significantly less in women (P=0.04).

The principal clinical tools for assessing iron availability include serum iron, transferrin iron-binding capacity, and ferritin levels. Serum soluble transferrin receptor, ferritin, total iron binding capacity, and iron were assayed in 965 Icelandic subjects (RLS subjects and their relatives). Ferritin index, a measure inversely related to body iron stores, was increased by 5.5% per A allele of marker rs3923809 (95% CI 1%-10%, P=0.02). In line with this observation, serum ferritin was decreased by 13% per A allele (95% CI 5%-20%, P=0.002) (FIG. 5). This shows that the A allele of marker rs3923809 can be used for quantitative prediction of iron stores in humans, and thus has utility as a diagnostic marker of iron stores.

### Example 4

### Association to Markers in the Meis1 Gene on Chromosome 2p14

Investigation of haplotypes over entire haplotype blocks between recombination hotspots revealed significant association to a haplotype within the Meis1 gene (Table 10). This haplotype is significantly associated to PLMS, with OR value in excess of 2.

Further analysis revealed that a two-marker haplotype comprising rs2192954 allele A and rs2300478 allele G captures the association (Table 11). The association appears to be strongest in PLMs in the absence of RLS, but is also significant in the RLS and PLMs combined group (Table 11). The Meis1 gene is thus implicated as a susceptibility gene for PLMs and RLS.

TABLE 10
Association of the haplotyped defined by (allele-marker): 4 rs4387782 4
rs12713568 4 rs3890755 2 rs6728018 2 rs4480973 3 rs10865355 3
rs9789535 4 rs2216120 1 rs2300477 1 rs2192954 3 rs2300478 3
rs6711787 1 rs2284706 2 rs2300484 2 rs1000756 to PLMS

| Orig. Pval | Corr. Pval | OR | Naff | frq. aff | Nctr | frq. ctr | Info | Chr | Mb |
|---|---|---|---|---|---|---|---|---|---|
| 2.08E−12 | 7.09E−11 | 2.1483 | 431 | 0.151242 | 19572 | 0.076594 | 0.939377 | chr2 | 66642261 |

TABLE 11
Association between the haplotype A-rs2192954 G-rs2300478
and RLS with or without Periodic Limb Movements in Sleep among
Icelandic subjects. For increased information content, the calculations
take into account the genotypic status of the subjects for marker rs10865355.

| | | | Allele Frequency | | | |
|---|---|---|---|---|---|---|
| Phenotype | Case/Control | OR (95% CI) | Case | Control | info | P Value |
| RLS and PLMs | 342/19572 | 1.75 (1.4, 2.2) | 0.1843 | 0.1142 | 0.98 | 6.6 × 10⁻⁷ |
| RLS not PLMs | 49/19572 | 1.07 (0.6, 2.0) | 0.1209 | 0.1142 | 0.98 | 0.84 |
| PLMs not RLS | 67/19572 | 2.22 (1.4, 3.5) | 0.2228 | 0.1142 | 0.98 | 0.00049 |
| RLS total | 391/19572 | 1.66 (1.3, 2.1) | 0.1763 | 0.1142 | 0.98 | 2.9 × 10⁻⁶ |
| PLMs total | 431/19572 | 1.82 (1.5, 2.2) | 0.1901 | 0.1142 | 0.98 | 4.5 × 10⁻⁹ |
| PLMs or RLS | 480/19572 | 1.74 (1.4, 2.1) | 0.183 | 0.1142 | 0.98 | 2.7 × 10⁻⁸ |

TABLE 12
Key to sequence listing for flanking sequences of markers identifying
the Meis1 LD block as associating with PLMs and RLS.

| SEQ ID NO | Marker |
|---|---|
| 329 | rs4387782 |
| 330 | rs12713568 |
| 331 | rs3890755 |
| 332 | rs6728018 |
| 333 | rs4480973 |
| 334 | rs10865355 |
| 335 | rs9789535 |
| 336 | rs2216120 |
| 337 | rs2300477 |
| 338 | rs2192954 |
| 339 | rs2300478 |
| 340 | rs6711787 |
| 341 | rs2284706 |
| 342 | rs2300484 |
| 343 | rs1000756 |

1. A method of determining a susceptibility to a sleep-related movement disorder in a human individual, the method comprising determining the presence or absence of at least one allele of at least one polymorphic marker in a nucleic acid sample obtained from the individual, or in a genotype dataset derived from the individual, wherein the at least one polymorphic marker is selected from the group of markers set forth in Table 4, and markers in linkage disequilibrium therewith, wherein the presence of the at least one allele is indicative of a susceptibility to a sleep-related movement disorder.

2. The method according to claim 1, wherein the at least one polymorphic marker is selected from the group consisting of marker rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3), and markers in linkage disequilibrium therewith.

3. The method according to claim 1 or claim 2, wherein the at least one polymorphic marker is selected from the group of markers consisting of rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11).

4. The method according to claim 1 or claim 2, wherein the at least one polymorphic marker is marker rs3923809 (SEQ ID NO:1), or markers in linkage disequilibrium therewith.

5. The method according to claim 4, wherein the at least one polymorphic marker is selected from the markers set forth in Table 5.

6. The method according to claim 5, wherein the at least one polymorphic marker is rs3923809.

7. The method according to any of the preceding claims, wherein the at least one polymorphic marker is associated with the BTBD9 gene, the GLO1 gene, the DNAH8 gene and/or the TEX27 gene.

8. The method according to claim 6, wherein the at least one polymorphic marker is associated with the BTBD9 gene.

9. The method according to any of the preceding claims, further comprising assessing the frequency of at least one haplotype in the individual, wherein the haplotype comprises at least two markers, and wherein the presence of the at least one haplotype is indicative of a susceptibility to a sleep-related movement disorder.

10. The method according to any of the preceding claims, wherein the presence of the at least one allele or haplotype is indicative of increased susceptibility to a sleep-related movement disorder.

11. The method according to claim 10, wherein the at least one allele or haplotype comprises at least one of rs3923809 allele 1, rs9357271 allele 4, rs6923737 allele 4, rs7770868 allele 1, rs4711546 allele 1, rs6904723 allele 1, rs12208647 allele 1, rs4236060 allele 2, rs10947739 allele 2, rs6920488 allele 1, and rs13219518 allele 1.

12. The method according to claim 10 or 11, wherein the increased susceptibility is characterized by a relative risk or odds ratio of at least 1.4, including at least 1.5, at least 1.6, a at least 1.7, at least 1.8, at least 1.9 or at least 2.0.

13. The method of any of the claims 1-9, wherein the presence of the at least one allele or haplotype is indicative of decreased susceptibility to a sleep-related movement disorder.

14. The method according to claim 14, wherein the decreased susceptibility is characterized by a relative risk of less than 0.9, including a relative risk of less than 0.8, a relative risk of less than 0.7, a relative risk of less than 0.6, and a relative risk of less than 0.5.

15. The method according to any of the preceding claims, further comprising analyzing a sample comprising genomic DNA from the human individual or a genotype dataset derived from the human individual for the presence or absence of at an additional at-risk variant for the sleep-related movement disorder, wherein the addition variant comprises at least one at-risk allele of at least one at-risk variant for the sleep-related movement disorder that is not in linkage disequilibrium with an at-risk marker for the sleep-related movement disorder associated with the C06 LD Block.

16. The method of any of the preceding claims, further comprising analyzing non-genetic information to make risk assessment, diagnosis, or prognosis of the human individual.

17. The method of claim 16, wherein the non-genetic information is selected from age, gender, ethnicity, socioeconomic status, previous disease diagnosis, medical history of subject, family history of a sleep-related movement disorder, biochemical measurements, and clinical measurements.

18. The method of claim 40, wherein the biochemical measurements represent a measure of iron stores in the individual.

19. The method of claim 42, wherein the iron stores are represented by serum ferritin levels and/or increased ferritin index in the individual.

20. The method of any of the claims 15-19, further comprising calculating combined risk.

21. A kit for assessing susceptibility to a sleep-related movement disorder in a human individual, the kit comprising reagents for selectively detecting at least one allele of at least one polymorphic marker in the genome of the individual, wherein the polymorphic marker is selected from the markers set forth in Table 4, and markers in linkage disequilibrium therewith, and wherein the presence of the at least one allele is indicative of a susceptibility to a sleep-related movement disorder.

22. The kit of claim 21, wherein the at least one polymorphic marker is selected from rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11).

23. The kit according to claim 21 or claim 22, wherein the at least one polymorphic marker is selected from rs9357271 (SEQ ID NO:2), rs4711546 (SEQ ID NO:5), rs12208647 (SEQ ID NO:7), rs6904723 (SEQ ID NO:6), rs3923809 (SEQ ID NO:1), rs7770868 (SEQ ID NO: 4) and rs6923737 (SEQ ID NO: 3).

24. The kit according to claim 23, wherein the at least one polymorphic marker is rs3923809 (SEQ ID NO:1), or markers in linkage disequilibrium therewith.

25. The kit according to any of the claims 21-24, wherein the reagents comprise at least one contiguous oligonucleotide that hybridizes to a fragment of the genome of the individual comprising the at least one polymorphic marker, a buffer and a detectable label.

26. The kit according to any of the claims 21-25, wherein the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size.

27. The kit according to claim 25 or 26, wherein the at least one oligonucleotide is completely complementary to the genome of the individual.

28. The kit according to any of the claims 25-27, wherein the oligonucleotide is about 18 to about 50 nucleotides in length.

29. The kit according to any of the claims 25-28, wherein the oligonucleotide is 20-30 nucleotides in length.

30. The kit according to any of the claims 21-29, wherein the kit comprises:
d. a detection oligonucleotide probe that is from 5-100 nucleotides in length;
e. an enhancer oligonucleotide probe that is from 5-100 nucleotides in length; and
f. an endonuclease enzyme;
wherein the detection oligonucleotide probe specifically hybridizes to a first segment of the nucleic acid whose nucleotide sequence is set forth in any one of SEQ ID NO:1-343; and
wherein the detection oligonucleotide probe comprises a detectable label at its 3′ terminus and a quenching moiety at its 5′ terminus;
wherein the enhancer oligonucleotide is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid;
wherein a single base gap exists between the first segment and the second segment, such that when the oligonucleotide probe and the enhancer oligonucleotide probe are both hybridized to the nucleic acid, a single base gap exists between the oligonucleotides; and
wherein treating the nucleic acid with the endonuclease will cleave the detectable label from the 3′ terminus of the detection probe to release free detectable label when the detection probe is hybridized to the nucleic acid.

31. A method of assessing an individual for probability of response to a therapeutic agent used for preventing or ameliorating symptoms associated with a sleep-related movement disorder, comprising: determining the presence or absence of at least one allele of at least one polymorphic marker in a nucleic acid sample obtained from the individual, or in a dataset derived from the individual, wherein the at least one polymorphic marker is selected from the group consisting of the polymorphic markers set forth in Table 4, wherein the presence of the marker is indicative of a probability of a positive response to the therapeutic agent.

32. The method according to claim 24, wherein the therapeutic agent is selected from entacapone, levodopa, carbidopa, combinations comprising entacapone, levodopa and carbidopa, rotigotine, safinamide, pramipexole, ropinirole, gabapentin, gabapentin enacarbil, istradefylline, aplindore, lisuride, radafaxine, SEP-226330, sumanirole, nitric oxide donors and dopamine D1 receptor agonists.

33. A method of determining susceptibility to abnormal iron stores in a human individual, the method comprising determining the presence or absence of at least one allele of at least one polymorphic marker in a nucleic acid sample obtained from the individual, or in a dataset derived from the individual, wherein the at least one polymorphic marker is selected from the group consisting of the polymorphic markers set forth in Table 4, and markers in linkage disequilibrium therewith, wherein the presence of the marker is indicative of susceptibility to abnormal iron stores in the individual.

34. The method according to claim 33, wherein the abnormal iron stores are characterized by decreased serum ferritin levels and/or increased ferritin index.

35. The method of claims 33 or 34, wherein the presence of rs3923809 allele A, or a marker allele in linkage disequilibrium therewith, in the individual, is indicative of decreased serum ferritin levels in the individual by at least 10% per allele copy.

36. A method of diagnosing a susceptibility to a sleep-related movement disorder in a human individual, the method comprising determining the presence or absence of at least one allele of at least one polymorphic marker in a nucleic acid sample obtained from the individual, wherein the at least one polymorphic marker is associated with the Meis1 gene, wherein the presence of the at least one allele is indicative of a susceptibility to a sleep-related movement disorder.

37. The method according to claim 36, wherein the at least one marker is located within the Meis1 LD block, between positions 66,580,000 and 66,660,000 in NCBI Build 36 of the human sequence assembly.

38. The method according to claim 36 or claim 37, wherein the at least marker is selected from the markers indicated in Table 12.

39. The method according to any one of claim 36-38, further comprising assessing the frequency of at least one haplotype in the individual, wherein the haplotype comprises at least two markers, and wherein the presence of the at least one haplotype is indicative of increased susceptibility to a sleep-related movement disorder.

40. The method according to claim 39, wherein the haplotype comprises rs2192954 allele A and rs2300478 allele G.

41. The method according to claim 40, wherein the haplotype is selected from
T-rs4387782 T-rs12713568 T-rs3890755 C-rs6728018 C-rs4480973 G-rs10865355 G-rs9789535 T-rs2216120 A-rs2300477 A-rs2192954 G-rs2300478 G-rs6711787 A-rs2284706 C-rs2300484 C-rs1000756; and
A-rs2192954 G-rs2300478.

42. A method of assessing a susceptibility to a sleep-related movement disorder in a human individual, comprising screening a nucleic acid from the individual for at least one polymorphic marker allele or haplotype within the C06 LD block, between position 37,816,141 and 38,797,853 in Build 36 of the National Center for Biotechnology (NCBI) Build 36 sequence assembly, that correlates with increased occurrence of the sleep-related movement disorder in a human population;
wherein determination of the presence of an at-risk marker allele in the at least one polymorphism or an at-risk haplotype in the nucleic acid identifies the individual as having elevated susceptibility to the movement disorder, and
wherein the absence of the at least one at-risk marker allele or at-risk haplotype in the nucleic acid identifies the individual as not having the elevated susceptibility.

43. The method of claim 42, wherein the at least one polymorphic marker is selected from the markers set forth in Table 4, and markers in linkage disequilibrium therewith.

44. A method of identification of a marker for use in assessing susceptibility to a sleep-related movement disorder in human individuals, the method comprising
a. identifying at least one polymorphic marker within the C06 LD block, between position 37,816,141 and 38,797,853 in Build 36 of the National Center for Biotechnology (NCBI) Build 36 sequence assembly;
b. determining the genotype status of a sample of individuals diagnosed with a sleep-related movement disorder; and
c. determining the genotype status of a sample of control individuals;
wherein a significant difference in frequency of at least one allele in at least one polymorphism in individuals diagnosed with the sleep-related movement disorder as compared with the frequency of the at least one allele in the control sample is indicative of the at least one polymorphism being useful for assessing susceptibility to the sleep-related movement disorder.

45. The method according to claim 44, wherein an increase in frequency of the at least one allele in the at least one polymorphism in individuals diagnosed with the sleep-related movement disorder, as compared with the frequency of the at least one allele in the control sample, is indicative of the at least one polymorphism being useful for assessing increased susceptibility to the sleep-related movement disorder.

46. The method according to claim 44, wherein a decrease in frequency of the at least one allele in the at least one polymorphism in individuals diagnosed with the sleep-related movement disorder, as compared with the frequency of the at least one allele in the control sample, is indicative of the at least one polymorphism being useful for assessing decreased susceptibility to, or protection against, the sleep-related movement disorder.

47. The method according to any of the claims 44-46, wherein the significant difference in frequency is characterized by a statistical measure.

48. A method of genotyping a nucleic acid sample obtained from a human individual, comprising determining the presence or absence of at least one allele of at least one polymorphic marker predictive of increased risk of a sleep-related movement disorder in the sample, wherein the at least one marker is selected from the markers set forth in Table 4, and markers in linkage disequilibrium therewith, and wherein determination of the presence or absence of the at least one allele of the at least one polymorphic marker is predictive of increased risk of the sleep-related movement disorder in the individual.

49. The method of claim 48, wherein genotyping comprises amplifying a segment of a nucleic acid that comprises the at least one polymorphic marker, by Polymerase Chain Reaction (PCR), using a nucleotide primer pair flanking the at least one polymorphic marker.

50. The method of claim 48 or 49, wherein genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis.

51. The method of claim 50, wherein the process comprises allele-specific probe hybridization.

52. The method of any of the claims 48-51, comprising:
1. contacting copies of the nucleic acid with a detection oligonucleotide probe and an enhancer oligonucleotide probe under conditions for specific hybridization of the oligonucleotide probe with the nucleic acid;
wherein
e) the detection oligonucleotide probe is from 5-100 nucleotides in length and specifically hybridizes to a first segment of the nucleic acid whose nucleotide sequence is given by SEQ ID NO:50 that comprises at least one polymorphic site;
f) the detection oligonucleotide probe comprises a detectable label at its 3′ terminus and a quenching moiety at its 5′ terminus;
g) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
h) a single base gap exists between the first segment and the second segment, such that when the oligonucleotide probe and the enhancer oligonucleotide probe are both hybridized to the nucleic acid, a single base gap exists between the oligonucleotides;
2. treating the nucleic acid with an endonuclease that will cleave the detectable label from the 3′ terminus of the detection probe to release free detectable label when the detection probe is hybridized to the nucleic acid; and
3. measuring free detectable label, wherein the presence of the free detectable label indicates that the detection probe specifically hybridizes to the first segment of the nucleic acid, and indicates the sequence of the polymorphic site as the complement of the detection probe.

53. A method of predicting prognosis of an individual diagnosed with a sleep-related movement disorder, the method comprising determining the presence or absence of at least one allele of at least one polymorphic marker in a nucleic acid sample obtained from the individual, wherein the at least one polymorphic marker is selected from the markers set forth in Table 4, and markers in linkage disequilibrium therewith, wherein determination of the presence of the at least one allele is indicative of a worse prognosis of the sleep-related movement disorder in the individual.

54. A method of monitoring progress of a treatment of an individual undergoing treatment for a sleep-related movement disorder, the method comprising determining the presence or absence of at least one allele of at least one polymorphic marker in a nucleic acid sample obtained from the individual, wherein the at least one polymorphic marker is selected from the markers set forth in Table 4, and markers in linkage disequilibrium therewith, wherein determination of the presence of the at least one allele is indicative of the treatment outcome of the individual.

55. An apparatus for determining a genetic indicator for a sleep-related movement disorder in a human individual, comprising:
a computer readable memory; and
a routine stored on the computer readable memory;
wherein the routine is adapted to be executed on a processor to analyze marker and/or haplotype information for at least one human individual with respect to at least one polymorphic marker selected from the markers set forth in Table 4, and markers in linkage disequilibrium therewith, and generate an output based on the marker or haplotype information, wherein the output comprises a risk measure of the at least one marker or haplotype as a genetic indicator of the sleep-related movement disorder in the human individual.

56. The apparatus according to claim 55, wherein the routine further comprises an indicator of the frequency of at least one allele of at least one polymorphic marker or at least one haplotype in a plurality of individuals diagnosed with a sleep-related movement disorder, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with a sleep-related movement disorder.

57. The apparatus according to claim 55 or 56, wherein the at least one polymorphic marker is selected from rs3923809 (SEQ ID NO: 1), rs9357271 (SEQ ID NO:2), rs6923737 (SEQ ID NO:3), rs7770868 (SEQ ID NO: 4), rs4711546 (SEQ ID NO: 5), rs6904723 (SEQ ID NO: 6), rs12208647 (SEQ ID NO: 7), rs4236060 (SEQ ID NO:8), rs10947739 (SEQ ID NO:9), rs6920488 (SEQ ID NO:10), and rs13219518 (SEQ ID NO:11).

58. The apparatus according to any of the claims 55-57, wherein the risk measure is characterized by an Odds Ratio (OR) or a Relative Risk (RR).

59. The method, kit or apparatus according to any of the preceding claims, wherein the sleep-related movement disorder is selected from Restless Legs Syndrome (RLS), Periodic Leg Movements in Sleep (PLMS) and Periodic Limb Movement Disorder (PLMD

60. The method, kit or apparatus according to any of the preceding claims, wherein linkage disequilibrium is characterized by a particular numeric cutoff value for the linkage disequilibrium measures r² and/or |D′|.

61. The method, kit or apparatus according to any of the preceding claims, wherein linkage disequilibrium is characterized by values for r² of greater than 0.1.

62. The method, kit or apparatus according to any of the preceding claims, wherein linkage disequilibrium is characterized by a values for r² of greater than 0.2.

63. The method, kit or apparatus according to any of the preceding claims, wherein linkage disequilibrium is characterized by a values for r² of greater than 0.2 and/or |D′| of greater than 0.8.


SEQUENCE LISTING (343 sequences)

SEQ ID NO: 1 - DNA, 599 bp, Homo sapiens
ctcttctgcaaaataccaagattttaaacactgtattcaaaatctaatattgtaagcctgatactatggttggatcctgtgaagtatgtgaaaacaccatggaagctcctgaaatttgtaatttagatatttttccaaaataatggaagtgaggattggctggggaggtgaggaaggaagaaaaggtccaacttctgtttctcttccaaacagaacaagaagtcttcagggattcagtaatagagatgacagaatgccatgtcttcaagtgaaaataaaatgatagctttctctcttctrtttatccatctgcaattcagtaggacaaatgcaatactctttttaaaaaatgggctctggaacctcaataaaaagaagtgagttctctgattcataaataatgtccccaaataagaaataaacaatagttttgggttatggttttgttttattaaggcaggagattggtactggcatgctgattttttccaatggattatccagttttcatccttaaattccacatgcctaaaattggattcatttctttcccaatctgacttatctctttgcctatttctgtcagtgctattttaatt

SEQ ID NO: 2 - DNA, 599 bp, Homo sapiens
atagggctgaggggttagttaagtttggggataaatgagatgggagtaaggaagtatactggacaaacaaagcattcttgtggaggaaacgtcaagtgcaaatgcagagagttataaatagcctgacaggtttaccgaaggagcaggagtgcggtactattggaatatataatagagtagaaggaatggagcaggagaaactgcatgggcacagagtgggcagatcatgaaaggctctgcattttaagctgtggatcttggactttatgccataggtgaagaggagccctgagaagtttyagtagaggagtgacatgacttcgttcgatttttacaactttcatcaagatacatgttcctaactacaaggagccccacctacttggttctgacatatcactgggttctatgaagcacaattttaaaaacactatattaaatgacttctctaggcctttaaacctaaacagtctatggttaaaagatcagtgagagaaatataaaattgtacttgtgccaggttcacaagtctctctttcttctctgtcccaatcctcccctcattcacactcagataactgtgtgcatggcaagcctga

SEQ ID NO: 3 - DNA, 599 bp, Homo sapiens
aaaactcctcaaatcacacaggaactaatggcatcatgacattgaacaaagaagtgttattttcttactactaagcatagaactatccagcattaaataatttatttcaatatcctctctccatactcctctcctacaactaaaagataataggggaagagatttactcttttattctttctgggcctgcatattaagagagacaacagagatattatgtctattattcttggtttagacaaaataagcaatgggaggcctcgagacatagtcactagctactaagctctctctcttctyagcctttacacatgaaaagcacagaataaatcataaagtactacttgacgcaccaaaaattaaaaatggagacagagggtggatcaatgtgcactgtctcacaaacacagtttcaaagataattggtaatgttctctttttccaaagaagtgagggttaaatatagtcactctgtacagagactgatgatcttcaggatgtgtaaattatacattgtagttacataacagactaaatcacagtcatcaacaaaagaaaattcggcacaattcttgaatttttattattctagctcataa

SEQ ID NO: 4 - DNA, 599 bp, Homo sapiens
gggcaggaggactgctttagcccaggaggttgaggctgcagcaggacatgatcacaccactgcactgcactgcagcctgggtgacagagcaagaccctgtctcttaaaaaaaaaaaaaaaaagagagagagagatggaaatcagcttcaggaaaatgagagggtaaagggaaaaaggactcaatggacaattttaaaaattcgggcttttggctaatggtcattccaattaaaatgcaaatgaatgaggcttctagcttttgccagcttgccaagtaccctatgacttactagtggcaamagccatcatgccttcccttgctacgttagtgtctaaaacaatgccctaaaagcttaagagagctggtggcagacagcaaagtagccagggcctagccacatgctctcaccaagatccaccctgaaggtgaaaactttcaagcagggaagcaaaggtaccagcatgcccccttatcaacatgggaaatacctttttctcaaaagacaaatgccacaatacagaactatgaccatctagcaagcaaatgacagatgggtctagacacataaggtaaaggacaaaattggttttggtgactc

SEQ ID NO: 5 - DNA, 599 bp, Homo sapiens
catgacttcgttcgatttttacaactttcatcaagatacatgttcctaactacaaggagccccacctacttggttctgacatatcactgggttctatgaagcacaattttaaaaacactatattaaatgacttctctaggcctttaaacctaaacagtctatggttaaaagatcagtgagagaaatataaaattgtacttgtgccaggttcacaagtctctctttcttctctgtcccaatcctcccctcattcacactcagataactgtgtgcatggcaagcctgaatgttaggaatatraggccataccaaggaatcaatgtcacatattcagacaaaatacatgggaaggaggaagtaaataccctcttgagggctgtcattgaaccatccaaactaaaaagaaacaatacaagtcacagcctgattattctgcagtggcctcatccaggccctttcgccatcctctgtctacctgctcactggctattaatgatttcattgcccactcaccctcccttcataggcacacaggtccagaaaagaaggaaacccccaaagaagttagctatgacaacaaaggagaagaaaattagcta

SEQ ID NO: 6 - DNA, 599 bp, Homo sapiens
atcaactatcatgtaaattcaacccttgttgcatggcatattcaaagttctattctgaccccaacctatatatcaaccttatttcctaacattccctacatacctcgcacacaccttatattgcagccaaactgaagcacttgtcatttcctaaatatgtcctttctcactcctgtaactttttccagaaatacatttctctgcctcttctgtctaaagaaatcccaggcatctttcaaggcctagtataaatatcgtaaggaaaccttccctgcaaatatgggcttccttcctctaaamtcctagagcatttgtacttctattacctcacttaatacatactgctttattattttgcatctatcaaactgtaagccccttgaaggcagaaatcatgttttattctcctctgtaactctcaagtttccctaggatagtcctttaaaattcaactcaaaagagatcaaatcatctactatatagagtatacccagaaaagatactcaaacatttggtgaatgaaccaatgaatgaatatcacaaccttagaatataaaactttcttaataaaataacatttacagacagaaatacagttt

SEQ ID NO: 7 - DNA, 599 bp, Homo sapiens
agatatagtttaaagcccttgagacttgattgaggtcaccaaatttgagcatgcttatgtatagaaaagaaaagagggtcacagatgaaggccaaggcacttcagttttagaggctggaggagaaggaggaaccggcaaaacgcactaagtagaaacagccagtgggataggaggaaaccagagaagagaggtgtcctggaagtcaagtaaatgacatttttcaaggagcagggtgttaacaactgaatccagtgctgctgctaagtgaagcaagacaaggactcggaagagactactgrgctcagcactgtggtaataattggtgaccttgacaacggcagtttgagggtgtaagatgagggaaggcctgtttggaataggtttcagaaagaacacaaccacttctggacaactttctttttcaaacattcttagataagctttttaaattcttccatcaagtcccttacaattgtaaaaagcaagcaacactaatgttaattaaatgtttagtgtatattatagtaacatattacttttaggtatgtcaaatttatggcataatagacatattttaatgtgttgaatgtctacttta

SEQ ID NO: 8 - DNA, 599 bp, Homo sapiens
agaactactggtatagagatagcaatctgcaatatcctggtgtcaatgagaagctggtttctattgtcaagaactctgctaatccctagagatgctgatgctggacacctgccagatggggatgttgtaaaggggattcaaatgttggcttgggtggctgggccagctgctcaccatggtctgttctaacagaagattctataattctgtctgagagggaacctaaagaagtctttcagagctttggctactgacatggaatatgaataacacttaatttaccttcattttgctacccaygaaacaatatgagtacattttttttccatggtgtattctcttcattttggtataatatttccaacattttggaagcaaggtgatttttaaattttgcttgttgagtttttattaaaagaaaagctttctcttccatgtccagcctcctaccatgttcaaatcacaaagaccagtaccatttgctctgaagggaaagaatgctaagggaagaggactctgagaatgtcctatagaccaaggtcaataacgaggcaaacattctgctgacagaaatccccaaggcaaatacacaaatacca

SEQ ID NO: 9 - DNA, 599 bp, Homo sapiens
ctgacttttttgagtgccaacatgatgagcaaagaaaatgctcattggagcatttcagatttcaggttttcggattaggaatgctcaaccagtaggtataatacaaatattccaaaatcaaaaaaagttcaaaatccttaacacttctggtcccaagcatttcagataagggacactcaacctgtatcttcagtgctttccacttgggcacatagtaaacactcaatacttgttgatcatagatgtgagtacatttttcttggcccctttttgcatctagtttttctttcactgcagtcyctgccaatttacatcaccaatcccatcccttggtgtataagagagcaacagttctcaaaatgtagtctggggactcctgggagtccctgagacccttctgggaggtctgtgagatcaaaacttctgaattcagagaagttatttacctcccatgtctgtgcacttgagctttccacaggctccatcacctgttctgtcacaacagattgaatgagaaatggagaatccagcagtcttctctttattatgccagacattaagatttgcaaggctactctctccattaaattaatttttaa

SEQ ID NO: 10 - DNA, 599 bp, Homo sapiens
ccaagagctcttctccctaactcttcaaagaatgtaaactggctaattaagtctggaaatgtccactcgatttcctgaatgtccatttggcaggctggaaaaaattaatttggggttgccattgtgtttttcactctgtataatttaaccgttaaatatcaaaggttcactgaagtaaatgaaagtagtttttaagcttaacggttagacactttaaaaattgttttcaaggcagcacagcacaaaggccagaatcatctgatcacaaattcctcccagggatataaaattaaaagcatrattcagtgtacttgtaaggctacatcttcccttctcacatccttggaataaatgaatgcagacctgagtgaagccagggtgcataacatgtgcttatgagccagcctgaaaagtacacatttcacaactccaggaagacatattaaagtcactgatgaataattattctgataaagctctaagatcctatgaggttaggctgtgatttgagttgacagctttgcttataaacattggttctaaaaaataccaattaatgattcatttgaatcctgctttgctctgatactgtccaaatt

SEQ ID NO: 11 - DNA, 599 bp, Homo sapiens
ctcggctcactgcaacctccacctctcgggctcaagcaatcccgctgcctcagcctcctgagtagctgggactacaggtgtgcaccaccatgcccagctaacttttgtatttttggtagagaaagggttccgtcatgttgcccaggctggtctggaactcctgggctcaagcgatcctcctgcctcggcctcccaaggtgtggagattacaggcatgagtcactatgcccagatgagcactaaaatctttaaataaacgctcttttctatagactgctaagagaaatagaagggaaaaawgcaatacaatacttaagatttactggagaagtactactgggaattgaggggagagtgtactctttggaaacagataaacgtagctctgaaaccctgccttgctccttgctagctgcatgattctgagcaagcttcttgatactcagcttcctcatccataaatacggggagcaatgcacttcctttgcagggctcttgtgggctgagagacagcatgcattcaacactcagagtacctctcctttgtcggtgctcaataaacagtagctcatctctgccattcctggagttaaggcaga

SEQ ID NO: 12 - DNA, 599 bp, Homo sapiens
ttgacagtttgccagtctgactcccatggcctgggcactgcttgagggtcatgactgtaccttctatttctgtattcccagtgctcagcatggtgttgcacacacagcagatgctcagtgctgtactgaatgagtacagacattggggtggggagagaggacaggtatttattagcacatgctatgaatgccatgctctttttgtatgttaccctccttgatcctcctcagagaattactcttaaaatgttatctccattttacagaccaagaaactgagacttacagggtatattaggwataggttctgttgcaagtatcagaggccctgagagaagtttatttctcacttaagtgaaagtccagaggtgagctgtccaggtattgcagctctgccgcatcaagtcctttgggctcattgctccaccgtctctaggacgtgccccttgttatcatggtccaagatggtagttagagttccagccatcatgtttgttccaggtagagatgaagggaagaatgaagaagggagcaaagggtgaatgacttttttttttttttttgacagagtctcgctctgtcgccaggctggagtgcag

SEQ ID NO: 13 - DNA, 599 bp, Homo sapiens
cacaggggatggggactcgcctctgtcctccctctccctgggcaaattcattgtcattgcctctgtcaccaggactttgaaccctgggagataatgctcctcgtgcttcctggctttctcttgtcccagctcgaggaagcagaatgtggaagttttccaccagcccttgccctgccctgccccttctgggcgcgtgggcagttgaatactagaaagatatcgacaaattctaatagcccactcagtcagaagtgttgaggccctacttctgttagaaaatagcctcctacttggaatttscttctttcagaaatctcctcttggcaagaaatttctttctccttggagaatctcaatgtattgaaacctccttcacctggggcatctctgctaatggagaatcccaaaggaatctcctttcttagctttttggttcttgaggctgagctgggtacattgcgggactggagtgggagagggtggaggaggctgctggagggaagagttcctggtgtttagtccaaaggcttagatggccatttattacttatgttagaacaatgttgatagattttagatgccatcttgggggcaggcta

SEQ ID NO: 14 - DNA, 599 bp, Homo sapiens
tacatcccacagggagtagaggatccagctggggtgtccatgctgctatcttctcttggccactgcatattgaccacatctggtggcccagtctttacacttaactctccttgtcacttctcattctccttgtaagctcccctggaacactcagctcagagtccactgacctcaagactttctactccacagcctagaaacttcctgaggctcagactatgtccccgtgattagaagagaagccccataaaggtaggcccagtggtgctttctttctctaaattcctctcagcacctggyaaggtttttcttttcattttgaactcctactgagatgaaacattcatacagaaaagtgtacgaaccatacctacacagctcaatgaattcacacaaacttaatgcacccatgtaactagcaccaggtcaagaaatacaatattaccgactggatgcagaattgctggagcccaggagttcaagcctgagcaacatagtgagatctcatctctaaaagaaattttaaaattagcctgacatagtagtccatgcctgtggtcccagctactcgggaggctgaggtaggaggatcacttgag

SEQ ID NO: 15 - DNA, 599 bp, Homo sapiens
ccttgcaaggctaaccattctcctaactcttgatatcatagattagttctgcctgtttttgaacttgatataaatgaaatcgtgtagtaggtattcttttctgtctggcttcttttgctcaacattaattttatgcaaatcactcctattgttgtgtgtagctgtagtttgttggttcattctcattgagaagatttaggagaggatgaaaattaaatatcatgtactgtggaataagagacatggtcttgaaaccgggctctgccacttacgaattgagtgttggacagctcatttagmctcacaaactttggtttctcatctgtagcataaagaagataaaatttcttttgagtggcacttgtaagatttatattaactacaataaccaaagtgtataaagcacctactaagttactccatgtggaaagcatttaaaaaacagaagcattattaattattaaggacttatttgctcaatgatctctgagaaggaaattgaacgcaatagttttgaattctcaatgttcatgtttcccccgtatgtgcgtgcgcatgcacatatataaacacatctagcttcacacctgtgtactcac

SEQ ID NO: 16 - DNA, 599 bp, Homo sapiens
ctcgtgatccacccacctgggccccccaaagtgctaggattacaggcgtgagccaccgcgcctggcaatgaatacaccagtcttaacctttcgacttcatcctgtacatctgagcttctttccaggtcataagcctttccttgagccactaattctgtctggcattccaaatgtctactcctgactctcaagcccaacctccccaactctggctccttccctaatttcaccctaataatgaattctaaccgttttatcctaaaactctctttttcacaaacagccttggttcttctcctygagtcagttcttggagtccatcccagccacacatggtgcattcacccctagaaatgatctgtacaaacaagcaagggacttgataaaaaacttcaggccagtctctgggatctcagagttacatggcagcctggctgtggcttctggctgcttttttaaaacctctagacaagaacggggaaggctggggcacgtgtcctggatggttcaccccagggagctcagggctggcatcaagcctgatgggctgggccaattcatgaagttcttggccatttattggtctctaagctgagccc

SEQ ID NO: 17 - DNA, 599 bp, Homo sapiens
tgtttgttgaataactggcaaacaggaaagcagtttggaaaatgacaatcactttttatgttgaattctccttttagttgtcgagtttccttttctgtacataaaagaaaatcatcaagatgccaagagagataatcccacttaatctttgttaaactcaaaggctgttaagtatctcacgcatgtgggaattttatgtcggcttaaatgaagatttaagggtaatatgaatccaatgagagcaaggtgtgctgtatggctttatcctttctatgcattttttctttctttctttctttwtttgagacagagtttcgctctcgctcttgttgcccaggctggagtgcaatggcatgatctcggctcactgcaacacccgccctccgggttcaagcgattctcctgcctcagcctcccaagtagctgggattacacgcgtgccaccatgcccggctaagtttttgtattttttttttaactagcgacggggtttcaccatgttagccaagctggtctcgaactcctgacctcaggtgatccacccgtcttggcctcccaaagtgctgggattacagacgtgagccacagcgtccggcttt

SEQ ID NO: 18 - DNA, 599 bp, Homo sapiens
cctgggcatagataatgtacttgtggctctgtggttgggagtggagacaaggaactccccaacaccggtgggggagaagagacaaggaagttgccccagacaaggggctgggggaggggcatttcccaaaagttcctgtcaagagaccagtataccgggtggaactataagagttgtttgggtgtggcgggaccaggaagagagagagagatgcccagggagttggcaaattggggagaatggctccccggaatcgcccaggagccagagcggcagctccagggaccttgactccgacgyccccgggaagccagagctccacttccgcgtttggcattgctcccccaggcccctcctcgcactcggttacgggttctacgggcgcatgcgccgccactctgggactggcccaatttaccctacacaactaatctgctcctccccctcccgcttggagcgcaccattcgcgtttagggggaggcaccgcgccccggagtactcagaaacctgctccccttcttcagacagggggaagataagtttgcttcagattctaaaagttctaagaatacgaaggcctcagccgcccgtcgccggg

SEQ ID NO: 19 - DNA, 599 bp, Homo sapiens
cagccttttacctttagccggtagggttctgataatgccaatagagtttggtggcagcagacaggaaaagttacactaatgttgtacttgacctataagcaaccagggcttctttttagatattttcagtctgatactttttatgaacttcagtttacctagcaggagtagtgaccacaaaactagtatttttatcgtgaaaagtgttctttttggtgcaatgttttaacctttcctaaggacgtttgtagacaattgtgtagtttgtattttataccaaagcatagcaacattgccgcyttgtttccatcgtatcacaactgactttggacatgtttcccttactgtatgggccacattcatattttgttcactgagtaaacttagaatatatttaacagatatttgaatgcctgtgatataatggtgggcaaggaggtgctggccccatactctggggcagggggcacagggaagtaaactgacagttgcaatacgttgtacaggtgctctaggggaggggcacctaccctagctggggtggaggtagaggaaagtagcctggagaaagtgacactttagcctagacccacaggaag

SEQ ID NO: 20 - DNA, 599 bp, Homo sapiens
ttcctatcttttctgtaatattctaggtcttcacatttgtgaagaatctacacacctcagcactttttaaatgtctaaataatttaaaatacagcttcaactaataccacttatatctgtatagtttaaaagtgttttcgtgtatgtgtatatttcatctaaagaaagtgtactaattgactatttcatttcagatacaacaattatcaacaaatacgttttttgcctgagtactaaatgcctaaaatgagccaagaaaatgttcatatttcacttaaagttttaagtccatgtctccargaaaaatttgttttaatcctaaaaatatgacgttaatatataccggccaggcgcggtggctcacgccggtaatcccagcactttgggaagccgaggcgggaggatcacctgaggtcgggagttcgataacagtctcccacgtctctactaaaaataaaaaattagctgggtgtggtggcacgtgcctgtaatcccagctactcaggaggctggggcaggagaatcacttgaacccaggaggcagaggttgcggtgagccaagcttgcgccattgcactccagcctgagtaatgagagca

SEQ ID NO: 21 - DNA, 599 bp, Homo sapiens
gaagagaattctaaactttaaatcatactgaaaagttttgactaaaaatctctttataatccttaatggtatcaaaattaacattagttttcacaaattaacctttccttgctctaaaaatcagagaactaagaaggctttgaaaagaataaagtccttaaaaagaaaccaagaacagtatataaaagattatttatttagaacagaatacttagagggtgagaactaatatcatttggattttttattaggtcatagggaggttctgtccatgcaaagtgatgtagtattttgaattcytgcaataatcacagattaaaattgaggttaaatattgttgttttctttttcacccaggcctgggactggcaggctgcatggctttgtggctgaggctggaagcagagcctatcaaagtcccctctcttgcaaaaaaaaaagaaattcttttctgtttttgaagaaaggttaaggttgatatctcagcaaagctacttgaaggaagtgattggtcttgacccagcacttgtaatgcagtacttacaaaatcatccagattctggctttttggatttcgtgggactttgtgtcagagtggt

SEQ ID NO: 22 - DNA, 599 bp, Homo sapiens
gaatgggaattaacatttcagtaatgggaattataatgtagtctgtaatttgaaccttttttgaagtgttacttttgagtcagacattttagctcttttagcactgttaaactggcaacctcagcggcttgtacagggaggtattggtctaacaatcagtttctttttggttcaaagaatttttgaagctactcacctttttgaaagttgataggttgtagataggctctagttagcttttatgacttttgtggaggaaaaatatttgttttcacaaatgtaaatctcatggcagctttytatatgtgattagggataagtgcattggattttctggtaaattcaggttaccatatatagagaaaggtggggtatattggaatttccagcagtagtggacaaaaccaaactgatctgtgatgcacccaggctaatagaaataagtgaatgaaaatgcatgtcatccatgaggctgagggacagagctgcaacagtgccaggcacctgaagttttggctacagctattaactcgagggatgatgtactagctaactacatctgttcatttagctgtaccagatgtcccatatcataggca

SEQ ID NO: 23 - DNA, 599 bp, Homo sapiens
ccttggatggaagaggagggccaagccacaccagagcattttgtttccaggttgtggataagctctagttagctttattgatactttagtcattacctggcagagtgaggttggtgatgtacttaggacctttttgggagtgtcttttctcatttatttctgctgaagtagagtggtgtggaggaacagctgttgatgtatctgcttcttggactcttacacactgataagttgaagtgcagttgcataaaataatcacactcacagccacacttcctggaaggagaaacattctgaccrttcaagaatatcacaaacactcttcctgtagtcagcttctttgtccaggcacttggttttattcatttattttttacaagaaagtaaaaaaaaatttctgtggcttatggtacctaactggaagagtggaggcaggaagttgaggaacacagagtgggtggtgggctttgctgtcagttctgtgctttgaaagcttttatctatttcagatcataattttttaatggcagccctttctaggatgctttcggcagaaaaatgtactggcaaaatcgtctcctgaaaagatttacgatgag

SEQ ID NO: 24 - DNA, 599 bp, Homo sapiens
tttaaaaaaaaaaaaagtggaggatgccagacttggtggctcatgcctgtaatcccagcactttgggtggctgaggtgggcagatcacctgaggtcaggagttcgagaccagcctggacaacatggtaaaaccccatctctactgaaaatacagaaattagctgggcttaatggcctgcgcctgtaatcccagctactcgggaagctgaagcaggcgaatcacttgaatcaggaggtggaggttgcagtgagccaagattgcactactgcactccagcctgggcaatagagtgagactcyggctcgaaaaatgaaatgaaataaaataaaataaatggagaagtgtttttgtatattgacccacaaaaatacttgcatgtagaaacttttaattttttttattgatttgggaggacatgttcatgtagtctctgaggcctgagttccctgtttctgtggatcagtcactgttaactagctcaccctgttcccccaacctcacccatgctgtagcacttccaatatgtgcttaaaaccgtgcattttatttgtaaggctgatgatcttcttcagggaatattgagcaggatctattaagg

SEQ ID NO: 25 - DNA, 599 bp, Homo sapiens
gatttaatgagctctgatgcctctcaagcccttgccacagagcctggtcatggtaattacatttattaaaggccagcttcccaacttgaaagaaggagcactgatggcagattagtaaatacagaactatcttacctcatatgggaaatttgcatttatagtgatctccagactgatgccaccttattaattggtgaagtttctgaccagcttttggcccctttggccattttccagagccagtcctttttccagcaagtgttaagcactctattgtgtgtcttgagctatgctaggcargagataccttggtctggcttctgccctgagaaagctcacggtctagaaggggagatggatcaataaactgacaaaacagtgttcgagaagtgctctgggagatcactgagtattgtgagaactctaatgggcagcttacctagatgggaactgagggcggccaggagagcttctcatgagttaggtttgagctagatatgaaagacgagcagaagttaaccacatgaagatggtaaggaggggatttctgggcagagggaacagcatgtacagagaggcacagaggaatgggaaagtgt

SEQ ID NO: 26 - DNA, 599 bp, Homo sapiens
tgcattcctatcagcagtgaatgagagttcctgttgctccatatccttgtcagcatttggtggtgttgaatgctgacactatatgatacatatgcatatatcattatgtatatataatgatatatgctcatatgcatatatcattatgcatatataatgatatatgctcatatatgcatatatcatgcatatataatgatatatgctcatatgcatatatcattatgatatatgtgtgtatgcatgtatgtttcaaaaaaaaaaactagatactaccagttttcaaaaatgattatatcrgtttacattcataccaccagtatatgagagttccggttagtccacattttgtcaatagttgctgttgatggtcactttcattttggttgtataaagttattgcatttttcttttaatttatatttctctgatgactaacaaaatagagcatcttttttcattggccatttggctaaactttgttgtgaggtgcctgtttaagcattttgcttacgtttctattaggtcatctgcttttttctcattcatttgtaagaggtttttgttttgttgttggagatagagtctcaccactctct

SEQ ID NO: 27 - DNA, 599 bp, Homo sapiens
tcttttctaaaaaacacattactcatgctttccctagccccagtttagtgggtccacgataaggtttggatagttcatccttgaccttttttctttcatctcttgaaaaagtgtcactttcttttgagacccagtggtatagagtgaaaagataactaaggatggaaccctgaggaacacaggtagaaaaagatgagtttggaaaggagttggatacactgagataggaggtaaatcaggtgagtataaggttagtgaaaccaaaggaggtgatatttcaatgaagaaatggcagtaggktcatgctgcaaagaatggtcaagttaagatgataattgagacatctctgctccttgaaggtcctagaagaacttgagtaatgcttttaaagtcatgttagtgcctctcagcagtattctggcccagcatggtaccagggtatgttttgtgaaaaattgttgtaattacatcctatgtgtaccagaaatgaacatctttcattaatcattcatgaatctaaccatcatgaattgaaacttctcttgaatctatattttcacttctattaacacttccaataatatatatgtttttagggt

SEQ ID NO: 28 - DNA, 599 bp, Homo sapiens
ctgaaaagatactaatactcagattccatgtctctccccctcccccgtataggttgagatgtcatagaaagaaggcgttcatgtgggagataatgaaatatatttcttaagaaaagctcagtatatatataggaaaaattaggtgaagtatgaggtaagatgaggggacagacttcttggctgatactggtttatgctattttattcatgacatgtgctgtatgtgtcaatatgacacaagaagaaaggacatattggaattattagaggagacagtgtgaggattaaggtcaagtagtwtttttcagatattctaaaaatcttattttgctgtgtgtgattttctgtaaacattcttcagcgtattttgctatatgtaactgctctgttgttattttagggaattttaagcatttcagcattggaactgtgtaatcagagacaatgggtccttttaatgtatagatagtcctttttttttttttttgagatagaattttgctcttgttgcccaggctggagtgcaatggtgcaatcttggctcactgcaacctctgcctcccaggttgaagcgattctcctgcgtcagcctcccgagc

SEQ ID NO: 29 - DNA, 599 bp, Homo sapiens
tcaacacagaggaaatttagagatcttgagtaaatatttcactttagttatgaaagagggtaatgaatgaaagagggtattttaagaagggtttccgaataagtttcagttctttgagtaataactttatggaaagagagactatttctctagtgtctgattttctcctcccccataacattttatcatgagaaatcaagaattgtacatccatttatttactacctagattctgtaatattttgctttatttgctttccatccttctatccatctattaatcttattttttattttccragtaaattgtagactcaatacatttacccttctgtacttcagcatgcatatcattaactagagtttaacatttgtttttaagtttttgttttgttttgttttgtttttgagatggagcctggctctgttgcccaggctggagtgcggtggcgcgaacttggctcactgcaagctctgcctcccgggttcacgccattcccctgcctcagcctcccgagtagctggggctacagggcctgccaccatgcccggctaatttttttgtatttttattagagacggggtttcactgtgttagc

SEQ ID NO: 30 - DNA, 599 bp, Homo sapiens
tatcattactgttttgagaactttatcctttctttactctttgtcacttggctttgacagttgcaacctggattgggcactcaagttttattttgttctaacatgctgagtttgactagtccattgtatttggcactttgaaaagtactttttaaaaatagcttacattgagatttgcttttgagaagctactaattggcagttccaaactggacagattgaggccaggtaaagcctgtaggtgcctgtcaggcagcggtgaagttaaaaatcacctgctgtcaacaccagggaattctrtaatcattttcatttttgaataaatggagaatcattagattcctggagctgcagcactgaatggggaaaatggttattctgcctatgtgcactatttcctttgtctctgtagcctcatcataagttactctttgctaacctctttctggctttggttaaaaattatttttctggtaagtttttaattaggaaatgcagaaatagagctgcttgagttgtcgtaacttaaactagccatgaaacttacaaactggagagagagctgtctcgcttagaatgtaactgagtttattgttgaa

SEQ ID NO: 31 - DNA, 599 bp, Homo sapiens
aagtcttaaggcttataagccaatagtgtcttctattattctggtaattatgataaactgctttgcaagttgagatgaattagtttggattctatattcctgctgctcatcaacgggggctgcagggggccagaaggttggagtgaagatggaggagaatgggagagacgggggaggaccatagacacactgcattctcaagcgagagctccccttttagagttgcattgttctattcaatttttctttttgactttttcccctctcttcttctttgggctatggggaggtaagtaattyataaacacctgtcttccccagatgaattgcgtcatataatagtttgtgcttgtgccattagtatttttattatttttcttcttattatttttgagacggagttttgcttttattgcccaggctggagtgcagtggcgtgatcttggctcactgcaacctctgcctcctgggttcaagtgattctcctgcgtcagcctcctgagtagctgggattacaggcacccaccactatgcctggctaattttttgtatttttagtagagacggggtttcaccatattgaccaggctggtctggaa

SEQ ID NO: 32 - DNA, 599 bp, Homo sapiens
aaatggattatattctaaaagggaactgctgatatataaaatataatatggttttagtaaatgcattttgtttgcaagcctttgctcatgagcaatgtaataggtacagaagtaggaagaaaatactaaaacccctaagaatctaaactattttattaggcacctgaataaataagtagattgtataataattcttgattaccagtaaactatttcggggggaaaacactaatcttacaatgtctattgtattttctgattataataagaatgtatttttagtacagaaaactgaaacasataaaaatagggcttttgcctttttaaaaaattttttttttattttttagatggagtttcactcttgttgcccaggctggagtgcaatggtgctatcttggctcattgcaacctccgcctcctgggttcaagcgattctcctgcctcagcctcccgagtagctgggattataggcatgcaccaccacacctagctaattttgtatttttagtagagatggggtttttccatgttggtcaggctggtctcgaactcctgacctcaggtgatccgcctgcctcagcctcccaaagtgctgg

SEQ ID NO: 33 - DNA, 599 bp, Homo sapiens
actgtgtcgcccaggctggctggcatgcagtggtgctcaagtgatcctccagcctgtgcctcctgagtagctgggacaacaagcacaagccaccaggtctggctaatttttatatttttggtagagacccagtttcgccatgttgcccggccggtctcaaactcctgagctcaaagtgatctgccccactcggcctcccagattgctgggattacaggcatgagccactgtgcctggctggttctgtttcttctctagtcttacttctgtcaggctcacttacctgacttcatgtagaarcattcttcctggatccatgttattctcttttctgctaacctgatattctctttctagcagtgctagttacttgtttctttcagtattcagcatatatagcttttcgtgactgcttcagattacactcaaaattccacctgcacaatgaagcagataccacctacggacttccaccttcccttatttccccttccatttagtcaggggcaccctccactccacctgtgtatggtttgtatttactttaggttgtctttaagttctaaactcctcaaggcacaatttgattttgttctctt

SEQ ID NO: 34 - DNA, 599 bp, Homo sapiens
tctattaaagacagatgatcctggcacacttttgcttaaattttatttcagtgttctgtgtttggcaatttggacttggcctgtaatatcttctgttgctgaaccctttcttctcttcatcgctcttccataccatctgtagctatgattgtctgtagttgtcaccagagactttgttgctctttaaatagtatattgtatgttacaaaatacataatttctacctaccaattttctgatcttatggtctattttcatatcttatggtcatgagctaaaaattaatgtttgcttattggrgaggggcatgatagcagcaggctggagaccttcaggtgaggctttgatttcagtgataggtgaggattgaattcatgaatggcttgcaaatattgtgggactggccttgtacctcaatcagtttgtgtaacattttagaatgtagtagcttttctccattgtcatttgaattttgatgaataatgatttatttttctgtctttaaaaagggtttttttttttcctgagatagagtctagctctgttgcccaggctggagtgggttttttttttttttctttctttttttgagatagact

SEQ ID NO: 35 - DNA, 599 bp, Homo sapiens
ctttatctgttcttacagataaacttctccataattgcacacttatctggggatctgagataagcaaaatgatggtttcttaattaaatatgagtactgtgcagtagtaaccataattctaaatgaggattatggatttttctggaagattcttttttcctgtggaacatgatgagaaatgtttaggagaggggacatagccatttttgtatgaagaccaattcaagaaaaaaatatatgtatgtgtgtgggtgtatatgtgtgtatatatgtatatatgtgtgtatgttcatatgtttrtgtatgtttatatatgtggttatacacacacacacacacacacacacacacacacatgcacacatacacgcacactctcactctatatttattctttggtattcctgggggattgattccagaacctcctgtagataccaaatccgaggatgctcaagtccctgatgtaaatagcttaatatggctgggcgtggtggctcacatctgtaatcccagcactttgggaggccaaggcgggcagatcacttgaggtcaggagtttgagaccagcctggccaatatggtgaaaccatgtcttt

SEQ ID NO: 36 - DNA, 599 bp, Homo sapiens
taggttacccagtggttttcacaggacactcccaaagattgttgaaaaagacattttgagtgtagaagtatctgagatactggtgcactgtacttggtgtagtgtattatccagtgacctttgaattttctgtgtcacttaaagaaaagaggttcttatttttacttcaatcactgtaaatgtcagtgagagatagctctgtggtcactgactttcaacagtggtaaggtcaggtaagaacttcacgaagccttttaggaatttgtctgtaaagggcactgtgttgtcatgaaacatacrgactgctttactaaggacctgggatgctcacacagaaggcaactccatttgtggagaaggcttattttgtctgaattgtttaatgtcttgtctcagacagatgatgactcaaagtcaaatgttcacctttaggggacccaaaaggatacaaaagagaatgcatagtaatgattggcctataggcggttgagtggcaaagctagtttaaagaagaaaaatccttcctatctttttttgtagttctgtaagcattatttgaagaaacttggatttccctgtgtgctttaaagtgaagtgca

SEQ ID NO: 37 - DNA, 599 bp, Homo sapiens
acctctccatttctggctttctccttttcttgatttccccctccactttccacctgatagaatcactctgaactctgtcatctggttcttcatctcagtaagacagtagtttctatctggattctattacctaacgtggcttacgtaggccttccctcaggcaaaaagccatttagaaaaacaaaaggaaactttctcaatgcaattcccattttccggtgcagactcacagtttctgcctggttttgattgctgttcagcgccttcaggtagttactttttatgttttgtccagagttwatggtttttacgtatggaacagtggggccagtaggaacatattcagttccacttctagtaatcttaggaagtatctcctggaagtggaacttgttttttgttaacctttttttgttgaaatatatcatacacacccgcagtagggaaatagaatattaccaaaacctcagaaacccctttcatttctcttcatgtcccttctcagtcattactcccacttgcctctccagagtaccactctcctactctggcaccatagattagttttgcttgttactgaaatttacgtaaatgatatc

SEQ ID NO: 38 - DNA, 599 bp, Homo sapiens
ggcttacgtaggccttccctcaggcaaaaagccatttagaaaaacaaaaggaaactttctcaatgcaattcccattttccggtgcagactcacagtttctgcctggttttgattgctgttcagcgccttcaggtagttactttttatgttttgtccagagtttatggtttttacgtatggaacagtggggccagtaggaacatattcagttccacttctagtaatcttaggaagtatctcctggaagtggaacttgttttttgttaacctttttttgttgaaatatatcatacacacccrcagtagggaaatagaatattaccaaaacctcagaaacccctttcatttctcttcatgtcccttctcagtcattactcccacttgcctctccagagtaccactctcctactctggcaccatagattagttttgcttgttactgaaatttacgtaaatgatatcattcataggttttgctttttgtttggctttcatctttgtgtttatgaggttattctatgttgcatgtaatttgttcattctcattgctgggaagtatttcattgtgtgaatttactgtgattagtttatcccttcta

SEQ ID NO: 39 - DNA, 599 bp, Homo sapiens
ttattgatagaatctttattgtgtaatgtatattattaggactttcaaatgtgtttcatttttagaagctacataatacatattttaagcagcgatttatttctgattatttttgcagtatgttagaaaagtaaaataatttgacatgttaagacgtgttccatccatcttcagggtacttctcatgacatttcatttgagcaactaggctttgtattttgttactgcttgcatttagcactcaattcttacttttagataataagaaaaaacaggtattttaaaaaaggacatgtagcwgctaataatagcatgagtcctatggttccttctctgttcatatcctagctgacaacacatacaagcattgctttgctggaatattctgaaattaacaattttgaataagtttatacatgtatgtttataaatcctttcccagttaacattttttaattttgtgtttttaagtaaatagcacttcagtttgagtccctataggttgactattatcaaatttgagatgcttacatgagtgatccccgattgctgatatttggacatttgggtctcaatagagagattgcaggttgaatgga

SEQ ID NO: 40 - DNA, 599 bp, Homo sapiens
tgcagtggtggatctcggctcactgcaacctctggcctcccagattcaagcgattctcctatctcagcctcccgagtagttgggattacaggtgcatgctgccataccctgctaattttttgtattttagtagagatggggtttcactgtgttgcccaggttagtctcgaacccctgaactcaggcaatccactcgcctcgccctcccaaagtgctgagattacaggcgtgagccatagtgcccggccaaagtatacattattcttaagggcacatgggacagtctccagtatagacaaygtagtaggccataacataaacctcaataaatttaaaaggatggaaataatacaaatcattttctctgaccatatgggatgaaattagaaatcaatagcagaaagaaatttggaaactctaatacgtggaaattacagcatactcttaaacaattagctggtcaaagaaatcagaaaatactttcagatgaattcaaatgaagacatagcatgtgaaaatgtataggatgagcagggtgtggtagcacgtgcctgtagtcccagctacttgggaaactgaggtggaaggatcccttgagc

SEQ ID NO: 41 - DNA, 599 bp, Homo sapiens
ctgagattacaggcgtgagccatagtgcccggccaaagtatacattattcttaagggcacatgggacagtctccagtatagacaacgtagtaggccataacataaacctcaataaatttaaaaggatggaaataatacaaatcattttctctgaccatatgggatgaaattagaaatcaatagcagaaagaaatttggaaactctaatacgtggaaattacagcatactcttaaacaattagctggtcaaagaaatcagaaaatactttcagatgaattcaaatgaagacatagcatgtsaaaatgtataggatgagcagggtgtggtagcacgtgcctgtagtcccagctacttgggaaactgaggtggaaggatcccttgagcccaggaatttgaggccagcctggggaagcaacatagtgagatgctcttaaacattttttaaaaaatgtattggatggagcttaagtagtgcttacagagaaatttacagctgtcaatgcctatgttaagagggaagaaagatctcaaatcaataccctgtccttctaccttcactagaaaaagaagagcaagtgaaatctgaagcaaacagaaa

SEQ ID NO: 42 - DNA, 599 bp, Homo sapiens
gtaaggttcatctttgttgttgcttatggtagtaattctcatcccattgtgtgaatacagaacagtttatggatttattcacttgtgggtagacattggcttatcttccgtctttgttaggtgtaatgctgctgtgaatgttgctttatatgtctcttggaagaaattcagcttcgtatttcaagagtatatagatgagagggaatcggtgagtcataggttatcaatgctaagcttttgcggtagtggctataccagtttactctcccagcatcagcggattagtatttcagtgttccrcacctttgccagaatttggatattttcagtcttctagcctctggtggagttatattggtgtggcattttaattttaatttgcattgtctgatgtctactgatgttgaactgttttccatttttttattgaccgcatagatgtctgcatttttgaagtgcctgttcaagtcttttgtccaatttaaattgggaatcaacagtcagcaagtagtcagaaaagaatagcaaaaacacacaaaataaataaattgggagtcagtcttcttattgatttatagttctttttatagtttacgcac

SEQ ID NO: 43 - DNA, 599 bp, Homo sapiens
tagtgtcatattatactcaatagattcagcatactttagtctctttttatggacatttaggttattaccaatcttgttttaactgtttaactgtgatttagttatgtaactattttaaacaatactgcagaaacccttgcaatacaagtttttctgcgtatgtgtgggaatttcttcccatattatcttaatgtttttatttataacttcttttatactgtgttctgagaacattacatagcaaagtactctttcagtactcttgagaattagaaattactattcaaaattttactctcytggaataggcttgcagaagacatctctttttcaagaggagaaactcatcttacaaatggtgtttgagcaaacttccttttgtgtagaaattcagtgtaaaattttaatatcaaataatgggcaatattgagttgttgcaattttcattacttttaaactgatttagttttctaagtagagtgcactagttctgtagatttttttcccctctttttggaaaagcactagtattttgtaaagaatacattacttgcttgttgttagtatttgtagtaacaaggttattaactcattcaaca

SEQ ID NO: 44 - DNA, 599 bp, Homo sapiens
atgtgtttgtctttctaatatggaaatgaaatcatcaattactaacttccagtatgtttcagttaagccttttagaggggcttcagccttttgaatgaaagcagtgaggttgagtgaaggtttgatactcacttccatgttctgtttgctgcatctgaaccactagcagactgacttgaggtttctctcacaaaagctgaagggattcacaggtgcttaggttaagtactggagagaccttcttgctgaggcctttaacaaaattgcaaaagacaaaagtaatttaattccttacttggwtctctggtgctgtatataaaaccaattgacatgtaacacttgtagctgttgagggttaattttaaatattctgttagtttgttgagtgtcctctggtaattctgtttttaaattcagcgtttgtatttgtatatttggaaagtatagatactgcctgcaaagggtaaaggtggggttaatttggaatctgaatataaaatatttgttataaaatgaatataatagcattatggccaggtacagtggcttactcttgtaatcccaggacattgggaggccaagccaggaggattgcttga

SEQ ID NO: 45 - DNA, 599 bp, Homo sapiens
tatatacttggtaatataaaaatttatttcggaatataaaataaaggactggacttcattactttttaagtaaacttttattggatgcttccttttaatgtatttttttttgtagaaatgaaattgttttaattttatagcttagcctgatgcctgattccatgcttgcactgactctctgtctgcactaacaaattatcagatttattcctttccaaaatatcattgattttttttcagatcatagaatattcctctcccaacttttttgttgacagctttactgaggtgtaatttacrtgccataaaatacacccattctaactgtacgtagttcagtgatttttagtcaacttaacagcattgtaccacagttaacacaatgcaatattatagcatttctatcattctgaaaagttttcctcagaccttttggaagtcattttctgttcgcgccccttggtgaacactgatgctttctgttactcttgatttgccttttctgagtatttcacataaatggaatcataatgtatagtcttttgtgactagcttctctcactgaacgtgtttttgtggttcatctgtgttgtcgcatg

SEQ ID NO: 46 - DNA, 599 bp, Homo sapiens
tttcctaaaataagacaacaatgaagtttgctgcatcaattgcctcttttttatgaaatatttctctatagaatgtaatgctgtttgatagcattttacccacagtagaacttctttcagaattggaatcaatcctctcgaaccctgctgctgctttatcagctgagtttatgtaatattctaaatcctttgttatttcaacagtgtttacaacatcttcaggagtagatttaactcaagaaaccattttcattcattactcattcatttcaattttattatgacattgcagtgattcayatcttcaggctctacctctgattctagttctcttgctgtttccaccacatctacagttatttccttcactgaaatctggaacccctcaaagccatccatgagagttggaatcaacttccaaactcctgtgaatgttgcttttttgacctcttcccatgaagaggtatcacagatgttcttaagggcaactagaatggtcagtccttcccagaaaatagtctattatttactttgcccagctgtctatggcagctatattctttttttttttttttttttgagacagagttttgctcttg

SEQ ID NO: 47 - DNA, 599 bp, Homo sapiens
ggagtagatttaactcaagaaaccattttcattcattactcattcatttcaattttattatgacattgcagtgattcatatcttcaggctctacctctgattctagttctcttgctgtttccaccacatctacagttatttccttcactgaaatctggaacccctcaaagccatccatgagagttggaatcaacttccaaactcctgtgaatgttgcttttttgacctcttcccatgaagaggtatcacagatgttcttaagggcaactagaatggtcagtccttcccagaaaatagtcyattatttactttgcccagctgtctatggcagctatattctttttttttttttttttttgagacagagttttgctcttgttgcccaggctggagtgcaatggtgtgatcttgactcactgcaacctccgcctcccgggtttaagcgattctcctgcctcagcctcccgaatagctgggactataggcacaggccaccatgcctggctaattttttttattttattttttcatttttagtagagatggggtttcgccatgttggccaggctggtcttgaactcctgacctcaggtgttctg

SEQ ID NO: 48 - DNA, 599 bp, Homo sapiens
attacactgctttttgttaggtagcgagtagttgtatgcctgtgattagcttcaagtttcccatgttcaacatgagaaaggatcaaagttcccggaagtctctgccagttcttttggattcagtgttcttgacccatgtgatccaagctggcattgtttagaatgaccattatttcggtagccaaagtctggtttattttagatttaaaacatctctgttttgagtaataatatcagtagttatattatttcttttataacctgattgacaaccatgttgctgaaaaatttctatccagwatgtggaatggatgtcatttaggtaatacataggtatggtaggcattttaaatacataaattatctgaagagagatcaagggactctgagaaaaaggccattttgccatttttttaaaccaggtttctgaaactcagatgttaaaactggcacactggttgccaagttatcctctgaaggctttccatgattcctgctgggcaattgggagtagcagaagttatatgagttatatctggctttttgtggcattgcgtggacaagagatagtgaccgcatcagaagggcgcctgattctc

SEQ ID NO: 49 - DNA, 599 bp, Homo sapiens
tttgtgacctagaacagtctgcccacctatgtaatggggatgagactgtatgtctttgatttctaagacccccttctaagttgaataatgccttggcagaggaaggggtaacatagtgttccattattggaacactaagcatgtgggaattatttatatcctgctcaaggtcattgccctggtctcactgcaaaaattcaaaaaattccaacctctggcataaacgggttaaattattcatcgcttgtaaccaattctaaggtatttccttgttacagaaatgctaatgtgatttttttwaaagtgcgttttaaaagcaaaacaatatggtaatacccatcttagtgatgttgggacgattaataagataatgtatgcgaaatgcccatccaagttagtattcagtgaattgtagctattattaatggatgaaaggacatgaaaactaatttgattggcgtatctgaagaaagaattttaaacaagaaggtcataatgtgattaggatctgaaataggaaagatcaaagttcagctaatgttatagagataaatgactgtgtatatgaggagaaagttttacccatttacgaagtattt

SEQ ID NO: 50 - DNA, 599 bp, Homo sapiens
ttttttttttttaatttttattttgttgaggggggagtggtggctagctagggacttgttattaatttatcagtttattagttcagttgcctgaccagaaagttctttgcctacgtttctgacatcttatgttttgattagagtgatatggagtttttagctcttctttctttcttttctttttgtttgccccaggtccagcaagactatgaatctctgttccaaatgctttgctggtaagtgcagaaaaagggttttttaatttactttcatttttctttctttttttttttttggttstttttaagactaaatcatttgaccctaaaggatttatgcatggggttttgttttcttagcttgtgtgagaaagcagtttcaccttaccaaggtgcttcatatatttgcctaggacggttgaaacagttttccttgaatgaaatcctatctcctatgtacttgcaccaaacaaactttccttggtttactgttgttggtatacagtagaactgtaaaaaatgcgtgctcatcttgacttgaatgattattggttaatgttgctctatcaatgagaatgtaacagaatattttcatgttgg

SEQ ID NO: 51 - DNA, 599 bp, Homo sapiens
cttctttattcattttgtcaaaggtaaacaaagctgcatactagctaaagttctaaggacaggttttagtcagtaatacacttttgcaatagggaagagagtccagtatgaaatgaagtcaatgttgatttgcagagatgactgggagttttaaagggagaatggcaggggacttggcagggtggataaacggaatctgtagactcaaagaagtgaagaatttaaaaagattggtaaaatatggtgattaggccagctgtgcctgttagctgacactgtgactggggaatagataccctrtttttcctgatggttacatttttaaaggaatggctttcaggtccttgagaaagacactcctgagttgtaggagtacagctcaaagggacagaggaaggattcacagttgtaagccctttttagtaaatgatctaagaggatagttagggcctatcttcagatgctggctagaacaaacagtagattctttggcagccttgagctttaagaggcagacaatttgagagaggctagggtcatcctagggaggtaaccttgagctgttagaaatgttagtatttaaaattagctggatgtgg

SEQ ID NO: 52 - DNA, 599 bp, Homo sapiens
gaccacgaggtctggagagcgagaccatcctggccaacatggcgaaaccccgtctctactaaaaacacaaaaattagctgggtgtggtggcccgtgcctgtaatcccagctactcgggaggctgaggcgggagaatcgcttgaacctaggaggcggagtttgcagtgagccgagatcacgccactgcactccagccagaacgagactctgtctcaaaaaaaaaaaaataaataaataaaaggactgtattctgctgcttaccttatgtatctacaattacagatatgaagaaatgcttcytcactgatgaaaggttaactttatagtgaagacgaaagtagcagtgagacaaaatgtatacaggttgagaaccccaaatcccaaatctgaaatgctccaaagtgcaaatgttccaaaatctgagaaaaacaaatcccaacacttctggtctcaagcattttgaattaggggtactcaacctgtattagattatatatttgtccccacatggagaccccattgtaatttagtaaagtatggctttgggagaaggccagacacctttgaataataaggtggttcacatcaaatactcctga

SEQ ID NO: 53 - DNA, 599 bp, Homo sapiens
tatcagtgtattgcacatagtaaagttattttgtgaaacctttgtttgacttgcattgagttgaaaataatgtgtaatgggttataatgtaaatatgtgtatattactattatgattttaagtttgaaacccagtgaactgcaggagtatggctttggaaaatcttggaatctaatttgctttgtaaaatagggaatatttcatttgtgtcttcaggcaagaggttaatagttgatttcttgtgatctttgtcagttctgagctgttgagtagtttagaaatgaagcttaaactagaccygatagcctactacagtgttaaaatacatatgaaaagtcaagcatagagtctaatgaatattcctgcctcttacaaaggtagaaatgatactgcctatggtatttttttttgtttgagtgcaaatccaattcatgaatttgtgcattttagttgaccagtgtttaatatttaggaatagttagtacctaattcatgatgacctcttgttctagcatactgaaggccagctatcattaaagcagtgcttttcacagaatggttttgctgacctcctaaatagaagtgtggatggcagaagc

SEQ ID NO: 54 - DNA, 599 bp, Homo sapiens
tttgccttttttcttatcacactaaaaagggcactaaaatgtcacagttttcttttaccctagtcatcatcttcccaactcagagacaactactctccttcttatccttgtatttacactttttattatgaatgggcatttatataaatattgttatgattctgtttttaaaatgtttaaatggtattttgtactagtctacaacctgtattcttttaaataatgcttttagaaactaatcatgttgacacgttgctcttgttcattttaactataatattccattataattaatgtctrgatttacctagaacaatctccgtttatgcattttgtgccagtgaaaatactaatagtacctctttgtaccaaactggtactattgtaagtgtaccaatttgtttgatacattaattggtcaccttcattataatgtgccctaatttatttatctagtcccctgtagagggacatttgttttcagtggccatttttataccacagttgcaatcctgtgttttctctctctctttttttcagcacttgcttgactatgttaaaagaatgtttctgtaggaaactctgttctttatggtgta

SEQ ID NO: 55 - DNA, 599 bp, Homo sapiens
ctaagattgctgcagcccttttatattatgaagtcctttaatacaatattatcacatttgattcttacaatgttggtgattttatagctccattttacagataaggaaactgactcaaatgggttaactcatgttcccaagattaaataaattttaaagaactgagatttgatcaatggaagtttagacttggatctaatctccactgtttgatgttattaggatgaaagtagtagacttagatggaattgtactgtgaaaaccaaagctaaatagattagtcgggattgtactgtgagratgtaacagtaacattgttttgcatttgtgtgtaggagtattctcttttcttctgctcttttcttttagttcagttgtagtttgcctctagatcccctcataatcagagaaacagtgctgaaagcttcacaagagtagctaatttaggaaaaggtgtgcatatgtggtggggggagtgtttttgtctaacacattttctgttttggtggtgagatcttcatttcttaacatctttcttgttcttgttcatatgacttttagctcattaaccttgctctctgtgctgcgtagtgaagcac

SEQ ID NO: 56 - DNA, 599 bp, Homo sapiens
cctgtgggcttatacatttcattaaatagaaaaccttttttctattttttaaacacctcccttccctctccccatcacttgaaggagataactgatagttatcccacagctaactgatgctcttatttttttcttgtttctctgtgtttcattttggatagttgttatagcttcatgttcactaatcatctgcaatttctaatcttctgctaatcccatccagtgtattttcaatctcacatgctacagtttccatctctagaaggccagttgggtcttttatattgtccttgtctcccrtctagctttttgcacacagaatatagttattccgtatgttttaatgtttgtatctaccaattatctgtgccagttctgggttagttttgattgattttcccccttcatttttggtcatatttttattactgtattttgtctcgttgcattggatatttttgtattccttaaaaacttcccatgctttattctgggatacagttaagttacttggaaacagttttatcttttgtggtcttgaatttattatttgttaggcagccctacaattcacagtctaggactaattattccatgct

SEQ ID NO: 57 - DNA, 599 bp, Homo sapiens
agtgtggaagccagatatgaagagagggaaagcgtgggaagaaggaagtaattgtggatatgaacagtccttcgggattgcaaagccttttgggagtggactgataattgtccagtttccttacttggaggtctaagcctgtgacagtcttcacatctcgctgagtttctagggatattgtgaaaaaaaatgtactcatttatgaaaactgaaatgacccatggctttcctgacttcctggtcacttgtttctttgctgctcttcagctctggtgtggtcagagttactgtgtttgaaaygattcagtcttttagtctttctgtccatgtgttaagcaagttccccccctttcttcctctcttttcaggaataacgcaaattatctttttctagaagaactattctataacttgccaattctagttgaaacattttaccatttcttatatccttagtctcatgctctggatttggcatcaattttttttttttcattgaattccaagactctttttgttgattgtgtaaccttggacaagtgacataaatgtctcggagtctcagtttgcacttgttcttggtggggatggtgataacc

SEQ ID NO: 58 - DNA, 599 bp, Homo sapiens
gagattctatttgtttgggagaaagtacaggaagaaaacaagagtctttttctagtaatccagagaactcttggattttgtccaagaccgtcaaggtggtacctcttaagagtctgcaagaaacacagtcctgctgggcttgaggtcccccctaaagcagatacagcttagatcacaacacctaagtcctttagaatatctggaaggcctttccaagaaggatgggtacaaacaagcccagactgcaaagactacaataaatacctaactcttcaatgcccagacacagaggggtgtctrcaagtatcaggatgattgaggaaaacatgatcttaccaaatcaactaaataaggcacccgggacaaatcctgaagaaatagagatatcagacctttcagacagagaattcaaaacatctgttttgaggaaactcaaagaaattcaagataacacagaaggaattcagaattatatcagataaatttgaaagattgaaataacaagaattaggcagaaattctgaagttgaaaaatgaaattggtgtactgaagaatgcatcagtcttttaacggcagaattgaccaaacagaagaaaga

SEQ ID NO: 59 - DNA, 599 bp, Homo sapiens
gtttagaccagtaactacttattgaccagttactggtctatttttttagaccagtaacaatttccagaccggcactgatctgtggactgtgagtagcactgtcctggtgtatacagacacagtctcatggatgtcagaatatatatgcgcacacatgtaattggggaaaataccccaaccatttctataaggttaatagcgagtagccgggagttcatatatccatttcttgacccggacagtatacaagggacatctgagacctctggaattccttccttatatgattgttattccacygcttcccaggtgagggttgtcattcttttttttttttcctttgagttgtgctgttcttcccttcttgtccttcacttcatgaggaaactatactccctgaaggccaggtaaatatatgttgtcttagacaaatcagctgtgctaagtagttgagaataggatactgaatactgctgccttatggactgaagtgaagcaatcactggcagtgtggccagaagaataactttttaaatttgagacaggatctctgtaacacgggctggagtgcaatggcatgatcatggctcacttcagcc

SEQ ID NO: 60 - DNA, 599 bp, Homo sapiens
accacctgacagaggatcagtaggaagcctgaagctgggtgttaatatgggggtgtggtattggtaatatttggtgtgtgctgctccatggggaaaataagcagtaaggatggctagcagttactacgagagaggctttaaatcttttcagagtgagaactgggacagggagtgaccagggttggcacactcattcccaagaccacatactttctcatgttttctcatcaggccaagctgatcaaatttattcaaacccttcccccttcccccttccgccttcccgcttccctttctctrtgttgctggggctggtgttaaactcctggtctcaattaagtgatcctttcacctcagcctccctggttgctgggattactggcgcaagccaccatgcccagttgaccctgtgtttttgaaacacccgagtgtgtccagtaatctgcgaacattatgaagtcctacccctaaaattcttaaagatatatttcagtgcagttaaactgcagtcatgtgtcacttaacaatgaggatatgttctgagtgaacttcctagagtgaacttccacaaacctagttggtgtagcctactacacacc

SEQ ID NO: 61 - DNA, 599 bp, Homo sapiens
agtgaagattccacttaactgaaagtagacctgaattattcatgcaatttgccaacatttatatccattaataaagtggtagtttattttgaaaaaaactttgcacttactgagtttttgttggcatttccctccctcgagttcatttgtcaaagtcatcatgaatatactgttagtatattcatggcttttgattgtttcactgctttttcttagtttctgagttgctgatttggtaaaactgtcatgattttctttgtcagttttcaaatgttgcaaagggggcagaacaactttgartcccatagatgattgatggtgctttgcaagtagttaaatacacccaaggtaaaggaaattttatgggattaaaaaaattctatgctccattctaggttgatagtatttgaagcaaatttcatttcattggtgggtggcctcagtacctaaactttaaaaagcatggattcataaaactgttacaacaccacttatgaaagtttctgaagaactaaagagttgttaaaaagctactgtctttctctaaacttggttactgtggagtagcactgtctgatgcaactttttgtaatgataga

SEQ ID NO: 62 - DNA, 599 bp, Homo sapiens
gaggaatcagtggtctgagcctaaggcaaccatacctagctggtagttccagcctagaggctgatagggatgcttgtggtgatagatgtataggtagaacaattttggctgacttaggatggtattttcttcataggaatagcgtgtttcttgtatttcaagacatttggggcacttaaacaaaaactacaacatggtgaggtcaacagttataaatcacagtacgaccttgttcaggactctggaatgagaagacagctgttatctttgtttcagatttggactaagtgatgctgttcwgtgaatattctcttttaaaaataactgttttcacccatattgaagccaggatcatcagcttcatgtctgtgaccactgtagtgagatcttagggtattgtgatttttggtcatggttaggtaggtgctttagaatgctcattgttactgtgggaggatcatggaattctcagtaagttttgttataaattctaaatttaaaatttgtaatatgtaaatatatatgtgtatgtttttgatgggaaacaccaaaatatatagtgtttgactctgggtggtggcttgtggggaaataatatt

SEQ ID NO: 63 - DNA, 599 bp, Homo sapiens
ttgactttttcctttggcttagtgattttggggtcccgagatatattttcctttcacagttgtgcaataaaaatgtagccaattatatatatttttatatttgtaaatgacataagcagtggagcagccttctagagggtgaaagatgatgcaaatatagtcatgtgtaacaatgtttgcatcaagggcagaccacatatgcaacagtggtcccataaaattatagaggaactgaaaaaactcctattgcctagtgacgtggtagccatcctaacacaatacattacacatgtgtttacratgatgcttgtgtaagtatatctactgtgctgccagttgtataaaagtatagccagtacaattatgtacaccacatagtgcttgataatgacaagaaatgactatgttactggtttatgtatttattatgctatctatccttagagtatggtatttatttttttaaaaagttaactgtaaagtagcctcaggcaactccttcgggaggtattccagaagaaggcagtgttgtcactggagatgatagctctatacctgttactacccctgaagaccttccagtaggaaaaaatccttgt

SEQ ID NO: 64 - DNA, 599 bp, Homo sapiens
gtggttagttcttcactccccaaggaagatgtttctttgcacagcaggtactggtagtggcttactgaacataatttctggtgcctataacatgcaaccactgccaccaccccaaaaatccattcaaatactcacctaggtaacagctggtgaatcccatgagagctcagtcacttaacagaccacaaactcaaatgtctaaaggataaggcaggtaatgtaagtgagtaaagtaagctaggctcaggacaacaggcagtagtttagactggcaaactcaaggatacctgccagtctaaygggcaatatctactcaactcctgccaaccagagccacaaagaaatgtaaaaagaaaaaaaaaattatattttgtaaagagaacccaaactcagaatttatgtgacatctcccctactttgaaaacactgcaggccaaatcaaacgtatctgcagacaatgtctacttttgtgttttccttttaaaatacaaatgtccctgagagagctggttatctgtttcagtcttgggacagcaacaaaatgcttttagcacaggaggtgcttgataaagacttgttagatggatggatagatctga

SEQ ID NO: 65 - DNA, 599 bp, Homo sapiens
tctttttataagggcactgatcccattcgtaagaactctaccttcatgacctaaatagccccacaagtcaccactttctaataccatacattgaggaataggctttaacatacgaattttggggggacacaaacattcagtccgtagcaatagataaagagcaagccacataagttatctggggaaagagtgttaggcagaggtgacaccaattgcaagggccatgaaactgggagtgttccaagatgggttcaggaacagccaagaagccagtgttggctggcacggtagtttttgtgrttttagtttttgtgattttatttgcatttaaatctggaatccatctggatgttaatgaaatgaaatgctgtttctagaagaaggttgttgaagtaatactctagaaagttaggatgcagagccagaggcacagtgctcacctgatctgggacatggagcagttttctcgaagagtttcaagaaaagataccataacggagacgagccttgtctcatgcttcttatatttatcctctgggaggatttcgttggctgttactggtatagcccaggacagtggttctctaactttagggctg

SEQ ID NO: 66 - DNA, 599 bp, Homo sapiens
tttaattttaacctctttcttaaaataaggaaaactaatcttgggtgtcagtcacattcataattgtaggggcaagcattttagaagaacttaactgttttcaagcactaaattctcaacctgagttgtcttcagcaacctaaggtattaatatgtctaaagagcccggtctcttggtttcttgtgcacgtagtttttgggtttctattaatgaaaggttctggaggttctagaataaaacaggtatacccttttggaaattgcattattaacatgcagtgatcattatttcttcaccaygttgttggaaaatttaattttgcagtgggaataaaaaatgtttaagaattcaattgatgtttattaattgttgcctatatatgttatgtactgtgcctggggaggggaaatacaaagatgagtaaagcaggacctcttccttaaatgaaattttagtttttaaaaagccttttaatgagcacaaaaagccctttagtgagcaaaacaaacacagttagtaagtaaaatgagtgcatgtgattttttttaaaggaaactatcaaattattctaaatataaaaattttaaacgaaatttaa

SEQ ID NO: 67 - DNA, 599 bp, Homo sapiens
gagatggggtttcaccatgttggccaggttggtctcaaactcctgacctcaggtgatccgctcacctcggcctcccaaagtgctgggattacaggtttgagccactgcgcccggcccaacttttttttttttttttttttaaagagatgaggactcactgtgttgcacagcctggtcttgaactcttgggcccaagtgatcctcgcacctcagcgtctcaagtagctggcacttacaggtgcatgccaatgactggctcattttgctttttctaaaaattcatattaatggatgtatacrgttgtgtctggcttgtttcaattcgcataatacgtttgggactcattcatgttgttttcatgtagtttgttccttttcattgctttccattgcataaatttaccacagtttgttgagctgatggatatttgggttatttccagtattggagtattttaagtttctgtagacctttgagtacaggtctttgtatgaatgtatgcttttattttcttgcataaatacacacagtagaattttggggttgtatgataaagtacagcagctgtatgcttaactttataagaaactgccaaact

SEQ ID NO: 68 - DNA, 599 bp, Homo sapiens
attcgcataatacgtttgggactcattcatgttgttttcatgtagtttgttccttttcattgctttccattgcataaatttaccacagtttgttgagctgatggatatttgggttatttccagtattggagtattttaagtttctgtagacctttgagtacaggtctttgtatgaatgtatgcttttattttcttgcataaatacacacagtagaattttggggttgtatgataaagtacagcagctgtatgcttaactttataagaaactgccaaactgttttccagagtggccgtacygttttacaaattcccatcagtagtacatgagcgttccagttgctccatatccttgccaacacctggtgttgtcagtctttaaaaaattttggtcattccactgagtgtggaatgtagttttaatttgtatttttaattttaatttgtagttttaattttcatttccttttggtttgtggatttgtactttgtggcatttatgctaaaagtaccaaggaggaagaaagttgtttttttcttcagattgttatcatcttggtttgtaacttgtatgttctcagaacattatttgattctcc

SEQ ID NO: 69 - DNA, 599 bp, Homo sapiens
tcaagaagaagaaatagcttttacaaaaaagtgatgtataataatttaagtggtatttccgcttccttactcagtttagtctctgatacgtcctatcactgtctaaattactccattattaggggaaaaggccaatttttaaaagcaggttgggtgcagtggtatgtgtatgtagtcccagctactcgggaggctgaggcaggaggcttggttgagcccaggaggtcaaagctgtagggcactaagattgagcctgtgaataaccactgcacaccaacctgaacaaaacagtgagatacygtctctttaaatcaaacaaacgagacgaaaccaggttactactattgaccaactgtagaaatcactgggttcagtattcctttctttaaaaatgtatgattcaacccagcagttccaatactagttattgaaaattcaaaacaggtgtataaaaaagtacatatatggccaggtgtgggggctcacgtctgtaatcccagtactttgggaggccgaggcgggcagatcatgaggtcaggagatcaagaccatcctggctaacatggtgaaactgtctctactaaaaaatacaaagaatt

SEQ ID NO: 70 - DNA, 599 bp, Homo sapiens
taatggatcttctccccaaatagggacttctaggcattttaaaaagttgatctagacaaaagaggaattaagttgagtcctcagcaggaattgtagcttgttcattgaatggtatttattttcccataggcctttttgattaatacttcaaatttgacttttaatttgatacatacaattgttctcataccacttcccaaaaagggttttatttttatttttattttttctaagtatggaaatagcagcacataagaaggttcctattacatgtataggattgttgatggttgtcttgcwtctaatttgtgctgttaaatagtcacagtttactggaaatttaagtttttacacttcctgggcctagtgtaaagttgatttcctgttgagctttcggggttcataaatataggcctgcttttctttcttggtggcttttttgatgcatgggattatggaagttttatacatgcattaaagttctttttccttggtgaactatagctgaagtgtgtaactcgagagcagtaagaaatacttaaaagtattttgcttaagaaaatatcttcacatgtgacatcccgaatcttctattatgt

SEQ ID NO: 71 - DNA, 599 bp, Homo sapiens
cctgtttgcttggcacttctctcctgccaccttgtgtagaaggacatgtttgcttccccttctgccatgattctaagtttcctgaggccttcccatccatgtgaaactgtgagtcaattaaacctcttttctttataagttacccagtcttgggtatttcttcatagcagcatgagaacggactaataccctgggactataggcgtgcaccactgtgcccagctgacattcttaaaatataacatattgtatttcttgaatttaagctgaacatttgcaagttttatcttaagggaaaaygtgtctcaaaagttaatatcaccactctaagttgaatttactgaaacagatgcagtggtcagcttagtagcagagcttaaatgaaggagacctgaagattggtggtgtgtgaccctggtaggagatgatgaggctgagaactgagtagaaatactcagggagtattgagggagtatttaggggtaggaatagccccttaagtcaggagctgcctttgtgaatagagggaagcaagaacgaataggaccagattgtaagagcatacataggtgggcaagagcagtcccaccaccgctcta

SEQ ID NO: 72 - DNA, 599 bp, Homo sapiens
tccacaatatgccaaaccctatctgtaattttgatggctgaactgtatgtatatatgcgcacacagcataaagtgcatattttgttagaactagattaatgccacaatattcagaaaacttctccctgtgttttcaaaaaattgacatattttgtggttagactgaaatgaggtaaatgtacatgttattgtagtactaatggattgttagatatttgctgtaatgttaacagttaatttatgccctgtaaatgaatattgaatatgaaacactgttacagggaaaactgtatagtctgmacaataaatgctgtataggaaacaagccttactccaaataattttttttgctagttcacacctctgagtagttggctctataggtgtgaagaatctagagaatgtataatttcagtgataaactagattataaagtaagtttaattgctgaaaagagtaagctttgccagcttcatatattctcttgttgcctgtttttgcaagacacatttcaggatatgcttttgggagccaatctaatgttacctgtatttgcctctcgtaaaaatcttaagctttgtgatttaaatctaagcaag

SEQ ID NO: 73 - DNA, 599 bp, Homo sapiens
tgatgttatgacaccatcaccctgtgcaggtgtagaaaaggcatagaaggagtatgggttaaaagaaggtacactgtgtcaactgctgattcctaaaagaaaacctgaagtttttgaactgccagttcacaaaagagaacttgaaagtacctgagcatcaaagtagcatggcaagcatgggctggagagtgtgtgggtgggacctggtattcataattgtctttagataccaacatccttcaccttcaaaggcttattgtgcctgagtgagtaagtttctgtgtgtgcatctgtctccargcattggggattatgctagatggtacagtgatgactcagtttcagttctttcaggatcatataacatttccaattaaatggtagacagatggttatgggagggacacaatgattattgacataggagagctaatgagattacatgacataaaaaagaagtatcattattgcagtatttttattccattgatatagtacctgtttcttgaaggtgctatacaaataacactgagataataagccatttttaaatgtcaaaacatggaagaacaagaaagtggctaggagaaataaataca

SEQ ID NO: 74 - DNA, 599 bp, Homo sapiens
gcgggcagatcacctgaggccaggagttggagaccagcctggccaacatgatgaaactctgtctctacaaaaatacaaaaattagccgggtgtggtggtacacgtctgtaatcccagctactcgggggctgaggcaggagaattgcttgagcctgggagacggaggttgcagtgagccgagatcatgccactgcacttcagcctggccaacagagtgagactctgtctcaagaaaaaaagaaaataattcattctctaattgttggatacttggtctttgtatgcttattaattattttrtgtgagcgttacattaaaatcgtcccctgtaggaaatttgtcagtttctcatcaatataaccatttttggtttaagtacagttggcccttgaacaacacaagtttgaactttgtatccctgcttatatgaagcttttcaaccaaacccagattgaaaatacgggatgctaaacctgtgtatatggaggaccagtatttaatatacgctggttcagtagggctgactgtggaacctgagtatatgcgggagttctgaaaccagtcccctgtgtatactaagggacgacagtattttgtgg

SEQ ID NO: 75 - DNA, 599 bp, Homo sapiens
aggctggtttcttctagtaaagcatttagaaaatattgcctattaaaaaatattttatgacttcctgctgggctggttttaagtgaaatatggtggggagatttatttacagttctctgtgtgtgtgtctttctctttgcttttctctaatgaagctcaaagcatcaggccatttatcagagataattatacaacagatggagtttttccgatatataatagtctcaaagtggaagattctttcccataatgaagaatgcccaaactttaaatgtctcttttgattttaatttaacggtktttttttccccagtgcatttattcattcatcccaaagctggttctgcaaagcatgctgcttttattagggctgtgttctgaaaagattgatactttgcagtacagtataataatgtacagttatatgctatattgaagtgaagagccataatttatcaaaaatcaatgtgtgagatcattaagttaatctctgtcaaaatacaggagttgagcagttcagcagtgttttcttatagaaattctttctgttccataatgtcagttggataggaacttgtttaagtactggtagtatcatc

SEQ ID NO: 76 - DNA, 599 bp, Homo sapiens
gactaaggtaatttctgtttagtgtcactcatcatattccagctgtggtacagctagctatgggaaaggctcagcaaagaggcagaagagaagggcaccggtgttccaagttgtttttacaagtccacgcaacagtacctgtggcctgaatagatgtctttttttcattagtcctgaagcctcttaggaagaacatgcaccagtcatttccctttctaaggaaagtgttgcagccttttttcagtagctgctgcaggctgagatttatgagaatagtcttgctagctgaactatgtttgraaaaaaaaaaaaaaagctcttccagtgctgagtggtgtctccattaatgttgttatgagaatcatgcttgtgttgatggtaaaatgcaaatgatgcaaatctagaaatcggagttgagccttagttaagcctgtaaatagtcaaaagtgaagcctaaattggagaggtgacagcccctaattttgtattcgtattaaatatgctttgtcacaagagaaaaacaaaacagtgtctaaaattagtactgattttattagccaggtgatattttgacctctaataaaatggggattattcta

SEQ ID NO: 77 - DNA, 599 bp, Homo sapiens
ggaagccagtgcttatttatccttctgaaaaatcctagggcacttaagaattctgctacatccgctctgcctgtgttctgtaaatggaacaacaaagcctggatgactgcacatctgtttatagcatcattcactgaatattttaagcctactgtatagacctactgctgagaaaaagattcctttcaaaatattattgttcattgacaatacaccttgtcacccaagagctctgatggagaggtacaaggaaatttatgtttccatgcctggtaacacagcattgattctgtagtccayggattaaggagtcattttgagttttaagtcataattatttaagaactacattttacaaggctatagctgccatagatactgattcctctcatggatcggccaaagtaaatggaagatcatctagaaaggactcaccattctagatgccattaagaccatttgtgattcatgggagaaggtcaaaatatcaacattaacagaagtttggaagaagtttattctaaccctcatggatgattttgaggggttcaagacttgggcaggggaaataattgcagatgtggtggaaatcacaagag

SEQ ID NO: 78 - DNA, 599 bp, Homo sapiens
agcaatggacattaaaggtcatcaagttaagccagctgtacttcttttcaagaagactgttctgtgccctcttaggctagccagactgcctcagccttaaatattggccctgactctaaaggacagagcttcctgattttgagaggtaaattgaaatgatttattttgagaaccacaggttgactgtctttgtctccatctttctctgtgtcctgcagtcagcaggttgcccatggtggggatttcaatcatttttcttctgaaccaccaattgttattgttattttgctggaatgttcyttgtagcctttatgtctaggctcttcaagttaggattcatatctatgacatgtgctgtacagtgcttctactgtgaggtagtctcccagacagaaaccacatgggccttcaggcatagatggtcagtaaataattactttacagtggtgtcatttcttaggagacacagagtgagaccttaagtgagatcttacctacctcctcccatccaatctatccatacaaggttggacctaaagcagccttgagcttaataatgatgtgtgttagaacaaggatactgagattagactaaggtg

SEQ ID NO: 79 - DNA, 599 bp, Homo sapiens
gcactcatcatcttgggtcctgaaggagttcatttgattcctggggcaggggacaaaaggaatatcaaatatttggcaagattgcaaaaatttctctaatcaaaaaggtgccatctgaccatgttttgccacagaaagcagttctcaccaaagtttatcatggcatgtcatagtggctgtttgctgacagtgtgtgggttttggccattcagtggatgactgggaaagggaaataatctactaattgctttcccttgggttttttgtttccctggaagaactttaatcagtctgcttctrttgtttatgttgaagaatagaatgaaggagtgaggtaaaatatactaataattagatgatttgatactcaatatgtaaataataggtgggtttaaaggcacctcacacaatgtatagatttcagataagatgagggaaaattaactgttttgtaattggagccctaaatgagagttaggtcccattgccacctacgtttgtgttacagcacaatgtataatgtgtcagtttgctatacttaaagtcaaaaacttgtatccaaggcctcgtttgggctttactcaggagaagcaggggct

SEQ ID NO: 80 - DNA, 599 bp, Homo sapiens
tagggatactcaacctgtatttacagtcaggctgtaaccagaagcaaatcctccatgtatgttcataggttgttcctaaaaaattgaaagcccaatatatgacatttacattattatgactctgtaaacgttccctgtcaggccaccgtgatacatgctaagaatatatttctttttctatgggtctaatgaaaacagttaatggtgaactatttagaaagcaagctttcatttacttttcagggctagcatctgtgagccattgcataagaatctctgagccatggaaaaagaatgttmctacagcaaagccaaattgatcctttattatattccatcagttgctcaaaaatcatgccacatcttagtttgccttatatttgcattatagctttttaatacaggtccccccacccctggcatttataattgcctttctttttttttctacagtatttggttttacgtcaccacttacttcctaactctgtatttacacctttcacctgaaacctttttcctagcgcctaccggtctttcctctccaatctgaactagattctgtaagcccgcggtgacatcctagaatttcctttcac

SEQ ID NO: 81 - DNA, 599 bp, Homo sapiens
tgaagagaaacttgagggagtgtagaggaaagtgagaaagaacttagagatggaatcagaagctgataataagcacctgaggccagtcagattggagaatgcagacatggaaaggaggtagaagtcagtggagatagggaaggagggagagccatagcagcccttgtttgaacaggtgagcgctgtgaatgacaggaagctcagaaagcccctttgtcctttcttaagaaaattgagaattataattgggaaaatcagacttccttttttgttgtattgcttttttctagcctggccccrccccgctttttgagatagggtctcactctgttgcccaggctggatacagcagtaccatcacagctcactataactttgacctgttgggcttgagtgattctcccacctcagcctcccgagtagcttaggactacaagcgtgtagttgtctcactgcaatccctgcctcttgggttcaagcaattcttctgcctcagcctctggagtagctggaactacaggtgcatgccaccgtccccggctaatttttgtatttttagtagagacagggttttttcaccatgttggccaggctggtct

SEQ ID NO: 82 - DNA, 599 bp, Homo sapiens
tatatacacacacacatatatatacacacacacgtatatacacacacatatatatacacgtgtatatatatacacacatatatacacgtgtatatatatacacacatatatacacgtgtatatatatacacgtgtatatatacacatatatatacacacacacatacatacatacacacttcacggacttctcaagtttctctcttttggcagatgaaaaatcttggctgattttgctcttctgataaatactgcaaaagatagtagaactgaagttctatctagtcttaacactgtacraacactgtacaaatctttgaattcttatggttatagactgacttggggccaaggaacagattaaaattaggggacttgggcatgtggactttgagaagtagatttttctgttaggtttttcagttttgcttttctgtctgttttgtcttaacttagagtactaccttattcggtactccactgatgctataaatgtagtatttttaaacatcaatgcattgtaatgttgttgaactccaaaatgcccaagcatgttataaaaaggaaaggttatctattgaggttcatttgtcatttac

SEQ ID NO: 83 - DNA, 599 bp, Homo sapiens
tggacaagataattttatgaaatagaggagcttgttttcattcctatatttgccccaggatctgtcccctgccctcatcccagagacagctgggtctttgtatacgcagaaagtagcagaaagttgtcttcgtaataaggtactcttatgggaaagcagaagtagcatctttaagtgtagcaactgttggagacaggatatatagtagttaagagcaaggactctaggccaggctactcttcacttatttgttgtgtgactgtgggcagttcacttaaccaatgcctcagtttcctcatstgtgaaatgcaggtagtagctacctcatagccttgttgtaacaccaaaatgagttaatatatgtaagagcatttagaaggtgctgataaagaatatgtgctttgtgttagtgttcattattgcaattattaggaagcttaactttaaggaatctcttttgtcatatccaccttccatgataagtggccttaaaaataggcttgtacaagtccaggcatggtgggtcatgcctgtaatcccagctactccagaagctgaggggaggatcgcttgagcccaggagtttgaggctgccgtga

SEQ ID NO: 84 - DNA, 599 bp, Homo sapiens
gtaactacatgggaacctacagttacctcaaaataaaacatttaataataaaaagaaaatgggctcggaacattcctaatagagaaaaattggcagtgaaagccatgtgtatatctgaacagacagaccaatattaggtaaataaggcaaataaatatcacagaaggaaaactgagctgaccattgacattttagtgagatgttcatgggcccccaagaaaaaaattgagccccgtgggttcagagggagggagaagacagacaacagcttcatttccctgaaaactcatcaaactggcrttctccaagggcctttgcttcccagactgtgaaatgttctatttagtggatggtttttggtaaagagaagaaggtagatgagtagcaacttacatacaaatgacattctttttttttttttggagacagaattttgctctcactgcccagtctggagtgcaatggtgcaatctcgggctcactgcaacttccacctcccaggttcaagcgattctcctgcctcagcctcccaagtagctgggattataggcatgcaccaccacgcccggttaactttgtatttttagtagagatagggt

SEQ ID NO: 85 - DNA, 599 bp, Homo sapiens
tgtgatatctttctcattctattaaacattggcagagaaagcaggggtctggtgagtaccaagagttgtgaacgtggaatgggggagcggcagctctgtgtgggtatatctactacactagattatgttctcaagtaaaagcatactggaaaaaaagaatctgctttgttggccctttatgaaggaccttaattagaagtcactgttaactcctggggtgggaatggtttccagcttggcacgggagttcctcctgctactaatgagcatgctccttgcaaggactcgctgcttctgacmgttaaagctgcattgtaagagttatttcttgtcacctctctctccacatttaactgttactgcccaagttcagagtatgtaagtagactgttaggtgttttcaggaaattggcaatgcttcacggtaacagggaatgatatttgtgagtaaaagtaaatcattacaggttaatttattcaaatatgggtagtagatggttgctttaatgtcaaccatgtttggatttttttttcctggtagtctttttctaattgacataaaacttacaaatgaatagttgaattatttaattcattat

SEQ ID NO: 86 - DNA, 599 bp, Homo sapiens
gaccaaggtgggggtgacagaggggtaagaggtgtggcatctggattattttgaggaagactgtttaggagactcccttctggaagtaatttagcacaaacaatttggaacttggtaagaggtacatcgtgggtatgcaagcagctgtttcaggaatacagagccccaaagagctgcccagtttattgaaggcaagagtcgtttacagccttgcctgtgataagagctttgttgcgtcttaattacttttttgaagttgtgcaagacacagttttcacagagtacacccccctccacctrccattttggctccacctcaaggtggggtgcctggtgctgctgctgctgcaccaccccatggtggagttcatagggcagcttgcatgtgtaccagcagaggtgccccttcattctgaatcaaattgaactaattgtcagtattagaacctgacttctgtgtctttaagagtagaaacgtgagtaattttactataaaagattacagatcactcatcaaacgctggctttgggaatggaattgtgaaagcatagtgatacagagatacattagacatagtcccatctctaaagaattacgg

SEQ ID NO: 87 - DNA, 599 bp, Homo sapiens
acctttccttctgtattctgtagtgtaataccttttagaattgctgcacagggatgcctacccacagtggaatgaaacttaagtcaaccctttctctcttcttgaccacatgtatcatgacacataggaacgaaattgtgtagcagcattattttaggctgatttacagtttcctgtacttgtttttatttcctggttttaaaacctaatattataaaattgtttgaggaccagttcagctgtcatcaggttgactttgagacctgtctgtagcctctgagaatcctatttctttctttstgagaagtcagctttagaagccccctttcacttctgggacagccgaagatatcctacaagtactattgctgacagtcaggactaaatagttcactcagcagttataaagtaatggtcactggaagtttactatcttctgaagcggcagcctttgctgctacggttcctcaattatctgccctattggaaatgggaaggaagagagaggttattagcttttcatagaaactacgttggaactcctattcctgttttgaccagatacctgtttgttacacattctggtgtatgcggtactc

SEQ ID NO: 88 - DNA, 599 bp, Homo sapiens
gaggatgtcattttcattaatttctaatgaggtgccaattaaaggcagggaagacgctccaaagggaagagtggccaagctcccctgaatcctccgccccagggctgtcgtccaaacactgccagtgaggccttctctaaaaattccattaaattacagtctcatcgagagcacacacagacacattcagcttccctttgatcttcaaaagagcaatttgtaaggcgagttctgagccttcttacatagggaacagctagaggtgataggaattgttaattgccaagacacttcatgggmtttcagtaaggaggaattttgaatacgaattctcctctgcctccctgtgactagataacctttaaatcaacaataccaaagtaacacccttttcctgtgaaatctttcagggtgccaattagaaaaggcatgtggttgcgaggctgggattatatcctgcatcttagagcatcttcaatagtttaacttctcaagatagaaaacagttgcctgaaaatggatctatttgtttcattttctactcagtaagcaaattgatcagctcattgtcattgcaggagtcttgtctcctgaattga

SEQ ID NO: 89 - DNA, 599 bp, Homo sapiens
ctatgccgttttggtgactacggccttatagtttagtttgaagtcagataatgtgatgcctccagatttgttctttttatttagacttgctttggatatgtgggcttttttttttttccatgtgaattttaggattgttttttctagttctgtgaagaatgttggtggtactttgatgagaattgcactgaatttgtagattgcttttggcagtatggtcattttcacaatattgattctacccatccatgagcatgggatgtgtttccatttgtttatgttgtctatgatttctttccrcaatgtttgtagttttccttgtagaggtctttcatctccttggttaggtatattcctaagttttcattttgatggttttttttgtttgttttgtttttgcacctattgtaaaaggggttgagttctttatttgagagctactgatttgtgtacattaattttgtatccagaaacttgccgatttatttaatcagttctaggagctttctagaggagtctttagagttttctaggtgaacgatcatatcatccatcagcaaacagtgagtttgacttcctccttaatgatttggatgccc

SEQ ID NO: 90 - DNA, 599 bp, Homo sapiens
tagtctctgattcctgcctaacaaacttgcgattccttgacatataatccaagtaattaaggtatcttagaagccatccatctggtgccagtccagacccctaaactgacgctgcataaaccccatcagcagtttcctggcaggtagtcccactgtgtacatctgcatggagggagtgtggcatctcctgcatggaggatgtatggcatctcctgttgctgggcaacttagattgactaggtgcttcctctacccagcttaaactggcctccctctagaacacatactcattccccctcwctctgccctccggagcagtctgtcacaaatcctcttcttcaagcctgttcacagatttgagcactgtgatgtaagctttccagcagcccagctcccgcagtaacaaattcattctcagagaccacagaactgatattggagcatgaaggacagggtcagaaacaggactggactcagtgtttgtctccacagtttttctcagtgaggccctcacaaaacctggtgtcttttttaacaaaacccttcatctggtgatctcccctgagacagctcaacagtcttctcttctgtggagtcca

SEQ ID NO: 91 - DNA, 599 bp, Homo sapiens
ttgagcactgtgatgtaagctttccagcagcccagctcccgcagtaacaaattcattctcagagaccacagaactgatattggagcatgaaggacagggtcagaaacaggactggactcagtgtttgtctccacagtttttctcagtgaggccctcacaaaacctggtgtcttttttaacaaaacccttcatctggtgatctcccctgagacagctcaacagtcttctcttctgtggagtccatggttggcctctgtttatgatatatgtctcagagcatattgaaattgtctgtttacwtgtctgtcctttctccagaccatgagcacttaacgatattcttctgcagttctcggctcttactgcagtgcctggcgtgcggtgacacattaatagacacttgaatacactgtgagatccctgcgtcttgcagttgtgatcatctgggtgaaggaaaaagaggaatagctttattgaacagctagtatatcctgggtacctgtacttgagttatatcatttgactcttaagagcattgttggacaaggccgggcgcggtggctcacgcttgtaatcccagcactttgggaggccgaggc

SEQ ID NO: 92 - DNA, 599 bp, Homo sapiens
ttctggagagggtaacagaatgagatgggagacttactttctgctaaatatcattttatacaatttgaatttttattatattcatgtactacctttaaataggcaaagtaagaatgaacaaatacaaaacaagtacatctcaggtcaattttttttttttaaatccagaagttccctccaactgctgggagggggacggcttggagggcagcaaaggcagaagcaggcaacacgcctgagctctagcagcctggtggagccatcggcctcaccgcattccagccctgctggctgcctttstcttccatcagtatgctgagcttattcctgccacaggacatttgcatgagaagaggtgcagagccgattcaggacatcatttagatctaactagacctctaataaagtcagtgtgaggagtaaggaaaagagaggaatcaagaattgagcccacagtttgacttgaacagcagctgggtggatatattgccactcacactgaggcagagaagatgaggagataaactaacagaggtcaagaagatgaagagttgtttcaaacatggtacagttgagattcctagcattattctagtcag

SEQ ID NO: 93 - DNA, 599 bp, Homo sapiens
tgaggcagagaagatgaggagataaactaacagaggtcaagaagatgaagagttgtttcaaacatggtacagttgagattcctagcattattctagtcagacgtgtttgtcaagtaggccattggatatcgggatttggatttgaggggaattcatagatggaaatataagttcaagaggcatcagcatggataccctatttggatctggatgaaattacctaaaggagaaaatgcaggaggacgtgatgctgggtgattttagaggagaaaccatgaaggattccaaggagcagcagtyggtcaggtgggaagaaaggagtgtgacatcacagaagatgaaaaataaaagtattttaagaaacagaaaagtggacaaatgctgtagaggttgaggtgaggatattggatttcgccccttgaatacctgattggcctttgcttgctccctagggttcagagaacacagtctttctatacctaacctatcattcatttacctgaccaaaatattttgagtgaaccaagcagtattttagggatagaggatatgtgataaattacagaagcatggtgcttgattttatgaaatctagccaa

SEQ ID NO: 94 - DNA, 599 bp, Homo sapiens
aaatgctgtagaggttgaggtgaggatattggatttcgccccttgaatacctgattggcctttgcttgctccctagggttcagagaacacagtctttctatacctaacctatcattcatttacctgaccaaaatattttgagtgaaccaagcagtattttagggatagaggatatgtgataaattacagaagcatggtgcttgattttatgaaatctagccaaggagacaaattaaatgaacaaattccacaaatgacgctaaattaaaattgtgactagtgattgaaggaaaagttcargatgccttgaaagcatccaactgggccctaacctagtctgagggtccaggaagatcttcctgaggaagtcattgaagctaaggcctgagtaagcaagaatttcctttggaagtcagtgtgtctgggacatagaatgcaagggagtaaatggcacaaggggaagcggcagccagggttgagatcgtgcttcagtggggaaatttttattgtaatcagtggagcacggtaatcacagacagggctggatctcatcatctcatcatgttggctgccctgaggaaggtggctttgatgtgggg

SEQ ID NO: 95 - DNA, 599 bp, Homo sapiens
tggtctctttgactaggtggtggcttatgacgctggctgctctgaggaaggtggatttgatatggggcgaccacctaaaggaggctattgcagtagccctggaaaaaaagattctggcggctttgactaggtggtggcagtagagatggcaagaagtgaatggatttgatttttggaaatcgtatccacaagactcggtaattgaatgtgaagagtaagtaaggtatcagtgatgactccaggtttgtgtcatgagccactggctgcatggtggccctattctcttagaatgggagacaytgaagaagaagcagatttgtgaggaagtagcaagtgtcctatgttagacatctcaagtctgaaatacctgagatgccaagtagataattgaatacacagatatggagctccaaagagaggtctggggcagagagataaatttcagaattgttagcatacatagttagaattttaaccttgaagaagagtgtgaagagtgtaggattaagacaaaaagaaggtccagcactgaataatgctaatatttcaaggatagcagatgaagagctatcagagaaagagaaggaaaacaaggtaag

SEQ ID NO: 96 - DNA, 599 bp, Homo sapiens
gtagggctttcactatagtgtgtagtctgacttcagatttgaactcatgtctgcagaaaatatttttctatactgaataagcctcttccccttcagggcaggtgtttttaaataaactaactttaaacatggagaatcaagaagccagcttctctgcccgcccccacccccacccacaccacacagtcttataacagtggacagcagacattgtaggacttctgaccttgcagttcagattttgtggaaacggggcaaaagcatgcttgtacttctaagctcctcggaatgacctttttsagttatcgtggtgaaaacaagaagcccttagagaaacttcattcttaaaacaagcacataaaacaagtgcatgtgatgctggcacagtttgggtgaaaacatagattgtttgcagaatgtctacagcccattcctttctcctgctcttcctttcactacagctcccagcctgacagtcttggggaaatagacatgaggcttttcaacaaacaatcagtacgtgaccaaacttggcaacacatgctctgcctctgtttgtgacagaggcttgcagaatactcagctgtacctgtagagga

SEQ ID NO: 97 - DNA, 599 bp, Homo sapiens
cagtggtgaatttttttatacttttaaaatggcatgtgtacaaactgcatggggagcttcgttagctggactgtggcacagttttcttgcctgcactgctgacggctctctgagagcgatgtgacggcacatgccaagggagggctctcaatcaccgctgactgcacacagctgccttatgagttctttgcacaaggcttttgtatctttgcctctatcatactgcttttggttctaacatttagctggcatgtaactgatattttaatttatttgggggaaagcagatgattgcttctsttttaaagctgtctcgatatgatttagtgtttcttatgaaattgttgaggaggagtgtgggcttttctaaattgcaagttttccttctctttgaaaagcagacagactgatgcccagcttgttgattcactgtggctttggttccagtgccctgggcagctgacttctgtccatccagtggttctcttctcagcccagcagagcactatgtcagccaactcagcaagctgggatccagttctgtttattggagtctaatgattttccccctaccatgcaaagccagggatggaggagaa

SEQ ID NO: 98 - DNA, 599 bp, Homo sapiens
aatcttgtgtctttggtagcaacagtcagggaagtgattgctctacccccatcactgtgccaagccacaggcttgggcggggccccgggatgacagccttcggtggttcagccactgtggctgtctggacaggcatcctcttcttccattcctgttccatgtgccccctcgcgatgctgtctgctcacttgaaggagatcttctcagatggagggtgtagctggtggagaccctggggaaaccttccccccagatcacagggggtgtctggtactacttgctgtggttgggcctcctcastagtcctgctctctctcttcccatgcccagggcagcctgtcagaagccagaatggtagcaggcagagagaggcctcagtccagagggccatggaaagtggagtcatggagcacagtgcctgcagagctgctgtttttccctccctttgaaatcgagggctcatcctaaattgctcttcagtgccaacaagcaaagggacgcccacacaagttcttcagcattttgtacagatttactgaacgcaactcaggcatccttaaggaagctctgcagcttcctcctggaccagtatttcatga

SEQ ID NO: 99 - DNA, 599 bp, Homo sapiens
tccttacccagcttggatcccagaattcaatcagagagcaacccaaggttcatgacacttgtagtggtttctggaaacagatctgtgagtaccaagaaaagaggataaagattcatcccatccaccagtcattcccatgcacctctacccgccatcccctgtatccaggacaacccccttctgacaccaaaatgcatttcaccattggctgctgtcggtagataatacctgctcagcatttgggacaagttccagacataacttcctcttagtgaatgatcctgacaggagaaagaattkagcttaatttatgccatctaataacctcagtgcagctacttgggaagttagccctccagagtttcccccaaagttttctccagtgaattacagtgccatatattctcattgctaccagcgctgctcccaaaatctatctgctgtttaatagtttttacctttcaaaaatgcaagctggctgggcgtggatttttgaaagcattcctcctgccttggcctctcaaagtgctgggattagagggtgccttctaatcccagcaatccagcacttggaaaggtcaaggcgggaggaatgcttg

SEQ ID NO: 100 - DNA, 599 bp, Homo sapiens
aagttaaggctattgaatcttaaccttaaatcttaaggctgttgaaactcttcatcctgtggccacttcgtccttgcttgggctcttgctgttcttctaaaagcacagcctctacatctctgaaaagaagttgaggatgtgaggagggatgagtcagtttgcaaggggtggctgcctgttctttatggcctggatgagggcttaaaagaaagaagtctgaaaagagatgctgttggttggaaaacaagtaaggagagtgtttgcttgggcaggctgctgtgggatgcctgctccagacasggagaacaggtttaggaatggatctagatttacctggaagcttgctcctgtggacctgatccaggcccacggggcctggaggtgtggtgagataatgctgatgggatgagaaggtgatgaggagccagccagcactggcaggtgtcttcctagccctgatcttgttctccccagcgttttgaaagaggcggccagcagactctggggacctcagacgtctgtgctccacatgcgtatcggccgctctagagattgcctgggaagctggagggaatgggagacaccccggtgcagctgcg

SEQ ID NO: 101 - DNA, 599 bp, Homo sapiens
ctctgaaaagaagttgaggatgtgaggagggatgagtcagtttgcaaggggtggctgcctgttctttatggcctggatgagggcttaaaagaaagaagtctgaaaagagatgctgttggttggaaaacaagtaaggagagtgtttgcttgggcaggctgctgtgggatgcctgctccagacagggagaacaggtttaggaatggatctagatttacctggaagcttgctcctgtggacctgatccaggcccacggggcctggaggtgtggtgagataatgctgatgggatgagaaggtgrtgaggagccagccagcactggcaggtgtcttcctagccctgatcttgttctccccagcgttttgaaagaggcggccagcagactctggggacctcagacgtctgtgctccacatgcgtatcggccgctctagagattgcctgggaagctggagggaatgggagacaccccggtgcagctgcgccctgagttctctgagcctggctcctggctccctgcagccctcccctccttgttcctgacccagagtgactttacctgtaccggctccctgctcgataggcctcttgatctttcatc

SEQ ID NO: 102 - DNA, 599 bp, Homo sapiens
tgccagatccctgcactggcgaatggcagaccagagccggcggaggcggggcactctggctgcttctcgtgaccttggatcctgtgtagaaaaggcgggtctcgttcgtggactagtcttgagtggagctaattttcaaagtgaggttactgtttctcttcttgtgtgatgagttttttcatagcaagtcaggtaaaaaaatttccgtaagaatctgtcccccaccccccatttccagctctgtgggagagtgaggccaccacccagcccagccacgtggccaatctcagcagcctcccrtggccaaggcacacgtggccatcaggtatcctggctactgggactaaggataagtgaagaggtgaaaggccgatcattaaacagagtttctggcaaagccatgtgcgtcgtgggaactttaacaccagcttctactcctctattaactcagagtctccgtctggaacactttccacttaaggctttttataagtttgactccagcagtgacaggtatgtgttgttttaaatccactagccatgctcaaaaaaaaaatattcttcatgttttaattttatacaatggctagcaggcacct

SEQ ID NO: 103 - DNA, 599 bp, Homo sapiens
cccagccccacctgactggtagtgatttcctaacatggggcaaccaggcccacccccacccttccccacctggcgtgcgcagttgcagctgctgaaatcctctgtgaacatgagggggcacaggtggagaaatgtaccctcaggccctcacctaccagagcaaatatcactctcggagctgggccacagccacaaaccgctgttctagacagatccaaacccactgtccctgggacgtatgctgccttcccttactaaacttgctatatggtagatgtggactgggtgtccttggactayggggctgcatagaaacgagaatggaggccacgacatcatcctcttggcccctggaggcacgggcggctccagctggaaggggcagagcccgtgggcggcagcttccccagctgctgctcccagcacttctactgattcttgttggctatgaaatgtctttttaaaaaactcccaatatagaaatctggctgcagaggccagtgtgcagacccagccaccgcgctgtgctgccattcgccacatctggtccgtgccagatccctgcactggcgaatggcagaccagagccggcggaggcg

SEQ ID NO: 104 - DNA, 599 bp, Homo sapiens
taatgtaaacacaaatactagtgaatattgctctggtaaaaatatatgctggatctcagtaggagactgggagagggaaagaatgtgcctgtgtattggtggcagaggataagagagccaagttcttgttcttagcaggaaatcaacaaaggatgtccaaagctgaaaacttgagagtagtatgttatttagaaatatggaggtaaaagaggaggaaaaagcttagaagttaaaattggttttctttggggtaggagataggaatgcagtaggccttatatttacgactacagttaaacrtataacattgattgaaaaattgaaatgctttaataagggaatgatggaagcccttcctgggaactgaggggccaagtcctcagaaacctgccttactgttctctgaaagtgggtttcagggagttcaggtctctgagatgcaacatagagtgttggttttgatcagagctgctttggaagatgcaggcagctggcacatcacaacacagcacatgtttaaaaccctgaaggctgagggcggtggcttgagcttataatgccagcactttgggaggccaaggtgggaagatcccttgagt

SEQ ID NO: 105 - DNA, 599 bp, Homo sapiens
aatcaacaaaggatgtccaaagctgaaaacttgagagtagtatgttatttagaaatatggaggtaaaagaggaggaaaaagcttagaagttaaaattggttttctttggggtaggagataggaatgcagtaggccttatatttacgactacagttaaacatataacattgattgaaaaattgaaatgctttaataagggaatgatggaagcccttcctgggaactgaggggccaagtcctcagaaacctgccttactgttctctgaaagtgggtttcagggagttcaggtctctgagatrcaacatagagtgttggttttgatcagagctgctttggaagatgcaggcagctggcacatcacaacacagcacatgtttaaaaccctgaaggctgagggcggtggcttgagcttataatgccagcactttgggaggccaaggtgggaagatcccttgagtccaggagttcgagaccaggcaacatggtgagacctcatctctacgaaaaaatattttttttttcttttcaaaccctgcatgtggcatgtgatgcaaaaaaaaaaaaaaaaatttaattgctgcggggagcgggtggtggt

SEQ ID NO: 106 - DNA, 599 bp, Homo sapiens
ggctcccctcctggccacctcttccaggcagctctatcaatcctagcagtgaggagtgcagttgaaggggcctggggcacacatcacagggttcgatgggagaacttgctgacagggagctggagtgtctctggagtcctcaggcccacatccaccaaacaccaggaggagctgaagaggagggactcccagactaggaggagagggatctgctgtgtatgacaaggtgcccaagttcggggtgggggtggggaccaagacatagaccccagaggtaaagctctgggtctgcaccgtggsagccccattaggggcatcttccagcccacagccctggctagggtgaccttcattcaacacctgctatatgccaggcagcatcccagacactggggatatgatggtgacccaaactacagctatagggaacaaaataagggcagtgcaaagaatattagaggggtgacaggcatacacgatacgcagggggaagggagcagggggagaagcatagggtctgagggtctttgtctgcgatgggcatggagggccctgcctgccgcttgggggttcggtcaggctgccactgtgcgaggaac

SEQ ID NO: 107 - DNA, 614 bp, Homo sapiens
ccacttctgtgagctgctgcccaggcaccaaccgtcccggcgctggggaccaattcctggggagggagactggaaaggttgcccagctggacctcttcttcaagcagttttgctacaatggaagcaaatgggcaggagagtggatgtgggggcgagggagtttacttttgaataaggagaagttgctgcatgtttttatgctgttggaatgatcccgtagcgagggagaaatttgggatcgatgcccatgatggggtgtagagcgcagggtggcaggtgtgggcacagggcaggcaagcgggggtggtatccttrtgctctgtgttctcacggacatatggaacagggtcgtgaggagtgagtgaggttgggtgagggggtagcaacagtttggtggggcactggcatggatttaccgggagacaaagccttagcttgaggcccctcactcgtgattgggcctcggcaatgtgtttgcatgttcccaagattttataaaatttgcaaaagtgagatactttaactgcagttggttaaaaccattccctccttccattctaacttcccttccttcacacttcctgttctgttgggtgatgttgaggccacatggt

SEQ ID NO: 108 - DNA, 599 bp, Homo sapiens
acagggcaggcaagcgggggtggtatccttctgctctgtgttctcacggacatatggaacagggtcgtgaggagtgagtgaggttgggtgagggggtagcaacagtttggtggggcactggcatggatttaccgggagacaaagccttagcttgaggcccctcactcgtgattgggcctcggcaatgtgtttgcatgttcccaagattttataaaatttgcaaaagtgagatactttaactgcagttggttaaaaccattccctccttccattctaacttcccttccttcacacttcctsttctgttgggtgatgttgaggccacatggtacttttgggttctggctaagggtaagcagagttagggacacgattcgtttgaggttagtgggatatatttgtgcgttcacagtcacttctgcatttagtaggtcattgccagtcatcctggggtagggctggcttcaagaacatgcttcttgtccattgcctgacacctggtgtcgtaaccagaaggagcaaggccacggttgtactgtgataggaacgtgtcctacgggacctggaacagggagtaccggttgtggaggagagatgag

SEQ ID NO: 109 - DNA, 599 bp, Homo sapiens
acactcctgtcagagcattaaagatgagtttaggaatgctgggggtgttctagaatcttacagctcagatatgaaagaaacttggtaacagttgccccacatttgatgacagttctaaaaatttacatgactatgccaataagagttgtgaagatgacagaaacttttctaaagtatcaataattcaaaacagagatcgaccatgctcaaggaaagattgaggaatctttctattcctacatgatattacaagatcattatcatgggaaagggtgatgaaagaggacacagccaagcaawgtaggaacaaaatgtgacagatgtgtcaccaccagtgagggtgaactgcagacatctgggtgtttgttgactttaaaacgtgtaatttgttgggaattcttttcccatgttgtacctaatttgggagtcagaatttttactctttttcttaaaggggggctcccaaactgtataaacttccggccctgcgtaacctgggggaggctcagagtacctggggtcttccgctgtgtcccaggaccctgagaggcagcgcagttgggtcagctgacaagactgtcctgacgaaggggcagcat

SEQ ID NO: 110 - DNA, 599 bp, Homo sapiens
atatgaaagaaacttggtaacagttgccccacatttgatgacagttctaaaaatttacatgactatgccaataagagttgtgaagatgacagaaacttttctaaagtatcaataattcaaaacagagatcgaccatgctcaaggaaagattgaggaatctttctattcctacatgatattacaagatcattatcatgggaaagggtgatgaaagaggacacagccaagcaaagtaggaacaaaatgtgacagatgtgtcaccaccagtgagggtgaactgcagacatctgggtgtttgtygactttaaaacgtgtaatttgttgggaattcttttcccatgttgtacctaatttgggagtcagaatttttactctttttcttaaaggggggctcccaaactgtataaacttccggccctgcgtaacctgggggaggctcagagtacctggggtcttccgctgtgtcccaggaccctgagaggcagcgcagttgggtcagctgacaagactgtcctgacgaaggggcagcatgctgatgtctcttctggtataagctccttgtcttgtgacgagatgacacgttgagtggcactggtcag

SEQ ID NO: 111 - DNA, 599 bp, Homo sapiens
actgtcctgacgaaggggcagcatgctgatgtctcttctggtataagctccttgtcttgtgacgagatgacacgttgagtggcactggtcagagagcgaatgagaaatgttgtggagatgtcgtgattgttaaacatgttctttccctcaattctggcgctcctcccgtggaaaagcggagcctctccccttggacacgggcaggagcgcgtgtggtggaggtgaccttcccgtccatgaggccacatctgaaagtcgcgtgagaagacccatctggcgctctcaggcgacatgagcctwaacacgggctggcgcgcaagaagtctggctattcacatcagaaggctggcgataatcgaagagtggaaagtgccagcgaggatgtggagaggctggaacgcttgcgtgtcgctggcgggaatgtgaaacggcacagccgctgtggtagaccctctgctggcttgtcaaaaactccaaaaggaatcaccacgtgacccagcaatgccactgctaggcatacacgagagagagctgaaagctgggactcacacaagtcctcccatgttccaggcagccctgttcatgacaacccaaagtag

SEQ ID NO: 112 - DNA, 599 bp, Homo sapiens
cctggagtcaggatgcggttctgggccagatcagcctctcctaggctgggagagctctaacggggccccggcctctcgttccctagctggagaatgtggaaggttccacaatctcagacccttgcctggtggctctcagccatgactgcctttagaatcacccgcggagtttttaaagatttccgtgtctgaatccgcccaagaccaaaaccaagtcagtctctggggtggccggtcaggggtggttcctcacaaagctctgcaggcaattccagggtgcagccagatcaaagccaccaycctaatgcagccttgctgctgccctgccgcgcgaaggcacgaaagcctgaatctggaacctgcgacaccctcattctccaactggagcgccccagtgtgcagacggagcgagtcctgggtggattttctcagaaatggcttcccattttttttccccttaaacttgcaaggcccatggaagctgggcaatagtgtgtgatggaggacatcggacttggccccacatgatgtcattgtcttccgtgggtgctaggtgcttttcataggcccctgggcacctccacctccaagcagggccc

SEQ ID NO: 113 - DNA, 599 bp, Homo sapiens
tgctccaaggcaaagaacgccatggggcttccttccttccccgcacctgctcagcagccttggttttctcatctgtccagtggagctaacagtagcaccttttgggactgtgagggttaaatgagcctggggcaggggttgggggaggtggtgagtgtgtgtgtgtgtgtgtaagagagcgagcgagagagacgccagccgttgtgatgatgttaacattctctgggtctacaccagtggccctttctgcctgcatttttattacaacatagtcctaacactgcatttcactgtcaggcrcctccgtccactccactcctgcgcactcctcccggctgtgtctcctgtcttttttgggtcccttttggaaatgtgggctcctcagagtatttggagccaccttccccccaacacctccacctctaggggcctggtcctttgggagtgggcagggccctggggcaccctgctggcctccagaagctgttggggagatacgagggaagtctgaggggcagcagcgctgggtacagggcaacaggcccgagccgggaggcccttccatgggaccacaagcgcccaccccgattgacgacact

SEQ ID NO: 114 - DNA, 599 bp, Homo sapiens
ctcagggtcctcgccgctgactgcacatctagtccagctgtcctctgaagcgctgggtgggttgtcagaatcagaaccaaggagagcacttaagaatgccaacccctgggtccacagcagccccccaatcagagcctgtaggggcgggtggtgtagacatgtcacttggtcaagttcccaggtgattcttacccaccacacagtttgagaactgttctagtagtcatcgagcaaaacagaaacacattacaacctgagccaaaccaaaattatcaggtcatcctcactcgttctggagcycaaataataaaatcttaatgatttcctacaaactgggactcccagtgtttttggttttttgttttgttttgttttgttttgagacggagtctcactctgtcgctaggcgggagtgcagtggcgtgctctcggctcactgcaacctccgactccctggttcaagggattttcctgcctcagactccgtagtagctgggatttacaggcacgcaccaccacgcccagctaaatttttgtaattttagtagagacggggtttcaccatgttggccaggatggtctcgatctcctgacttcgt

SEQ ID NO: 115 - DNA, 599 bp, Homo sapiens
agcgggcgcccaccgttccttacaggagcctcctcccattatgaaatgcctggcatcccaggagtgttccactctcccacacaacagggaagtggcagagatcagagctgaggggataggaagcagctcagccctgcagctggagctaccggggaggatggagagctgcagaccctggcttctcctcaggaacgtgtccaccctgccccggtagccctctgatgtctgccatcctgtgttcacacggaaagacagtcttccaatttaatcacgcagtttgatgctaccctactatttccmtatggacaaatcactcaaaatcaagttaaactaggaggaagcggctgacccgttggctctttctagagcgcagctgatatgtctttgacacacctgaaaggaaactcccacgccctcaccctcctttgatcagggttgcaggttggcgccgaagacccaaggactgcagtgctgctcacgtgccatgccgtcctggggagactcccaggctgatgctgatggcgacagaggccggagctctggggacagcggaggggcaggcaatgttgtgcgcagttacccagggcaggcagcactgg

SEQ ID NO: 116 - DNA, 599 bp, Homo sapiens
acacgagtgactattaactccagtgtccacaggttgatttgcttgtgtgcatttgtggcacttcctctgtggcccaagtgccaggatgagctgcatccgtgtctcatttaggagacatgcacgatgggtctctgtccccgcaagctgaaccgttcaaaaccaaggaaggaaaattatagcatcaaaagggattaaaactagtgatttgctgtttgagcatttcaaatagatccggaacacctatgattcactgagtggagaataactaaaacaaataaaaaatcatttagttgccagggyaatgtattccagcagaatgaacactctatggaggcccattcctaaagggtgcctccaggtagcaaaggcccaatggctttctcctccgcctgagtgtttgcgtgccatttttgtattccaacacaggcctgtctatgacttcctcctgttccctgcgcctgggctagattagagaggtagggtgttaatggtggacttgatgaagattgaagacccattttctcccccttgagacctgcctgatttggataaattgagaagaacaaagcagcccctttctgtccttgggagaaaacctg

SEQ ID NO: 117 - DNA, 599 bp, Homo sapiens
atacacaggcaggagtgagttacctgtattaaatcacaaactgtgtggcgaagattctaagtggaaaagagtgtttttacaaatggataggcgggtgaagacacagaacctgccatgggtgggggtgggcaagctacaggtgacaggtggggagggaacagcctctgcacaccaacagagacaggatgaacatcacatatgcagcagacacggccagaggatgacgctgcacacgtgagcagggtcaggaggtacacacgtaccacaagcatgaagcacctcctacgtgcctgactgggygaggtcccagctgccctcagagggtgtacggtccagtgaggccagcagacacataagccagggagaaggagccccagggcatagaggagtgggacccagccaggcaggcggtcagggaaggcttcctgggggaggagacatctgaggtgagtctggaaggaagaggcagccaagctagagaggatggaggagggggggacttggtgtggaagaaaaacaggaagaactacacaggttctcaagccaaagggtggcaacagagcaaggagggcctgggagcagcagggtgtgggggggag

SEQ ID NO: 118 - DNA, 599 bp, Homo sapiens
cagaggatgacgctgcacacgtgagcagggtcaggaggtacacacgtaccacaagcatgaagcacctcctacgtgcctgactgggcgaggtcccagctgccctcagagggtgtacggtccagtgaggccagcagacacataagccagggagaaggagccccagggcatagaggagtgggacccagccaggcaggcggtcagggaaggcttcctgggggaggagacatctgaggtgagtctggaaggaagaggcagccaagctagagaggatggaggagggggggacttggtgtggaagawaaacaggaagaactacacaggttctcaagccaaagggtggcaacagagcaaggagggcctgggagcagcagggtgtgggggggaggggtggggcagaggccacacccaatgggacagggcgagggcctggccttaagctgggcagggatcctgatactcttggacaaaggaggctatttatttatttatttatttatttatgagacagagtctcgctctgtcgccaggctggggtgcaggggggctatctcagctcactgcaacctccgcctcccaggttcaagcgattctcctgcctc

SEQ ID NO: 119 - DNA, 599 bp, Homo sapiens
ggctacagagaggaaggtgtgcacaggtgaggccttgaggggttgggaggcagggaggggagctgctttccaggcaaagggaacgaggagaagatttccacataaagatctgggccttggcacctcactcctggcagcctgtctgtaattcactctggtcacccctctgtggagcagtgtgcctgcctcagtcatctctgttgatgatttaagcctagcagccttcagccaaatgtggcaactgtgtaagccaatacccagctagctcctggaccactcggatgcccatcaacatgtacsaggctggggttacttctcctgggatcccagaaatcctcagatttgcacgcaatgcaaatgggcattcgggggggcctgtgggtggtcccgattctgttcttgggagcgggagtgaaaagcaagcctgtcgtggggaaactggcaccttcactggtggccacgctgctggggctgggcgtgaagctgcctccggcccttggagaggagtgtgagggaagggaggcggcagtgctttttatacacaggagctggaggagggtgatggcagcttggaggaagtcatcactgccatggtaggg

SEQ ID NO: 120 - DNA, 599 bp, Homo sapiens
cgaagccagtaaataaattctgcctcactccaggccctgagccgagtggtcctggagggcagctcaagtagccacttgcagactttctgtcctgctgctgtcggcccatgcagatgctcctctgacacgctcgtgggctgatcactctccttggaataaggagcggtgagcctgcagagtgctctgcaccctcccaccctcccaggtcctctaagacataaactcattcatggctcccaaactccaagggccaggatcactcgatccgttttacagatcaggactcaccctcctccttcmgggctggctgctgttttcattcactgatgtctctaccatcaatcaggtacagccatttgaaatctcacccagagttaaggggctttttaatggggattcatttctaccaggtggtgaagctgccatccccttaaggaagggtcctgcctcagtttctccccaagatctagcacaaactggtgtacacagcatgctcagagccacctgcttatcaatgcctgactcatagagttctgggccagggtgcagctgggctcattttgcacattgccgtctattatgcctttgtgggagacgca

SEQ ID NO: 121 - DNA, 599 bp, Homo sapiens
aaggtcctggtgtgtgttccgctgtgccgtggagcttcatttataaaacacaattcaaaggtaaaaagaccctaagcatttcaagatggtgccacagagcattaaacctcaagcactgggtcccatgcgactgtcctggtggcacaccatagagccaatcctgcataaatctgagaaagatcacagaaaggtaaaattgcagaaatgttgctacttttatgtttctcatctacctgttgcatttcgacccatcatttcctatgctgcctctcttaaaccagaggttcctcggaggcacaygcttgacacagtgtgactacgaaacagcaggggagtgataagtctgcagcacggccgcctgctagacgccacgctcagaggggcaagaaacctcatacaactgctgtcacttgggagggctgaggactcagagacaagggatggggtgtttgacctctcaagccctctgtggttggccgattccacagagagctgtaggaattagcaaagcaataggggctggctttcatttgctaatgaagagatatccaaaaatgataatgataaccccactgtgcaactgcaggactttctccaca

SEQ ID NO: 122 - DNA, 599 bp, Homo sapiens
agccaccggatgggaggaatgtacggtcgccatcagcaagggagggtttgcgtgagtggctctggatgataaggacactgcacaggcccataagtgacgagagaaagggtgaccgtagtgctcagcagagagcagctctctgtcctgagccttgttttgccttctctgaggattcaggatcctgtggcaagggtgagggtgcctagtcttcttgaatctggaaaactatgtgactctactgcacatactataaagagaaagcacatatcagaacttttctgcagcagacctgttgaacaytggggagacagagacaggaaagtatttggctagggccagactggcaagtaaacacagcaaactcaacacatggcacagaatgcattctttccactaaaatggcactaaagaaatatttgtttcccatcaaatatatgtgttatatattaattaattaatacatgcagacacatgcatgaccacaaaacaaagaggaagaatgaggaggtatgactgtctctctgcatcggcacctcagagaggaaggcacacagaaagcaagtggaatcacaccacagaaccctagaaaggctcagatc

SEQ ID NO: 123 - DNA, 599 bp, Homo sapiens
ggacactgcacaggcccataagtgacgagagaaagggtgaccgtagtgctcagcagagagcagctctctgtcctgagccttgttttgccttctctgaggattcaggatcctgtggcaagggtgagggtgcctagtcttcttgaatctggaaaactatgtgactctactgcacatactataaagagaaagcacatatcagaacttttctgcagcagacctgttgaacactggggagacagagacaggaaagtatttggctagggccagactggcaagtaaacacagcaaactcaacacatrgcacagaatgcattctttccactaaaatggcactaaagaaatatttgtttcccatcaaatatatgtgttatatattaattaattaatacatgcagacacatgcatgaccacaaaacaaagaggaagaatgaggaggtatgactgtctctctgcatcggcacctcagagaggaaggcacacagaaagcaagtggaatcacaccacagaaccctagaaaggctcagatcctcaagtcaccagctacctttgaaggtggaggtgtgcagggggctgaaccaggaagactggttgaaagaagg

SEQ ID NO: 124 - DNA, 599 bp, Homo sapiens
tctataatctaactgggtatattcataaatgtgagtcagaaagtcacacttagtttaaggtaactaacttctgatggaattcttgctggtagatggtgaatgtgggtcttaatgagctctcctatgaaaggggcacttaccatggacaggaagatatgaaaaagcatgctggctctgtgagtgcccctcaaggcagcctttgcatttgtaccaaaattcaaaggacggaaatgttcagtcagaagttctcttacctacctcactccagctagggaaccaaggccatctatctcttttccyacaatgccatggacaaatggaatagaaacaaatcagcaaaggtagctgccacatcttgcccaatgatgtgtcctagaggctctgagattcacttgaatgaagtcaccagcagtgaaattcacagttaatgggaagatgcaaaatgctcaggagacattagaaagctagccccatagaccctgaaggtgaagaccaaaagcttccccagtggtaagatataggagagaaggggcattccctggccctaaacttactaatgtcctcctgtggaaccattcctttagtaactattaataaaa

SEQ ID NO: 125 - DNA, 599 bp, Homo sapiens
agatatcagataaacccataatatatcagagcagttctttgcggcacatatttaacttctgtgaccatagcctccaaattctctaacagtgcggtattctggaaagagcacacaccccttatgcccattctactgcatccagcacacaaggctgggcagaaagcaaggttctctgcagcagcctaccctggaagtttcaacacagccactgcactatgactgtggccttacttggcctgccagcgcctttcagtgcttaccttttggcaggtagagtttaacaggaagcaatagccctgmtaccaaatgacagaagaacacaatctgagggttgatgacctttgtattaatttaaacatctcaacctttctcaaccaggacaggcagccacctccacttgtgaacctgaattcggctgccacaatttgcatgcataatgtactgaaaagccttctttataatgtttacttctctgtttcttccaaatgaaatcactgaagggcaaatttgattttcaaatcagttttaaaacatctgccttctaaaaaagtaagagtaaagagataaggcttttatattacaggcaagggctaattatt

SEQ ID NO: 126 - DNA, 599 bp, Homo sapiens
ggcagaaccatggctaagagccaggcctcctgattttcagcccagggatttttattttgccttttttacttcactcatgtacagataatgctgccatatttcacacttttcttgctggtggcaaatactagtaaagagtctaataacacaatccctagtataaaggctaagattgaaataattttacattaaacagaaacaattccaacacagattttcactttcatacatgcaccccttattccagcaattacactgtacttctcctggggacaggctctgagccctccaccagttaasttctagtacatagtgggtacacagtaactgctaaaccacaggaggtttgcagctcttgttccacagcgagcctgcactttacactctgacaatcaccaatcaccgtgggctctttaattttgtggtcaggcagatttttgcctgccacagtactgaactcatagtaggtacttgataaaaaggggttgaatgagtgaagaaaggtctgcatacgtttcctgcaggtagacactgcattttcttatcagtaaaatgggagtcttgagagaaggtagtcaagactggagggtcagagcaag

SEQ ID NO: 127 - DNA, 599 bp, Homo sapiens
ccaggaactagcaacactactatagattttaggtcctctctgaaaccacaattctcagttctccagagctcccatttcccactttacttttcatttgtagtaaaattaacaaatcatatgacctagacatttctaaagactactggtctgtgacatcatttcaaaccatccccaaacctgaaatccaaatagccaacatcctaacagaggctaaagaacaaagcacaaacccagtgaaggggaaaccacttctgttttgaaggaaagactcatttggattacgacagttagccaacacaygcaggtctgtaatgggggatatctccatataaaattcctaagtaaaagggtcacaagaactataaatcctaatttaattcagccaatctaagtaaatcccaaagaaacatgctgggctagaaatcaaatcccagtaaacaactggaaaatgtgggcacagtaatatgaggactccagagatttctgcagcaaatggattgttctaatgacatttgattaaaaatctgcttccacctgggtcagtgcatttcatgtgcagagtggattcagttccgcatgcagaataaaaatttcaatgg

SEQ ID NO: 128 - DNA, 599 bp, Homo sapiens
ggtggggagggttacaatgtaaagcatttgtctgtttccttttcaatggtattttccaatgttttaaaaaaaaaaaatcagccagatatggtagctcatgcctctactcccagcactttgggaggctgaggcaaaaagactgcttgaggccaggagttcgagaccagcctgggcaataaagagagtccctgtctctatttaaaaaaaatagaaaaattagttgggcatggtggtggcatgtgcctatagtcccagctacctgggagatggaagcaggaggattgccggaggctaggtttrgaagcctaggtgacagagcaggactttgtctcttaaaaaacaaaacaaaatcagcaagcacctcattcaatcttttttaaacaaaaaatttgaagaaattgattctgagccaaatatgagtgaccaatggcctgtgatacaaccctcaggaggtcctgagaacaagtgctcaaggtggtcatttggggcacagcctagttttatacattttagggagacgtgagacgtcaatcaaatacatgtaagatttacattggttccacctggaagggtagggcaactcaaggacgggtgcttcc

SEQ ID NO: 129 - DNA, 599 bp, Homo sapiens
aaaaatttgaagaaattgattctgagccaaatatgagtgaccaatggcctgtgatacaaccctcaggaggtcctgagaacaagtgctcaaggtggtcatttggggcacagcctagttttatacattttagggagacgtgagacgtcaatcaaatacatgtaagatttacattggttccacctggaagggtagggcaactcaaggacgggtgcttccaggtcattaaaaattttctgattggcaattggttgagagttattatcaacagaaaggaatgtatgctttacagtaagctgttgyggagaccaaggttttttgtttttttttttttgagacggagtctcgctctgtcgcccaggctggagtgcagtggcacgatctcggctcactgcaagctccacctcccgggttcacaccattgtcctgcctcagcctcccgagtagctgggactacaggcgcccaccaccacgcccagctaatttttttgtatttttagtagagacggggtttcaccgtgttatccaggatggtctcgatttcctgacctgttgatccgcccacctcggcctcccaaagtgccaggattacaggcgtgagc

SEQ ID NO: 130 - DNA, 599 bp, Homo sapiens
aaaaagcacagccaggagacagctaaaaggagcttaagaatgtatatagtcattttattataaatggctactctggagccaatatacttgactttgacttttttaaggcaagattaaccatgacacattttggtcaagcttaggaaacaacttgctaataatctatcttgtgaaaaaagatgcctctggaaattcatcatattggtggagggagaaaacacctacattttggctgtcactaaaagccaaacagaaacttcctgttttcaagggtcagtggaaaattactgcttattatayagtattacatttacagaaactgcaagtgctcatcacaaactgaacgtcacacttaccttatcagaaagagcagcctggcactggcacaggagtgcaacaaaacacggtcaatttactaagcctcccgatagccaaggttattgttggcaatacaaaaggtaacaaaggagtggactggctgagcgtgcaaggctgctatcctgccatctctgggaagatctgagctcaaggcacacctgaacttccctccagcaggctgaagagagaacgagtgattttcttgtatccaccaaggccag

SEQ ID NO: 131 - DNA, 599 bp, Homo sapiens
ggagcttaagaatgtatatagtcattttattataaatggctactctggagccaatatacttgactttgacttttttaaggcaagattaaccatgacacattttggtcaagcttaggaaacaacttgctaataatctatcttgtgaaaaaagatgcctctggaaattcatcatattggtggagggagaaaacacctacattttggctgtcactaaaagccaaacagaaacttcctgttttcaagggtcagtggaaaattactgcttattatatagtattacatttacagaaactgcaagtkctcatcacaaactgaacgtcacacttaccttatcagaaagagcagcctggcactggcacaggagtgcaacaaaacacggtcaatttactaagcctcccgatagccaaggttattgttggcaatacaaaaggtaacaaaggagtggactggctgagcgtgcaaggctgctatcctgccatctctgggaagatctgagctcaaggcacacctgaacttccctccagcaggctgaagagagaacgagtgattttcttgtatccaccaaggccaggcccattccatctaggtagtaagtactt

SEQ ID NO: 132 - DNA, 599 bp, Homo sapiens
gaatgtatatagtcattttattataaatggctactctggagccaatatacttgactttgacttttttaaggcaagattaaccatgacacattttggtcaagcttaggaaacaacttgctaataatctatcttgtgaaaaaagatgcctctggaaattcatcatattggtggagggagaaaacacctacattttggctgtcactaaaagccaaacagaaacttcctgttttcaagggtcagtggaaaattactgcttattatatagtattacatttacagaaactgcaagtgctcatcacwaactgaacgtcacacttaccttatcagaaagagcagcctggcactggcacaggagtgcaacaaaacacggtcaatttactaagcctcccgatagccaaggttattgttggcaatacaaaaggtaacaaaggagtggactggctgagcgtgcaaggctgctatcctgccatctctgggaagatctgagctcaaggcacacctgaacttccctccagcaggctgaagagagaacgagtgattttcttgtatccaccaaggccaggcccattccatctaggtagtaagtacttccagattta

SEQ ID NO: 133 - DNA, 599 bp, Homo sapiens
actaaaaacttgattacttcttaatgttattgtggctctgtcttcacagggcaattgaggctttcaattagcctaccagatccacgctcaaacgggcagaggctgtgataggaggggctcagtggtgctccgagaacaccaactctgaagtcctgttggtttaaatctgtgaccctgacactagattaatgtagatttgggttttgatcttaattaggaaaaatgaattctgaagattcagaaatgtgaaacaaacaaacaagcttccaggagcccctccttctcccaccctcagcataycctaaagaaaagactatacatttcaaagctccagcatcagaatttctattaagcttatatgtccactgctaaatcctacagagaattcagtaactctccagagaatcaggaaggaaaaaagaacgatataaacaagttctctcctctaatagagcagtaccaacatatgctatcttcctttgcctaagaatcacctcacagcagaaatttacactaaggctctgagaaccgcagatgaggaagcaagcagtattatccagtcttcctcatctgaggaaaaccacgaactctctgggaca

SEQ ID NO: 134 - DNA, 599 bp, Homo sapiens
tgctccgagaacaccaactctgaagtcctgttggtttaaatctgtgaccctgacactagattaatgtagatttgggttttgatcttaattaggaaaaatgaattctgaagattcagaaatgtgaaacaaacaaacaagcttccaggagcccctccttctcccaccctcagcataccctaaagaaaagactatacatttcaaagctccagcatcagaatttctattaagcttatatgtccactgctaaatcctacagagaattcagtaactctccagagaatcaggaaggaaaaaagaacratataaacaagttctctcctctaatagagcagtaccaacatatgctatcttcctttgcctaagaatcacctcacagcagaaatttacactaaggctctgagaaccgcagatgaggaagcaagcagtattatccagtcttcctcatctgaggaaaaccacgaactctctgggacaaatttaatgtgcaacattgcctcaaggtagaaggccgaacaagatgaccactcatcaggtcaagattgctaagccacattactaggaatacaaacattcttttgctccatttaggtttattacat

SEQ ID NO: 135 - DNA, 599 bp, Homo sapiens
tctggaattgcacacatttaccagctgctttggagggagtttgcgagtgctttctgctcatacacttttcacttctttatccaaatttatacaacagtatggagcccatgtaataaacctaaatggagcaaaagaatgtttgtattcctagtaatgtggcttagcaatcttgacctgatgagtggtcatcttgttcggccttctaccttgaggcaatgttgcacattaaatttgtcccagagagttcgtggttttcctcagatgaggaagactggataatactgcttgcttcctcatctscggttctcagagccttagtgtaaatttctgctgtgaggtgattcttaggcaaaggaagatagcatatgttggtactgctctattagaggagagaacttgtttatatcgttcttttttccttcctgattctctggagagttactgaattctctgtaggatttagcagtggacatataagcttaatagaaattctgatgctggagctttgaaatgtatagtcttttctttagggtatgctgagggtgggagaaggaggggctcctggaagcttgtttgtttgtttcacatttctgaatctt

SEQ ID NO: 136 - DNA, 599 bp, Homo sapiens
atgtgcaacattgcctcaaggtagaaggccgaacaagatgaccactcatcaggtcaagattgctaagccacattactaggaatacaaacattcttttgctccatttaggtttattacatgggctccatactgttgtataaatttggataaagaagtgaaaagtgtatgagcagaaagcactcgcaaactccctccaaagcagctggtaaatgtgtgcaattccagatgatcaccactctgaaactatactttagaactttgtaagaaatgtttattactgatactcagtacataatttayaacaccacggtgaaattcacttacattccatttcaagggatggtgaaaagaaaatgtcaactgtggggaaaatatgctttatagagcttggcttaaaacagagggaaaaaaaccacacatgcaagtctcttttcgtggaaggaaacagtctaataaatggcctaatgaacagtgtaatgattttttttaagtaggcaataggaaggaaaagggaagaaaaacaggctaatatactctacaacctgaataatgaacccctagataatctagatttgaccactggtgtagaagctatactt

SEQ ID NO: 137 - DNA, 599 bp, Homo sapiens
ctatacttttaaaaccaattttaaagtatgaattatgatacagctaataggctggacaaattaatattctgtggaagtagtactgacagcaaggtagggaatcatataaaaataaaggtggatttctttgtatccatagtcttcatcatacttctggaaggtggatagttgacttgtacctttatgtgattttaaaatctgaagttcatagcctccttgggttgggaaatcaccagtcgtcccaggcacaacatgagccagtagcagatgggtaggctcaaaatgcatcttttcagaaaratttccctaactccatcccatactctgataatgtttatatacttacagtacttacatatctgatttttcatgtttactgtggtcttataggccatgcaaaattctgggaaaaagggactcagaatcaggagacctgagttctaatccagggcttgccattagatgagaacttgggaaaggcacctgtgactgctgtctcatctgtaaaatgggaggcttgggctatgatttccaaggtacttctaccagttacagtattgtttgattctaggatcttgttttgtattttacactgtgtg

SEQ ID NO: 138 - DNA, 599 bp, Homo sapiens
agccagtagcagatgggtaggctcaaaatgcatcttttcagaaaaatttccctaactccatcccatactctgataatgtttatatacttacagtacttacatatctgatttttcatgtttactgtggtcttataggccatgcaaaattctgggaaaaagggactcagaatcaggagacctgagttctaatccagggcttgccattagatgagaacttgggaaaggcacctgtgactgctgtctcatctgtaaaatgggaggcttgggctatgatttccaaggtacttctaccagttacartattgtttgattctaggatcttgttttgtattttacactgtgtgtgcatatgtgtgtgtgtacacaaacaagatggttcccaaatgttctgtataacctgtaagttccttcatagaagaaatgtcttcctgtctcctcagaattttccttcacaatgtattaatagtatgagcctctacctactagggtcactgaacaaatattagactgaatcaaagttgtgcatttgactacagacctacagagatgactctgggaaaagctgctcctatacttccaaggggataaatcccaatgta

SEQ ID NO: 139 - DNA, 599 bp, Homo sapiens
attctgggaaaaagggactcagaatcaggagacctgagttctaatccagggcttgccattagatgagaacttgggaaaggcacctgtgactgctgtctcatctgtaaaatgggaggcttgggctatgatttccaaggtacttctaccagttacagtattgtttgattctaggatcttgttttgtattttacactgtgtgtgcatatgtgtgtgtgtacacaaacaagatggttcccaaatgttctgtataacctgtaagttccttcatagaagaaatgtcttcctgtctcctcagaattktccttcacaatgtattaatagtatgagcctctacctactagggtcactgaacaaatattagactgaatcaaagttgtgcatttgactacagacctacagagatgactctgggaaaagctgctcctatacttccaaggggataaatcccaatgtaccttgaaaaatggctagatttttaaaaagtaacctttctcctggactaggcccttatttcttcccttttctgaaacagacccaagaaacagtaaatgtatcaagttatatgcatgccaattacagtggacattcacataatggag

SEQ ID NO: 140 - DNA, 599 bp, Homo sapiens
agaacttgggaaaggcacctgtgactgctgtctcatctgtaaaatgggaggcttgggctatgatttccaaggtacttctaccagttacagtattgtttgattctaggatcttgttttgtattttacactgtgtgtgcatatgtgtgtgtgtacacaaacaagatggttcccaaatgttctgtataacctgtaagttccttcatagaagaaatgtcttcctgtctcctcagaattttccttcacaatgtattaatagtatgagcctctacctactagggtcactgaacaaatattagactsaatcaaagttgtgcatttgactacagacctacagagatgactctgggaaaagctgctcctatacttccaaggggataaatcccaatgtaccttgaaaaatggctagatttttaaaaagtaacctttctcctggactaggcccttatttcttcccttttctgaaacagacccaagaaacagtaaatgtatcaagttatatgcatgccaattacagtggacattcacataatggagcagtcacttgtgtttgggaggagaaagatcaaggaagaactcatagagcatttggctgggtcttg

SEQ ID NO: 141 - DNA, 599 bp, Homo sapiens
gctatgatttccaaggtacttctaccagttacagtattgtttgattctaggatcttgttttgtattttacactgtgtgtgcatatgtgtgtgtgtacacaaacaagatggttcccaaatgttctgtataacctgtaagttccttcatagaagaaatgtcttcctgtctcctcagaattttccttcacaatgtattaatagtatgagcctctacctactagggtcactgaacaaatattagactgaatcaaagttgtgcatttgactacagacctacagagatgactctgggaaaagctgstcctatacttccaaggggataaatcccaatgtaccttgaaaaatggctagatttttaaaaagtaacctttctcctggactaggcccttatttcttcccttttctgaaacagacccaagaaacagtaaatgtatcaagttatatgcatgccaattacagtggacattcacataatggagcagtcacttgtgtttgggaggagaaagatcaaggaagaactcatagagcatttggctgggtcttgaagggaacactgaataggtggagacttgaagatcagccagagcaaataaaaataat

SEQ ID NO: 142 - DNA, 599 bp, Homo sapiens
gttccttcatagaagaaatgtcttcctgtctcctcagaattttccttcacaatgtattaatagtatgagcctctacctactagggtcactgaacaaatattagactgaatcaaagttgtgcatttgactacagacctacagagatgactctgggaaaagctgctcctatacttccaaggggataaatcccaatgtaccttgaaaaatggctagatttttaaaaagtaacctttctcctggactaggcccttatttcttcccttttctgaaacagacccaagaaacagtaaatgtatcaarttatatgcatgccaattacagtggacattcacataatggagcagtcacttgtgtttgggaggagaaagatcaaggaagaactcatagagcatttggctgggtcttgaagggaacactgaataggtggagacttgaagatcagccagagcaaataaaaataattagcttacctaaagcatgggctcatttaacaatagtgattaggccaggctgaaaggaaagtaggaatataaggactgtaaagagagaattacagaaataaaggttagaaagtctggttagagtcagagagatatggt

SEQ ID NO: 143 - DNA, 599 bp, Homo sapiens
attaatagtatgagcctctacctactagggtcactgaacaaatattagactgaatcaaagttgtgcatttgactacagacctacagagatgactctgggaaaagctgctcctatacttccaaggggataaatcccaatgtaccttgaaaaatggctagatttttaaaaagtaacctttctcctggactaggcccttatttcttcccttttctgaaacagacccaagaaacagtaaatgtatcaagttatatgcatgccaattacagtggacattcacataatggagcagtcacttgtgtytgggaggagaaagatcaaggaagaactcatagagcatttggctgggtcttgaagggaacactgaataggtggagacttgaagatcagccagagcaaataaaaataattagcttacctaaagcatgggctcatttaacaatagtgattaggccaggctgaaaggaaagtaggaatataaggactgtaaagagagaattacagaaataaaggttagaaagtctggttagagtcagagagatatggtaattttatatttgaaatctggacatgtggtttaacaggaggcaggatatttaaaa

SEQ ID NO: 144 - DNA, 599 bp, Homo sapiens
atcaagttatatgcatgccaattacagtggacattcacataatggagcagtcacttgtgtttgggaggagaaagatcaaggaagaactcatagagcatttggctgggtcttgaagggaacactgaataggtggagacttgaagatcagccagagcaaataaaaataattagcttacctaaagcatgggctcatttaacaatagtgattaggccaggctgaaaggaaagtaggaatataaggactgtaaagagagaattacagaaataaaggttagaaagtctggttagagtcagagagayatggtaattttatatttgaaatctggacatgtggtttaacaggaggcaggatatttaaaaatgagatgtttagggagagctggtcatgtagagatagaagatgacccaggtaaggatcggaggctttcctcactgaaggtccctgtacagggattaagaaatcacagcaacgacaacctagcaacagtgttctagactctagtacagcaacagacgtagagtatagaaattctctgcaacagtattgagggaaactcactagaagctactataaatactccaggaaataagtatgaagg

SEQ ID NO: 145 - DNA, 599 bp, Homo sapiens
gaaagatcaaggaagaactcatagagcatttggctgggtcttgaagggaacactgaataggtggagacttgaagatcagccagagcaaataaaaataattagcttacctaaagcatgggctcatttaacaatagtgattaggccaggctgaaaggaaagtaggaatataaggactgtaaagagagaattacagaaataaaggttagaaagtctggttagagtcagagagatatggtaattttatatttgaaatctggacatgtggtttaacaggaggcaggatatttaaaaatgagatgyttagggagagctggtcatgtagagatagaagatgacccaggtaaggatcggaggctttcctcactgaaggtccctgtacagggattaagaaatcacagcaacgacaacctagcaacagtgttctagactctagtacagcaacagacgtagagtatagaaattctctgcaacagtattgagggaaactcactagaagctactataaatactccaggaaataagtatgaaggtctgaatacgaatggaggcaatgggaatggaaaaaaaagatgaaagggtataacagaaaggatatagca

SEQ ID NO: 146 - DNA, 599 bp, Homo sapiens
attgcaaactgcaacaagggctgtgaaaaaaagtagcttggttttatgagaggggataacacagtgatttgatcaagatgggtggtcagggcaaactttcctgaaaagtggcccttgagctgagaagggtaagtgggggtggttaatgagacaaagggcagagggtgggctccagacagagaccagcatggcatgcgcaaaggctctgtgtgaggacagagcatgatggatggtgggaaaacagaggatgccagggagatgggaacagggagagggatgcagagggagtatggagaggcrgggggccagattgtgcagggcactctaggccacgcaaagaattctgttcttgattcagagagcagcaggaagcagtgcagggtcctaagcaggacgatactagagttgtgcttgaaaagtgccacctgcctatagtaagtaaactcatgggagggcacagagggaatgtgggggggtcagtcagtagagtggagtggaacaggtaagtgatgatggtaggtgaactaaatgtgatcttgatttatttttaatcatttggaatataacattaataagaaagttatttccataataaaata

SEQ ID NO: 147 - DNA, 599 bp, Homo sapiens
ctgtgtgaggacagagcatgatggatggtgggaaaacagaggatgccagggagatgggaacagggagagggatgcagagggagtatggagaggcagggggccagattgtgcagggcactctaggccacgcaaagaattctgttcttgattcagagagcagcaggaagcagtgcagggtcctaagcaggacgatactagagttgtgcttgaaaagtgccacctgcctatagtaagtaaactcatgggagggcacagagggaatgtgggggggtcagtcagtagagtggagtggaacaggtragtgatgatggtaggtgaactaaatgtgatcttgatttatttttaatcatttggaatataacattaataagaaagttatttccataataaaataaaaatttctgaccatgtacattttgtgtgctcacacagctcactgagaaagttttattgaggtatgcttcattttttttataacaggcatggaaaaaattattaccctcacagaataacgtggctcctgggttaagtaagaaagaaggaaataggagctctcagattcttcttcctgatcatttgctacccaagaggacttcatt

SEQ ID NO: 148 - DNA, 599 bp, Homo sapiens
gggtagagaggagcctcccactgggggtccacaggatctgctggcagctctaaatttagaactcaggcaattcacatacttagcatcaaaatctactttccacatgcctcccaatgtttgggtaaatttataataaaaacttcctgcaatacaaaagataagtcccctttaaatgtgcaggtatgactggctgactccttattgctattttctattctaaaataagcatcagctttaggtacttcataaccccataaacaagtgtgagtaactggtgtctccaagtgtggcctaacgtayagatgacttctatagggcaagcctgctgatatctcatcattaacacaaggatctgcctagggtcagacttggcgcacgtgcacgtacgtgtgtgtgtgtgtgtattgtctaaaatctgctattttcttcatttttcattttttgaaaccattcctcttacaggtacatgtttcttcattgttcaagttctaaagctcttgacaaaattaacaccaatgtttgccctttaaaaaaaaaaaacagaaaagaacatgactggcttttttcattactgtcaaatgctgtgtagaaaaacggaa

SEQ ID NO: 149 - DNA, 599 bp, Homo sapiens
cgtatagatgacttctatagggcaagcctgctgatatctcatcattaacacaaggatctgcctagggtcagacttggcgcacgtgcacgtacgtgtgtgtgtgtgtgtattgtctaaaatctgctattttcttcatttttcattttttgaaaccattcctcttacaggtacatgtttcttcattgttcaagttctaaagctcttgacaaaattaacaccaatgtttgccctttaaaaaaaaaaaacagaaaagaacatgactggcttttttcattactgtcaaatgctgtgtagaaaaayggaattaagtatgaaaaaagaaaaataaaatcttcatcgagcatttcaaaagtgacaattacacaaccagaactcattaattggttcacgcttctagaaggaacaagcaaacatactcctccaacctggtagggcagaaaaatctgaggacaacagccctgtctgttattaccaatatgggatttatgcgacagtcaagtgcttcaatcttcttggaaactaacacgtcacccaactatgaactatgtctcatcatcattagtatcaatgaggaagacatttcctcctttatatcctgc

SEQ ID NO: 150 - DNA, 599 bp, Homo sapiens
agggcaagcctgctgatatctcatcattaacacaaggatctgcctagggtcagacttggcgcacgtgcacgtacgtgtgtgtgtgtgtgtattgtctaaaatctgctattttcttcatttttcattttttgaaaccattcctcttacaggtacatgtttcttcattgttcaagttctaaagctcttgacaaaattaacaccaatgtttgccctttaaaaaaaaaaaacagaaaagaacatgactggcttttttcattactgtcaaatgctgtgtagaaaaacggaattaagtatgaaaaragaaaaataaaatcttcatcgagcatttcaaaagtgacaattacacaaccagaactcattaattggttcacgcttctagaaggaacaagcaaacatactcctccaacctggtagggcagaaaaatctgaggacaacagccctgtctgttattaccaatatgggatttatgcgacagtcaagtgcttcaatcttcttggaaactaacacgtcacccaactatgaactatgtctcatcatcattagtatcaatgaggaagacatttcctcctttatatcctgctttctttctgttgcatct

SEQ ID NO: 151 - DNA, 599 bp, Homo sapiens
cttggcgcacgtgcacgtacgtgtgtgtgtgtgtgtattgtctaaaatctgctattttcttcatttttcattttttgaaaccattcctcttacaggtacatgtttcttcattgttcaagttctaaagctcttgacaaaattaacaccaatgtttgccctttaaaaaaaaaaaacagaaaagaacatgactggcttttttcattactgtcaaatgctgtgtagaaaaacggaattaagtatgaaaaaagaaaaataaaatcttcatcgagcatttcaaaagtgacaattacacaaccagarctcattaattggttcacgcttctagaaggaacaagcaaacatactcctccaacctggtagggcagaaaaatctgaggacaacagccctgtctgttattaccaatatgggatttatgcgacagtcaagtgcttcaatcttcttggaaactaacacgtcacccaactatgaactatgtctcatcatcattagtatcaatgaggaagacatttcctcctttatatcctgctttctttctgttgcatcttatttgtgacatcaagttctttacacaaaactccaaatcacatggttagcagtg

SEQ ID NO: 152 - DNA, 599 bp, Homo sapiens
gaaaatgacttggggtcactggggactatcaacagctgactacaagtcagcacaggccgtggtgcttttgggaattcaggtccaagatggctgacattgtaggcacagttcgctctcccttcccggaaaccactgacatgccatcaaagaaatgcaaagagaatacattcaaaaaggttgcaaattggagaggacattagtaaatgaaaaatcttggtgaatttcgagaaaatgatatgcacatgaaatattactcatttataaatcgaggagaactgggcagatttccataccgaagcraactgagcaggatgaatgacaaatgacccacatccagccatgtcactgtgaaatttgagactaccgaggtgaagagaatatcctaaaggcttctatagagaaaaataaattactgcaaagaacatgagtcagactgacatcagatttctcatctgcaacattagatactagagggcaataaatccaagctgtcagacttctgaaaaaactgaattctatacccagtcaaactatgattcagatatgctggggttctgaaaatttgttttctatatcccttttctgaaaaaaattactta

SEQ ID NO: 153 - DNA, 599 bp, Homo sapiens
tgttctgccatctaggtcctcctcagcatcagccacatttagctggcaggagaaagaacagagaaggtacacctagttctgaaaagccttgctctaggggtagcatacatgccttccactcacatgccactgagatgaactagtcacattgacatactagatgctactatggctgggaaatgtagtcagttcctggttgggtagccacttcccagtggagcacagatcttgttaccatctcagcctcttcttccagaagcagagctgagatttaaatccagcttcttgccactataccayattgcctcttaggactcaaaacactgctaaagaggactgtaccgagctagatgatgaaaagggggaaacaaagtggttaatttatcccaagctcctggaaccctttttatacaatacgagggcctccacggcccatgaacaactatgcatttgtctttccagagtctttgcttattgggtccacagctcccagaccagaccttgtacttcctcaaagcttgtcatcttgtacatgtcttacccaccacaggtctcttttgtcatttattgttgtaaactggattcctcttctcttttat

SEQ ID NO: 154 - DNA, 599 bp, Homo sapiens
ttttgagtaacttttaaaagtgtattttttgctcctatgtttgtctggttcttattttgattataagaactttagaaagatgtttggtctttctctttttaaaaactctgcttcaaacatgtaggctctgttcatggcatatgttccatatatgttctggattgaaatcattcgtaactctgacacagtagagtaaaactaggggaaaacttcaaatcaattaatcattctcttcatttaacagttctgttaccataactgttgttggaagataatgttgaggttattttccccagtggratttttaattatttatggagagctaggtttatcatattccctatccttctagataatcataagctagtgtttctacagaggactgaaatgaaaatggtcagtccgttttgggatagaacctaaccacatctcaaaacatttcatggaaaatgacagccttgaagaggatatgggatatggagaagataaaaaaaagaaggataaattatgagtatcatatatgttctagtagatattcttggagtgataacatatttgaggaaaattttaagttgtatatatcatgctctgtaagtagc

SEQ ID NO: 155 - DNA, 599 bp, Homo sapiens
gtaaactagctgggaatgtttgcatggtgagctcatccaggacagacactctctatgaccttaactgaaatgagctccttaaacatacacagcacacttccacccatagatcagaagccattttcaaaaggggctgaataaaggaagttgctggccaagatttattgatgcagatcaggtcggtgtttagctgttgccacaaacctctctaactaatgagcacagcatgaaatgcctggtgatattatatatgttgtctcctccatcttcccttattctcattgtgcagtttccctaaargcagtgaagtgctgggcttttttttttttttaattgattagcagatgtgattttgcttacatcttccataaatgctctggctggctgcataccattgattaccaggggttcagtttacagcaactaattgctctccttgctggtattttgatacaaaattcaatagcgaatatgttttcaaaactcttttctttgtggcttgagtcagaagtgactcaatgcaaacccagtggactgaggtttccagtctttaaaatagttgctctctcatcagtctgtgaagaaagtttccaaattgt

SEQ ID NO: 156 - DNA, 599 bp, Homo sapiens
tctggcactgaatccccaagagccagcattgatattcctgtggtggggcagaagaggaccccagcctatctgagtcacacatgagagtgtttgggtgtcaggggctgggaaatatccctgaggtaacctccaaggcgctggtgctggaactgtgtacaaaggatacatatgaatagcctgggggaaatgaggagcttggaggctcctagcagccaaaaggaagaggtagcatgagcaggtaaaaggaaaacagtgaagttactagttatttccttagcctattaggaccacaaagggttscaatgactctccttgggcctctgatggacacaaggctactgtccacaattccttggaatgtgtaatgaatagcagtagtttcagatgggaggcaggagtcccaactctgactctgtcttgcttcacaactgtatcctctcagatttataatctgggaagtgggtggtagggggataggggaaaggagggagatggattttaatgtacgtgatttctgattcctttttcaagtctatttttcccttttaattacaaagcgatatatgataaatctttggcacttttataaagtatccatt

SEQ ID NO: 157 - DNA, 599 bp, Homo sapiens
aattaaaaatgaaaaccatttccattcccaaaccctcgttactccctgaactgatcaagtgcccaggagctacattctcaaactgataggatagtcattgaccactaccctctagagccctactaaaggggatggaagtagtagggtagggtgaagaaattacagagacatttaaagtcaataaacaatatgtcaaaaaaataaagttttatagtctttgataaggcagttaatatgacagtagtaaaataataatatatcagttatagaggaagatgacactatagactaaaacacacyaattaagtacacagtgctgagttgtttgttggtgaccaggagaaaatggatttgtacagatctatcctgttgccagctctgtctactgtaattataaaaagcattttacaagatagctggccagggcagtctatgaccacagaacaataaaaaccttatagtacttggcattttaacagaactactcatttagactctttaatgaaaccaattttaaatggatgactctgttaaacacgttcttctcaaatagatgcaactaatctccatgaattttctgcttgtctctgagtctaatg

SEQ ID NO: 158 - DNA, 599 bp, Homo sapiens
agctctaattcctttagacagttatataccatttaattgtcctctgatgtccaatgcaaacctcttgtcatttcttttcctgatatctgacctttctgtattcatctcctttctgaggttaaaagataataaagagcatataatcaagtactgtttttattagaaggttcataggaaccctgttagaaatagataaaccatcatctataaagaattgtttgagaattggtggtaaccagataaataatatcttgttgataatagccctactagtaaaataggaaaatgactttgtcacgrttgtgttcactgtagtgagattttagctgtatacattagactcagagacaagcagaaaattcatggagattagttgcatctatttgagaagaacgtgtttaacagagtcatccatttaaaattggtttcattaaagagtctaaatgagtagttctgttaaaatgccaagtactataaggtttttattgttctgtggtcatagactgccctggccagctatcttgtaaaatgctttttataattacagtagacagagctggcaacaggatagatctgtacaaatccattttctcctggtc

SEQ ID NO: 159 - DNA, 599 bp, Homo sapiens
aaacaaaacaaaaaataaaactgaaaaaacaagtctttcttaatgagattatttgttatttgcagccaaatgcaagctcctttggaacaaatgacctctagtaaaacaaatgtaaagtctcagttgcaacatataatggcagtttctataatttatgaccacagaaatcaagggtggattgaaccataagatgtgatggctcctttcattttctttgtaaggctataggaggttctgcaggatcacgaaacaaattagaatctttggagctggagttttaccatgggttgtgttgtcctrctcctgatggagagaaagggatgaaactcaaactatgctttgatttcttcatcttgtaaatgaaaacagtgagagcagctaaagctccagaaccactgtaagacatggtgaccaaaagaaccattagccatttctaaaaattaaactgtacagaaggtctaaacactcttggctggtgtttggttacagtaaaagaataatgtggcaaggcaggtaatatcataaccattgagggatatggtttcaggcataataaggtaaaaggaagaaagaagatcctgaccattaagtcttaaggg

SEQ ID NO: 160 - DNA, 599 bp, Homo sapiens
acatggtgaccaaaagaaccattagccatttctaaaaattaaactgtacagaaggtctaaacactcttggctggtgtttggttacagtaaaagaataatgtggcaaggcaggtaatatcataaccattgagggatatggtttcaggcataataaggtaaaaggaagaaagaagatcctgaccattaagtcttaagggcagcaaggaaagaaaatccagggagaagtaaaagcatactctgcatttatctatgtaaccaatcccccttgcactgaatctgatacattgctgaaagaggcartgcagcaggcaaataatgattcctcagctagagctaagaccctgagtgtccaggaaagttgctgaaaggagtccttgacgggcaccaaccagcctgtcatcataatcatctttcctagaagaagaatgcagaaatggaggtagacagagaggagaggcaagaagaggagatgaaagagtttttcttggtctcagtgcttctgcttgttcttaagtagatatacaagcatgttttggtcaagtgcctctgtggttaccaatgtaaggggctaagctttttaaaaagagagatacactgac

SEQ ID NO: 161 - DNA, 599 bp, Homo sapiens
ataaaacatctgccacagaaggaggaggctgatgctttccactcatttcgttttccaaggtgaacaattctggtttttaaaatgattgctataatcactaccggtgaagtatttgttttaaagagttcttaacaattctagataattactaaacacacacgtgtgcatgaaaaaatgggtcagatggggccgaaatcctctgtaaaaatgtcttattgatgggttaaaatcttggaaagctaaaaggattcttttcatatatccagctatatcttatgatgactacttgccaagttctcmaatcactcaacagaggtaaatggttgacatgtgaggtaaatcacacacatgaacacacaaacatatttttttttttggcatttaacttttcctgcttttccaaagaacatctgaattctagtgctcctcctgtatttttaacacacaacttagttgccatagcaatactgtctggtcttgaaatacctctaggatgaaactggctgaggcatatttaccatgcccccaaactatcctgctatctgtttccaggatccaggatttctggccctctatgcattcacacagttcagacttca

SEQ ID NO: 162 - DNA, 599 bp, Homo sapiens
ttcttaacaattctagataattactaaacacacacgtgtgcatgaaaaaatgggtcagatggggccgaaatcctctgtaaaaatgtcttattgatgggttaaaatcttggaaagctaaaaggattcttttcatatatccagctatatcttatgatgactacttgccaagttctccaatcactcaacagaggtaaatggttgacatgtgaggtaaatcacacacatgaacacacaaacatatttttttttttggcatttaacttttcctgcttttccaaagaacatctgaattctagtgcycctcctgtatttttaacacacaacttagttgccatagcaatactgtctggtcttgaaatacctctaggatgaaactggctgaggcatatttaccatgcccccaaactatcctgctatctgtttccaggatccaggatttctggccctctatgcattcacacagttcagacttcagcacctactgggaaccatagataaaattatttgctgtatgatttttctgggaaccagagctctgaggtctgagggtgataaaagaagactttagagtgatgcagataggctcagagagaaacaac

SEQ ID NO: 163 - DNA, 599 bp, Homo sapiens
tcttgaaatacctctaggatgaaactggctgaggcatatttaccatgcccccaaactatcctgctatctgtttccaggatccaggatttctggccctctatgcattcacacagttcagacttcagcacctactgggaaccatagataaaattatttgctgtatgatttttctgggaaccagagctctgaggtctgagggtgataaaagaagactttagagtgatgcagataggctcagagagaaacaacacttagtaataaatcatggatcagtgagacaaggaagaattccttggttcmgggctgtgacagacgacattcagcatgactgatatgaagcaaattttgtaaaatatctttccaattttgactagaattataagcaatcttaatccaagccagaagagtcatatatttagatgtttgctgtattcttcaggctataaaaaggaaatttctagctctctaggttgcaaagaaggctgtcttaatgtaaatagatgtactgcttgcctgtgcagcactcagacagaatgaaatggaagcggcatggaacaagtgaggcaagcatttttacctgtgctaatgtcatcacctgt

SEQ ID NO: 164 - DNA, 599 bp, Homo sapiens
ggtagaattgttttttccttctgcaaaacaaaaagagcttcaccccacagactaaagagattatgaatacccatcaagaaactattttgggctagacatcttttgtcctaacatattgcatttatataatattttccaactgaagaactcaaaatgtttttgtgtcattaacttcttaatactcacaacacccacagagacaatgggtttagttagctgcatctatctaaacagtcttatcttttgacaccctatttactattatttgttatttaaaatattttgggtatttcacttacwctattgcacatatcaattgcaaaaacatcgttcaaaccaatgtaaaaattgttaatttaactgactatcttataattagggtttcaacaaagtgctgtcatttattagctggaaaagactggatgataggtatacttaatgcatagcatatttgagttctcatactagcctgaaaacactaaaaaggatatcacaattagcaagtagaaagataataagtcttaaactaaggcgacagagggactataaactaagacattaggtggatttggttagttattatgactctactctggtct

SEQ ID NO: 165 - DNA, 599 bp, Homo sapiens
tttaaatgatttcaagtctcctgactcagatgagttacaccccaagcctactgaaagaaattgcagatgtaatctcagagacacgattggtaatcttcaagaaattgtggaggctatcaggggtgccagaagacaaaagaaagacaactgttttccagatttttaaagtaaaaagggataggttgtaatggaaaatatagaggtttcacatcatctttaacaaaaattctagaatagcttactaaatagatggtttacaagtactttagttaatgcatacagatgacagaaaggaaggtsgggtattatttgctattcgacagaagtttatttgtacaggtagctgatgtggcttgtctctttccctccctaagcacaaagtaagctggtctggcctgaggtacaaactcgaggcttctctatctgtgatttgttgcctatggtactcagcctgtaatgtggccctttataaaaaagggtcaacaatttaaatgaagaatccacagtctgaattctccaagaaacccagcatcaactccttagggtgccctggggaaccagcaagcagcatcaacttctatggttattagcctaaacat

SEQ ID NO: 166 - DNA, 599 bp, Homo sapiens
gcgggctgatcacttgaggtcaggagttcaagaccagcctggccaacatgatgaaaccccatctctactaaaaatacaaaaattagccagccatggtgacaggtgcctataatcccagctacttgggaggctgaggcaagagaatggcttgaacctgggaggcagaggttgcggtgagccaagatcgcaccattgcattccagcctgggtgacaatagtgaggctccatctcaaaaaaaaaaaaaaaaaaaaaaaatatacacacacacacacacacacacacacacacacacatatagyagcctaaatgtacaatgtttaaaagtctacagtacagtaatgtcctaggccttcacattcacccatcactcactcactgactcacccagagcaacttcttgtcctgcaagctccattcatggtaagtgccctacacaggtgtaccattttttatgttcttctttttttttttaattgagatgagaccttgtaatgttgcccaagctggtctcgaactcctgagctttcaagcgatccgcccacctccgcctcccaaagtgctgagattacaggcatgaggcaccgtgcccaggcaccat

SEQ ID NO: 167 - DNA, 599 bp, Homo sapiens
aactcaagtctatatcaacctgctcatttataaaagtcaagaacttccacaaaatgatggatgctcactatgatgtaagttctctaaacactgagaagagaataaaaggtgagggccttttaagtcccctgggaccttttctgcacgtttaaaacaaaaacctttaattttcttcagacatttaagttatttaaccaaaacctacttacattcaaggtttgtagcttacatcagataaattaaaatagattggaaaagaaaaaagtaattttacatatatcttctatgcaactcatttaytttccaaagttggacatgcttgtacttaagccctcatctcgtatgccttcactaattgctttgctctctgtagctgagtaaagccacccacagagaagaaaggaagggagaggagggagagagagaggatgaatttttctggtgatgaattccttgattatgaagcttaattccaaagcaacaagtaaaaacaccggtgaaattacctgtaaggacagtttcctggcagagaaggagctcagagagaagctccctgtgactttggaaagaaacagaagtggactatatttagaaggtaa

SEQ ID NO: 168 - DNA, 599 bp, Homo sapiens
tacgttttcaactttctaaacatcgtttttcaagttgtctttctgttactgatttttaattaaattgcaatgtggaaaaaaaactgtggtgtgtccgatgtgacttcgtaaaatcttagatttactttgtggtctagtacatggctaattcttatgaatgtcttatgtgtgcctcaaaagaatgtatattctctaatttgggggatgtatggttctatatatatttattagatcaaaattgccaattgtattattcgaatatttatatgcttactaattactgggagatgtttgttaaawtctcctgcggtcaggcgcggtggctcacgcctgtaatcccagcactttgggaggctgaggtgggcggatcacaaggtcaggagttccagagcagcctggctaacgtggtgaaaccccgtctctactataaatacaaaaattagctgggcatggtggcgcgcacctgtaatcccagctacccaagaggctgaggcaggataatcgtttgaacacgggaggtggaggttgcaatgagctgagattgcgccattgcaccccagcctgggcaacagagtgagactgacttgaagaaaaaaaaa

SEQ ID NO: 169 - DNA, 599 bp, Homo sapiens
gaaacaatgcctagcacatggtaggcgctcaataaatatttgctgagtaaatgaatcaaagatagaaaacgatgcctatgtaaggttgtggcgcttaaccttgcttaacatagttctttacggaaagctaggaaaatctaaaagttactgaataaagcagaaaaaaattgctttatcagtggaagttttcttagaaaagtaaaatatgatcaatctctgggtcttgacctaaacattaaaaagaaaataaatatgcccatggtagaagagttgctatctaagcactacccacaccttctrttctctcaacttctcccctaaaagtctgccccacatgcagaaagtttggtcactcatggtagactaaaatattgtttgatggagaaaacattttgaggggcaaaagtgagaagaagaatgcagataacaatgtagcttttaaagactataaaaggttattgtttttgtttgttttgtttgtttttgagatacagtctcactctgttgcccaggctggagtgcagtggcacggtctcaactcactacaacctccaactctcagtttcaagcaattctcctgctcagcctcccgagtagct

SEQ ID NO: 170 - DNA, 599 bp, Homo sapiens
tataaaatttgattttttctaaaagtagtactaacagtgaggtgtttaaacattaaatggagctgggtatgctatagagaaaatgtccccatcagatctgtggcaagtatgctcctacaatcccactttggcagaatcttcctgccagagcctttggaggcagagagaaaaaggtgagacataacaccaaatctcttgtgccttaaccctcacaaaccatcctggattgagtgagaggtgtgagaaggaaagaatctccgcagatctcatgaaagtaagactggaatgtcgaagaaacayaccagggggaaaagtgagtacgaacttctgaatactgcattcagtattgagactccttaatcaaaccacaaagggtgcctgagggaacaacttgtcccactgggcactctcagttctaccctgaggcaccagtccaggtctgtgggtaaattgggcaaatccctgagtgtgaaggacgcctcctgagctgtgaacttggagacctggcgcagtttggcttcaaaggcagccaaagtcttccaaatcacctggaatgaccctaatcagacttctgaagtagttatcagaaaaaaaaaaaa

SEQ ID NO: 171 - DNA, 599 bp, Homo sapiens
gtcaatattcataaaaaggcagggagaagaagggattattatctttataggtagagaaacagtcacagaatggttaaatggcttccttgacgttgcctaggaagctgtttacagaagtgaggttagttctctgaatgcctgctttctagtccattgccttgtgcagtggactttcaagcagaagctgggaagggaagagcagctgttttcctcccgcataattaatatgtagatatgatggaaagagagttaaaatgcagaaataagatattccttaaaaggcagatacgaaaatatttrtagtaaagtgtcatttaaaaatacttgtattgagaaggaggcagagtaagatggccaaatagaagcctccactggtcatcctccccccaggaacaccaaatttgacaagtgtctaccaaaaaagcaccttagtaagaatgaaaaatcaggtgagtggtcacagtacctggttaactcaatatcactgaaagaggcactgaaaaggataggaatgacagtcttgaattgctgatgccatctctcccccagtagccacacggcatggagagagaatccgtgcatttgggggagggggagag

SEQ ID NO: 172 - DNA, 599 bp, Homo sapiens
acagactatggcaacagggagaagaggtgatggtagcttgtactagggtaacagcagtagtggtggtgagaaactgaccaggaatcacagcagtgatatactactttatggccttcataatggcaaagggtacgtgggaggaaaagggctcttttgtactcctggccctgggagtatgaactgacaggaagacaatctggaagtatgaagcaaaattaaatatgtgcatactttagtactcaggcatctcacttttggataaaggtcccagagaaactaacacacaggtgcaaaggagayagacacaaggaaattcattgcagtcctgttttggagaacaggaagctgaagacaacctaggtttccatcaccaggggaatcaatgaatgaaacacgagaaaaataatggcacctaccttgtgagactgttgcaagggttaaatgactttatgtaaattgcttagaacagtgatgacatacggtaaggactatataaatttgctaataatatatttgtaattattatgatggaacattaagtaacattaagacacatgatgtataaatcttaatactgggtgagaaagtataaaaaagaa

SEQ ID NO: 173 - DNA, 599 bp, Homo sapiens
aggctgcagtgagctgtgattgcatcactgcactctagcctgggtgacagagaccctctcttggaaaaaaaagcaaaaaaagaaatattttacttatgtacttctgtattgtttgatttttgatgagacatgactttgtcttatttatatacatataaaacatttttaatgaaaaatatttgccaattaaacaagccaaaaaatggtattttgtttcgataagcacttaattattagcgaggcaaaacgctttcaagtttatggactattacgtttttctttctttttaaataactgctrtttatatacattttctgtaagggaatcatcttttttttgtacttattttgtcatgtatgttgcaattattttttcccagtttgtcaaacacctttgaattctgtttatggtttctgcctttgcttttgtgctgaggaagttgttcctcaccttgagataagataaatacgtattaatacagtttcctacagctcttttggagtcacattgtaaaatctacctctttaacaagaaatgcttttgtatctagcatgaggaaaaaatttcatgtaactgtttgcaattgttaactgtctcac

SEQ ID NO: 174 - DNA, 599 bp, Homo sapiens
gtttttctttctttttaaataactgctgtttatatacattttctgtaagggaatcatcttttttttgtacttattttgtcatgtatgttgcaattattttttcccagtttgtcaaacacctttgaattctgtttatggtttctgcctttgcttttgtgctgaggaagttgttcctcaccttgagataagataaatacgtattaatacagtttcctacagctcttttggagtcacattgtaaaatctacctctttaacaagaaatgcttttgtatctagcatgaggaaaaaatttcatgtractgtttgcaattgttaactgtctcacttgctgtgttatccttcggttcttaactgatgtgagatgtcacctttatctcacattatattcttacacatactgggatctaattcagagtttggatgattgctttatttatctaccagtttgttcttgcattaatgccacattactatgcagcactaattttaacacctgctgggaatagacccctcattcattttttattttttatttttttcaggcttttttaaggggatggggagaaggaaaagatatttttacttatttattctctt

SEQ ID NO: 175 - DNA, 599 bp, Homo sapiens
agttgtcaccttctgtttaagtgtctttcttctctattagctcttaagatccttcaaggcagaaactacacccatgtgtctttctctcccctgtgtctaagatacactcagcagcctcttaataaatgtctgcttaatgccaagaaatttacagtagctaaagaagttaagctttactgacaagcagaaccagccttaatatctggttagagaaataggacttggaaaagcacagcctcctggtctagactagttcagactcactagtgccactgtaggaaactatctgcattaaggccyttaaggcccctctgccaagccaggaagtttgcttctttgccacattctaacaccttcatctgctactgaacttcctcacacccaaatctctgtttctgctccagaggacattcactttactaaacacagttgtgaagcacagaagttgtgttatacgtctaggtcactggatgccaaaaacccttaacagcccagatttccaactctgtttactttcccaaaaattttacagcattctaaacatactgttacaatctcaaaatagttttaaatgacagtagtaaccatagacaacctat

SEQ ID NO: 176 - DNA, 599 bp, Homo sapiens
ttacccagtgcaaacattaggaatgcgagaaaatgtgcaaccctcccctcctttgccccgcagttataagcaactgagaataaaaagttatactggaatacattttatacccttatgatagtatttgtgatcacaaacaaaataatatatatggtgggaaaagcattgagagatgcctccagataaaaagttctgtgtaaatgaagataatgttatctctgctgcataatcctgaagtcaaattagcaattcaccattatatctcacaccttttgatcagcctgttatatgtaatgctcrtaacaagtttaattattctagttctacaatgctaagtttctagaaaaaaattgtagcataatttgcaaaaatggcataaagtccctactcaaatacacattttaaaaaatgtgacatcaaaagaaagaagccaagctcctttttctcaagcaaaaagttataaatatcctggaccttccccatataaagatctagcctagaaatggcaaaaggcttcaattaatggcccaacatcaatgactggtaggagcagcctgaagctctacattaagaattcagggccaggcgtggtagccctg

SEQ ID NO: 177 - DNA, 599 bp, Homo sapiens
gcatgcataggaaaacatacagtatatataaggttcagtaccatgcatagtttcaggcatccactgaaggtcttaaaatttatcccctgaggataaggaggggattactgtattactgactttagatgttaaaatggtgcatctatgagaggcaggccaaggagaagacttcaggaacacagggaggggcttgtccttccacttaggacaccacagtatggggtctatgggccatagaaagcaagcgacattcagaatgtagatttcctagctctgtggcacttctcgctttaatcctcwagcctttaaaaaatgttcttgacctgtcttagctaagcatgaggaagcaagggaacaatcttttgcccatatttgggtttaaagtttaaagtatcttctcccttcctccaacccaggatcccttcttggcagagaatgtgaaatccagggagactgctcagggccatgtggtccagctaaccatacccagtgacgtggttctttttttgtaagccctggaagttaggaatttgaatttgttaagaaaaatgaatttctttacagagagacagcatagtactatgacctaaagaaatttt

SEQ ID NO: 178 - DNA, 599 bp, Homo sapiens
gactttagatgttaaaatggtgcatctatgagaggcaggccaaggagaagacttcaggaacacagggaggggcttgtccttccacttaggacaccacagtatggggtctatgggccatagaaagcaagcgacattcagaatgtagatttcctagctctgtggcacttctcgctttaatcctcaagcctttaaaaaatgttcttgacctgtcttagctaagcatgaggaagcaagggaacaatcttttgcccatatttgggtttaaagtttaaagtatcttctcccttcctccaacccagratcccttcttggcagagaatgtgaaatccagggagactgctcagggccatgtggtccagctaaccatacccagtgacgtggttctttttttgtaagccctggaagttaggaatttgaatttgttaagaaaaatgaatttctttacagagagacagcatagtactatgacctaaagaaattttgctttggagatttcacagcattctctgcctgttctgggaaggtacgagttccttttcttaatctagctttccgtcattgcttatgaggatgctactagatttaatttctaaggcaca

SEQ ID NO: 179 - DNA, 599 bp, Homo sapiens
atgctactagatttaatttctaaggcacatacagtacacctttaacatactaaagacaatatagaaaaacagcaacttctgcattccaactttaaacccgtggtattccgcagtcttcagttctagtggagcacaagcctacctgtggttacagttaatccttaacctttgccttagttatgtgatcacctaacttctgtactgagcacattttattacaacataaaattgaatttggtaagaaatatttaaatgagctctcaagggattaactaaagctaaaatccatctgaattccaytctttggtaaacaaatatttattctcaaattgaactttatattcttaggttaaacaaagaatcttgaaaactagagttagcctgagattggtacaaaagaagggaaccttaacattttggctcttccttcagtaaccatagaggactctgcggagaacattgttgtttgttgtggccagaggcaggcacctaacaagaacttaagctacagcattcactagagatttgagatcacccaaggtttagatgcaccatttccaagattcctatttgaccttttcctttaaggagatactaag

SEQ ID NO: 180 - DNA, 599 bp, Homo sapiens
ctttggtaaacaaatatttattctcaaattgaactttatattcttaggttaaacaaagaatcttgaaaactagagttagcctgagattggtacaaaagaagggaaccttaacattttggctcttccttcagtaaccatagaggactctgcggagaacattgttgtttgttgtggccagaggcaggcacctaacaagaacttaagctacagcattcactagagatttgagatcacccaaggtttagatgcaccatttccaagattcctatttgaccttttcctttaaggagatactaagawtatacacaaataattctctaacccaagaagcacgtaatgcctgtggttttccccagtgtaaaattttcttctagttctgtgtataatgttagtgctcttataatcaagccactgactggagggggtgtgacttcaggtcttgttgctaaggcagcaagtggaaaacaagtttactgccagaatgacaaaaagcttctagaaaagcatgttgcaaagaaagatttgttactcttagaaaaccttccctctggctggtctgtggagctatagattctcttgtactgacttcagcaaagggg

SEQ ID NO: 181 - DNA, 599 bp, Homo sapiens
ctttatattcttaggttaaacaaagaatcttgaaaactagagttagcctgagattggtacaaaagaagggaaccttaacattttggctcttccttcagtaaccatagaggactctgcggagaacattgttgtttgttgtggccagaggcaggcacctaacaagaacttaagctacagcattcactagagatttgagatcacccaaggtttagatgcaccatttccaagattcctatttgaccttttcctttaaggagatactaagattatacacaaataattctctaacccaagaagcasgtaatgcctgtggttttccccagtgtaaaattttcttctagttctgtgtataatgttagtgctcttataatcaagccactgactggagggggtgtgacttcaggtcttgttgctaaggcagcaagtggaaaacaagtttactgccagaatgacaaaaagcttctagaaaagcatgttgcaaagaaagatttgttactcttagaaaaccttccctctggctggtctgtggagctatagattctcttgtactgacttcagcaaaggggccaagcagcatttgcttgtttgggagaggaagg

SEQ ID NO: 182 - DNA, 599 bp, Homo sapiens
gttaaacaaagaatcttgaaaactagagttagcctgagattggtacaaaagaagggaaccttaacattttggctcttccttcagtaaccatagaggactctgcggagaacattgttgtttgttgtggccagaggcaggcacctaacaagaacttaagctacagcattcactagagatttgagatcacccaaggtttagatgcaccatttccaagattcctatttgaccttttcctttaaggagatactaagattatacacaaataattctctaacccaagaagcacgtaatgcctgtggktttccccagtgtaaaattttcttctagttctgtgtataatgttagtgctcttataatcaagccactgactggagggggtgtgacttcaggtcttgttgctaaggcagcaagtggaaaacaagtttactgccagaatgacaaaaagcttctagaaaagcatgttgcaaagaaagatttgttactcttagaaaaccttccctctggctggtctgtggagctatagattctcttgtactgacttcagcaaaggggccaagcagcatttgcttgtttgggagaggaaggattattgaaaggtt

SEQ ID NO: 183 - DNA, 599 bp, Homo sapiens
ctatgtggtagctattgctactgggtgctgggatatgggactgaataatacagataaaatccctccctcacagattttctatttaaagttgaaataaacaacaaattaaaaaatcaaataattatccaatgattgataggtgatctatttagcactgaactttatagacaaagttgctaaattagaaggataaacagacataaaacttctaccttctggaataaaagaagagtcaaacgggaaagtacaactagctctgtctaagaacacggctataatgtaaatgagtctaaggtgtaracatagtacacctattatagctatttagcaggatacaggcaaaaatctaattaaataggcttcttagcatctggaaagaggaactgatttgaatttcaagcaatgtcttgcttatttcatggtttagttttctttttgttctttccatttttttcagattgtaattttcaagggattatagagagagaaaatgagatgtggagtatataaaatgaaaaatgaataagggagaggggaagaagagagacgggggagaaaaaagggagggggatagagaaagagaggagcaagggagaaag

SEQ ID NO: 184 - DNA, 599 bp, Homo sapiens
tcacagattttctatttaaagttgaaataaacaacaaattaaaaaatcaaataattatccaatgattgataggtgatctatttagcactgaactttatagacaaagttgctaaattagaaggataaacagacataaaacttctaccttctggaataaaagaagagtcaaacgggaaagtacaactagctctgtctaagaacacggctataatgtaaatgagtctaaggtgtaaacatagtacacctattatagctatttagcaggatacaggcaaaaatctaattaaataggcttcttarcatctggaaagaggaactgatttgaatttcaagcaatgtcttgcttatttcatggtttagttttctttttgttctttccatttttttcagattgtaattttcaagggattatagagagagaaaatgagatgtggagtatataaaatgaaaaatgaataagggagaggggaagaagagagacgggggagaaaaaagggagggggatagagaaagagaggagcaagggagaaagagaaaaacgaatatgaacaaaccaaccccttctccgtctctgttatgtgctaatgtgaacaaattcc

SEQ ID NO: 185 - DNA, 599 bp, Homo sapiens
tcaaaatgtctgcattatcttttccagtttatccaataagctatgcgtttccacgaatgtagacaaatgagtctaatggaattgtgaactagagaaaaagcagaacaaatttaaatttgttagtttccctatgaatctataagaactcatttggcgactatgacctgtaacatgcctgctacttagtgtcagggcaagggaagtttctcctctttttaacctaagactagggtgggcatatgccgttaattgtaacttaaaagtttaaggtttggaagaatctgtttttctccttttttyttcttttagagtagagagttaaatagaggaggaagagaggcaaaagaaggaaaaagaattagtctctccccaaagaaaaatctaagaatacttctaagaatgtaaacattatataccctgagaaatcagaacaggaattgcccaagatggcttaaaaaacagaagtgaaaactaagcatcaaaaccaagagagtgataaagagagcacagggagtgaggaaatcacaaatccttactctagactcctgagaaactagcactggaattaaaataaaagctaatcacttctgcatattgta

SEQ ID NO: 186 - DNA, 599 bp, Homo sapiens
atagtaggggctgagcagacatgggattcaaacctaacagtctacccaatcatttaaaaagacatttccccagatgtacactttatgtgttaggagtcctatgcactgttgaagttcaattttgaagcctgtctccaatccatatttctggtggattagcaagcaggttgggagggggagtagtttggcaaggctgtttttttcccctccccaaagcttcttgagagttttaccattcatgacctagactctgctggtacagcattagtgctaagccagtgatcagaaagaaaatgtcartcctcacacttgtttcagaaattcagaaaaagaaaaacttaacgagtggaatgaacagaaagtaaactgtgtactttgtaagaaaggacgttttttaatatgcaagaagattctatcatattactaatagtcgttgctactgctgtgctgcttggcctcttcattaaaacaaaacaaagctggtttcactatgaagcaagctatcatcctgggatcaaccttcaccatcactgtagggattctttcatctctctcctgtgttaaacagcctgcacactgtatctcacactttcttcttt

SEQ ID NO: 187 - DNA, 599 bp, Homo sapiens
tgcttgaacccaggaggcagaatttgcagtgagccaagattgtgccattgcactccagcctgggcaacaagagtgaaactctgtctcaaagaaaaacaaaaaacaacaccacaggaaaaaaaaaaaacaaaaacaactccacaagtatttcaaaagcaaagattaattactttgaggttaaaggtcctttggattaacagactgctggtctactttttcaatgtagaggacccaacacacctccacttgcttttctggctctattactagctacttctatcatatgctctatgtttgaawcatgggtaattccccaaaagtacttcaggtgtcttggcttactcacatggattctgttcctaggtatgcctttctctccttactcccatagtgaactataactttcgcccagctcaaatgtcacatattttatgtaatttctcttgagtactgattcaccccaagaagcattaatttctccctcatcagtgctcatataacttctattatggtgtgtctcacgttgtatcaaagttaagtgtctacttttcagcttcttctactggacctgatctttctgaaagcagagaccattcctt

SEQ ID NO: 188 - DNA, 599 bp, Homo sapiens
atgctgacagggaatagtttaaatccctagctcccaaaatgatttccattgcttcctcctttggttcagtgatgatgatgtcatcatgcatctgcagtttgttatttttatatgaacacaaaaacagacagaaaatacattaaagtgatatgtaattttctatgcaaatgctttcagactaacaagagttgtgttttatgattaagtggaaattggttatttgctttaaatatttatgataaagtatttagaattaacacttaagcaattcaatccaggagatttaaaaaactgcaataytccttgcctgaccttggcagctaaggtgatatagcccatgttaccttagctgtggagggactttatataataaatcacaacaaatagtagtcaaaatggttttctgcgtagttctgctaccaaacccaggtaacctaaagcaaatcctttaatcttactttttagtgtgtctaacaggataaaatggaaaatactaacatatcatacactggaaaaaaactaaaccaactagattgacgaacaagacaaggccctgtagatcttgagaaagagagatcttctctgttctgagacttctg

SEQ ID NO: 189 - DNA, 599 bp, Homo sapiens
gttgtgttttatgattaagtggaaattggttatttgctttaaatatttatgataaagtatttagaattaacacttaagcaattcaatccaggagatttaaaaaactgcaatactccttgcctgaccttggcagctaaggtgatatagcccatgttaccttagctgtggagggactttatataataaatcacaacaaatagtagtcaaaatggttttctgcgtagttctgctaccaaacccaggtaacctaaagcaaatcctttaatcttactttttagtgtgtctaacaggataaaatgraaaatactaacatatcatacactggaaaaaaactaaaccaactagattgacgaacaagacaaggccctgtagatcttgagaaagagagatcttctctgttctgagacttctggagatcctcaaaatcattcagccaggtattagttttgtgtaaatgaatcacatcctaggacatgggagtcctatgatagcagatgttccaaagtgcttgggggagactgtgagtaatccagtctccaaaggtatttttaggactaggctaaatagtaacacttataacattgaggcaatgttgaatg

SEQ ID NO: 190 - DNA, 599 bp, Homo sapiens
agtatttagaattaacacttaagcaattcaatccaggagatttaaaaaactgcaatactccttgcctgaccttggcagctaaggtgatatagcccatgttaccttagctgtggagggactttatataataaatcacaacaaatagtagtcaaaatggttttctgcgtagttctgctaccaaacccaggtaacctaaagcaaatcctttaatcttactttttagtgtgtctaacaggataaaatggaaaatactaacatatcatacactggaaaaaaactaaaccaactagattgacgaayaagacaaggccctgtagatcttgagaaagagagatcttctctgttctgagacttctggagatcctcaaaatcattcagccaggtattagttttgtgtaaatgaatcacatcctaggacatgggagtcctatgatagcagatgttccaaagtgcttgggggagactgtgagtaatccagtctccaaaggtatttttaggactaggctaaatagtaacacttataacattgaggcaatgttgaatggtttgtaataatatattataatatttatgacaagttttaaaaaagtaattaacat

SEQ ID NO: 191 - DNA, 599 bp, Homo sapiens
acattgctggtaccatccataccagcaagagtaacaggctactcaagatactgaaggtacaggctggatgtggtggctcacacctgtaatctcaacactttgggaggccaaggagggaggactgcttgagtccaggagttcaaggaccagcttaggcaacacagggagacaaaatttttgtttctaccaaaaattttaaaaataagaaaatttgcagggcgtggtggcacacgcctataatcccagcttctcagaagactgaggtgggaggattgcttgagtccaggaggttgaggttgyggtgaaccaagctcatgacactctaccttcagcctgggcaacagagcaagatcctgactcaaaaaaaaaaaaaaaaacatactgaaggtacaatctcccaaggccaaatgctaccgataagtaacatttctgcatggtccacaataaataatttgaaaataattatggaaaaataatttctgtatttaaaaactggaaggagagactgctcacaaagtagacaactgctggttctcaaaaacaaggttcagaagaaagaacagaaaacagagattttcatttccatgttaactggatgc

SEQ ID NO: 192 - DNA, 599 bp, Homo sapiens
taaaagaagtaggttcaattagatgtcctcctcccaccactcctcccaggtcccatgggaatcacaaaatgcacataattcaccatctcctgggccatcagtgccacatgtgccaacaatgaaaccaatgtctttgggccttgaatgagagaatttttagatgcacctcagctcactcataaaccaaaatctaaagtgattaaaatttaagtatcaccccaattcctggaagataatatctttgtacaaactgctagtttattctcttgccatgtggagtcacattcattattaaaagaygttaagcctaatcgcagtatgatgaggaacactaatttgtcacatcctcaggctatttctgcagctgtgagttggggttttagtgggttggtatggactgacataacacattttctaaaaatcaaacagtgctacagcaactaaaagaaaatcccgctccaccaatctacacatccttcaagaaaaaaataacacttttaagtatgaagcagggcaggataaaactcttttcatccatatttgcaggaacaacaaattaatacttttctgtgtttactttccctttttaggggctatgt

SEQ ID NO: 193 - DNA, 599 bp, Homo sapiens
atggtgaaacccatctctactaaaaatacaaaattagccaggtgtggtggcacacgcttgtaatcccagttacttgggaggctgaggcaggagaatcgcttgaaactgggaggtaaaggtcgcagtgagccgagactgtgccaccgtactccagcctgggcgacagagtgaggccttgtgtcgaaaaaaaaaaaaagtatatatatatatgtatatatatgtatatatatatatatatatgtatataaaatctgtactcaaactatataagagtaaggagagacagcaccgaaaagttayattctgtaaacttcagaggtacattaatattcagaaatttctaaagaatcacattatttttaaatcaaatattttaacttattgtatcctaataaaacaactgtctctctgacacttcagcctcccacagttctgggattaagggtgtgagccactgcccccagttgagacatctcttttttagcataatcaataaaatgaacaaaccatttaatgaagtgttttcaatttaaaacaaattatttggggaaaaaaagttctgcatacattcagacattacccacctcccccagctcttg

SEQ ID NO: 194 - DNA, 599 bp, Homo sapiens
ctaaattgcttgtatcttgctttttattagcatcaaatatttttttcttctgtccctgcccccagcgtcaaatatttactaagcaatcataaaatgcatgctattgtgtgaggggcttggcattaagaagtcttagtgtgagttagaaagtctgcaccctgaagatgcttacagttttcaggaccaaattctagtcattgcatccattaactacagaggatgaaaagtcagcaagtattaaagtcagacagctgagtctgggaacatcttccacatttttgtctatcacttgctaaccayagtatgaagggttaccagatatttgaactctgacaacaatatcagatcttttcttgctcagaggaatttaactatctgaattattgaatagatttcttcaaagaagacaaggtctgtggaaaagggcaagttggaaaatccagctatggtttggttgcgaaggataaacacacaaacactgcatactgtgcaacattaattcttttaaaatagaagtagtaggttttggacatcagtttattactaaaataaaaatgtaaatttggacttaactaaaactttcccaaagcaattaacct

SEQ ID NO: 195 - DNA, 599 bp, Homo sapiens
ccaaaacattaattttccacatagtattttttttcttgttaatagaattactactttttctaatacaggaattgttatataatcaatagggtggtataagaaatcaatgtaccaacttcttccttaaaaagaaatctttagattccttcttgaaaaagatattttaaaagatacaggtgctgcagaattttgctccttagttcagctaaaatcctgctctactcacccttcaatatgtctgcgttgtctaaattttcctggtcatgtgattcttcccctggcaaacagtgtgaacttccraacttcccatggctccgccccgtcctcccagtggaggacgactctcggggaaccctcccccttatctgcctcctgcatctatcattgacaggatttccttatacagcaatatagctaacttatgtgcagagaaacccctcctcatgaagaacatctaaaaatcataggcaaaacttttcttttttttttttttttgagatggagtcttgctgtgtcgcccaggctggagtgcagtggtgtgatcttggctcactgcaacctccacctcctaggtttaaacaactctcctgcctcagcct

SEQ ID NO: 196 - DNA, 599 bp, Homo sapiens
taagaaaaaaaggaaacagagtgctgggagaaaactgtatttagatgctactagaaggtgtaaaaaagattatcaataagtattaaaaactaacttcaggaaaaacaatcaaacaagaagggaaacaatcataccacatatgctcggatattaattatatttgcatgaccacagtgaaaattacttttactacaaattgtaacataactatattgggaggatagggaaggaatgcacaggagacggcagtgtaaaagagccaaacagtcatctattgtcatattaggaaattaatagaargtgactacaattgattaatcaaaagaaagatgtcccacatagaaaatataggtatgtataaatgcccaaagaaagagctaaaagagtggtaagtcattgcctttggagatggagactggagtaaatgaagaaaggtgggtgtgggaattgctgtatttgttatatactatatgctctttagtactatttgactttttaaagtatatgcctgtattactttaataaaaataaaaaaattgtaaacatatatgaaatgaaattattttcaagaatatgggtgaaataaagatagaactttc

SEQ ID NO: 197 - DNA, 599 bp, Homo sapiens
aaacagtcatctattgtcatattaggaaattaatagaaggtgactacaattgattaatcaaaagaaagatgtcccacatagaaaatataggtatgtataaatgcccaaagaaagagctaaaagagtggtaagtcattgcctttggagatggagactggagtaaatgaagaaaggtgggtgtgggaattgctgtatttgttatatactatatgctctttagtactatttgactttttaaagtatatgcctgtattactttaataaaaataaaaaaattgtaaacatatatgaaatgaaatkattttcaagaatatgggtgaaataaagatagaactttcataatttcaaaataaggaatttccagacaaagaaaaataaaagaattcatcatcaacagattcttactaaaggaaattctgaaagatatacttcaggaagaagaaaaatgatcccagaaggaaaatctgagatgcaagaaggaatggtgagccaagaataggaaatatttatggataatgctaaacacacattgactagataaaacaataatattgtcccacttgtgggttaaaaatcaaataaaattctaggaaagaaca

SEQ ID NO: 198 - DNA, 599 bp, Homo sapiens
aatacttaagatgctaaagagattaaaaagataagaaattataaataactttaagtcaataaattcgaaaccttagttgaaatggacaaatttctaggaaaatctgaattatcaaaaccaactcaatgagaaatagaaaaccagtgtggtgctataaacattaagatatttaatcaacagttaatagtcttcccacaaggcccagaaagttttataggtgaattctaccaaacattcaagaaacagacaattttaatatatacaaattcctccagggaaagaaaaagaatactcttcaaygcattttatgaggctgatacaacctgaaaacaaaattagacaagcacaagaaaattctagttgacctcataaacacaaatgcaaaatcctagaaaaatttcagccaaacaaattcaatgatacataaataatcatcatgaccaaattaggtttattccaggagtgctaggtatattcaacattagaaaatcttacaatataattcacacattaactttaaatgagaaaaaccctgtgattgtcacaatatttccagtaaaaacatttgataaatccaaccactatttaaaaatttaaca

SEQ ID NO: 199 - DNA, 599 bp, Homo sapiens
aatcttacaatataattcacacattaactttaaatgagaaaaaccctgtgattgtcacaatatttccagtaaaaacatttgataaatccaaccactatttaaaaatttaacaaacactcagcaaaccaggaatataaggcaattttcttaacttgttaaaaggcatctatcagaatcctataacaaatgtctttctcagtggtaaaatgatagaagcattccctttaaaattaacaaataaacttcagaaggatgatcaccattgttccttctattcaactgcatttctatacattctgyagcaagcaagcagaatacattgaattaaaagacactgtacaaaatggcaacaaaaatagatgttacatctagaaataaacctaacaaaagatgtagcagatatggagaaagttataaaattttgttgaatgacggtaaagaagaccaaagtaaatggaggaacacaccatgttcacgtatacaaggatcgaaccttataaagttgccagaagaactggtaacgttccatctgttacactaaacggtgagtaatgggtcttcattttattgaaactcattgctatagattgaatgtttgc

SEQ ID NO: 200 - DNA, 599 bp, Homo sapiens
atagaagcattccctttaaaattaacaaataaacttcagaaggatgatcaccattgttccttctattcaactgcatttctatacattctgcagcaagcaagcagaatacattgaattaaaagacactgtacaaaatggcaacaaaaatagatgttacatctagaaataaacctaacaaaagatgtagcagatatggagaaagttataaaattttgttgaatgacggtaaagaagaccaaagtaaatggaggaacacaccatgttcacgtatacaaggatcgaaccttataaagttgccaraagaactggtaacgttccatctgttacactaaacggtgagtaatgggtcttcattttattgaaactcattgctatagattgaatgtttgcgtccacccctcctccaaattcatatgtcgacatcttaaccccttgaagctgatggtatttaggaactggggaggtgattttggatagaattttgcccaagtccagggatgactgagtgggctgtggcagacagtgccttttgtattggatagctgacaagctgcatgggaatacgcactcctcctcagtcatacattatcttcctttgg

SEQ ID NO: 201 - DNA, 599 bp, Homo sapiens
ttgcccaagtccagggatgactgagtgggctgtggcagacagtgccttttgtattggatagctgacaagctgcatgggaatacgcactcctcctcagtcatacattatcttcctttggcaggctggcagcatagggctcggtgggaaaaccttaagcctcactttaataatggtagcacataaggcacagtgattctgactcagttgcaagctgagtgcaaaattggagtatattctacccttaaatctcacctttaaaaatttcctttttcttttaagcttgcaattgattctttctgygaagtatcttgtgatactgtgaattaggctatataaatataaaacacactgcattatataaagttttactgcttctctgctataacatcaaatatgcttgattctctaggcctaatttgtgtatctttagactcactgagtaggttttacctgatgaagaattcaataaaatggtattaaaagagacaaagattgctatcagatataaatccctttttatctttcctctgagcttaggcctttggcaaatggttcttttggatatgcaaaatgtatcccacaaacaaaagcctgcttag

SEQ ID NO: 202 - DNA, 599 bp, Homo sapiens
catacattatcttcctttggcaggctggcagcatagggctcggtgggaaaaccttaagcctcactttaataatggtagcacataaggcacagtgattctgactcagttgcaagctgagtgcaaaattggagtatattctacccttaaatctcacctttaaaaatttcctttttcttttaagcttgcaattgattctttctgcgaagtatcttgtgatactgtgaattaggctatataaatataaaacacactgcattatataaagttttactgcttctctgctataacatcaaatatgcktgattctctaggcctaatttgtgtatctttagactcactgagtaggttttacctgatgaagaattcaataaaatggtattaaaagagacaaagattgctatcagatataaatccctttttatctttcctctgagcttaggcctttggcaaatggttcttttggatatgcaaaatgtatcccacaaacaaaagcctgcttagttcatacagaagttagactagtcagcacttataaactgcaataacccatcactgtgaaatatatattagaaggtcttcacaactagtatttcctaaga

SEQ ID NO: 203 - DNA, 599 bp, Homo sapiens
agtatcttgtgatactgtgaattaggctatataaatataaaacacactgcattatataaagttttactgcttctctgctataacatcaaatatgcttgattctctaggcctaatttgtgtatctttagactcactgagtaggttttacctgatgaagaattcaataaaatggtattaaaagagacaaagattgctatcagatataaatccctttttatctttcctctgagcttaggcctttggcaaatggttcttttggatatgcaaaatgtatcccacaaacaaaagcctgcttagttmatacagaagttagactagtcagcacttataaactgcaataacccatcactgtgaaatatatattagaaggtcttcacaactagtatttcctaagagtcacataataaggaaaaccacagtgctttcaggcctcagaagactagaaaacaatttgattggggtaatatgtgataactcctaaataacctaacatttcacaagaagacaatgcttaaatatggtggcctagaaaaacattcccagggtaaaaggcctctagggtgcacagggaattttgtctcatcctgtatgtcataaac

SEQ ID NO: 204 - DNA, 599 bp, Homo sapiens
aaagaggcctgaaaaccaagagagaaaaataacggcccgcttcattagcattctatgttctcagaactaggtagcatcaatttgaaatgcatgtactcacttcacactgataggatataccaattctaccttctcagataccctttctaatcacagtccctgggactgtagtctagagtgaaatgtaatctccaagtgaaaaataaaaaatgatggaataagtaatttcctcactcaagtgaaatctggttttatgatgctcttgctcaagagacatctgttagcacatctggatttaawtgttcagatatgcactttgttccaagaaaatgcttctggtgtaactgcccctgacaaaaaaaaaaaaaaaaagtctactttattacaacagctttgaggttcacagtttaactggctggagccagaccatgttgttggtaactatggcccctctcctatttgacttgtacagtaacagcagcattagctctctaacattaggggctgttttgttctgtttttaaaagaaagtttggtataacttagtgcctgtattagctgttaggattacagtctacttagacttttataatgatttg

SEQ ID NO: 205 - DNA, 599 bp, Homo sapiens
tattttctctctggcttctggtgtcttaagctttatgttggtgatttatgatttatattgtcagtcaaagctcttaataaaaatacagctttatgttatgcagctgggaatagaatttcttcctttctctctccgtagagtcccacatacatattgaatatattctagtggtaattataaaatacgaggttatagggctatcactgattgtcatcatatgccactcaaacttcttcctgatggtacaatgaagtgactgaaaatacctctctgctggtgccgtcacatatctcagagtgygctgcgctacaacacagaaggctttcattctcctcgggatgtagagatacttccaaagattttctttcttttttttgagacttcctcagccccctgagtagctgggatcacaggcacctgccaccatgtccagctaatttttgtatttgtagtagagatgaggtttcaccatgttggccaggctggtctcgaactcctgacctcaagtgatccacccaccttggcctcccaaagtgctgggattataggcatgagctacctcacctagcccatgtgtaaatttttttactaatgggtac

SEQ ID NO: 206 - DNA, 599 bp, Homo sapiens
gatttatgatttatattgtcagtcaaagctcttaataaaaatacagctttatgttatgcagctgggaatagaatttcttcctttctctctccgtagagtcccacatacatattgaatatattctagtggtaattataaaatacgaggttatagggctatcactgattgtcatcatatgccactcaaacttcttcctgatggtacaatgaagtgactgaaaatacctctctgctggtgccgtcacatatctcagagtgtgctgcgctacaacacagaaggctttcattctcctcgggatgyagagatacttccaaagattttctttcttttttttgagacttcctcagccccctgagtagctgggatcacaggcacctgccaccatgtccagctaatttttgtatttgtagtagagatgaggtttcaccatgttggccaggctggtctcgaactcctgacctcaagtgatccacccaccttggcctcccaaagtgctgggattataggcatgagctacctcacctagcccatgtgtaaatttttttactaatgggtacatgatccaaaaaagttgtgagacccctggcataaagttctct

SEQ ID NO: 207 - DNA, 599 bp, Homo sapiens
tcattacaaagaggtagatggtgatgctttagtcacttctttaatacaatcgtaagcaatttaaagaactgtacttttttgcaaaaattatatccactctacattttggtgttatagaacaacagtgacagtcaacatctttacttagcaaaattccttttggaaattgcactggtctgtgaaatccaaaagtctgggaactcctgctttagtacataccttccactctagaaaagaccttaggtctccctaatttactctatcattcctgcttttatatgcttatacttattgtctccstaatcctggaatatcaaagcttcaaatcttacccatttttaaagacttcacgtcttactcattcattcagtcagtttactcaaatatttactgaatatctaagtggaaggcatcaggctgaggggaacagaatttgggtgaggaatgtgtgccaagatgattagctcatgaaccttactctcaaaggttttacatactatcctttttatcaagtcttaagttacaaatttggttaacaatttcctaatcacatcatctgttaatagaatgatgaagggccacactattttaaatcagag

SEQ ID NO: 208 - DNA, 599 bp, Homo sapiens
tcacttctttaatacaatcgtaagcaatttaaagaactgtacttttttgcaaaaattatatccactctacattttggtgttatagaacaacagtgacagtcaacatctttacttagcaaaattccttttggaaattgcactggtctgtgaaatccaaaagtctgggaactcctgctttagtacataccttccactctagaaaagaccttaggtctccctaatttactctatcattcctgcttttatatgcttatacttattgtctccgtaatcctggaatatcaaagcttcaaatcttayccatttttaaagacttcacgtcttactcattcattcagtcagtttactcaaatatttactgaatatctaagtggaaggcatcaggctgaggggaacagaatttgggtgaggaatgtgtgccaagatgattagctcatgaaccttactctcaaaggttttacatactatcctttttatcaagtcttaagttacaaatttggttaacaatttcctaatcacatcatctgttaatagaatgatgaagggccacactattttaaatcagagctgctctggaaaatctagcacatataggtcac

SEQ ID NO: 209 - DNA, 599 bp, Homo sapiens
atttaaagaactgtacttttttgcaaaaattatatccactctacattttggtgttatagaacaacagtgacagtcaacatctttacttagcaaaattccttttggaaattgcactggtctgtgaaatccaaaagtctgggaactcctgctttagtacataccttccactctagaaaagaccttaggtctccctaatttactctatcattcctgcttttatatgcttatacttattgtctccgtaatcctggaatatcaaagcttcaaatcttacccatttttaaagacttcacgtcttaytcattcattcagtcagtttactcaaatatttactgaatatctaagtggaaggcatcaggctgaggggaacagaatttgggtgaggaatgtgtgccaagatgattagctcatgaaccttactctcaaaggttttacatactatcctttttatcaagtcttaagttacaaatttggttaacaatttcctaatcacatcatctgttaatagaatgatgaagggccacactattttaaatcagagctgctctggaaaatctagcacatataggtcacagtacccattacatgatacacagtct

SEQ ID NO: 210 - DNA, 599 bp, Homo sapiens
tatatgcttatacttattgtctccgtaatcctggaatatcaaagcttcaaatcttacccatttttaaagacttcacgtcttactcattcattcagtcagtttactcaaatatttactgaatatctaagtggaaggcatcaggctgaggggaacagaatttgggtgaggaatgtgtgccaagatgattagctcatgaaccttactctcaaaggttttacatactatcctttttatcaagtcttaagttacaaatttggttaacaatttcctaatcacatcatctgttaatagaatgatgargggccacactattttaaatcagagctgctctggaaaatctagcacatataggtcacagtacccattacatgatacacagtctcttggagaaggctttttggatgagtcacagctttaaataggaaagaaggcaataatgaaggtcatacctgaagtcagatcagtgaagcagagcttcctatcttcttacatctaagaactaatgtcaacaaatcttctgattattttaccaagaaagaaaatgctgtaatgattaattctcttttaactaatctttttgatacctcaaacaccagagt

SEQ ID NO: 211 - DNA, 599 bp, Homo sapiens
tattttaccaagaaagaaaatgctgtaatgattaattctcttttaactaatctttttgatacctcaaacaccagagtggttgtttctgaataaaaacttgcttgtttttcttttttttttttttttatagccattctctctctaatccttaccctgctgaatttaattgccttggcaatgccaaaacaatgtctgatcctaataggtcagcattgatcactctaaaccaattggttccttcattagcaagaaaatgaagtaaagcgaaaaggcaggtggctcccgaacaggcagctgggyttttgctcctcttacagcgcctctctctcattaaacagtagctgatcgatcggcaacttgctcaaaatccacaagaactattcatcacttgtgacacagacctgttttaacaggccacacaactcaagatttggataccccaggcagtctatttgtggggcccatcacattcttcccagacagatgtctctttggatcacatggaggaaaaaatgtggacacatcatgcaatagaccactttctggcaagacaccaaacaaagtccagaatggcaaatcccagctctgagtttctggag

SEQ ID NO: 212 - DNA, 599 bp, Homo sapiens
aggcaatgatttttaaaattttatttattttgctcatccttaagacaatgatttttttttttttttttgagatggagtcttgttctatcgctcaggctagagtgcagtggcatgatcttggcttactgcaacctccacctctcaggttcaaacaattctcctgcctcaggctcctgagtagctgggattacaggcgcgtgccaccatgcacagctaatttttgtattttttagtagagacagggtttcgccatgttggctaggctggtcttgaactcctaacctcaggtgatccgtccayctcaggtgatccgtccgccttagcctcccaaagtgctgggattacaggcgtgagccactatgaccagccaagacaatgatttttaatgggaataaaaagaataagaggaacatgtagccatatttaaggctattgctttcattctggaggttcttgcaaattgactctgggttctgcatattctgaggggaggaggcagggccaacattgtcgacaagtctcccttttgcttgctgcctttccacaaagctagcagagccagaagagaactggccatagaaacaactgaaaccacaata

SEQ ID NO: 213 - DNA, 599 bp, Homo sapiens
gctaatttttgtattttttagtagagacagggtttcgccatgttggctaggctggtcttgaactcctaacctcaggtgatccgtccacctcaggtgatccgtccgccttagcctcccaaagtgctgggattacaggcgtgagccactatgaccagccaagacaatgatttttaatgggaataaaaagaataagaggaacatgtagccatatttaaggctattgctttcattctggaggttcttgcaaattgactctgggttctgcatattctgaggggaggaggcagggccaacattgtygacaagtctcccttttgcttgctgcctttccacaaagctagcagagccagaagagaactggccatagaaacaactgaaaccacaatagtaggtcaattttctcttaaatttcaaatccagaaacagaaagaacttattcaacttctatgccagtaagtaattccttggtctaataaaaaaaaaaaaatctacgaaaaacttctcacttcctatactagccacaacacagttactcaataaaaagcacttagcaatatgttgaatttattcaccaatcattcacaaaacacctttcatgg

SEQ ID NO: 214 - DNA, 599 bp, Homo sapiens
tcatgagtgaggagccctcatgaatgagattagttcccttataaaagaggccagagagccaggtgtggtggttcacacccataatcctaatactttgggaggttgaggtgggaggattacttgagccccaaagttagagaccagcctgaacaacatagtgggaccctgcctctacaaaaaaaaaatttttttaattaacagggcatgttggtttgcacctatagtcccagctactcaggaggctgaggtgggaggattgcttaaacttgggaggttaaaggctgcaatgagccatgatcrtaccagcttgtctcaaaaaaagaaaaaaaggccagagaaagcttttttgtcccttctgtcatgtgaggccactgctagaaagcgccacctataaatgagtaaatggccctcaccagatgccaaaattgttggcaccttgatcttggacttctcagcctccagaactgtgagaaataaatttctgctgtttataagctacccagttaatagtatttgttagtaacataagacagcgacctcaagccagaaccactcagctaaactactctcaaattcctaatccctagaaactgtgagat

SEQ ID NO: 215 - DNA, 599 bp, Homo sapiens
agaggccagagagccaggtgtggtggttcacacccataatcctaatactttgggaggttgaggtgggaggattacttgagccccaaagttagagaccagcctgaacaacatagtgggaccctgcctctacaaaaaaaaaatttttttaattaacagggcatgttggtttgcacctatagtcccagctactcaggaggctgaggtgggaggattgcttaaacttgggaggttaaaggctgcaatgagccatgatcataccagcttgtctcaaaaaaagaaaaaaaggccagagaaagcttytttgtcccttctgtcatgtgaggccactgctagaaagcgccacctataaatgagtaaatggccctcaccagatgccaaaattgttggcaccttgatcttggacttctcagcctccagaactgtgagaaataaatttctgctgtttataagctacccagttaatagtatttgttagtaacataagacagcgacctcaagccagaaccactcagctaaactactctcaaattcctaatccctagaaactgtgagataataaatgtttactgtttttttttttttgtctttttgagacagag

SEQ ID NO: 216 - DNA, 599 bp, Homo sapiens
tgaggcattctgttgtgaatttcaggtgcctgtgcatcactctagtgaagctatgcagtggccatacttcagcgtgctatttgcaagcacaggagagagaactaggcaggagatgaagacgtgttattaacagatgccatgtgagcccatgaaattgctaagaaagagtgtgcagagtgagaaaagtggaagacacaaacaaacacattaagaatatcattctagattgtgataggtgttatacagaaaataaagcagaatcacaagacagtgatagatagggctgaggggttagttaartttggggataaatgagatgggagtaaggaagtatactggacaaacaaagcattcttgtggaggaaacgtcaagtgcaaatgcagagagttataaatagcctgacaggtttaccgaaggagcaggagtgcggtactattggaatatataatagagtagaaggaatggagcaggagaaactgcatgggcacagagtgggcagatcatgaaaggctctgcattttaagctgtggatcttggactttatgccataggtgaagaggagccctgagaagttttagtagaggagtgacatgacttc

SEQ ID NO: 217 - DNA, 599 bp, Homo sapiens
gaaaataaagcagaatcacaagacagtgatagatagggctgaggggttagttaagtttggggataaatgagatgggagtaaggaagtatactggacaaacaaagcattcttgtggaggaaacgtcaagtgcaaatgcagagagttataaatagcctgacaggtttaccgaaggagcaggagtgcggtactattggaatatataatagagtagaaggaatggagcaggagaaactgcatgggcacagagtgggcagatcatgaaaggctctgcattttaagctgtggatcttggactttaygccataggtgaagaggagccctgagaagttttagtagaggagtgacatgacttcgttcgatttttacaactttcatcaagatacatgttcctaactacaaggagccccacctacttggttctgacatatcactgggttctatgaagcacaattttaaaaacactatattaaatgacttctctaggcctttaaacctaaacagtctatggttaaaagatcagtgagagaaatataaaattgtacttgtgccaggttcacaagtctctctttcttctctgtcccaatcctcccctcattca

SEQ ID NO: 218 - DNA, 599 bp, Homo sapiens
ttatgccataggtgaagaggagccctgagaagttttagtagaggagtgacatgacttcgttcgatttttacaactttcatcaagatacatgttcctaactacaaggagccccacctacttggttctgacatatcactgggttctatgaagcacaattttaaaaacactatattaaatgacttctctaggcctttaaacctaaacagtctatggttaaaagatcagtgagagaaatataaaattgtacttgtgccaggttcacaagtctctctttcttctctgtcccaatcctcccctcaytcacactcagataactgtgtgcatggcaagcctgaatgttaggaatataaggccataccaaggaatcaatgtcacatattcagacaaaatacatgggaaggaggaagtaaataccctcttgagggctgtcattgaaccatccaaactaaaaagaaacaatacaagtcacagcctgattattctgcagtggcctcatccaggccctttcgccatcctctgtctacctgctcactggctattaatgatttcattgcccactcaccctcccttcataggcacacaggtccagaaaagaagga

SEQ ID NO: 219 - DNA, 599 bp, Homo sapiens
tgtgccaggttcacaagtctctctttcttctctgtcccaatcctcccctcattcacactcagataactgtgtgcatggcaagcctgaatgttaggaatataaggccataccaaggaatcaatgtcacatattcagacaaaatacatgggaaggaggaagtaaataccctcttgagggctgtcattgaaccatccaaactaaaaagaaacaatacaagtcacagcctgattattctgcagtggcctcatccaggccctttcgccatcctctgtctacctgctcactggctattaatgattycattgcccactcaccctcccttcataggcacacaggtccagaaaagaaggaaacccccaaagaagttagctatgacaacaaaggagaagaaaattagctaatggctgaaatagagtttaacattatctgtttatttcaactttacttcatcaaagcacttaccattttttcctaatttattcttatacccatttaaacatataattatttaatttaatttaatttgtggccagaagaagtgacttgtggagatatacactccctgccctcagttcagagccttttgccatcacactact

SEQ ID NO: 220 - DNA, 599 bp, Homo sapiens
ccatagattcaatgcaatccttttcaaaatcccaacacacttatttgtagaaattgacaacctgatcctaaaattcatatggaaagaacccagaatagccaaaacaatcccccatccccaaaagagttagaagaatcacatttcctgattttaaaacttatgacagaccacagtaatcaagatagtgtaacactggcataagggtagacatataaaccaacagaataaaattgagagcacagaaataaacccatacatctatggccaactgatttttgacaaggctgccaagacaattcraaggggaaaatgtgcctgggacaactggatgtccaatacaaaagaatgaatttagactcttgcctcacactgtatataaaaagtaactaaaaatggatcaaagacctaaacataagagtcaaaactataatacttttagaagaaaatagaggtgtatgttatcataaccttagattaggcaacaatttcttagcaatgaaacctaaatcagcaagacttggtggctcatgcctataatacctgcactttggaaggccaaggagggaggattgcttgagctcaggagttcgagaccagag

SEQ ID NO: 221 - DNA, 599 bp, Homo sapiens
gaaaaaagaaaagaagagaagagaagaaaaggaaaggaaaacctaaatcacaagcaaccaaagaaaaaataaattggacttcatcaaaatttaaaacttttgtgcttcaaaggacattatcaagaaagcaaaaagacaacccacaaaaggagagacaatatttccccacaaaattaaatcatatatctgatagggttctggtaattatatacatatgtatatcttaaaactcaacaattttaaaaaatccaattaaacaatgggcatataaacaaaaaccaactcaatttaaaaaggcayaaagaatctggataagacgttttccaaataaaaccctgtaaatccccaataagcatatgaaaagacactcaatgttattagccatcaggtaaatgcaaatgaaaaccacaactgatatacccacaaggatggctataatcaaaaatgtctgataatgacaatgagtgttgatgagaatgttgagaaattagaaccatttcatactactagccggaatgtaaaatggtacagtttctatggaaaacagtctggcagatcgtcaaaaagttaaagacagaatttccatatgacccagcaat

SEQ ID NO: 222 - DNA, 599 bp, Homo sapiens
tggcttctatccagaatgctaaatagacaatctatctgaggaagggcctgatccctcatacctggcctgggcaggtctgtgactacatgtgtatcactcgctttgaacctgataccctataagacaagggaagaaaatctggaaactggcaaaacagttaaaaaaaaaattggacagtcattgtagggctcctgccaacttctggtttcttgccatagagagtaatgattcctgccagtcacttagaaaaagaatagcttctagaaaagctaaattgctatgacctgaaagcagcaactstgcatcagcacataaaaacttataggaagttggaagtctgaaaaatcagattcatattcagaaatttaatctctcctccaatactgaagctataatgagaaataagctcctgcaaaaacacatattaatcattttattggttctgttgttaaaaaaaaaaacaaacaataacacccagctagtgttacattctcagtgcaaatgatataaggttttgtgatggcaaaaccttcaaattattttatccatatttgttctataactttttaatactaaatattcccaaataaatagaagtc

SEQ ID NO: 223 - DNA, 599 bp, Homo sapiens
taagaagaatcacattggggaaactaaatttctacaggtatcgtgtcatctgaaatcaataatcaccttggtgaggaaggtaggacaaagtactttcttattatgatattttaatggggatcacactttgctgcccaggctggagtgcagtgcctattcacagacacgaccacagtgcactgccgcctcaaactcctgggctctagcaatcctcctgcctcagcctcctgagtagctgggactataagcgagtgccactatgcctggctatgattatcaccatgctcaaggaagctcaaygaccagtctgagcaggattcacatggtaagtagcagaaccaggtctggaagtgaagtcagagttcaaattccttgttctgtttccactacctggctgtcttttgtaaggagaatgggaggcagaaatgggagtgaacaagaagaaagcagaataaaaatgtatgttgcacctgagtatttctcaaatagattttaattttattttcctacaggtttggaaaagtatttgatgacatcttaactgtttaaacttttattttccttttgaatccgacttgcaacacatctgttgatcacat

SEQ ID NO: 224 - DNA, 599 bp, Homo sapiens
aataatcaccttggtgaggaaggtaggacaaagtactttcttattatgatattttaatggggatcacactttgctgcccaggctggagtgcagtgcctattcacagacacgaccacagtgcactgccgcctcaaactcctgggctctagcaatcctcctgcctcagcctcctgagtagctgggactataagcgagtgccactatgcctggctatgattatcaccatgctcaaggaagctcaatgaccagtctgagcaggattcacatggtaagtagcagaaccaggtctggaagtgaagwcagagttcaaattccttgttctgtttccactacctggctgtcttttgtaaggagaatgggaggcagaaatgggagtgaacaagaagaaagcagaataaaaatgtatgttgcacctgagtatttctcaaatagattttaattttattttcctacaggtttggaaaagtatttgatgacatcttaactgtttaaacttttattttccttttgaatccgacttgcaacacatctgttgatcacatataaaataaaattgccagagattaagcaatgagttgttttcaataaattctaacttt

SEQ ID NO: 225 - DNA, 599 bp, Homo sapiens
tgatattttaatggggatcacactttgctgcccaggctggagtgcagtgcctattcacagacacgaccacagtgcactgccgcctcaaactcctgggctctagcaatcctcctgcctcagcctcctgagtagctgggactataagcgagtgccactatgcctggctatgattatcaccatgctcaaggaagctcaatgaccagtctgagcaggattcacatggtaagtagcagaaccaggtctggaagtgaagtcagagttcaaattccttgttctgtttccactacctggctgtctttygtaaggagaatgggaggcagaaatgggagtgaacaagaagaaagcagaataaaaatgtatgttgcacctgagtatttctcaaatagattttaattttattttcctacaggtttggaaaagtatttgatgacatcttaactgtttaaacttttattttccttttgaatccgacttgcaacacatctgttgatcacatataaaataaaattgccagagattaagcaatgagttgttttcaataaattctaacttttgttcccagtccaattcaaatggcaactttcagaatgaggccaaat

SEQ ID NO: 226 - DNA, 599 bp, Homo sapiens
caatgagttgttttcaataaattctaacttttgttcccagtccaattcaaatggcaactttcagaatgaggccaaattgaaagatcatttcagagcaataaaagaggaagttctttcagtagggaatcacttttgagttctagttctttgcaatgttatatttaagaccaattacttatccagtatgcctgtgtaccagcctggctatgcccggatcaggacgagaagcagaccagccacagttcttcctgtgtgcaacctgcagcagccccttgcagagtctagctggggttttgaagrattctagtcatgcatttaataaaagtcattttcccaatgtgctagacttcagttctaacttggaaaaagggagctaagttgggtaaagttaaatgtggggaaatatgcaggacaaaatcagctaagtgactgctttcaacaaaaaccaaaaactatttcattttccatgggtattttcacaatgttttcctatgttcatgactataaagatgatcaattgtaaatcctgaagtagcaatggaaataattatcaaaactggattcaattagaagactaacaaaatccatcatgcttgcct

SEQ ID NO: 227 - DNA, 599 bp, Homo sapiens
ggtcctcaatggaacaactctgatgtataatcagatcaaaggaaagctgtttttgctttctttttggagataatttatttttcctaaaaaaggaatcccaatgaaacagctgatacataacctaaaattaggccactttacttactaacacttgaaggacacgatttaaaatcatggcactggcaaagcgtggtggctcatgcctgtaatctcagcactttgagaggccgaggcaagaggatcacatgaggccaggagttcgagaccagcctgggcaacacagtgagacccccatctcamaagaaaatagaaaaattagttaggtgtagtgacatgcacctgcagtcccagtttcttgggaggctgaggcagggggatcacttgagcccaggagttcaagtctgcagtgagctataatcgtgacactgcactccagcaacagagcaagaccctgtctctaaacaaaacaaaacaaaaccaaaaaaacagttctcatattctcttacttcctttgcctactattctaatgcttaaaggttaaatacattgaagagaatccaaaaagaatccaaagctttgtgatctggataggggatgaa

SEQ ID NO: 228 - DNA, 599 bp, Homo sapiens
tgtgcctataatccaagctattcaggaggctgaggctggagaatcacctgaacccaggaggtggaggttgcagtgagccgagatcagaccactgcactccagcctgggtgacaaagcgagactccatctcaaaacaacaacaacaacaacaacaacaaaacagtcggaaattttgtaaactgccttagaaattcccagtctagcccactgtgtatagttgtatatgatgagtagttgcacatggactatataaagtggtacttaacggaactgttcaatgtgtggccctgctaccaaccrcctaacccttaggaaatcatttcttcctaccaaactcttcttttgattcaaattaagcttagtccttccatctccatcctttccttctttccttccttccttcttttttcttaactatgcccttagggaggacaataacatccctcttttaaaaagtattctttcccaaacttagactcctcttcagtaacgtggcttaaactaggaaagacaaactgaaatgtccacgtgggtggcatgcgagctggatgaggacagactggagaacatatgcttatactcactgcagtgaggcagcg

SEQ ID NO: 229 - DNA, 599 bp, Homo sapiens
acccttaggaaatcatttcttcctaccaaactcttcttttgattcaaattaagcttagtccttccatctccatcctttccttctttccttccttccttcttttttcttaactatgcccttagggaggacaataacatccctcttttaaaaagtattctttcccaaacttagactcctcttcagtaacgtggcttaaactaggaaagacaaactgaaatgtccacgtgggtggcatgcgagctggatgaggacagactggagaacatatgcttatactcactgcagtgaggcagcgggggytgtggatctgatgttgaaaaagtttcctatttttcaagagcagctggtgatttggatatttttaagagaaatttcccaatttttaagagctggcatccaatttttatttccttacactgtataggccaaacaaaaatgccactgtttagacctctagtttgcaaggcctgttttacatcttttctcagcaggcttattttttaaccaattaatcatctagattgctcttctctagaccagccagttattcatcactttcaaaatacagagatcaaaaagggacatcattttctaagaat

SEQ ID NO: 230 - DNA, 599 bp, Homo sapiens
tgggtggcatgcgagctggatgaggacagactggagaacatatgcttatactcactgcagtgaggcagcgggggctgtggatctgatgttgaaaaagtttcctatttttcaagagcagctggtgatttggatatttttaagagaaatttcccaatttttaagagctggcatccaatttttatttccttacactgtataggccaaacaaaaatgccactgtttagacctctagtttgcaaggcctgttttacatcttttctcagcaggcttattttttaaccaattaatcatctagattgytcttctctagaccagccagttattcatcactttcaaaatacagagatcaaaaagggacatcattttctaagaatgtgatgtgactggtacatctctgttttacatgtcattttcctgtacaaacattctaggacttcatgctttttttaaagagtagtgtatcccatcctatgctggaacttgctcatggtcacacaactactattttttttaaacaatattctaattagaaaccaattactttgatgcctggttcagcccttctgtctcactgacctactgtgtaaccttcaagtttc

SEQ ID NO: 231 - DNA, 599 bp, Homo sapiens
ggacagactggagaacatatgcttatactcactgcagtgaggcagcgggggctgtggatctgatgttgaaaaagtttcctatttttcaagagcagctggtgatttggatatttttaagagaaatttcccaatttttaagagctggcatccaatttttatttccttacactgtataggccaaacaaaaatgccactgtttagacctctagtttgcaaggcctgttttacatcttttctcagcaggcttattttttaaccaattaatcatctagattgctcttctctagaccagccagttaytcatcactttcaaaatacagagatcaaaaagggacatcattttctaagaatgtgatgtgactggtacatctctgttttacatgtcattttcctgtacaaacattctaggacttcatgctttttttaaagagtagtgtatcccatcctatgctggaacttgctcatggtcacacaactactattttttttaaacaatattctaattagaaaccaattactttgatgcctggttcagcccttctgtctcactgacctactgtgtaaccttcaagtttcccaaccataaaatgagaataata

SEQ ID NO: 232 - DNA, 599 bp, Homo sapiens
tgtcctaggcaactcatgtatatcatgattttagctactctcctaaaactactgacacccaaattaagaattactaagctccagacccatactttcaattgcctactagacacttcccctaaaaggttctacaggtactttaaaccctaaacatctaaaataaaagttatcatctcaatgttcccactctaagttgcttttttcccctttctaacgtagcaaatggcatcattatcaatatagacctagctacctacaaaggcatacctttcgtaggtagcataatgaccctccccaaayatgtccatatcctactccccaaaatctgtgagtatattagactatatggcaaagagaattaaagttgcagatggaattaaagttgctaatcagctgactttaaaatggggagattatttaccctggattatctgggtgggaacaatgtaatcacaaaggcctttaaaagtggaagaggaaggcagagattgctagctttgaagagggaagggagccatgagtcaaggaacatgggtagcctctagagtctggaaaagcaaagaaaatgctttctcccttagagcctctagaaaggaatg

SEQ ID NO: 233 - DNA, 599 bp, Homo sapiens
tttttttttagtacagacagggcttcactttgttagccaggatggtctctatctcctgacctcgtgatccgcctgcctcagcctcccaaagtgctgggattacaggcatgagccaccacgcctggccccctctctgtactcttacattgcctggagccactctctaatgtcacacttaccttattcacatctttcagtttccgcatttaaatacaaaacatggctgaggaggctctgcaatttggggcacagtcaccaaagggctcagcccttgtcctcctctccagccctcagtactaytgaacatttccagaaagggtgccaaattactggtctaccctagatctggaagctacattgctctatgtgtgacatgcaggtcccagcctttgcccaaggacctcgtggttttgttttgttttgttgtttgtttgttttgagaccgagtcttgccctgttgcccaggctggagtgcagtggtgcaatcacagctcaccgtaaccttgtcctcctgggctcaaacaaggtgctctgcacctcagcctcccaagtagctaggactacaggcacatgccaccacacctggctaattattcttt

SEQ ID NO: 234 - DNA, 599 bp, Homo sapiens
ctcgaggagagagagagagagagagagagagaaaaaaaatattgacacataactggtatttttcaaataaataagtatatgaatattagttttagttgactttcattttcccataatatgaagcagtctttgttttggatagccaatcatcacagctaactttttcccaaaggggaagattttttaaaaggcacaccttttaaaagtcattcatttatcagctttctaccatgggaaagaaagcagctgaacaatgtaccctagaattcttgcctcctataattgaagaatgcttttttytatcatcatttatgtcttgcttatggcccataaaaagtcagatttaaccgcattggaaatttaagggaaaaatataaaggatcttgatgaaacaaaagcattggtatgagatgagccttccaagagaaataaattacacaaaatcactctttctgactctaaatacaggaataaatatgtttagggttgcacaactaccgtgtaaatgtgttaaggctcagatgcagaaattggcctaattgtatgtaaggctatcctttcacaaatcagacagtctggacttacctactgaagtgtat

SEQ ID NO: 235 - DNA, 599 bp, Homo sapiens
gtaatatagctagtcataatcatcctctatgtctaacatttcatccatgaccagataaatggacgagaggccgactctactgaattttgagatgataacaaacagtaaggcattagcagacactagaagttctgaccaaggttaaattatcaatgaaataattaagttcattaatgacaagtacagggtacatcagtaatgcaaaagaaataaagaaagaaaagtaaatggagagtataaatatgaaatgcagaaaacttggcaaaaaatatgcatgtttgtgactgaaagtaatgatcwgttactcttggattccattaagttcactgataacaggttcagaatgaagcataagggatttaggataactaattaaaaacagtttttcaattcattaaacaaaaagccagtgccttcaaatgcaattgtaaacctccgtacaaagttaattttctaaaccaggggttttcatggggggtagtatcattctggggagcattctgggttctgctggagcacttcacccttctagtctaattatttcatttctcctttatggtacagctagaacataatattgatttagtcaaacacccttt

SEQ ID NO: 236 - DNA, 599 bp, Homo sapiens
gtctaattatttcatttctcctttatggtacagctagaacataatattgatttagtcaaacaccctttctgtgatagaaccaatatatacaaaaatggataaagtataaaggaataacttagcaagctactgtaaaatgaactcctacccagcatctcagaagccctcatgtgccccttcctacaacccattcttttccttcataggtaacttggctacttttaatctttatactttatacattttagaataagctgattaaattccactaaaaacagttggggttatattgattttttwaaaaaagtttatgtgtgtgggcaggttatattttctataaattttatttcaggagagtaaggggcactggataatatttcttttaaaaagggaatattggtgctgatagggataagaaccaatgttctaaatgaaatgaagatgtagaatagggatccctgtagaacagggatctaacattctaagactatatcttctatgaaaaataaaactcaagataatcactgagaatatctccatttaaaaaactttctcagaggtcacgatttgcctccctaattattttacacagggttttc

SEQ ID NO: 237 - DNA, 599 bp, Homo sapiens
ggtcaaagcataaaagttagtactgggattttgtagttaaatcatatagtaaaaaatggtaaggcagatacaatgcattaaaagaatgtcttcagaattaaaaattcaattcagtcttggtggtctaagatgaataagcgactatatttttaagtagttcacgacagggcccgtgccatgttcagctttttcacttcctgtctacaggaccatgggcaagttacttgacctttcagaggcttggtttccttctccgcaaaatgaaaataatcctggcattcaactttttgtgtgaattaytggagatatgtattcaaagcatctaggctaggtacccaggataaactaaataagtgatggttattagcatgaattttatactcattcattgactaatgaaaacttgtgaatgactgaatgagtttcaagtggattatatttccaaggcatctgcactgtaggcttttacttctcattgcagaataaaagtctctggttcctttaaagaaacttcaggtatcagcaagcagatagcataagttcactgaaaattcatcatacaaggtcatatagtccattcagtttataagaaacaaatt

SEQ ID NO: 238 - DNA, 599 bp, Homo sapiens
tggtctcgaactcctggactcaagtgatcctccagcctcagtcttccaaagcgctgggattacaggcatgagctactccaataggattttcattcccatcactccactgaaatgtttcctgacaaggctgctatcaattaatcctgtgccaacccaatggtcaggtctgtcctggtcttactcttctcagcgtcatttgatgtggttgacttctgtcttttaatgctttctatactttgctttcacaatgcagtacccttcaggtttcccttctgtcctttgacagctccttctcatttygttgttgactctacgcctttgtcttgacctccaaaggtgaagggccccattcttcagtctttgtctctcttctcttctctcctctctccataggaaatctcacccagtcccatggttttcaatacccatctgtatgctaataactctcaaagtttatctccagtcctgatttctctcctgagttccagactcataaatcctattgcctcttcaatatctcatgtggacatacactaatattatgattatgggtaatagtaataatattaacagtagcagcaacaatagctaacatttac

SEQ ID NO: 239 - DNA, 599 bp, Homo sapiens
catggcctacaggggtcttgctatgggttgaatgtgtccctcagaattcatgtgttggaaatctggtacctggtcctggtatggtggttttgggaggtgaggcctttaagaggtgattagatctttaagagggactaatgttgctctttcagatgaattctggtggaagtaagtgaattcttcctctcacgggactggattagctacctcaagagtaggttgttttgagggaaggctatcatgttttgtctcttttgcacaagctttcttgcccttctgcttttcaccatgttatgatgyggtaaaaaagcccttgccagaagctgccaccatgcccttggatttcccagcctccagcgctgtgagccaaatatagttctgaaactgcctttgcaaagatcatgacagtgagagaaatctcgcatggctgacttcatcttgcctctagcctcacaggctgactgtcctcactcattcctgagcgggaggaatttggaattaagtatgataacagtccctccctaaaacatcccctcctcatttggggctgagacaccatttgtaagactaatgagaggccacaagattgggattatggg

SEQ ID NO: 240 - DNA, 599 bp, Homo sapiens
atagcacagtctcagtgaattggttttctctgtgcagcaggtaggaacaacccactgggtgattatatttcttttctttataaattacccagtctcagatattctgttatagcaacagaaaactgaataagatagccctatatgatcaggctctcagccacatcttcagcctcacatactaccacttgcactagctcttgcacaaacaggccttcctctgtgccttgagtataccaagttcattccatctttgaggattctgcaaagttctccctttacttggaatgcttccccttgagmtttttacttgcctaattccttcatgtcagttaagattcagctcaaactttaccttctatgatgatccaactcaaaagtttctcatccctctactgtcaggcctctgagccctaactaagccatcatatcccctgtgacctgcacatatacatccagatggcctgaagcaactgaagatccacaaaagaagtgaaaatagccttaactgatgacattccaccattgtgatttgttcctgccccaccttaactgagcgattaaccttgtgaaattccttctcctggctcagaacctcccca

SEQ ID NO: 241 - DNA, 599 bp, Homo sapiens
tacaagtatatttattgagcatttactatgtaccaggtatagttctaggtgctagagagcttcattttagtggggaaagatagagacaacagattaaaaagacaaaataaattagtaaaatatatttggttatgtggtgatcaatactatgcagaaaaacaaaacagaaggaagatgggtgtttggccttactaaggccttactgagaaaacggaatagaagtggaggcctgaagggagtgaggaaataagccatgaagatatctgaagaaaaaagctgccaacagaggggacagcaagycctgcagcaaaagtctatcccttgtgtgttcaagaacagcaaagcgtgtcctggagtgaagaaaacaaggaagagagtatagtaagacagtccaagagaagacaggacacctgataatgtggggtcttgtaggctcgtgtagcaactgacttttttttttttttttttttttttggtgagatggagttttgcttttgtcacccaggctggagtgcaattggacaatctcagcagctcactgcaacctccacctcccaggttcaaatgattctcctgccttagcctcccaagtagctggg

SEQ ID NO: 242 - DNA, 599 bp, Homo sapiens
ttatgttggtgttggctactattattattacttggttttatattaagtgtatgccacaatttaaatgttaaaacagctttatggaaatgggctgacttgtcaaatacagtgctaaaaagctacttgcccccatggtaacttttcactaactaaaagaagatttattcatttattcatacataattagagtcattatttctacttactgatatctagatccagttctcttccttctggacacatggaagactaaatttcctattcctctttggctggctggggccatgtaaattgtgagtrggagtggcaggagtcctgtctgaactggaatattgaattgctagagtgagaccctctagagctctttttcccctgactctgtgatcatggaagcatgtattgagtctctgtcagcgtgggtccctgagtaactacaatgaacaaagaccctaccaattcttactgaatttgttgtatgagtgagaaatatggcactaagatttttagagtggtctataactacagtataacctagcctattctgactgatatagtaacatctgcaggctattactttcttaatcaatccataggagaga

SEQ ID NO: 243 - DNA, 599 bp, Homo sapiens
caatgtagtttatcatgtcagaaaaacatctcatcttaacatgattgatatttaagcttttccagggtaaagttaaagaagctggtgggatacaaaaagtaaattaaagttttagtcataggatgagtgaagactggagaaagatacgtggacatttggtcctcattcaatagctttaaaacggatctctggacctatattcacataactctccaaaaggcaatttatggtttaaattcagcttgttcagttgcagctctggttaactccagagctcaagccttgttacagtaatcaggrtgtgtcaagggacatacgggaggaaccaaagggatggaaatggcaaactttgcttcatccccagccaaagcctctcttccgcaaccaaaattctccacaatagtgtatcactttgtcccacctgaaagtacctggaatgaggggaaagcacagataacttctctaaccttacacatgcaaaatccttatcaaatgaggaaaggaaaggtaaaagaaagagaaggagaggggagaaagaatgcaatgtaagagggaaggtgacggtgggagataaatatttttgtaagccaaggtataga

SEQ ID NO: 244 - DNA, 599 bp, Homo sapiens
ttagacaagaaggtctgataaaccttgactcaaactaacttatgagtaactgacattttctttcagcgttctcaggtaacaagcaatttttacaaacagttgtctcagacagtacccattgttacctcttataactcccactcaccttaaatacatatcttaatactgtttgaaaatggcaaacattgttgcagaagagaggcaaaagagtacactcctctttcttctatagaacaaggatgatggtgaccacatatgaagacgatgatagtacccaagtgccatgtttactactggtawttgttgccaagtcttgattcatccttgataatttctgttccatccacctctctttttcattcccatctgacactgactgttctatgaactgcactctgaaaagtaaccatcaaatttctaataatcaaaaccagtggtcattgatccttgggtttgcaaagttttagttcatgtacctgaaaaatactgtctgtagtgtacttgaggagagaagactctgatatattttacaatgtgagcatcttaagaactacaaattttttaaagtcctcattctttgaaaactttaaaaagtcctc

SEQ ID NO: 245 - DNA, 599 bp, Homo sapiens
cacttctactaataataactatcattaattgatgtcactaaactatgtattttacaaactgtggttaatttttataagactatcatattattctccccattttcaaatgaggattcctatgccccaaaaaggtcaagtaactcagtccccaagagtacaccactactaagggattaatttgggattccaacccatatctatttagttccaaagccaatacagtataccactatactatgctgtctcttaacaacattaataacttcaattactattgcaatcattttgaaactttgaaaytgctaagtttaacaaaaaccgtgttttagtaagataaatctagtgatgatgttcaggatggatcagaaggaaattaactggatgttggaaaaatcaattaaagatgtaataatttaggcctgagtccataatgctctgcattaggatgctggcaatgagaatggaaaaaaaaaagatgaatataagagaaattatgagagatgaatgaaaagaacttggcaactgcttaaacatatgaagagtagaaaggaatcaaagctgatttaaattgtaggtctatgtgactaagaaacaaaagg

SEQ ID NO: 246 - DNA, 599 bp, Homo sapiens
tgtagtgaaattctcacctcacccttaaccctggcaaacactgatctgctttccattgctatcattttgcctcatttttttcttttgagatggggtcttgctttgttgcccaagctggtcttgaactcctgtgctcaggtgatcttcctaactcagcttccggaagtcttgggattacaggcgtaagccaccgtgcccagcctcattttgccttttatagaatgatatataaatggaatcatacagtatgtagccttttgaatcttgcttttttcacttagcataaaacatctgagatayatacatgctgttgtgtgtgtaagctgcaaattcctttttattgtgagtagtactccataatatggatataccacagatttttcacccatttatggattgaaggggtatttcagttacatccagtttttgtcaacttttttgtttgtttttttgtttctgagaaggagttttactcttgtttcccaggctggagtgcaatggcgcgatctcggctcaccgcaacctccacctcctggagtagcggggattacaggcatgcgccacaacacccggctaattttgtatttttagtagagatg

SEQ ID NO: 247 - DNA, 599 bp, Homo sapiens
ttgagacgtagtcttgctctgtcaccgaggctggagtgcagtggcgtgatcccggctcactgcagcctctgcctcctgggttcaagcaattctcctgcctcagcctcccaagtagctgggactacaggcgcctgccatcacgcccggctaacttttgtattttagtagagacggggtttcaccatgtttcccaggctgatctccgtctcctgacctcaggtgatccgcccaccttggcctcccaaagtgctgagattacaggtgtgagccaccacgcccggccccacagtctgggttttyttaaaggctggataacttagtaattaatgaaatattttatgtttgtcacctaactatgaaagtaattaaatttggttcttcaggaaattgcctccctggttctaaccaggcgtttcacgctcactctgtttaccctcttggggtaaagggaatggcacagtaagttttatagttacagacctcacattcccttcttaaggagagtgaagcttttgtttgcttttaacaactttattgaggattaatggaaacaaactgtacgtatttgagatgaacaatttgttaatttttgacctgta

SEQ ID NO: 248 - DNA, 599 bp, Homo sapiens
ttgttgttgttgttgaattgtaagaattcttcatatgttctttattagatatacgattgcaaatatcttctcccagtttgtaacttatcttttcatattcttaaaagtgtctttagaggagcaaaaggttttacatttgaagtccagtttttcaatttttttcttttatgaattgttcttttgatgctatatcttaaatacgtttgcctaaacaagagaaaacaaaggttttctcttacgttttctttttcctaaagaaagtttataattttaggttttacatttagatcgataatccarttggggtcaatttttgtctataatacaaggaatgaattttaagttcatttttacatatgaatatccatttgttgaaaaaactttcttttttccactgaattgcctttgcacctttgttgaaaagcagttgtccataattgtgcgggtctacctctaggttctctattctgttccattatcttttatatgactactatactgttttgagtaaggtaggtttataatatatcttaaagtcaggtaaacgttaagtcactagtttgtttttttcaaatccgttttgactaatttggcttctt

SEQ ID NO: 249 - DNA, 599 bp, Homo sapiens
ttttgtctataatacaaggaatgaattttaagttcatttttacatatgaatatccatttgttgaaaaaactttcttttttccactgaattgcctttgcacctttgttgaaaagcagttgtccataattgtgcgggtctacctctaggttctctattctgttccattatcttttatatgactactatactgttttgagtaaggtaggtttataatatatcttaaagtcaggtaaacgttaagtcactagtttgtttttttcaaatccgttttgactaatttggcttctttgcatttctacrtgaatttgaaaataagcttgtcattttcttcaacaatgcctgctgggattttattggaattgcactgaatctatggataaatatgggggagacccaaaaactttacaatattgagtcttctgacccatacacaatctctctctccatttatttctctaagctttttttgttttgttttgttttgagacagggtctcactctgtcgcccaggctggagtgcagtggcgccatctcagcttactgtaacctccacctcctggactcacactatcctcccacctcagactcctgagcagttg

SEQ ID NO: 250 - DNA, 599 bp, Homo sapiens
attagccaggcgaggtggtacatgcctataatcccagctactcaggcggctgagacaggagaattgctggaacctgggaggcagaggttgcacgaagctgagatggcaccactgcactacagcctgggcaaaagagtgagactctgtctcaagaaagaaaaaacagaacggaaaagaaaagaagagaagagaagagaaaagaaaaaggaaagagctgagagtaacctgaactaatcaaagccatcttcattcccagggcctagcacagctcctggttcatacaaatacatgaatcaatamcagcatcagttctcaaagctctttttttaatagtatttccacttgttccagcatagagatatcctcctgtctctttcttaaacataattacattcatttccagggccaaatttttagtccacatcatgataaaaaaaattagatgatctctaatgcctataatttgaagttcaaactcaactgccacattaaactccaaatgaaagtaaagtatacttttaaaaaatctcaatcagataaataagcaaaaatttatatacaagaatattcatcagagcgttatttacaacagcaaaaca

SEQ ID NO: 251 - DNA, 599 bp, Homo sapiens
gaactctgacaatttcctctttgcattaaaaaagataataagtgtcaaaccagttattataacacatgacagcttcagaatagagacagcgtatccagctttatattattcagtatagacacatgggaaaagagcaaacagtttttctaacagatttctctgcctcaatgccaatatggcaaataatcataggaaaatgttattactatacctaattttcccttggagcttttaaatagaaacttttgttaactgatttttaaaatatgcacagggagtgaggacagcaacaacactacrtggggccctgccagtgacattcaggcttcagagaggtgacacctgggatgggtggggttctgaagattaataagggctgaggaccaggggatgcatcacaaagacagatgtctatcctaaaagggaagagctcctgaaatggcatgaattccactgagggagaagttggggaaatgaaatgagaaagcttaatctgcgctttctttctctaattactggggatgacaatcaaaattagaggagagtaaagtaatccagatgtacaaggaacagaagcaaaatgaagtagttaaaagaca

SEQ ID NO: 252 - DNA, 599 bp, Homo sapiens
tgttacctagtacctactatgtgctagctgtaacatcgaaatacaattgaatagacagggcttattttgtaagagcaaactctcatctacaggacatgccaggggcagaatcatgcttacaataagatgctcagaagcaagactgtcttttcctttatacctaaaaaagcttgtttagcaaacagagttatctttatctgaaatgcttctaatcctgtgctttgtatttatatccataccttttagataagacctactttattgccaattctcacattttactgagctaacgagcagtayctatatttgctgattttattttctgtcaccctagtgttatcataacttaatgctatcagggaataggaaagaatcaatgctgattatagttaagaaagcgaggatgaaagcaatacgaactgggggtatttgaatttgtaaagcctctacaggaggtcttcttatcctcgagtcatttaagaattactcacttaactcaaactgagcaacgatgccacgaacacatcaagtaaagccatcagtggctctgtggtgaggcagcatggtttagtggaaagacaattcacacaaggtcttta

SEQ ID NO: 253 - DNA, 599 bp, Homo sapiens
ggcttattttgtaagagcaaactctcatctacaggacatgccaggggcagaatcatgcttacaataagatgctcagaagcaagactgtcttttcctttatacctaaaaaagcttgtttagcaaacagagttatctttatctgaaatgcttctaatcctgtgctttgtatttatatccataccttttagataagacctactttattgccaattctcacattttactgagctaacgagcagtatctatatttgctgattttattttctgtcaccctagtgttatcataacttaatgctatcrgggaataggaaagaatcaatgctgattatagttaagaaagcgaggatgaaagcaatacgaactgggggtatttgaatttgtaaagcctctacaggaggtcttcttatcctcgagtcatttaagaattactcacttaactcaaactgagcaacgatgccacgaacacatcaagtaaagccatcagtggctctgtggtgaggcagcatggtttagtggaaagacaattcacacaaggtctttagagtcaggtgaacccgtttttgaaatctggccccatcactttttaaaaacggtgcaac

SEQ ID NO: 254 - DNA, 599 bp, Homo sapiens
ttgccacctttaaaatattttggttttattttattgattttgactatcatattagcttactcatatatatcatctgacacatccatttactggtcttttaagcatctctgggcatgtggatgcttgaaaaatagtttattagtatgatttgactatctctatcttgtctcctattttttttagagaattacatggccggcagtgaggatggtgtcaaatctcatcagtcatctacaaccactttctctgaccctttctgcatcattgaatttgactccctttcttcccatactctccttrgaggacctaagaggaaatgtgcatgggcagacaaaacagaaaactaaaggagggaagataagtgggttcttggcttccggaaggattctgacctacttcacttgccttcaaattctgtgtgaatttatttcagaagctacagggaagagctaagaactcctgttttccttggagaagggaagaaacaaatgccagattaaaactcagaggaaactctggaaattataagcgaggctgctactatataatgaccaagtcgccttggtatgtcttgtgagctaaaattccatttcactttt

SEQ ID NO: 255 - DNA, 599 bp, Homo sapiens
ggttttattttattgattttgactatcatattagcttactcatatatatcatctgacacatccatttactggtcttttaagcatctctgggcatgtggatgcttgaaaaatagtttattagtatgatttgactatctctatcttgtctcctattttttttagagaattacatggccggcagtgaggatggtgtcaaatctcatcagtcatctacaaccactttctctgaccctttctgcatcattgaatttgactccctttcttcccatactctccttagaggacctaagaggaaatgtrcatgggcagacaaaacagaaaactaaaggagggaagataagtgggttcttggcttccggaaggattctgacctacttcacttgccttcaaattctgtgtgaatttatttcagaagctacagggaagagctaagaactcctgttttccttggagaagggaagaaacaaatgccagattaaaactcagaggaaactctggaaattataagcgaggctgctactatataatgaccaagtcgccttggtatgtcttgtgagctaaaattccatttcacttttatcatatataaaaccaggatt

SEQ ID NO: 256 - DNA, 599 bp, Homo sapiens
tcccatactctccttagaggacctaagaggaaatgtgcatgggcagacaaaacagaaaactaaaggagggaagataagtgggttcttggcttccggaaggattctgacctacttcacttgccttcaaattctgtgtgaatttatttcagaagctacagggaagagctaagaactcctgttttccttggagaagggaagaaacaaatgccagattaaaactcagaggaaactctggaaattataagcgaggctgctactatataatgaccaagtcgccttggtatgtcttgtgagctaaarttccatttcacttttatcatatataaaaccaggattatagctccaggaggagctacaatctgtgttctctttggtctatctgaacatcccaaatagtatcacagtctacctgaagaagtgctgctgaaatagactgacaatgcttcaattattttggctgcaggcaaagacaaaataagtctcaggagttctccaaaagctcaaaaagttctctgtaaactatgctgttatcaaatggaaactacaagaacgcgaccaacaccacaggaggagagatttccctgggctttgctctggct

SEQ ID NO: 257 - DNA, 599 bp, Homo sapiens
aaagctcatcttctttccagatggtagtgaggtgtgaggtgatcctttctctttcttttttttttttttttacttttagctacccctcaattctggtgttcagtaacaatgtattccacttaaaagagagaaatcatattaattattctaagatataaacacttaattttattcatgctcttgaattactttcctttctaccgcatgtttgtgtagctgttatatgagtcctttctactgatgtagctgtcattccttcaactcagttcttgccaaacactgagctatgtctggagatayaaagataactactcttgttgaagagatgccagtttagtgggggagacagacatgtaaacagatctgtatctgagatagatggcctgggtaggaaaagaatatatatagcacaaataactcagtggacctgcaaaaacagagcttgaggccatgatataccacaaagacccaccaaggcacaagcactgtcaacatgaaggtaaggcttccatttttgcctctttcaatctgggtacatctattctacatactctgcctttcctcctgttacaggaaaagaaggaacctcaattctacct

SEQ ID NO: 258 - DNA, 599 bp, Homo sapiens
ttatctgtagcatacgatgctgtttgacagcatttttaccaatagtagaccttctttcaaaattgaggtcaatcctctcaaaccctactactgctttatcaactaagtttgtgtgacagcctaaatcttttgttctagaatctaccatttcaacaatattcactgcatcttcaccaggagtagattctatctcaagaattctatcttcactttctttgctcatccataagaagcaactcctcaaccattaaagtttgcagcaatccagtcatatcttcagactacacttactgctgttartattttgaccacctctcacgaatcacaaatattcttaatggcctctaaatggtgaatactttccagaaggctttcaatttgctttgcccagatcagaggaatcaaaagaatttgccatcaaaggaatcactatctatagctataagctatagccttacaaaatgtatttcgtaaataagactggaaagttaacatgattccttgatccacgggctgcagaatggatgttgtgttaacaggcatgaaaacaacattcatctccttgtacagctccatcatagctcgtgggtgaccaggta

SEQ ID NO: 259 - DNA, 599 bp, Homo sapiens
caagtttccacagctaaaggctgaatgttaagtatgaactgatttggcattacaagaatataaatcctttatctttttactgagaagaaaaaatgaagagattcgatgacttctgacttgaataagtggtttgcagtagcacagcgccctccagtggccaaatttcaaaatgcagtttcaaaggacaaagttcagtagcaaatcttttcatgttgaatggtgttaagctggtaacattagctcctttttcttttctcttttaatataattgtttatctcagcacatattttcaatatcaraccatttttctattgccaaatttccctcaccaaattaaaattcttctattcaaatctgatttgttttccctcagaaatattctgaaaagtaaaaaagaaatcaaagcgaaacaaaacaaacctaatgtggacacagacaactgcaatgagagggaattctctatacgcctcagataatggcgaatgccagcactagagtagtagtaatgaattcttaagtgttaaaatccaaactccagagtaagattatacaaattatggaactctattttgcctgatatacatcatgatgagtacat

SEQ ID NO: 260 - DNA, 599 bp, Homo sapiens
agaattggcatttgttatgtgcttcatgtgctactggttgcacccagcttttctatacataatcgtattacttaaggcaccacagaactctatacagcagttagaactgtgatggaaccagtgaggctggcttcctcttatccaagtatgactggaggacagggctcaaatgaccgtgcaggtgcatctgggcgggggggcgggggtataacaatgaagaggaggtgcaataacagccccgtggggatgatgacagacagaatatgctcatgaataagaatgtcttacttatcccatctyaattactttcttctgtctctttcttcatgtttaagtattaagagcagtgaggtggtaagaaatggtatgggagtcatatcagaatcctttggagagaagagatgggcacatgtgtcatctaaaaaagcattttcaaaattataataaaatacacaacatttgcaaagcaggctttctgcccgttgtgaccgtgaaaacaaagcagctgaagaggcctgatgtgaaagtgacctcctctgtgctctgtcactggcaagaacaggaaaaacacttggggcaagcaagccagccccactttc

SEQ ID NO: 261 - DNA, 599 bp, Homo sapiens
tagctaattaattgtcaagcatttaatgtaacaggattctaatctccaacctctgaagcagggtatttatttgttataatggtaaaacttctgtttaaattgcttatttatgcaatatatattctctatcatctcaattctaatttttatatctgctttgtaagtaaaataaagaatcccatgcagaaagacacagacagtacacttcacaaacagtaatgaatctcttgtgacttcttttattcacactacaatagattctatattaccatgcagagaaaaacaaaacaaaacaccatmgattatttccctcgtttgctcacagtattctgatagcaacaagaacttgaaggtgcagcatgctgtcttaggactgtgagtaaatacgatattctgttgacatcttgttcctgctttcaaacaaccaggtatgttacatatgaactgtcagagcgctgaataagttttcctgtctaaatgtgcccacattcttttcagagacgtcatcttaagctgagctccgtatcttagttgaatactgcagttaacaatttcttaacttgcaatagtcatttcaaatcaatatttaaaaggtataa

SEQ ID NO: 262 - DNA, 599 bp, Homo sapiens
ctttgtaagtaaaataaagaatcccatgcagaaagacacagacagtacacttcacaaacagtaatgaatctcttgtgacttcttttattcacactacaatagattctatattaccatgcagagaaaaacaaaacaaaacaccatagattatttccctcgtttgctcacagtattctgatagcaacaagaacttgaaggtgcagcatgctgtcttaggactgtgagtaaatacgatattctgttgacatcttgttcctgctttcaaacaaccaggtatgttacatatgaactgtcagagcrctgaataagttttcctgtctaaatgtgcccacattcttttcagagacgtcatcttaagctgagctccgtatcttagttgaatactgcagttaacaatttcttaacttgcaatagtcatttcaaatcaatatttaaaaggtataatcattactacttgtctgctaaccttcccctcacacacaccatgcatactttccatgtggcaatggaaatggtctgaatccagcagaaaatagaaaggtgtcaaagaaagagagatagaatctctttctcaggcccttaatgaggtctccagagcc

SEQ ID NO: 263 - DNA, 599 bp, Homo sapiens
tgggtggctgggccagctgctcaccatggtctgttctaacagaagattctataattctgtctgagagggaacctaaagaagtctttcagagctttggctactgacatggaatatgaataacacttaatttaccttcattttgctacccacgaaacaatatgagtacattttttttccatggtgtattctcttcattttggtataatatttccaacattttggaagcaaggtgatttttaaattttgcttgttgagtttttattaaaagaaaagctttctcttccatgtccagcctcctaycatgttcaaatcacaaagaccagtaccatttgctctgaagggaaagaatgctaagggaagaggactctgagaatgtcctatagaccaaggtcaataacgaggcaaacattctgctgacagaaatccccaaggcaaatacacaaataccaaacctgcagagatgagcaggatatcttcatcttctgcgcaggtcttactcttgcgtaacacagaaataggatggcttatcattaatgagcacactgtgcccacagaaatgaataagttgtttagaatatatggcatcagattcatgaagg

SEQ ID NO: 264 - DNA, 599 bp, Homo sapiens
cttcagagaactgtaatttcctattaacttgtcagtaatcttacctacccaccttagaaccagtgagtcagaatctccaggacagggactctagcatctgtattttttaaacacttacttgtatagctaatgcacagccagcactgagggacaactgttttacttccctgaagacaagaagtgtgatgtttactgttgtaacccaatacccagcatagtgctgacacatggaggttactcaatagggggaagggggaaatgattatacatttaaaataatcctcaagaacaagtttggawgttcttctgcacaagggtaaaaggacatattaccaggagtgcaatttttaagaatgaaatttctattttactttcagtgttaagtttttctttgctactgctaaacaatgttctctttgcatcaactataagtaaagaatggcagctaagatatgttagtcctaaaaagttcagtacaaatcagctgtcacatctataattctgcatattttgccttcttgaccttataaatactggctggaaaggcacaggtcacagaaaaacatcagctatttgagcatctgtactttctcaaaacg

SEQ ID NO: 265 - DNA, 599 bp, Homo sapiens
cttacacgaactggatgccatagcctactatacaccaaggctagatggtatagcctattgctcctaggctacaaaccagcacagcatgtaactgaactgaagactgtaggtaactgtaatacaatggtatctgtatatctaaacacagaaaaggagagtaaaaatataattagcacttaccatgaacagagcttgtaggaatggaagttgttctaggtgagtcagcgagtgagtggtgagtgaatgtaaaggcctagaacattactgaacactacagctgactttataaatactgtgcayttaggctacacaaagtttatttttaaaattttctttcttcagtagtaattaaacttagcttactgaaactttaatgctttataaacttttaatttttaactttctgaatctttaaaaataatacttagcttaaaacacaaacattgtatagctgtacaaaaatattacctttatatccttattccataagtttgttttctatttacattttttaacttttgaactcttttattaaatactaagacataaacatatatattagcctaggcctacactgggttaggatcatcaatatctct

SEQ ID NO: 266 - DNA, 599 bp, Homo sapiens
attacatatgctatacttttatatatagggttgcttacaccagcatcaccacaaacacatgagtaacgtgtaatactacaatgttatgatggctatgacatcactaggagttagggatttctcagctcctttatgggatcactgttgtatatgtgacttgctgttgatgtaaagattattatgcagcacataattgttactgttccctctgccccaaataaccttcccacctgaaaaactcctacttatccttcaagacctagataaatgttaccttccttacaaaatattgtctgaatytcccagacataatcatttcctcatctgtgtctgttccacagtattttggaaatgccttttttttttttttttttttgagacagggtctcactctgtcacccaggctggagtgcagtggcatgatcgtgggtcactgcagccttgacctcctgggctcaagtgatccccctgcctcagcctcccgagtagctgggatgacaggcgcctgccaccacgcctggctaattattttattttttgtagagacaagatctccctatgttgcccaggctggtctcgaactcctgagctcaagccaa

SEQ ID NO: 267 - DNA, 599 bp, Homo sapiens
tgcaggatatgagtcttgactgtagaaacaaattgccagctgtcacataaaggacaacctcatttagtagaaggcaaatgctagcacagaacaaaagtaaataaagatggctacatatgctctagctttcaaaccagaaagtgacagtgatgagagaagcagaagagggaaaggattaaacatgaagaagaagagctgctaccaatagcagactatctgactattcaaccagtcttctggtgcagtaagaatgccaattaaaatggctggtataaggatgtgatccagagatctgatggytaaagaaaccatgggggtacaaccaacacactcctgttattgtataactaaactcagatgttatgtgtaaaagtgctttataaactacaatgctctacagaaagacatcgtactgctattattcctaatatgtgtctattcttcttttaattgtaaacttattttttaaagtcccaattctttgtccattttttcatttctaataaagagctgtaattgtcaataccactgttattttacctttattttttcaagaaccttcagataatatatgctgctcaaattgagtatcttatagg

SEQ ID NO: 268 - DNA, 599 bp, Homo sapiens
gagcacaaagcttatataatgatggcagagtctaaaaaacagaaaagcccagatccttgacaatatcattaagccaccaaattaaccaaccctgagctgaccaatctctgtgcttctatataaggacatgtataatacttccttactatttaaggctctttgagtcacagtttcctcttatttgtagctataagcacctttactaactgcataaagcatctggctggttggagaggcatgtgaatggctactataaggtgccattatacaagggataaggttagatttgaaaagaactgktcttcactctcaagagcttataatccattttttataagacatataaaacaattgggaaaaaagtgatataccaattaaagaaaaaataaaggagcacaggtacccagagtaaagaaatcatcagtgaaggacatcagaggcagttttctaaaagagataaaaccaatacatatgctttttgtgcagcactaaattcaattagtgtaattcaacttccaggaagctttctgctgcagatatgtatagttctatcacttgagcacagcatcctcttgcatgtgggaaatgtgacttccttt

SEQ ID NO: 269 - DNA, 599 bp, Homo sapiens
gaatggctactataaggtgccattatacaagggataaggttagatttgaaaagaactggtcttcactctcaagagcttataatccattttttataagacatataaaacaattgggaaaaaagtgatataccaattaaagaaaaaataaaggagcacaggtacccagagtaaagaaatcatcagtgaaggacatcagaggcagttttctaaaagagataaaaccaatacatatgctttttgtgcagcactaaattcaattagtgtaattcaacttccaggaagctttctgctgcagatatstatagttctatcacttgagcacagcatcctcttgcatgtgggaaatgtgacttcctttactctgagctcagggatgggcaccagacccaaggtgaactagccagagccattttcttagattttttaaaacttgggatcataagggctcataaggatttgggttacactgttgtatggacttgtcctaagacttgtgcatttctttgtaaattttgtatcaaaagaaaaagataaataagtattgaattctataatacctatgctgaagtatttaggagaaagtatactgatagctgcaa

SEQ ID NO: 270 - DNA, 599 bp, Homo sapiens
gcatggtagatccactgacagcttgcaccgtgcacctggaaaagccacagataccagacaccagcccatgaaggagctgtccaagactgtgggagtgcaccccttgcatcagtcagcatgccccagatgagagacatgcagctaaatatcatttcagagctttaagatttaatgactgccctgctggattttggacttgcatgaggcctgtagcccctttgttttggccaatttctcccatttggaaggggtgtatttacccaatgcctgtatccccattgtatctaggaagtaactaaygtggttttgattttataagctcatgggcagaaggggcttgccctgtctcagatgagactttggattatggactatggacttttcagttaatgctgaaatgagttaagactttgggggactgttgagaaggcatgattggttttgaaatgtgaaaagatggctgggtgcggtggctcacgcctgtaatcttagcacttcgggaggccaggcagatggatcatctgaggtcaagagtttcagaccagcctggccaacatagtgaaaccccatctctactaaaaacacaaaaaattagctgg

SEQ ID NO: 271 - DNA, 599 bp, Homo sapiens
ctaatggaaaccaaaaagaacaggagtagctatacttatatcagacaaaatagatttcaagacaaaaactataagaagacacaaagaagtcactatatacaataaaggggtcaattcagtaagagggtataacaattataaatatatttgcacccaacactggagcactcagatatataaagcaaatattaaagctaaagagagagactctattagaatcacagctggagacttcaacaccccgcatccaacaatgaacagatcttcaacaaagaaatgtcaggctcaatctgcacttayagaccaaatgacctaataaatacttacagaaaatttcatccaatggctgcagaatacacattcttttcctcagcatatggaaccttatcaaggatagaccatatgttaggtcacaaaaaagtcttaaaacattcaaaaaattaaaataatatcaagcatcttttctgatcacaatggaataaaacaaatcaataacaaaaggaattctgaaaactacacaaacacatgcaaattaaacaatatgctctgaatgaccagtgagtcaatgaagaaattaaaaaggaaattgaaaaatttct

SEQ ID NO: 272 - DNA, 599 bp, Homo sapiens
cataatctgacctgtccagtctcatctcctgcccctcccctgactcaccatgttctacctgtacaagccttctttcttgtccttgttaccaatctacaaaggctcttcgcacagatatccacatgggtgactcttccctgtcattcaggtgttagctcaaatatgaccttctaaaaaggtctcctctgaccacagaggtattcccctacttccccacccaagcctataacccaccctcaccacgttcactttctaccaccttgtgttattttcttcacagcaatcactgatctttaacawtaccttgcttactgatttatttacctattgtatatatcacccctctagaatgtaaatgatgcagggaccatgaatgtcttacttgccactgtaattccagtgattggcacttagtaataaatgtttgtcaaataaacaatgcactacaataagagctcttacaattttcaatgggctcggtcaatacccatttacaattcatttctcattttttctaatctagaggctagccttttttgcaggttggcatgccatgacagagttcatttactcattcaaaaaatatacattgcacctgt

SEQ ID NO: 273 - DNA, 599 bp, Homo sapiens
acgtcattctcctgcctcagcctcccgagtagctgggactacaggtgcccgccaccacgcccagctaattttttgtatttttagtagagacggggtttcaccatgttagccaggatggtctcgatctcctgacctcgtgatttgcccacctccgcctcccaaagtgctgggattacaggcatgagccattgcgcccagcctaaaattcattcttttaaagtgtgcaattcagtgtttcttagtatattcccaaagttctgcagccatcgccgctatctaaacccagagattgttcctttytaaacataagtcaaagcctccttaaaactgtccagtcatggccggacatggtggctcacgcctgtaatcaccagaggtcaagagatcaagaccagcctggccaacagtgtgaaacctgtctctattgaaaatacaaaatttacttggttgtggtggcacatgcctgtaatcccagctacttgggaggctgaggtaggagaattgcttgaacccgggaggcggaggttgcagtgagctgagattgcgcctccacactccagcctgggagacagtgcaagactctgtctcaaaaaacaaac

SEQ ID NO: 274 - DNA, 599 bp, Homo sapiens
tcgcctgaggtcgggagttcgagaccagtctgaccaacatgatgaaaccccatctctactaaaaatacaagattagctgggcatggtggtgggcacctgtaatcccagctatctgggaggctgaggcaggagaatcacttgaacctgggaggtggaggttgcggtgagccaagatcatgtcactgcactccagcctggctaacagagcgagactccgtctcaaaacaaaataaaacaaacaaacaaaacagtatcgtaacagatataataataatgaaaaggtttgaaatattgcaagarttactgaaacgtgacagagttatgaagcaagcatacactgttggaaatatagtgctaatatacttgctcaaaatggagttgctgcagaccttcaatttttgaaaaacacagtatctgcagtgtcataaatcaaagacaaatagaatgaagtatatttgcattaaaaaaatcaccatgtatatcaattagggatacattttagtacaaatcacaggaaattttggcttaaataattgaggtgtttattcttattagatgataggttggtataggaattcaacagtaccttcagaagccag

SEQ ID NO: 275 - DNA, 599 bp, Homo sapiens
ttcagtaattatgtgttaaatgaatacatgaatgaagtttcttttccattgtgaaaattttgctaggttctaaggagttaaaaagaaaaacattcccacccacacagcaatacttcaaattttcaaattttgcagtgtaaattacataaaacactattatacccttattttttatactcatttagttacctctgattcaaaattatgtttctttgcagcaaatctataagtatactattctctagaaacacatcaaaaacatttatatgttatattttcagtaagaagtgataggattayttcaatttcactcattttcactccaccaaaatggtacagattttccaggctgaaacaattcatttccctttcatttacacacttatctgtgaaggcggcatgaagtatgccattggaatcatctctcttcgaagtctattacgtatgctttgtaatttactgaaaattgaaatcatcagtaatcatttcttcctgttctcttttcgggctagagttctttttttgtagagctttcttttcaggttttgccaacacattttttcaagtcatttttccttttcctttaaaatgaaaatgtg

SEQ ID NO: 276 - DNA, 599 bp, Homo sapiens
ttaaaatgaaaatgtgcctacaaagttcaaaatcgtgcattcctagatggcatcttggaagaaagaatagagcaatttcaaagcaaaatatctattttttttcccaactcacgcattaatctttccctagagatatgtaaataggtaaagaagtgaggtccatacataaaaagaaacagtgtactaagatgtttacctgaaaaccacactataattcaaatattgttaagggtcattgtcaatatgtttaggccaagagcacaatctacttttaaactgttgtaacctgataaaagggayaaaagtttgacacagtattcctctaaagagagcaatgtaaaaagatgagcaataaatccataaggaaatggatacagccaggcagaaattatagaaaaggcaccaggaaaaaatcaataactgcagaaagggacaagagaacccatgggtaaaggtgacttgtaaattctaaggatttaagatcttttagaggattttcattagattgggctgtttttccccaaatctagcctctcagtataaataagtgaaggctatcttcttaaccagaagttcacaatattttttaaaagtaacct

SEQ ID NO: 277 - DNA, 599 bp, Homo sapiens
gttagaacaatcaaatcacaggattccttggctcaataccctccagtggctgtcaatctcactcaaccaagcgctctgcaacctggccccatttcttttctgacaccgtcttatactactttctcttcagcttcagccctagtgccctccttactattccaggagcataccagagccagttgctgttccctcagcctggaacactctttgcccacattggcttgttcattaatcttcaggtctttactcagatgtcacctttataatgaggccttgcctatttgaaattgcaaacccacsccataaacatcctcttccctgctttatttattttgttgtctctctctccccagtagaacataatttctatgaagacagacatgttgtccaatctgtacttcactgaatcttcaatacctacaatactcaggttattcagactggtctgggaatcttcagagtgaagacaatgctcaggcatcaggcagggcgatgttcactccgggcaaagggcataccatgtttaaagatacagaacaatgaactagcacagtgtacacttggaatctagaagcagtttggtttggttggagcttagg

SEQ ID NO: 278 - DNA, 599 bp, Homo sapiens
tacactatattgcctctgtgctatggatcttgggtttcatggtatagacaaggaggagccactcaaaggttttacaccggcaaacaataggtttagtctgcattttaggatcacagcaatgtggagaacggattagaaaagtgtaaagagacctgcaaggtccaagctatagagatgttgaaggcagggtttaaggcagcagtataaagaaaggaagactttacagacacccaggaagattttataaacacccagaatgtagaaattattagggattttaagagtgtggtggctgaagayccaggaattcctgctctgccatttaccattctctccatatgcatttacatttacagaaaataagaaatcacaagctgaatgaaaatcaaagtatgatactatgaggtggctcagcatctaaataccacagagcaggaatacagtaaaactggactggaacatttggcttaggatcaagatgcaattaagggcctctatcttaagtccaattctgtatcttgggattttcctatcttcaggccaacaacacagtatagaaatcggaagcagtgtccagcatacttaggcttacatgcaaa

SEQ ID NO: 279 - DNA, 599 bp, Homo sapiens
cacatattagagatacttcttttgtcaataaacaaccttacgtatgtatataaatgtttatttggatttatatgagaatagagaatggtgtggaaggatacacaccaaactttaatattgattaagcaagtagtagtagatgtttatttatcttttgttatacttggctatgcgatcatttttaaaaaataaatagggagttgaagttactttttaaaagttctgggagatgaggaaatgaagaaaaaaagaatagatcactttttttgagaaacttcgccatgaaggtaaataaaggtrcctggtggacactagaaatgcatgtactttttaaagacgagaggtgctaaagcatgtttaaatactgattaaacggattcaggaggaagaaactgaagagggaaggagaaagacagtcaatggaataagacacatctccattttgtgtaacagaaagaatgttatattttcgtattgtgatgacttctattttcctgtgctatcttcaagactgcaaagcatatgtttttaaatgtaccctacagaatctgggtctgaaggagtccacatcataatttgatctttgcagattttggctt

SEQ ID NO: 280 - DNA, 599 bp, Homo sapiens
aattcattccaagaaaataggtatgtaagcatgtttattgtagcattgcttatagcttcaaaaaactgaaaactaccaaaatgcccatcaataaggaattgattatatctgtatgaattgacctagagggattttaataaaaattgttagatgaacaacacatattagagatacttcttttgtcaataaacaaccttacgtatgtatataaatgtttatttggatttatatgagaatagagaatggtgtggaaggatacacaccaaactttaatattgattaagcaagtagtagtagatktttatttatcttttgttatacttggctatgcgatcatttttaaaaaataaatagggagttgaagttactttttaaaagttctgggagatgaggaaatgaagaaaaaaagaatagatcactttttttgagaaacttcgccatgaaggtaaataaaggtacctggtggacactagaaatgcatgtactttttaaagacgagaggtgctaaagcatgtttaaatactgattaaacggattcaggaggaagaaactgaagagggaaggagaaagacagtcaatggaataagacacatctccat

SEQ ID NO: 281 - DNA, 599 bp, Homo sapiens
tcagttttttgaagctataagcaatgctacaataaacatgcttacatacctattttcttggaatgaattttgccaacaaggggattgctggctcaatgggtgtatgcatgtttaattttattataaagtatacatttccttctttaacccactctaatctgacttccaccactactgtatcagtaaaactgctcctgccaaggtgaaccatgcactgagtgttgccagggctgatggacacttcccattcctcatctacttgatctgccactgatgacctgtccctctttcttgaagccrcacattcttgactgtccttttcttgctggctattctttccccatttcattttacaacttctcttctacctgagcaaattttggagcacttcagactcagtcctaggtcctcttttctttttcctctacactttactacattctatttcctttggatctaattggctcctatggtttcaaatatcatcttctaagacaacaactcttaagtttctatttctagcccagacctccttttccatcttcaagcctgtatctaacaatgctccctggaagtcacataagctgaagaacacatct

SEQ ID NO: 282 - DNA, 599 bp, Homo sapiens
tacttaaagcaggtatattctccacctttatttcgggcaaatatcctgagttgaaatattttttcttaaccatacttttgccctagagaaatcaatggtaaaaaggaagtttacagagaaaaatatacttttgtgcagacagcttaccggtgtgatgcagccatgaatcacactgaaaataagtcccttgaaatcaaagaaaacaatatgtttatacaaagaatgtgccccaaagttccccaacaaagaaaagtgtttaatcgaaatattcgttgacagctggttagtgctgattctcargtagaaaagaaaatcagtgtcattgtagtctattgttttgtgtgtaagagctgaatatgcaaatgagaccaaaatggagactcattatctgttttacacttcaagtgtgattttaagcactgtaggtatacatacacacacacatccagaatacacatatatctcaacaaaccatgcacatcatctgttcagaactgggaaacgcgtgcatgcttctacaacattaaatggaaggcagatggtagtcactgatcaacagacataaaataaagtccttctaaatcctactctctctaaca

SEQ ID NO: 283 - DNA, 599 bp, Homo sapiens
aatctcccaattagccattaattatatatcttgtatacttttgaaatcattttgcagttcctggaccttgctttatattcctttggtggtagtggctcatcaggtatcttgttgaatctctttagctctctaaaatccctgaatgcaggtgcctggataataatctgtttttctggaaaatacccttttaacactgctatttttttttgccagtgttagaaggaaataataggaaacaaaattttatattttttttccacaggaaatatgtttaaagaagactgtgagtacaagtcccayggttagtaaaatcatcagagctttctcagtcatcattataaatggtagaagttccctagatcagggtgctgtctgtggatacaaggttagctcaagggagagtagaccagaagtaaaaccagattgcttgtcggctgccagaaaatctgggcagagtgccaggcctggttcatcttctaaaaattcatctcccagtatgaaaatagagggctcaatccctgcaaggaacggtggtagcacgaacacttggacttccttcagcccaaagcactttattgatgcgaagaaaggtgggggga

SEQ ID NO: 284 - DNA, 599 bp, Homo sapiens
ccaagttcatctctcctatgccagactctaaattgtcaatttacacccatctcaacaccctgtctggccttgtctccagtaaacaacgctcctaatcctcctctaaactctgaccagcccctgaagtaaatacctctcccacctacgctcttgttccctctctcaacgggcataaccttgagaacgttgcttttagccctgctttcttctctctcaactcccaggttttagaccaagatcccacttgcaattccagccaccagtcacatgcacactttgaattctacttctatccatttwcgctcatgttctaaagttttctcatttttcagcaatattatatggctgccaaattaacagcaccatgttgaggaatagctcaaaattttaagagcaccccccagagtactcaatatccaccattcattactagtgttttctgtttagtactaggctcgatcatctaagaaagctgtggaaaggactgggagagagtttaccagataaccaagaagacaatttgaaggttacagattatgggtaaagcggaaattgcctttcctgagaaagaaaaggctgataagacataacatggttct

SEQ ID NO: 285 - DNA, 599 bp, Homo sapiens
acatcactgttgctcattttttttcaaccagaattgggagctggctataccaagacaaaaactgccttcatgaatacagccagtctctaaacacgtaacagctctaccaacaaagtcaaggccagacaggaaagacaaaataaggagagtataataatgctcactaatatataaaggtaaagtatattttctaaaagccagagatcatgatcaaaggcacgtttccatacatacaacaggagctttcaggtgggtaaagcacacaggctgtcatggctggtattattatattcataatgrtttgttctgggagtcatgttcctgttgtttcaaattaaatgcaaacacacagtccttcagtccatatcagacgctttgttatgctgcctgtgttttaatcagatacaacagtaataacctgagtcaatgcagactcaatctcctcctctatgtgggggtccctctgtcactctggaagtatccctaggcagctctgagatatggaatcttcactcaatttcttgtccctcttattttaatccattcaaaactcctctgataaaagagcaaccgctagacctgagacactttagtctgtc

SEQ ID NO: 286 - DNA, 599 bp, Homo sapiens
taacaatattgtgtccggtcataccaataaaaaaaatgtaggcaacgttctttactgtaccagaggctgaacggttttgtgaagttccttagtggaggtaaaaattcagccttttgctgttagaccataagtttcacaagtataagagtaacttctgaccaagagactaagtgctgtggtgccttcccatctgctcccaatcagccacagtgggagatcagatgggtcatgggtgaggggccagctccacattctccatggtgtacactcaccatatcccttcccttttcttcctgagcraacaaactaatcactttctacctttaatcttttgactttaaggtaaagaatatctaaaggcaatttccagtgagaaaacacagcagaaaaaagcaacaaaattttgggggcctttaaccacctaaacaaattcactaatttgtataggctttacaaaatttcagaaattttaaatgatggattatacctaaagggagaatatgaaatattaaaacagtagctttttgatacttttaaaagacaaccatatcgagggctgcgatacataacaaaaataaaagcagaggtgctctggctga

SEQ ID NO: 287 - DNA, 599 bp, Homo sapiens
catctcaaatttaaatttggcttatgacaggggaatatagatttgttcaggatgggacctgcatgtaatggttcggagtagctgactaggggaggattataggctgttttcgttactgccagattatcagctggcaagttttaagttgtacgaaacacgcattatctcttgactggtgtcattgatttttttgcaggtagcttgtatgtgggagggtaggagactgggaggcagcaggagataagctgccctctgtgagggagccattccaagttgactttcttccttagaggggtggcygaacaccctggccagggaaggtagagggagaaagcatacttcctgcttctccattttctgttctttctctctctcgtggcatcatttatcttctaatatggtataggtgagacatcctgaccatgtttgaaaaggagtaaatatcttgcatgcctgatgtgccaagcactgtgtaggaccctggggatatgaatataagtacaggatggcctttgttcttaagcagcttatgatttgagaaagcagacagacaacctgagagataatttcagtgcattgtaggtgaatgctatcataac

SEQ ID NO: 288 - DNA, 599 bp, Homo sapiens
gaattgtcaagaaagtcacttgtaatgctgcagttcttggggattattcacaggaagggagcatccatacattttctagggaagttgggatggggagtagaaaatgatttaggtgacagaattgggtaagtagattgctttgggaagagaactaagtctggatccaaataatccactggggcgaatgaccatgtcaacctacatgtaatagagagttcgataaaagataattaagaacataaagcaatgaaagaaaagcacaaggtaattaacccagcactcctctcagattttggcacygtgtagctaggggtacattctgcttcatttctccttaatttttccttctaataattagcaatctctacctagctgtagaatttcaaagcttaatattatgcattaaaaattatgtgtaaaaaattatgattatctactatcttgtttaatctttatcaccttgttttttcaagacagagtcttgctctgtcatccaggctggagtgcagtggcatgatcttggctcactgcagcctccacctcccaggttcaagtgattctcctgccttggctgcccaggtagctgggattatagacat

SEQ ID NO: 289 - DNA, 599 bp, Homo sapiens
agatagaaatatctccacagctaagacaaagctaaatgtttggcagaggtacaggtagttcaaatatcctaagaaaaaaaaattcattaaaaagttaattctctttccccagctgtatagaaaaatgttcatcattatatatgcataaaactagtctaaactaccttccagaaagaccatcacagataagtttaaaacactgtttggatcttttttttttaagggggaaaaaaaatccctgtaatttggtaaaagccttaggacatgcagctctaagaattaaactttacagtaaccaaygataataaattcaaagaaaaagcattagtctctatatcccagtcccaagataaaacctctctacacttcatgagaagtccccaaagacacagaatctacatccagattctgaacaacaacaacaacaaaaccaaattctgtcaataacttcaaaataaacagacctatcttttcaagtcgtcagcccaggaacacttctgaaattttatatcctttttttcccctaacagtgaagaaatttgataaaagaaaacaatgtcatgaatgttaagataacatttactggccaggcgtggtgg

SEQ ID NO: 290 - DNA, 599 bp, Homo sapiens
gtgctaatgatgcaacgagttttcaggggaaaacttctgcgtaatgttgaagtaacaataaagtataaagatgcagatgacgatcttataacaatttttgatagttctgatctttcctttgcaattcagtgtagtaggacactgaaattgacattatttgttaatggccaacgaagaccccttgaatcaagtcaggtgaaatatctctgtcgagaactgacagaactttgaaataaagtgaatcacttattggatagcttggaaccacctggaaaaccaggaccttccactaatattccygaaaatgttactgtgaatggtagggaagaaaagcctgcttcttcggattcttctggaaaacagtctactcaggttatggcagcaagtatgtcagcttttgacccttttaaaaaaccaagatgaaataaaaaataatgttacgtcggcatttggcttaacagatgatcaggtttcagggcaacccagtgctcctgcagaagactgttcaggaacatctgacagcattgccgcctcctcctcagtagcagctcacccaccaggagttcagccacagcaaccaccatatacagaagctaaga

SEQ ID NO: 291 - DNA, 599 bp, Homo sapiens
ccacactttatccaaggctgacacttaactagtaccattctctagcacctctgatttcttcctggacctctcccaacctgatctctctagctaccatactatttttttactaccttacaatccaattcacaaatcagacagacaatatgttatctctattatcttttcaaacatccatggcctaattccctggcagccaatcttctgtccagattacttatcaactaatcacaatttatatccaaaagttgtctctcaattttcttgacctccctatcgtgagctaccattgaccattgratccataggaaagctctctcctccggttgtcctctgtaactctattctatcccaaatgtcttccttcttccttctctaccttaagtcccctggcaataggttttctccaagtttcagactttgttttttacttctttttctatactctagcagctcaggtttcaactcttttatcaccagctttgactggtcctaaagagtaatgtgttcctatttcctattagacatttctccctgcattcattcactaagcattcaccaagcattcaccaattacccattatatgctatttggtgtg

SEQ ID NO: 292 - DNA, 599 bp, Homo sapiens
atttttttactaccttacaatccaattcacaaatcagacagacaatatgttatctctattatcttttcaaacatccatggcctaattccctggcagccaatcttctgtccagattacttatcaactaatcacaatttatatccaaaagttgtctctcaattttcttgacctccctatcgtgagctaccattgaccattggatccataggaaagctctctcctccggttgtcctctgtaactctattctatcccaaatgtcttccttcttccttctctaccttaagtcccctggcaatagrttttctccaagtttcagactttgttttttacttctttttctatactctagcagctcaggtttcaactcttttatcaccagctttgactggtcctaaagagtaatgtgttcctatttcctattagacatttctccctgcattcattcactaagcattcaccaagcattcaccaattacccattatatgctatttggtgtgttaaatgttgaggatatagagatcaataaggcctataaagatcatctctgccttcaaggatcaaagacaagtaaacaaaaatgtttaaaacaatgtgatc

SEQ ID NO: 293 - DNA, 599 bp, Homo sapiens
taagttttatgtgcaaaagagactgtgctgctgtgtggttgttttcaaggtagtgacaatcaaatagggtcctttttggtattgcatactaccttttggttttgtttttctctgacagagagtgtgactaataattccttagagcaagcaggcaaattcatagaggagatctgatgcaacatttgatgtctttaagtccctgctttttttgctggcttaaaattcagtattatagctagagtaaaactgttttgctcaaatcttcctttgaactctgggtagtgattgtctctccatttsccttgttgtgttccttctagaacctggatatttctaatgagaaaaatctattgaaaagctacatatcttgatttgagtgtaaattaagatctgtgttatcctatttcatttttttctacttaatttttaaagacaatgatctttgagggttgcaacattgttcctggaggtcagtttggagatctgagagccaaaagtatggtagtgaagtaatactgtggaacaaaatttgaattttaaatgccaaatatgtctttctaacttaacaatgtacactctcttgaagaatctgaatgtat

SEQ ID NO: 294 - DNA, 599 bp, Homo sapiens
tgtatgcagctgttgttggttacttgggtccttaatttccagggttataaatcttaagctgatctttaactactctacattttacctaatatttgtcaaaagggagtcatcactgaattaattactgagtgagttataatttatgcatgataatggatctttttctatttttaaggataaagatggttcacagagttatagatttatctgggaaacttggtatctaaaatggagattccattggattagagaaaatttcaattaccttgtagtgataactgcagtgtctggcttttatartcagaattaagcaagacagcaacacctagagtcagacatgttgtggtaaagtgtgtctcctggaaatatttactctgtagctagagctcctatggtttttcatgaacttccaatcttacacatgtaaaatgccactgtagatgaaaccgggagcttagaaaagtgaggagtctgttaaaagttctagcactctcctattcttcctagtggtcaactctgccaaggcatccgaccttggtctagccattatttgttatcagcctctgtcctctgcacacttttccttttatatttcttcc

SEQ ID NO: 295 - DNA, 599 bp, Homo sapiens
tttcggtaagtgtgtaatactttttttgaaaatttagttcacttgtagaaaacactggtattttctgctctatgaaaaatatataaccaagtataatgacaaagtcttgcagtctctcaattcaatatctatgaagatttcatccttgtagactttgttattttttgagaatcaccttttcctttaggagccaccttaaaattaaagggttagaaggattcccagaccaaatagtgcccaatcaaccatttttcaaggtctcaagaatgtttctcaggctaagtcttcgttgccctcacygtctcctcccagtagatggctaatgggactaaagcttctttttgcataggtagactgccctgtggacctgctaatggacacatataccccttggtttgaggtctaggagctgttagtaacccagcagatctaagacgtgcacttgtatgcagctgttgttggttacttgggtccttaatttccagggttataaatcttaagctgatctttaactactctacattttacctaatatttgtcaaaagggagtcatcactgaattaattactgagtgagttataatttatgcatgataatgg

SEQ ID NO: 296 - DNA, 599 bp, Homo sapiens
aactagcaggataatcccttaactgccaaggtaaatccttctccctcaactgcagcctccatttgaaagccctaaaaacagaataatctctagtcacttgtgtatgaaagaccaaattcagcttaagaggatctatttgagagtatgagcaagtaatgtgtgacacagtagtcttaatgtgtacacacaacaactgtgtaatataggagctggaaaactttctataaagggtcagacagtacatggtttaaagctttgcaggttatacaatctacgtcaaatgattcagctctatggtargagcctaaaggcagccacaggaaataagaaaatgaataaacatggctatctccagtaaaactatttctagaaaagacctagattagactcgcaggtcatagtttgctagcccctggcctaccccgtgatcaaggactgacgtcagggactgactgtaccctaggcaaaggatcagactgggagtttttttgtttgtttgtttcttgctgggttacccagttgatgttcctatgatcatttcctatgttggtcaggaaattcagaaagctactggtacctgacttcaaaacctcttcaat

SEQ ID NO: 297 - DNA, 599 bp, Homo sapiens
aactataaacaaatatgtcagtaagtttggccacaaaattacaaaataaattgctttgtttgtgagataagtgtttggatttgccataagcaatagaaattgatttagtgactaagacagaaatggcaagcagaagggctatgtattatctccacagagaaaaagtaattaacaacttgaaggcaaggacataaagttcattacattaaaactggcaagagaaaaaaaaagttcattacactaagacaggtaaatcagatacctgggtagcatggcctactaggacattccatctgatawatcccaactaaagatcttcacctaagagagtttcagtctgactttttctagacttaaggcagattttcccagatgcaaagtgttcttcaatttgcccctactgactgtcctggagtctaaacttgctgatcctcacagcatagggcaagggtaatgtattcactcaaactgttgcctcaaggtgggaggaagaacagtaggaaggcagggggacgaaggacttacactgagaatggacaggaatatctcaactaggaaatggtacctttcacggtaccatacacaattattagttttgg

SEQ ID NO: 298 - DNA, 599 bp, Homo sapiens
cacaaagattaataaaagtctctacttttgaagaactcattgtctactaaggaaagtaggaccagataacaagtaatgtaaattctaaaactcatagggtggctatgattttatgaaatttagaaagtcaaatacttccataagtaccataccaaaacaaccacaaagatgctaaatagaatactaaatgaacagaactcagaaaattatcagttatattcttatatgagagtaagggttaatattcaaataattggctatgtgctctccaaagtgggctgctcatattcgagggagackgcaatacaaaccacaggaatttggaaagaaaacattaacacttatattgatgtgtttgtatgtatttattttagacatgaggtcatgctatgttgcccaggctagactcttaggctcaagtgcttttcccgccttagcctctcaagtagctgggactacaggtgcacacaaccatgcctggattgatattaatttttaacctagaaaaatgagaataaaaatcagctttgctaattcttttttttttttttaaaggcaatcttgctctgtcactcaggctggagtgcagtggtgtgatc

SEQ ID NO: 299 - DNA, 599 bp, Homo sapiens
tatcccatcctcatatgtgtaactcttcactgtcagtgaagtgcagttatttgaaaaaggaccgcaataattactttaagcattcacacaggtattgagtacctctatgccaggtacttttctaggcactgaggtgaatagggcagaacccatgctctcatggaggttatcttctagggctaagagcagtgaagaaaatcaaatggagtagaaggttagaaatggtatgggagtgaagggggtagtagggtatgtagggtggttaggagccaagacctgaatggtgagtggccagtagtrtgaagctttgggaacaaaacattctggtagagggaaggtaatgataaggatcctaagtcagtaataagctttgactgtgtttaaggaacagtaaggccagtgtggctggagcagaatgtgccaggagaggagttggagatgagtttggggaggtggttgggttgcagagctgtagatcttggtggagtatgatttttattcagcatgagataggaaggcactgaagtgttttaagcaatggagtgactgatctgatttatattttcaaaagatttatgtggagaaatggcatgtaaggg

SEQ ID NO: 300 - DNA, 599 bp, Homo sapiens
taaaccagttaaaattttctagttattattgatcctttttagtaacagcatgagtctcactcctgtcaagcctgctaatactcatgctgcagtttttagttagtaccttgctaaagtcctggagatgaaagtcagcctaataaatctttgtaacatctcattgagcgactcatttgttaaaaaaaaatcactttttaaggaggtaatttataaaaatggtacaaaattttaaaatatgacagcatttacagtggaaattaagtcgccctcccatcctgaccccacagacatccagttccmcctctttgaaacagcttaccattggttcattgccaacaccggtgctgttcttgtgtttggggatgcaggggtgaacaaaataggccaagatctgccttaatggagtgtccattctactgggggagattgaaaataataaagtttcttgcatatctattcagaaacatgctgtgtatatgtggcatattctaatgtttattcttaagcccatatatattgtactgcatcttgcttttctctcattatatcttggaggctgttccatatcagtaccttaatgcctacagagcaccatgtca

SEQ ID NO: 301 - DNA, 599 bp, Homo sapiens
gtggcacgatctcggctcacagcaacctccgccccctgggttcaagccattctcctgcctcagcctctggagtagctgggattacaggcatgcgccaccacgccaggctaattttgtatttttagtagagacggggtttctccatgtaggtcaggctggtctcaaactccagacctcagatgatctgcccgccttagcctcccaaagtgctgggattacaggcgtgagccaccgtgcccagccaacaacaaacattttttaatcttatgatttctgtgggtcaggaattcagaaccagcktagctgggtagttctggctgagtcactgatgagttgcaatcaagcttttgaccattgctatagcagtcatctgaaggcttgactggggctggggctggggctggaggatctgcttccgagatggtacactcacatggctgttgacaggaggcctcagttccttgctggctgttggtaggggacctcagaaatttcatagggttgcatgagtatccttataacatgtcagctagcttcatccagagcaagtgatgcaagagagagcaaggaggaaaccatgtgccttttatgtcctgttt

SEQ ID NO: 302 - DNA, 599 bp, Homo sapiens
gttgggtgctgggaagacaaagaagttaagacacagtatatgctcagcatactaagtgttcatcagaagagataaaccactaaccattatgtgctgtgacaaattaatcacttttggaatgaataatgtgctgggggagtagagtgctttggctggagaaaaaactgatagggaacatgttgagaagaaggggatggagcttagctctgagggttaaataaaatttcgccacatgtagaggtggaagagagcattccaggggccagcataagcaaagttgctgaatattaaaattaaatrgcgcttttggaaatatcttgagttgtttcttgtgggcaagagaggggagagggctgaactcatctcaagaacccattcctgagataatgaacccattcctgctataatggcattcatccattcatgagggcctaatcacttcttattgcatgggggaagtgatgaggcaggaagctgtttttggagccaaattatgagggggtcttccacttcatcctaaaaagtctagactcctcccatccattggcagtggagagccatagaaggtagaataatcagaaagttgacagttcgtgtgc

SEQ ID NO: 303 - DNA, 599 bp, Homo sapiens
cttcttgtaaggtgggatagaggttgtattaatcagagtacagtctttgacaagaataagtgttatccttacttgaaagtaaaggatattacgcttgaaccaatatcaggttgttaatatacttttgatttcaattactaattggaatcgatgcctcactaatgtgcatgtattttaggtagtcagtggtgcatgtctcattatctaaaatctacctgatgtacttgggccattgtattggaaatttactgaaatgatgtaccaatagtcatccagtcacactcagtgacatagttttgyttgggattatctctctgaggtttgtctttactgctgatcctgtaaaatatacaaatgttagttgggaaaggagtctttctgatgaacgatgcttaaaaatactgaccaaagagatttcagaataattaaagaccctgctgctgaatattgaacccttggtctttatttataacatcttctgttatgtgaaaattcttttgagtggtatgtaaacctgaagttgaattgctaaccaagatgtgtaaagttacaactgcaaatgttgcattatttaacaactcagcagatacttactgggt

SEQ ID NO: 304 - DNA, 599 bp, Homo sapiens
gagtcagagaccacaaaaaggccaagtatatcaagaactggaaagcattcactggatttagcaacagaaaagtgattggtaaccaacaatagcaaacttcagtgggatagtggagatagataccaaatcactgagatctgaaaggcagatcaatatagtggattggcctctagactagtttgatgggagttttgagattctagtcttgattctggccattaactagctagataactttgaacaaatccctcaactactccaggctttggttttcgcatctggcaaaggatgagatcaagmtgagtctatgattttctcattaattcctagataatccccccagttttatttttttattttttgtcatatctgattcaatacttttatttatttttattttttatttttgagacagcatcttgctctgttgcccagtgtggagtgcagtggtacaatcttggctcactgcagcctcaacccccgggctcaatcaccccaatttcatatcagactcacttgcttataacagcacacttcaaatccttctcagaccattttccaaccactaagaatcagtatatttcatgccagcttctgtc

SEQ ID NO: 305 - DNA, 599 bp, Homo sapiens
attctcttgtgttcatatgaggaacacagagtgatatcacacacaacaacctgtcaactatgccttctagaattcctcagtataagggaaagggtacatagtatgaagcctggggggttaaaagcatggattcgaatcctagctttatcacttattaattgtaccttgagtaattacttaactttctatgccttggcttcctgttttataaaatattgtgaataatatcacctacttcaaagggcagttatgaggcttaaatgaactaatgcctacaaaacacttaaacatagataacayaggtgcaagtactatgtatctggtacatggtaaacatccttattattaaagtcaacgctaaaatgaatgtgtgtgcatatgctaatagtacagagagagggcacttaaaccaactaagggcctgagggaaggtttcctggaaagaggatgcttgtgctgggtccaaatcttggtctactatgaccttggccaaattatttaaactttgtccctatctgctaaaacagatactttgtcagtgatctatacaataagctaatgtttacaaaattaagtaagataatacatgaggattagac

SEQ ID NO: 306 - DNA, 599 bp, Homo sapiens
tgcatacccttttctcctaatctgcatattgtcagttcattttcagcacagcttcagagggcagaggggaagctttgcctatttgcccacacaatgcactggtgggctctgtggagttgtctcttccagaattgcttaacatatgcagaagacacaatactcaaaatacaagccatgaaaacactcttggtggtggatgaggaaagtttctgtgtgtgtgctcaggacaatatggtcaggtatgcagtgtcctgatggaccagctaatattgtgtggctgactaaaggcagatacatacygtttagaattcagtaatctaaaaagcagatggtagagggctgacagccttttgtgaagagtgagtcaccctattaagattctcacctaaataggaacattcctctccatatggtagtgaatagagagagcccctgcaggcaatctaggattttccttaaaaaaacaacacttcaaaaataaaaaaaataagtgttcaagaccatcaagttccatccaatagagcaagaggtctgcgaactcacagacaagtcatgtttttcttttctttaaaaaaaattcccattagttaccaatattt

SEQ ID NO: 307 - DNA, 599 bp, Homo sapiens
acgtcgatacctctttactgcacagtatgattggtcactcaacaaagaacacactctgttcattagaagaatttttttttttaattcaagggagcaaacaagatggccttccttgaaacactcctccctttgcttccatgaaactgtacccacctagttctactcctaccttcctgaccggcccttctgtctccatttcttcctcttccctttaaatacagatgagaaatactgagtggagaagaaacctcaactcaaccactcaacatgacaccctgagcaaattatatccgtgtgagraatcacgtcttaaataaggagtccagatagtgatactcactttacttcctaacagggttacggatagattcaagggagaaagtgggcatgaaccattttgaaaactgcaacataggaatgaaagacattcctccaatattctgtcctcagcagtagccttttcttttctgtaatatcttcctcctgggacagtttcacctactctcatggctttaactatcatctgtttgattattatttaaggagataaaaagattaaagctataatataggcagagcaaaacatgaatataatatat

SEQ ID NO: 308 - DNA, 599 bp, Homo sapiens
taaaaaatcacaacttcttgcctagttcccaagcatgagcctgttttcagacccagaatctactgactgagtgagaggccaggttaacagaaagaatgaccctgcaacaccatagaagtcatatcagtcacgacttccccccaatcattccccagaggaactacagtcatttactagggtagccatacgttggtgaaagaggaatactcagtattttgaagactgttggacacaaggctcaagttgtcattgatacctgggaacccgaagcatcatcatggcccctggggcttaagggartcagataataaatggagtcctggcccagggccagttcacatacccagccagtggtcatttcactcatcttctgctatataattggaacccactttatagttgacagaacacccatgttgtttccttgaactggggggtaagagctatcctagtggcgtaggttatgtagaaatttctgcaacataccccactctggccaaaataataaataaaaaatcagtatcagatcctgggaggaatggcaaaaattaataccagccttaaagatgtaagggatgcatgggtggtgaaccccgtct

SEQ ID NO: 309 - DNA, 599 bp, Homo sapiens
ttatatgaagatctgttcctgaactttatattctgtccctttgatctatttgtcaataccaccctgccttaattattgtaatgtcatgataagtcttgatatctggcagagaaagttatccaatttgcttgatcagttccaatttgcagatcagatgtgatccaatttgctttgatcagaccaaaaggactattttggtcctttgcatttccatatacattttagaatcagcttgttagttcccataaaaatctttctgtaagtttgattggaatttcattcagtctaaagatagatttytggagaatttacctcctgttaacttccaatttataaaaatgatatgtccctccattcctttaggtctttaatttctgtcattacagttttacagttctgtagtttatagaggtcttgcacatcttgttagatttattcctaggtatttgacatttagatgctgttttaaattgattttttttttttttgagatggagtcttgctgtgttgctcaggctggagtgcagtggtgtgatctcggctcactgcaacctccacctcctgggttcaagcgatttttctgcctcagcctctgaggt

SEQ ID NO: 310 - DNA, 599 bp, Homo sapiens
gcttgatcagttccaatttgcagatcagatgtgatccaatttgctttgatcagaccaaaaggactattttggtcctttgcatttccatatacattttagaatcagcttgttagttcccataaaaatctttctgtaagtttgattggaatttcattcagtctaaagatagatttttggagaatttacctcctgttaacttccaatttataaaaatgatatgtccctccattcctttaggtctttaatttctgtcattacagttttacagttctgtagtttatagaggtcttgcacatcttsttagatttattcctaggtatttgacatttagatgctgttttaaattgattttttttttttttgagatggagtcttgctgtgttgctcaggctggagtgcagtggtgtgatctcggctcactgcaacctccacctcctgggttcaagcgatttttctgcctcagcctctgaggtagctgggattacaggtacgttccaccatgcccagctaatttttgtatttttagtagagatgggggtttcaccatattggccaggctggtctcgaactcctgacctcaagtgaggaggcggtcttgc

SEQ ID NO: 311 - DNA, 599 bp, Homo sapiens
acaagcatcagcagcacctgggaacttgttagaaatgcagattctagggcccatcccagatccacagaatcagagtggggcccagacacctgtatttaggcacacccttcaggggattctgatgcatgccaaattttgagaactgtccatttaggagtgaatgcctcaaactagtccttcaggtgctataacttgccactaatagccccatgaagagtcccaaatagggtagttagtattgcttttaaacctactttattcttgtttatattttgctttgccgttgtgtcttagttttaygcactttgcttccatagattataatattctcagtatagaatccatgtctcatacttgcagtcctctggctctataagaacacttcatgtgattgtgtaaggatctctcgatcatgctgtgcataccccactaagtcctttatcctattattcacatgacaggatattttaaaatgttttctccgtgcatttctatgtaacacaatgcccatccccattcaggcagctagatcatgggtccaaggatgttaccaaaggaaaaacaaaaaaaggctttgacttccaaaaatctaaatctaa

SEQ ID NO: 312 - DNA, 599 bp, Homo sapiens
gggatacttttagttaatttaatatctatagaaacaatgctaatgactggtttgctgttaataaatacatgggtaaatctctgttcaggggtctcagctctgaaggctgtgagacccctgatttcccacttcacacctctatatttctgtgtgtgtctttaattcctctagcgctgctgggttagggtctccccgaccgagctggtctcggcaactttcctctgcacctgtggtttaagataaagagaaatcatggagtcctgagacttaggtgtctgggaatctaaaaggagggggtgrtcgggagcggggctcacaactgtaatcccagcactttcggaggctgaggtgggaggatcacttgagtccaggagttcaggaccagcctgggcaacatagtgaaaccccatctctacaaaaaaattttaaaaattaaccaagtatggtggcacacaccttagtcccagctacatgggaggctggggtggaagggtcacttgagcccaggagtttgaggctgcagtcagctaagatgatgccactgcactccaaccctgggtgacagagtgagactctatctctaaaaataaataggtaaa

SEQ ID NO: 313 - DNA, 599 bp, Homo sapiens
tgggaggctggggtggaagggtcacttgagcccaggagtttgaggctgcagtcagctaagatgatgccactgcactccaaccctgggtgacagagtgagactctatctctaaaaataaataggtaaataaataaataaaaataaagaagaggggcctcgtatttgacttcccaagtagaacccttggaactcagcaagatccctgagaagaaatggctgtgaaatgtgcctattaagcaagaccttttataaatttcatagagttagtttcccatgatcttgtttgatgttagagcaacygattcagtgctctgtgggtaaatgtagagaagaaatctaagctccaaagatttctctgtatgccaacatagcatggaaatagcagaatcaaatagcaagggagacttgaagtagcctcaacaataagaatgaatgatggtttaatagcagtctacaaagacttacaacaaaggaccaaatgggatatttgccattttgtcaaatgcccatagaaggggtcacctatggaagactagatgctcagtcccctccaaccttctagacattacaggaatttaagactacccaggacaggtgtg

SEQ ID NO: 314 - DNA, 599 bp, Homo sapiens
gtacaggggccacccttggagtagtgaggctgttcaaaatactgatgactctactgtgctggttttcaacgtgcactggctatattttcttaaataaacacagggcagtacttgtgccaaattatgtcataggtttatggtacaattttagactacagttttaaaatgcaaaatgaatattagcaacaaagaaagttccttcagaatcagctaagagaaggcagtgtagataaaaatataatgctaacttccttcatgggcttttacttacctgtgtcaacttaacaacaacaacaaaaraaacaaaaaactggggccaggcacctggtggctcacgcctataatcccagcactttgggaggccaaggcgggtggatcacgaggtcaggagatcgagaccatcctggctaacagagtgaaacccgtttctattaaaaatagaaaaattagccagccaggcatggtggcgggcacctgtagttccagctacttgggaggctgagacaggggaatggcgtgaacccaggaggcggagcttgcagtgagccgaggttgcgccactgcactccagcctgggcgacagagtgagactccatctc

SEQ ID NO: 315 - DNA, 599 bp, Homo sapiens
tccagcctggcaacagagcgaaactctgtctcaaaaaataaaaaaaaaaagtaccttctaatctttaatatgttaccatcctgaatagaagtttgggtagcagagatgttgttaaaatacctcattttactcaaaaggtaaaaatgtaaatgttagcaaattaagcaatatcacttaattattccgtgttataaaagtgacacttgttcattttagagaatttggaaaatacagaaaaatataatgaaggaaataaaatcattggtaatcccagcagccagcaataatcaaaatttggtrtatttccttctagtgatttctatgtgtacatgtatcttttctaacaaaagcataatgtacataaacttgctttttccactggatatctcataaccatttctctacatctttaagcattctttaaagacacaatttagtggtgttaaagtagaatagataatattctatcattgtgggtactataaaagttaatgatttcccaagttttggacatttgtttattttttgctattatgaaaagtgatacaagcaataaattttcaaggatacaatattttctaggatacagaattcccaga

SEQ ID NO: 316 - DNA, 599 bp, Homo sapiens
gtcccactgagtattcatataggtaaaaagcctacttttcccctcaaggagatggagcataactccccccagccagtgtgggctttacataatgagtttcttcctcatcactaccactgccaggtgattaaggttaacatagtaagatcagggacatgtcatgttgatagtatgtccccttgatgtgacaagagcactttgtctctgtggtattcctccaaaaaacaataatcccagttttatcatgacaaaaaaaatcagaagatcccaattgaaaaacgttttgcaaaatacctagtkagtactcctcaaagctgtcaaagtcatcaaaaacaaggaaagtctgaaaaactgtcacagtctggagtagcctaaggatacatggtgactaaatggaatgtggtttcctgaatgtcacctgtaaggaaacagcaggtaaaaatgaaggaaattggaataagtatggacttcagttaataatatcaatatagtttcattaattgtgataaatgtcccacactaatgtaagatttttaaaataggggaaactaggtatgtggtgtatgtgaattctgcactatctttgcaacatttctgca

SEQ ID NO: 317 - DNA, 599 bp, Homo sapiens
tcatgtgaatgtcatttcaggatagtggatgtgatagatattatgttacaatgttattataaacagggtcagctaagtctaatatgattgagaaccacaatctcagacctaagctctgagggcaagaaatgtttcatttcttacagtactctgcacctaatatctaattctcaatatctaatttgactctgggtgcggtttctgtttacacctacccagaagcccctatttgctcctccattctggcagtctcaattctgattcactgtccaccctcatgcagccagcttgggtaaataygatccatctaagtcagagtgttcccacccccttggccagtgattagacaggtaagtgacccagtactggttgagatatgacgggaattttgctgaggagcttagggaaaggtttttcactccttatcaaagacacatgaggaataaaatgatattttctttcactggacatttctattagtccgttctcatgctgctataaggacatgcccgagactgggtaatttataaaggaatgaggtttaattgactcacagttctgcaagggtgggaaggtttcaggaaacttacaataacggc

SEQ ID NO: 318 - DNA, 599 bp, Homo sapiens
cccaggctggagtacaatggcgtgatctcagctcaccccccaggttcaagtgattatcctgcctcagcctcccaagcagctgggattacaagtgcccaccaccatgcctggcaaattttgtattttcagtagagacagggtttcatcttgctggtcaggctggtctcaaactcctgacatcaagtgatccacctgcctcagcctcccaaagtgctgggataacaggcgtgagccactgcgcctggtcaaccaatccattttacaatctcttaagtgctggctttttttcttattgccaayagcaagcatccttactaacactcttcataatcagttggatggctgaggattatttatatatactgtactatggactgaatgtttgtgtcctgcggctcccttccatacatatgtttaagcctaattttcagtgtgatggtattcggaggtggggcctttgggaggtaatgtcatgagggtagaaccctcatgaatggaattagtgcccttttaagagatgatttatcttggatgtgtgagaacacagtgagaaggtgcctgtctacaagtcaggaagtgggtcctcaccagacactgga

SEQ ID NO: 319 - DNA, 599 bp, Homo sapiens
ggccagagattcctaggcacctagaaatgagacttaaatttatttcaccttccacaaaacacctcttcaggttggatcttcccacttcatccctcagggaagagtattagtaggagacacttccgatagtgttattccctagccctgggcgtggagagagggctggggagatcattcggccctaccagtttttctcagtttctcacaagcacaaggctcctgcactgcccttgtttactggtaaggacatctgaacaagcgagagggcaggtgccgatgcacacagcagtgagggaggarcaagagcaaaactctgttctctcgcagccattatgcactgccttctggtccaggccagaactaccacctcccacacagcccaagttaaaacagtcacccggccccatcctcacccattctccccagctgtcaccagccgggtttctttctttctttccttttcttttcttttcttttcttttccttttcttttccttccttccttccttcctctctctccctccctccttccttccttctctctttctctctttctctttctctttctctttctctctttctttctttttcctgcattt

SEQ ID NO: 320 - DNA, 599 bp, Homo sapiens
gtgcataatggctgcgagagaacagagttttgctcttgctcctccctcactgctgtgtgcatcggcacctgccctctcgcttgttcagatgtccttaccagtaaacaagggcagtgcaggagccttgtgcttgtgagaaactgagaaaaactggtagggccgaatgatctccccagccctctctccacgcccagggctagggaataacactatcggaagtgtctcctactaatactcttccctgagggatgaagtgggaagatccaacctgaagaggtgttttgtggaaggtgaaataartttaagtctcatttctaggtgcctaggaatctctggcctaggcttatccagcccattgtcctgagatcatgtgggttgttaaggacagggatctggccattctcctattcacactgcttgatgtgttagaatagcgaacagttgcaggggaaaaaagctcagaggcaaaaaataataatttgagaaacataaccataaaacacccccaaagcaatggactgtagattcaaacagaagaaatattagaataatggaccaagagctatttttgttttttttctggagacagggtcttgctc

SEQ ID NO: 321 - DNA, 599 bp, Homo sapiens
gagcaagactctgtctcaaaaaaaaaaaaaagtaataaacaatatgaaatattaccaatatgaaaaaaagaacaagaacatattaagcaaactaagcgaaaattagagatcaaaattataacaggtgtttaagaacaaaatagatgaattaaatatcagaacagatacatttgggagaaaattagctaattggtattccagtttgaggaaatctcccagaatgcaggcagtgacaaagaaataatataaaaagcttctagaataaaacagaagaatatacaaaggaacatgaattgacasatttttcaacagcaacatcatatgcaaaaagaaaatagaagagtattttcaagactgaaggaaattaaacctaggcttttccataaaactgtccttcagacataaaggcatcatcctcagttacacagaatctcaaaagttttgccttaaaaaccctttaaagggctgggtgcggtggctcatagctgtaatcccagcactttgggaggctgaggcaggcggatcacctgaggttgggattttgagaccagcctgaccaacatggagaaaccccgtctctactaaaaatacaaaattag

SEQ ID NO: 322 - DNA, 599 bp, Homo sapiens
ggccaaggcgggcggatcacgaggtcaggagatccagaccatctgggctaacatggtgaaaccctgtctctaccaaaaatagaaaaaattagccaggcatggtggtgggcgcctatagtccgcctactcaggaggctgaggcaggagaattgcttgaacctgggaggtggaggttgcagtgagccaagatcgcaccactgcactccagcctgggcgacagagtgagactccgtcacaaaaatatatatatatttaatatccaaatcttctccccaataagaaaggaatttgcttcactcygtcctcattgtgtcactattacctagagtgggatagtattgattttattttttaaagataataataaatatatcacggtgtttgatcactttttcttcatatatctcttgtagtgtctcctttgagacaggatctcactctcatccaggctggggtgctgcaatggcactgtcatagctcactatagccttgaaactcctgggctcaaacaatcctctgcctcagcctcctcctaagtagctaggaccacaggcacatgccaccacacccagctaattgttttatttttttgtagagat

SEQ ID NO: 323 - DNA, 599 bp, Homo sapiens
atgaaatataaagtacttagcacaataaatgagcattattattattcttattattgttcttgtccagtacctgtatttactgtctctttagtctcgttaaaatcatactttggccatttgtcctttctatagttgaaaacttaatatttttgtagcaggggttaggccaattatgtaaagtagcatataaacgtggtaagatttcaaagatgttacttgcttttcagaatatctattccctaggacattgtagctatgttttccttccttttgtagcaattggggcactgaagatgatgmgacccagagttaccacaatggcaattcagaccctcgaggattcggtaagtttgcctgtttttatgtgatatatgtagatttgtgtgattttgtattatgtatataattatgattttaataaacctaaaacattaatgtcctaattaagtatgatcaatttattagagatatgattatttttattttccacaaataatcttatttaggagttgtttaatttcagagtctcgatctaaaattacacttaccatctcaccatgatttacttttaagtctcagaattaatttccagtagactt

SEQ ID NO: 324 - DNA, 599 bp, Homo sapiens
cacacaaatctacatatatcacataaaaacaggcaaacttaccgaatcctcgagggtctgaattgccattgtggtaactctgggtctcatcatcttcagtgccccaattgctacaaaaggaaggaaaacatagctacaatgtcctagggaatagatattctgaaaagcaagtaacatctttgaaatcttaccacgtttatatgctactttacataattggcctaacccctgctacaaaaatattaagttttcaactatagaaaggacaaatggccaaagtatgattttaacgagactaargagacagtaaatacaggtactggacaagaacaataataagaataataataatgctcatttattgtgctaagtactttatatttcatttaaacccttaaaacagctccatgggctaggtgtgaacatctccattctattgatgaaactgccacttacttgtttagagtcacagactcatatctaagcctgcctgattccaaagctttattttgaatcaagggacacacgaaatcacacactctgaaatcaatctcttttaaagtatgcagataaacaatgtaaagaaattgcctacattt

SEQ ID NO: 325 - DNA, 599 bp, Homo sapiens
cgtagtggcgggcgcctgtagtcccagctacttgggaggctgaggcaggagaatggagtgaatccgggaggcagagcttgcagtgagccgagatcgcgccactgcactccagcctgggcaacagagcaagactctgtctcaaaaaataaataaataaataaataaaaataaaagacaaatgggccaggcgtggtggctcatgcttataatcccagcagtctgggaggccgaggcgggcggatcacttgaggccaggagtttgagaccagcctgggcaacatagtgagaccttgcctctaygcctctaccaaaaacaaacaaacaaaaactagctgggtgtggtggcacacatgtgtagtccaaggtgctcaggaggctgaggcaggaagactacctagaagttagaggcttcagagagcctgtctctaaaaaaaacacatctcatttgcccaagaacaatacaaagatcaaaagatgcatgaaaaacacttcaatatattattaaaaatgtgaagtcataggcagatctggtttgaattatactggtcagtaaacaaaacaaaaaagatttttaaaaactaccttaaaaaatctataat

SEQ ID NO: 326 - DNA, 599 bp, Homo sapiens
tcttaccacctatccttaatttcctttcctagatgttttggcatagtaagaactcaataaatgtgtttatttaactgaatgacatatttatatatgtgtttgtatgtgtgtatatatatgaaatagcaacaaggttctttatatttttcagtacaaataaacacagttctcttttggagatagccgtttctatttattaacaagtaccataatttcagctaaatgttactatatttacagaattgatgaattacagaagttttcatttttgtatttcaaatatttacagatgcatttttmatgatattttttcactgatttttcctcctgcttattccattaaaataggcttactacatatagctcttctttttagtaaaacttgaatattaagttatgttgattatagtatgaattagcctttcctgttgttaatgattcaggcttgttgttttttcattgcaggctaatccaaaaatgtgattttcccattatgaagttttcactctacttcttggcttatgaggataaaaatgacatccctaaagaaaaagatgaaaaaatagcctgggcgctctccagaaaagctacacttgagc

SEQ ID NO: 327 - DNA, 599 bp, Homo sapiens
tcagatgctacagtcctaaaggtctcgattttatcaagcagctgagaaagagaaaagtagctctgcacatccaagtgctggcctggtactcacagctgggccttggttttctcctgttgaacatatataaagtcactctgtggctgccatgatgtgataccaaaataaaaaaaaaaaatttaaagcagcaagaccatgtccacttcatattccggtctaagtatacattaaaacaaggcactatgaacactaccaaaataacaaacatccttctttcccagctaatatgagtggtagctrcttctctattgattacaatgtttgcttgaggaagccacctgcaggacaaagtcttatgtatgatctgtcaggcccgaccacacacaatcacacagactttagctggcaggcctcatgggggagatctgtcctcccagcactggttctcagtctcaggagggagcactgcagtccctggagggaggtgcaagcaaggactaaattctaaagatgtccttccttattctctcaaacactaacctaggtgctgctgtgagagactctgagaggaaattaaggtcactcatcagtggacttta

SEQ ID NO: 328 - DNA, 599 bp, Homo sapiens
cagaaaactagcttcatggaatactgccaaatgatagcaaccctgagaatgcctgagatacaggaaaactgtaccttccacagagttctcttgaggcttaacagaaggtaaaccccatgcagcacttatataagacattaagtgcccacgatcctgagtattaagtgtccccgaaaatacaatgagggaacacatataaaaacacttagcacagtagtacatggtaatggctaaatcaatgctagctgttatttgtagtagtttagtattagtgtgtgtagtgaagaaaaatactgaacyggtaatcagggaaactgagtcctgttactgtgctactcaacaaataaccgtttagtcgactaagcctcagattgtgaaactgctctattactggatgtccttatttgtaaaaagaaatgggggagttgagataatctctaagttcccttccagttcgaaaaatgtctaactctatgacgagtttggtttcaagcctcatcaattgagaggaagatgatactattgaccgaaataaaaatgtcaaaacaaccaattggaaatgtctaggtgttgtagggaatatgcaattaaggtgcaca

SEQ ID NO: 329 - DNA, 599 bp, Homo sapiens
agggcacgcaatggagaggacactcatgaccccatagatacttatgactcactaataggtcatgactctcagaaccctagggatgaacacccacaacctgctcctcttacctctccagcctcatcttgttttgctttcccaggttccagttcctccagccacacaaggccttggtatatgctgttcctttggcctggaatgctcgcctctgcccttttaggctgcccactacacgtagttgacttttctccgccctttagattttggctcaagatcactccagcctaagtgaagtcttcktgtttcacacactcatagatgtgtacttttttccaattaagagcttcagtttgcagttgtacatttattactgtaattattttgtttaactttagtgccttctaactggaaggcttgatagaaacagaaactgctgattttgttcaccatttatgtctccaagtgttggcctagtgtttgggacatcaataggttgttaaatgaatgaacgaataacaagaatgactttatgcaaaagatggttacatatatggggcattttttttttttttttttttttttttgagacggggtctagc

SEQ ID NO: 330 - DNA, 599 bp, Homo sapiens
tagacctccagctataaggaacacccgttacccttgttttgtaatccttcttgaaggaatgcatggactgcttaggtaatatttatagtaaggagctaattttaataaattctattttaaaaacctatagaatctaaagagagttttgttgttgtttgtctaaatgtaagtaaccactgacatctcagtactaattctttagtgactttatttcaagttctgggaggtcttcagggatgcacaattcattaggaagactgtacaaattttaaatggtcacgtcattgctatgtgattaaygcaacttatctaggaaatcctttctgaattcttgctagatgtaaaaaagccattgaaattctggctaattaaaacaatagcaataccaatttattagtaaactgtaaactagtctttttttaaatgtcaaaagacatattcgaaaacctctcacaccttttaaaagcaaattgctatttctttttaaaataaatggatcaatcctttcactgggtttattcagtgaaaaaatctgcccttgaaaaggctttaaagcatttctgtgttagaagagtagaataggccctgaattaaaaacc

SEQ ID NO: 331 - DNA, 599 bp, Homo sapiens
agaaggtggtggggaaggacaactatattctcacattcttgcccaaactgttcatcagggtatttgagaaaggcagtttcttcttcaaactagtcctaatctaccttaattgtcatgtgcctgagacaagggaagtggcttgtggagatagcaggtaactttgattccttcttcactacctgttatctgcagacagatcattaaattaagagccttgaaaactagtatgaaggtgcgagaagatttttttttccttgcagatgtccatgtaccctatcaaaaattagctcttttacatayataagaagaatattttcatagtttttactttttcccttacatgtaactgccattcaccttcacctctgaccaaagctaggctttgctgaaactaacccaggcttggatgacaaaatcctttatctgagttttaaaagatctctgctactttgtagatagcgtatttcaactttgactagataaggaccctgactatttttcactcaagtgggaatcatcagtctggggagggaaatggagtcgattatttaattacttgaggggaatattggaatgctctaatttttctgtgatataca

SEQ ID NO: 332 - DNA, 599 bp, Homo sapiens
cgcataggaagtagctgtaatcaccagctcataaagaaaccacagcagagcttttggatttttgcagagtgctcatgtaaaatttaagctgggaggcagaatccctgttaatgtaattaggatctaatttaccaccctttgcacacagatgtaataatgtctagatatttattctatctttaagcatgttacatatacctcagtgcagcagttactgaacagaaacaggtgtgagcagtttcctaattcgtttttacactgtaaacacatagcaatttaagggaaatatttaccaaaaaygaggggaaagaatgtctgcaaatgaggcagtaatggacagttgccgtttatgtactgatggatttctcaatggttaagtaaattaaagcaaatgctagaaaaatcaataggccagcatagtcttggttttgcctcatagacttcaggacagaatgacagatgtggtgtctatgacccatttaagtattaatgcttgaaaaactgcttctgcatgcaaggttgtaggcttagaaacattctcctatattaaattacagacaataatatttttaagtatatgtggaatttatatgtttcag

SEQ ID NO: 333 - DNA, 599 bp, Homo sapiens
attgtagactactctttgtttgggtgtaggcatttataatttatggtaagtttagtgaagaccaaattttcacaaagaattaaaatgacaaaccaagcattttaatttgctaaataaaataaaggcattgcttaaccattgtcaaacatatatatacatacatatagctaccccctcttaataagactgaagtacatttgtcggtatcagaagtggtgggcaagagatatagtcaaatcaattcatttacttctctcattgagccctgtgagtctcaggcccacaccttgaagtgaaaamatcatcaattaccaaaatgacaggtgctcaggacttacttcctgaaagatgttcatattggaccagacaccacgagcactcagcaacattcatcacttcgttcaaaagagaccgaaaaaactgtggggtgaatttgcaagggaaaaaaggccaaatatacatgcatgaaatagcatacgtttgaatttctaaaagtcagacagcaagcaagtatcctattttattttaaaagattattaattgttatttaatagtcaacttgcatgtgatcttacaattaataattgtatccccatttc

SEQ ID NO: 334 - DNA, 599 bp, Homo sapiens
caaaaatatctcctcaccaaatgtaccattttgcttctaacttacggatactttattctttagacaaggatttttcctgaataattaatgctagacagggcatctgtaaaggcaataatgattgctgtcaacagactcattccaaaatggcgttgtagtatttcatcttccttgtctttctacttaatggtttcaaaatcctttctctttgatttttaaagtggaaaagtgttcttcaggctggtgttaggtctccttttagctgacgtgctctctgaaattgtagagatgggctatacrtagtttgaaaaagatccttactttattggagtttattgtataaatcagtcatgaactttcttttcagaaaggtggtccagtatagtgtgccatgtttttgttgtgcatgttcaagtgaaattcttggggaaaagcctccatgttgaaacagtcttctttatcctttcttctgtcctgtgctaggcataaagatacagaacttgaattttgttgcattgaagttagagagtaggaagggaattttgtcacaaataaacccaagaacaacatagagtgaatttgggagaaactttggggga

SEQ ID NO: 335 - DNA, 599 bp, Homo sapiens
gggagaaactttgggggaaattataaattgacgctaagaaagaaaagcagaataacacagtaattaaaagcataggctgtgcaattgactacttagagtcaaatcccagctctattttacttgccagtaaattagattctgagcctcagtgtcctctgtaaaataatggtaagagtacttatcttagagacttgttttaaggattaaataagtaaatacttaaagctctgagaacgctgcccaacatacagtaaacattatataaatgtcaggtaaataaagtgagcaaaatctggactrggcactctactattctgggatttgttgaactaagtgagaaggatgaataattgttggaatggagctgctagttttgtgtttatctgaagtcatctactcctactcacaaatgcaggttgagggattttagtcatctctaactttaataacattcattcacagaaatatttattgggcacagaactggtagctgcatcatcactgggaatagcattcatatgtttttattagtagtctactaaaatgtaatgattttgaatgtcatttaactcatatttgattaagaatcatgtaataaa

SEQ ID NO: 336 - DNA, 599 bp, Homo sapiens
cctttgggagagatttcagatatattaaaccccattacagaaaatgtaatttctgtcaatataatcccatgtattcttcaatttagtattttagagataacaagctacttacaatatatttgcctttggaggattaatttacaaagtgcactatctgcaagacacagacagactcacataagaattgcttgtaagggttatcaatttctatgaccttactgctgccttgagagatcaatttttgcagcccttttcatcagtggatggtaaaccaagcaaaggcatactacatctaaatgygttactacgagttgtgtggctgctgaggttttttgtttgcctatttctacctgctactgtgtcttaattttcaaggctgttaatactacatattggaaatttcaaatatgtaataatttcatatttgaaatatgaaatttcaaatatgtaatatttgaaatataatatcctggatgatattataggggagaccatcaattttgcgtgcacatctagattcatatatctgtattttaaaacttaaagctgatgttgagacagttttaatacaatggaggctgcacaaatgtccgcatttc

SEQ ID NO: 337 - DNA, 599 bp, Homo sapiens
gccttgagagatcaatttttgcagcccttttcatcagtggatggtaaaccaagcaaaggcatactacatctaaatgtgttactacgagttgtgtggctgctgaggttttttgtttgcctatttctacctgctactgtgtcttaattttcaaggctgttaatactacatattggaaatttcaaatatgtaataatttcatatttgaaatatgaaatttcaaatatgtaatatttgaaatataatatcctggatgatattataggggagaccatcaattttgcgtgcacatctagattcatrtatctgtattttaaaacttaaagctgatgttgagacagttttaatacaatggaggctgcacaaatgtccgcatttccctgggaagtgttcctgtttgcgggcaaaggagtggcaggacaagctctctagttaagaaacgaaaagcgctgtcattcaggggatctatcctgtgagccatctctcttcctctgcagggttgctagcaaactccactcataaacacagttaccagagaacaaatcccaatacaagttgcttactatgggtggacggccttattatgggatatttcctgtgaa

SEQ ID NO: 338 - DNA, 599 bp, Homo sapiens
ttttaaagtatatgcagactttaccctaaacttcacactgctattcaattctgcacgggtaaataacttaaaaatagcttttaattagttatttgggacagaaacacaattctgtgcttaactgccactttgtgcagtgtgtggtttctctctacttgtatatatatggagttagaagtggcttttgcttgcaccacagtgctttgcatttggcaattgtttcaacatttaacaatgttgtttgctgtagaaaatgtccctcaagtaactaaaagttgtaagagagattgtttttctttrgtttgacaaataatgacaataccatgtttgatttgaatttttaatggtttattaatttttatagaaacctcttcctgttttgttttgttttgttttgttttgttttgttttgttttgttttgttttgataccgagtcctgctctgttgtccgggctggagtgcagtggtgtgaacatggctcactgcaacctccaactcctagctcaagtaatcctcctgactcagcctccccagtagttgggaatacaggcatatgccaccatgcccagttaatttttttttcttttgttgaggcagg

SEQ ID NO: 339 - DNA, 599 bp, Homo sapiens
gcattcaaaggcagttgctgagcttctcaggaggacaagacactagctggaagctgcaatccagctggtacaacctgaggaattatgtcgggaccctgtagggttggggttcaagtgaggctcagagttgagtgagttgggagactgatttacccagcagggacccaatgtttctgaatatccatgattcatttcaaggcaatgaaaccagtgacaacggaggttgactttgaacatggtatgcaggtcagcacaacttgttgcaaatcctaattaagccagtcttcttgttttcagtgkgtctgtaagtatctggtcagagaaatgcaaacaacaggctgtgggtcatgcaacccgatggggcaggacttgactcccatttccctggagagggcatagggatgaggggctgagaggtgtcacggtcaagggggaagagcttggctacgcctcatccataggtggctaaggtgtaagcctcatgacaatttaaaatttacacagggcaagtatgggaaggggaaatgcagggacaggtcgctgcaatcaaaacatttcatccctcatccctactccttggatttgggcgacagtgtcct

SEQ ID NO: 340 - DNA, 599 bp, Homo sapiens
aataaacaaaagaatattaatctgttccgacatatttgagaggtttcttttcctttttaccctcttaactaaccagtgcctcacaagattagatcctatggttgaagaaggggaaaagtatttcctttttcaaacacattaactcctccaggtctgggggagatgattggagctacatgtaacttagctcaaaaataaatgtcctgcctttggtgagatctggcttaatattgataaagtcacttttttttttttttcaaaaacaatatgttgcctacggatggaagttaatgcaccatrtaattatacacacatacacacgtgcatgtaagttttcagtatttttactctaggatgctaactaaatcaaatgcaaaccagtagtcttgaggtttgttgtctggttgaagggttttgttggcctatatcactaggacatctgcagtggttgactttgtttgtttgctagtttgggtcatagttcagatgtacttggaaagggccggggagtgggtagggggatgttttaaaagcttatggaaagaattgaacatgtacaaaaagatataggatctcagccccctggctggttacctgac

SEQ ID NO: 341 - DNA, 599 bp, Homo sapiens
aaacaatatgttgcctacggatggaagttaatgcaccatataattatacacacatacacacgtgcatgtaagttttcagtatttttactctaggatgctaactaaatcaaatgcaaaccagtagtcttgaggtttgttgtctggttgaagggttttgttggcctatatcactaggacatctgcagtggttgactttgtttgtttgctagtttgggtcatagttcagatgtacttggaaagggccggggagtgggtagggggatgttttaaaagcttatggaaagaattgaacatgtacaraaagatataggatctcagccccctggctggttacctgacaatactgtttacagaaaggaaagtatagtaggttatggcagagattcctatcctcaggacttggggcggggtgggagggggcgcagagagggggtagcggggcacataagaagcatgtcacaatcattttgagtgaatatagtatttaaaaagcatatcgagaattaaattttgtttttagtcatgctatttcttttgaaattgcaaataacaaaggaatataagatttttgatgtgtcaaaagggagcatggattaaaa

SEQ ID NO: 342 - DNA, 599 bp, Homo sapiens
cttggaactgttggatcctggaggaggttaagaaactagaaaaaggtgccaaaaacatgatttcataaggtttgacttgagaatgaatcaactttcttgaatatcttaaaatcatgtttcatgaaattttatttcacaaagaaagggagtgtagaagacttgaactctgaagaccaacagttatggatcattaaatccaatatgtaaatgtaaatctaaaataaagagttccaacagaaaaatttcagagtgatttttttattgttgtttaaaagaacagagagttacatgggagataaytagacctctttccctgagggtttgtttataaaacatgccttaaaagttattaagggatgctgactttaaaaaaaagtagtagatatgattttatttaaatgactatgatatagatgaaggaaatgctatggtgaggaaattcctggaataaaaaaataacaaaatgttgggatttgacaactaccttccaatgataaattttatatttgaagatttttcatgaggtttaaattctaaaatttgaaaatatttatagaaatcacaaaaatgaattatatatcaattatcatctgtgcatg

SEQ ID NO: 343 - DNA, 599 bp, Homo sapiens
tgcttcagaagtaagctgccttgggttattaaagcacacaaaagctctacctcattgaatgtctttgaactttactgagtcccacaagcaatcctgacccctttgcccttttgacaggtaataaagtttacagcaattccacaccaagccatgcaccagggagtggtcatagtagcagaaagagctgggaaggcaaactcagaaaaaaaataacattaatatattgaatgtgtacaaagaatattgtaactgagcatattatttaagagttacttttctctgttggctttttttttccaycttctccaaacgttttataatgatgttaaattgattagtgttgttatgaaacgaaactggtttaattttaattgccaggcgttcctatactcactcccagtttatttaccaggtagcggacctcccggtgaatggcttgaggatggttttcgcttgcgtgacttgaatacagtcactatgggggacttgcctgctcggttggactggtctatcatgggctgcactattcttctccgggcattaataaacctgtaaccaagaaagtgaaacaaatcgttattccttaaaaataaaagggg